(19) United States
(12) Patent Application Publication
(10) Pub. No.: US 2024/0158515 A1
(43) Pub. Date: May 16, 2024

(54) ANTI-FGFR3 ANTIBODIES AND ANTIGEN-BINDING FRAGMENTS AND METHODS OF USE THEREOF

(71) Applicant: Regeneron Pharmaceuticals, Inc., Tarrytown, NY (US)

(72) Inventors: Yan YANG, Mamaroneck, NY (US); Christopher DALY, New York, NY (US); William OLSON, Yorktown Heights, NY (US); Tammy T. HUANG, Cross River, NY (US); Drew DUDGEON, Montvale, NJ (US); Robert BABB, River Edge, NJ (US)

(73) Assignee: Regeneron Pharmaceuticals, Inc., Tarrytown, NY (US)

(21) Appl. No.: 18/509,136

(22) Filed: Nov. 14, 2023

Related U.S. Application Data
(60) Provisional application No. 63/383,686, filed on Nov. 14, 2022.
(60) Provisional application No. 63/587,538, filed on Oct. 3, 2023.

(51) Int. Cl. C07K 16/28; A61P 35/00

(52) U.S. Cl. CPC C07K 16/2863; A61P 35/00; A61K 2039/505

(57) ABSTRACT

The present invention provides antibodies that bind specifically to FGFR3 and methods for treating or preventing cancer, such as bladder cancer.

### CROSS REFERENCE TO RELATED APPLICATIONS

This application claims priority to U.S. Provisional Application No. 63/383,686, filed Nov. 14, 2022, and U.S. Provisional Application No. 63/587,538, filed Oct. 3, 2023, the disclosure of both of which is herein incorporated by reference in its entirety.

### SEQUENCE LISTING

The instant application contains a Sequence Listing which has been submitted electronically in XML file format and is hereby incorporated by reference in its entirety. Said XML copy, created on Nov. 3, 2023, is named 250298_000562_SL.xml and is 265,438 bytes in size.

### FIELD OF THE INVENTION

The present invention relates to antibodies and antigen-binding fragments thereof that bind specifically to FGFR3 and method for treating conditions such as cancer.

### BACKGROUND OF THE INVENTION

Several cancer genomic analysis studies have identified FGFR3 as a common mutation exclusive to non-inflamed bladder tumors. FGFR3 mutations including fusions are enriched in the luminal papillary (LumP) subclass of MIBC (30-40%), characterized by low immune cell infiltration.

CD73 or ecto-5′-nucleotidase (ecto5′-NT, EC 3.1.3.5), is a cell surface enzyme that catalyzes the dephosphorylation of extracellular AMP to adenosine. It is broadly expressed in many types of cancer and has been associated with a pro-metastatic phenotype in melanoma and breast cancer. Adenosine activates immunoregulatory responses through specific receptors, which protect tissues from damage caused by excessive inflammation. Extracellular adenosine level is kept low under physiological conditions, but it increases during inflammation and cell death. In T-cells, the high-affinity adenosine receptor A2A (A2AR) is activated by adenosine to increase the cytoplasmic cyclic AMP level. This suppresses T-cell functions such as proliferation and cytokine secretion.

Erdafitinib is a pan-FGFR tyrosine kinase inhibitor (TKI) approved to treat FGFR3-altered bladder cancer. Rapidly rising resistant mutations and the toxicity associated with TKI (hyperphosphatemia, ocular toxicity) presents a challenge in treating cancer by targeting FGFR3. In addition, the antibody B-701 (Vofatamab) has been investigated for use in the treatment of metastatic urothelial carcinoma (MUCC) and as targeted alpha therapy. Necchi et al., The Journal of Urology, Vol. 201, No. 4S Supplement, 2019, e840, abstract: PD47-08; Storozhuk et al., FGFR3 Targeted Alpha Therapeutic [²²⁵AC]-FPI-1966 Induces Regression in Preclinical Bladder Xenograft Model (Fusion Pharmaceuticals, Inc. (2020)).

### SUMMARY OF THE INVENTION

The present invention provides an isolated antibody or antigen-binding fragment thereof that specifically binds to FGFR3 (e.g., monomeric or dimeric FGFR3b; optionally with the proviso that FGFR3 is not FGFR3c) or an antigenic fragment thereof (optionally in association with a further therapeutic agent, such as, for example, an FGFR inhibitor, erdafitinib, pemigatinib, infigratinib, rogaratinib, dexamethasone, alkylating drug, altretamine, trabectedin or busulfan, a nitrosourea, carmustine, lomustine, a cytotoxic antibiotic, an anthracycline, doxorubicin, valrubicin, bleomycin or dactinomycin, an antimetabolite drug, methotrexate, floxuridine, clofarabine, pralatrexate, a vinca alkaloid, vinblastine, vinorelbine, vincristine, vindesine, a photodynamic drug, porfimer sodium, aminolevulinic acid, a platinum drug, cisplatin, phenanthriplatin, a taxane, paclitaxel, docetaxel, a topoisomerase inhibitor, irinotecan, topotecan, etoposide, teniposide, ziv-aflibercept, an anti-cancer antibody, rituximab, trastuzumab, cetuximab, cemiplimab, pembrolizumab, panitumumab and bevacizumab) comprising: a heavy chain variable region (HCVR) that comprises the HCDR1, HCDR2 and HCDR3 of a HCVR that comprises the amino acid sequence set forth in SEQ ID NO: 2, 22, 42, 62, 82, 102, 122, 140, 159, 169, 179, 199 or 219, and a light chain variable region (LCVR) that comprises the LCDR1, LCDR2 and LCDR3 of a LCVR that comprises the amino acid sequence set forth in SEQ ID NO: 10, 30, 50, 70, 90, 110, 130, 148, 187, 207, or 227 or an isolated antibody or antigen-binding fragment thereof that specifically binds to FGFR3 as such an antibody or fragment or that competes with such an antibody or fragment for binding to FGFR3. In an embodiment of the invention, the antibody or antigen-binding fragment comprises (a) a heavy chain variable region (HCVR) that comprises the HCDR1, HCDR2 and HCDR3 of a HCVR that comprises the amino acid sequence set forth in SEQ ID NO: 2, and a light chain variable region (LCVR) that comprises the LCDR1, LCDR2 and LCDR3 of a LCVR that comprises the amino acid sequence set forth in SEQ ID NO: 10, (b) a heavy chain variable region (HCVR) that comprises the HCDR1, HCDR2 and HCDR3 of a HCVR that comprises the amino acid sequence set forth in SEQ ID NO: 22, and a light chain variable region (LCVR) that comprises the LCDR1, LCDR2 and LCDR3 of a LCVR that comprises the amino acid sequence set forth in SEQ ID NO: 30, (c) a heavy chain variable region (HCVR) that comprises the HCDR1, HCDR2 and HCDR3 of a HCVR that comprises the amino acid sequence set forth in SEQ ID NO: 42, and a light chain variable region (LCVR) that comprises the LCDR1, LCDR2 and LCDR3 of a LCVR that comprises the amino acid sequence set forth in SEQ ID NO: 50, (d) a heavy chain variable region (HCVR) that comprises the HCDR1, HCDR2 and HCDR3 of a HCVR that comprises the amino acid sequence set forth in SEQ ID NO: 62, and a light chain variable region (LCVR) that comprises the LCDR1, LCDR2 and LCDR3 of a LCVR that comprises the amino acid sequence set forth in SEQ ID NO: 70, (e) a heavy chain variable region (HCVR) that comprises the HCDR1, HCDR2 and HCDR3 of a HCVR that comprises the amino acid sequence set forth in SEQ ID NO: 82, and a light chain variable region (LCVR) that comprises the LCDR1, LCDR2 and LCDR3 of a LCVR that comprises the amino acid sequence set forth in SEQ ID NO: 90, (f) a heavy chain variable region (HCVR) that comprises the HCDR1, HCDR2 and HCDR3 of a HCVR that comprises the amino acid sequence set forth in SEQ ID NO: 102, and a light chain variable region (LCVR) that comprises the LCDR1, LCDR2 and LCDR3 of a LCVR that comprises the amino acid sequence set forth in SEQ ID NO: 110, and/or (g) a heavy chain variable region (HCVR) that comprises the HCDR1, HCDR2 and HCDR3 of a HCVR that comprises the amino acid sequence set forth in SEQ ID NO: 122, and a light chain variable region (LCVR) that comprises the LCDR1, LCDR2 and LCDR3 of a LCVR that comprises the amino acid sequence set forth in SEQ ID NO: 130, (h) a heavy chain variable region (HCVR) that comprises the HCDR1, HCDR2 and HCDR3 of a HCVR that comprises the amino acid sequence set forth in SEQ ID NO: 140, and a light chain variable region (LCVR) that comprises the LCDR1, LCDR2 and LCDR3 of a LCVR that comprises the amino acid sequence set forth in SEQ ID NO: 148, (i) a heavy chain variable region (HCVR) that comprises the HCDR1, HCDR2 and HCDR3 of a HCVR that comprises the amino acid sequence set forth in SEQ ID NO: 159, and a light chain variable region (LCVR) that comprises the LCDR1, LCDR2 and LCDR3 of a LCVR that comprises the amino acid sequence set forth in SEQ ID NO: 148, (j) a heavy chain variable region (HCVR) that comprises the HCDR1, HCDR2 and HCDR3 of a HCVR that comprises the amino acid sequence set forth in SEQ ID NO: 169, and a light chain variable region (LCVR) that comprises the LCDR1, LCDR2 and LCDR3 of a LCVR that comprises the amino acid sequence set forth in SEQ ID NO: 148, (k) a heavy chain variable region (HCVR) that comprises the HCDR1, HCDR2 and HCDR3 of a HCVR that comprises the amino acid sequence set forth in SEQ ID NO: 179, and a light chain variable region (LCVR) that comprises the LCDR1, LCDR2 and LCDR3 of a LCVR that comprises the amino acid sequence set forth in SEQ ID NO: 187, (l) a heavy chain variable region (HCVR) that comprises the HCDR1, HCDR2 and HCDR3 of a HCVR that comprises the amino acid sequence set forth in SEQ ID NO: 199, and a light chain variable region (LCVR) that comprises the LCDR1, LCDR2 and LCDR3 of a LCVR that comprises the amino acid sequence set forth in SEQ ID NO: 207, and/or (m) a heavy chain variable region (HCVR) that comprises the HCDR1, HCDR2 and HCDR3 of a HCVR that comprises the amino acid sequence set forth in SEQ ID NO: 219, and a light chain variable region (LCVR) that comprises the LCDR1, LCDR2 and LCDR3 of a LCVR that comprises the amino acid sequence set forth in SEQ ID NO: 227. In an embodiment of the invention, the antibody or antigen-binding fragment comprises: (a) a heavy chain variable region that comprises an HCDR1 that comprises the amino acid sequence set forth in SEQ ID NO: 4, an HCDR2 that comprises the amino acid sequence set forth in SEQ ID NO: 6, and an HCDR3 that comprises the amino acid sequence set forth in SEQ ID NO: 8, and a light chain variable region that comprises an LCDR1 that comprises the amino acid sequence set forth in SEQ ID NO: 12, an LCDR2 that comprises the amino acid sequence set forth in SEQ ID NO: 14, and an LCDR3 that comprises the amino acid sequence set forth in SEQ ID NO: 16; (b) a heavy chain variable region that comprises an HCDR1 that comprises the amino acid sequence set forth in SEQ ID NO: 24, an HCDR2 that comprises the amino acid sequence set forth in SEQ ID NO: 26, and an HCDR3 that comprises the amino acid sequence set forth in SEQ ID NO: 28, and a light chain variable region that comprises an LCDR1 that comprises the amino acid sequence set forth in SEQ ID NO: 32, an LCDR2 that comprises the amino acid sequence set forth in SEQ ID NO: 34, and an LCDR3 that comprises the amino acid sequence set forth in SEQ ID NO: 36; (c) a heavy chain variable region that comprises an HCDR1 that comprises the amino acid sequence set forth in SEQ ID NO: 44, an HCDR2 that comprises the amino acid sequence set forth in SEQ ID NO: 46, and an HCDR3 that comprises the amino acid sequence set forth in SEQ ID NO: 48, and a light chain variable region that comprises an LCDR1 that comprises the amino acid sequence set forth in SEQ ID NO: 52, an LCDR2 that comprises the amino acid sequence set forth in SEQ ID NO: 54, and an LCDR3 that comprises the amino acid sequence set forth in SEQ ID NO: 56; (d) a heavy chain variable region that comprises an HCDR1 that comprises the amino acid sequence set forth in SEQ ID NO: 64, an HCDR2 that comprises the amino acid sequence set forth in SEQ ID NO: 66, and an HCDR3 that comprises the amino acid sequence set forth in SEQ ID NO: 68, and a light chain variable region that comprises an LCDR1 that comprises the amino acid sequence set forth in SEQ ID NO: 72, an LCDR2 that comprises the amino acid sequence set forth in SEQ ID NO: 74, and an LCDR3 that comprises the amino acid sequence set forth in SEQ ID NO: 76; (e) a heavy chain variable region that comprises an HCDR1 that comprises the amino acid sequence set forth in SEQ ID NO: 84, an HCDR2 that comprises the amino acid sequence set forth in SEQ ID NO: 86, and an HCDR3 that comprises the amino acid sequence set forth in SEQ ID NO: 88, and a light chain variable region that comprises an LCDR1 that comprises the amino acid sequence set forth in SEQ ID NO: 92, an LCDR2 that comprises the amino acid sequence set forth in SEQ ID NO: 94, and an LCDR3 that comprises the amino acid sequence set forth in SEQ ID NO: 96; (f) a heavy chain variable region that comprises an HCDR1 that comprises the amino acid sequence set forth in SEQ ID NO: 104, an HCDR2 that comprises the amino acid sequence set forth in SEQ ID NO: 106, and an HCDR3 that comprises the amino acid sequence set forth in SEQ ID NO: 108, and a light chain variable region that comprises an LCDR1 that comprises the amino acid sequence set forth in SEQ ID NO: 112, an LCDR2 that comprises the amino acid sequence set forth in SEQ ID NO: 114, and an LCDR3 that comprises the amino acid sequence set forth in SEQ ID NO: 116; (g) a heavy chain variable region that comprises an HCDR1 that comprises the amino acid sequence set forth in SEQ ID NO: 124, an HCDR2 that comprises the amino acid sequence set forth in SEQ ID NO: 126, and an HCDR3 that comprises the amino acid sequence set forth in SEQ ID NO: 128, and a light chain variable region that comprises an LCDR1 that comprises the amino acid sequence set forth in SEQ ID NO: 132, an LCDR2 that comprises the amino acid sequence set forth in SEQ ID NO: 34, and an LCDR3 that comprises the amino acid sequence set forth in SEQ ID NO: 134; (h) a heavy chain variable region that comprises an HCDR1 that comprises the amino acid sequence set forth in SEQ ID NO: 142, an HCDR2 that comprises the amino acid sequence set forth in SEQ ID NO: 144, and an HCDR3 that comprises the amino acid sequence set forth in SEQ ID NO: 146, and a light chain variable region that comprises an LCDR1 that comprises the amino acid sequence set forth in SEQ ID NO: 150, an LCDR2 that comprises the amino acid sequence set forth in SEQ ID NO: 14, and an LCDR3 that comprises the amino acid sequence set forth in SEQ ID NO: 153; (i) a heavy chain variable region that comprises an HCDR1 that comprises the amino acid sequence set forth in SEQ ID NO: 161, an HCDR2 that comprises the amino acid sequence set forth in SEQ ID NO: 163, and an HCDR3 that comprises the amino acid sequence set forth in SEQ ID NO: 165, and a light chain variable region that comprises an LCDR1 that comprises the amino acid sequence set forth in SEQ ID NO: 150, an LCDR2 that comprises the amino acid sequence set forth in SEQ ID NO: 14, and an LCDR3 that comprises the amino acid sequence set forth in SEQ ID NO: 153; (j) a heavy chain variable region that comprises an HCDR1 that comprises the amino acid sequence set forth in SEQ ID NO: 171, an HCDR2 that comprises the amino acid sequence set forth in SEQ ID NO: 173, and an HCDR3 that comprises the amino acid sequence set forth in SEQ ID NO: 175, and a light chain variable region that comprises an LCDR1 that comprises the amino acid sequence set forth in SEQ ID NO: 150, an LCDR2 that comprises the amino acid sequence set forth in SEQ ID NO: 14, and an LCDR3 that comprises the amino acid sequence set forth in SEQ ID NO: 153; (k) a heavy chain variable region that comprises an HCDR1 that comprises the amino acid sequence set forth in SEQ ID NO: 181, an HCDR2 that comprises the amino acid sequence set forth in SEQ ID NO: 183, and an HCDR3 that comprises the amino acid sequence set forth in SEQ ID NO: 185, and a light chain variable region that comprises an LCDR1 that comprises the amino acid sequence set forth in SEQ ID NO: 189, an LCDR2 that comprises the amino acid sequence set forth in SEQ ID NO: 191, and an LCDR3 that comprises the amino acid sequence set forth in SEQ ID NO: 193; (l) a heavy chain variable region that comprises an HCDR1 that comprises the amino acid sequence set forth in SEQ ID NO: 201, an HCDR2 that comprises the amino acid sequence set forth in SEQ ID NO: 203, and an HCDR3 that comprises the amino acid sequence set forth in SEQ ID NO: 205, and a light chain variable region that comprises an LCDR1 that comprises the amino acid sequence set forth in SEQ ID NO: 209, an LCDR2 that comprises the amino acid sequence set forth in SEQ ID NO: 211, and an LCDR3 that comprises the amino acid sequence set forth in SEQ ID NO: 213; and/or (m) a heavy chain variable region that comprises an HCDR1 that comprises the amino acid sequence set forth in SEQ ID NO: 221, an HCDR2 that comprises the amino acid sequence set forth in SEQ ID NO: 223, and an HCDR3 that comprises the amino acid sequence set forth in SEQ ID NO: 225, and a light chain variable region that comprises an LCDR1 that comprises the amino acid sequence set forth in SEQ ID NO: 32, an LCDR2 that comprises the amino acid sequence set forth in SEQ ID NO: 34, and an LCDR3 that comprises the amino acid sequence set forth in SEQ ID NO: 76. In an embodiment of the invention, the antibody or antigen-binding fragment comprises a heavy chain variable region that comprises the amino acid sequence set forth in SEQ ID NO: 2, 22, 42, 62, 82, 102, 122, 140, 159, 169, 179, 199 or 219, and a light chain variable region that comprises the amino acid sequence set forth in SEQ ID NO: 10, 30, 50, 70, 90, 110, 130, 148, 187, 207, or 227. In an embodiment of the invention, the antibody or antigen-binding fragment comprises (a) a heavy chain variable region that comprises the amino acid sequence set forth in SEQ ID NO: 2, and a light chain variable region that comprises the amino acid sequence set forth in SEQ ID NO: 10; (b) a heavy chain variable region that comprises the amino acid sequence set forth in SEQ ID NO: 22, and a light chain variable region that comprises the amino acid sequence set forth in SEQ ID NO: 30; (c) a heavy chain variable region that comprises the amino acid sequence set forth in SEQ ID NO: 42, and a light chain variable region that comprises the amino acid sequence set forth in SEQ ID NO: 50; (d) a heavy chain variable region that comprises the amino acid sequence set forth in SEQ ID NO: 62, and a light chain variable region that comprises the amino acid sequence set forth in SEQ ID NO: 70; (e) a heavy chain variable region that comprises the amino acid sequence set forth in SEQ ID NO: 82, and a light chain variable region that comprises the amino acid sequence set forth in SEQ ID NO: 90; (f) a heavy chain variable region that comprises the amino acid sequence set forth in SEQ ID NO: 102, and a light chain variable region that comprises the amino acid sequence set forth in SEQ ID NO: 110; (g) a heavy chain variable region that comprises the amino acid sequence set forth in SEQ ID NO: 122, and a light chain variable region that comprises the amino acid sequence set forth in SEQ ID NO: 130; (h) a heavy chain variable region that comprises the amino acid sequence set forth in SEQ ID NO: 140, and a light chain variable region that comprises the amino acid sequence set forth in SEQ ID NO: 148; (i) a heavy chain variable region that comprises the amino acid sequence set forth in SEQ ID NO: 159, and a light chain variable region that comprises the amino acid sequence set forth in SEQ ID NO: 148; (j) a heavy chain variable region that comprises the amino acid sequence set forth in SEQ ID NO: 169, and a light chain variable region that comprises the amino acid sequence set forth in SEQ ID NO: 148; (k) a heavy chain variable region that comprises the amino acid sequence set forth in SEQ ID NO: 179, and a light chain variable region that comprises the amino acid sequence set forth in SEQ ID NO: 187; (l) a heavy chain variable region that comprises the amino acid sequence set forth in SEQ ID NO: 199, and a light chain variable region that comprises the amino acid sequence set forth in SEQ ID NO: 207; and/or (m) a heavy chain variable region that comprises the amino acid sequence set forth in SEQ ID NO: 219, and a light chain variable region that comprises the amino acid sequence set forth in SEQ ID NO: 227. In an embodiment of the invention, the antibody or antigen-binding fragment comprises (a) a heavy chain that comprises the amino acid sequence set forth in SEQ ID NO: 18, 38, 58, 78, 98, 118, 136, 155, 167, 177, 195, 215 or 229, and a light chain that comprises the amino acid sequence set forth in SEQ ID NO: 20, 40, 60, 80, 100, 120, 138, 157, 197, 217, 231; e.g., which comprises (a) a heavy chain that comprises the amino acid sequence set forth in SEQ ID NO: 18, and a light chain that comprises the amino acid sequence set forth in SEQ ID NO: 20; (b) a heavy chain variable region that comprises the amino acid sequence set forth in SEQ ID NO: 38, and a light chain variable region that comprises the amino acid sequence set forth in SEQ ID NO: 40; (c) a heavy chain variable region that comprises the amino acid sequence set forth in SEQ ID NO: 58, and a light chain variable region that comprises the amino acid sequence set forth in SEQ ID NO: 60; (d) a heavy chain variable region that comprises the amino acid sequence set forth in SEQ ID NO: 78, and a light chain variable region that comprises the amino acid sequence set forth in SEQ ID NO: 80; (e) a heavy chain variable region that comprises the amino acid sequence set forth in SEQ ID NO: 98, and a light chain variable region that comprises the amino acid sequence set forth in SEQ ID NO: 100; (f) a heavy chain variable region that comprises the amino acid sequence set forth in SEQ ID NO: 118, and a light chain variable region that comprises the amino acid sequence set forth in SEQ ID NO: 120; (g) a heavy chain that comprises the amino acid sequence set forth in SEQ ID NO: 136, and a light chain that comprises the amino acid sequence set forth in SEQ ID NO: 138; (h) a heavy chain that comprises the amino acid sequence set forth in SEQ ID NO: 155, and a light chain that comprises the amino acid sequence set forth in SEQ ID NO: 157; (i) a heavy chain that comprises the amino acid sequence set forth in SEQ ID NO: 167, and a light chain that comprises the amino acid sequence set forth in SEQ ID NO: 157; (j) a heavy chain that comprises the amino acid sequence set forth in SEQ ID NO: 177, and a light chain that comprises the amino acid sequence set forth in SEQ ID NO: 157; (k) a heavy chain that comprises the amino acid sequence set forth in SEQ ID NO: 195, and a light chain that comprises the amino acid sequence set forth in SEQ ID NO: 197; (l) a heavy chain that comprises the amino acid sequence set forth in SEQ ID NO: 215, and a light chain that comprises the amino acid sequence set forth in SEQ ID NO: 217; and/or (m) a heavy chain that comprises the amino acid sequence set forth in SEQ ID NO: 229, and a light chain that comprises the amino acid sequence set forth in SEQ ID NO: 231. In an embodiment of the invention, the antibody or antigen-binding fragment thereof binds to one or more epitopes of FGFR3 selected from: a. an epitope comprising the sequence GPTVWVK (SEQ ID NO: 260) and/or an epitope comprising the sequence TQR; b. an epitope comprising the sequence ADVR (SEQ ID NO: 258) and/or an epitope comprising the sequence IGVAEK (SEQ ID NO: 259); c. an epitope comprising the sequence HCKVY (SEQ ID NO: 261), and/or an epitope comprising the sequence KSWISE (SEQ ID NO: 262), and/or an epitope comprising the sequence ADVR (SEQ ID NO: 263); e. an epitope comprised within or overlapping with the sequence GPTVWVK (SEQ ID NO: 260) and/or an epitope comprised within or overlapping with the sequence TQR; f. an epitope comprised within or overlapping with the sequence ADVR (SEQ ID NO: 258) and/or an epitope comprised within or overlapping with the sequence IGVAEK (SEQ ID NO: 259); and g. an epitope comprised within or overlapping with the sequence HCKVY (SEQ ID NO: 261), and/or an epitope comprised within or overlapping with the sequence KSWISE (SEQ ID NO: 262), and/or an epitope comprised within or overlapping with the sequence ADVR (SEQ ID NO: 263). In an embodiment of the invention, the antibody or antigen-binding fragment thereof binds to one or more epitopes of FGFR3 selected from: a. an epitope consisting of the sequence GPTVWVK (SEQ ID NO: 260) and/or an epitope consisting of the sequence IGVAEK (SEQ ID NO: 259); b. an epitope consisting of the sequence ADVR (SEQ ID NO: 258) and/or an epitope consisting of the sequence IGVAEK (SEQ ID NO: 259); and c. an epitope consisting of the sequence HCKVY (SEQ ID NO: 261), and/or an epitope consisting of the sequence KSWISE (SEQ ID NO: 262), and/or an epitope consisting of the sequence ADVR (SEQ ID NO: 263). In an embodiment of the invention, the antibody or antigen-binding fragment thereof binds to one or more epitopes of FGFR3 selected from: a. an epitope comprising the sequence SCPPPGGGPMGPTVWVKDGTGLVPSER (SEQ ID NO: 245) and/or an epitope comprising the sequence YSCRQRLTQRVL (SEQ ID NO: 246); b. an epitope comprising the sequence LLAVPAAN (SEQ ID NO: 247), and/or an epitope comprising the sequence VLERSPHRPILQAG (SEQ ID NO: 248) and/or an epitope comprising the sequence YVTVLKSWISE (SEQ ID NO: 249) and/or or an epitope comprising the sequence ADVRLR (SEQ ID NO: 250) and/or an epitope comprising the sequence LCRATNFIGVAEKAFW (SEQ ID NO: 251); c. an epitope comprising the sequence GQQEQLVFGSGDAVE (SEQ ID NO: 252) and/or an epitope comprising the sequence VLVGPQRL (SEQ ID NO: 253); d. an epitope comprising the sequence VLERSPHRPILQAG (SEQ ID NO: 254) and/or an epitope comprising the sequence HCKVYSDAQP (SEQ ID NO: 255) and/or an epitope comprising the sequence YVTVLKSWISESVEADVRLR (SEQ ID NO: 256) and/or an epitope comprising the sequence LCRATNFIGVAEKAF (SEQ ID NO: 257); e. an epitope comprised within or overlapping with the sequence SCPPPGGGPMGPTVWVKDGTGLVPSER (SEQ ID NO: 245) and/or an epitope comprised within or overlapping with the sequence YSCRQRLTQRVL (SEQ ID NO: 246); f. an epitope comprised within or overlapping with the sequence LLAVPAAN (SEQ ID NO: 247), and/or an epitope comprised within or overlapping with the sequence VLERSPHRPILQAG (SEQ ID NO: 248) and/or an epitope comprised within or overlapping with the sequence YVTVLKSWISE (SEQ ID NO: 249) and/or or an epitope comprised within or overlapping with the sequence ADVRLR (SEQ ID NO: 250) and/or an epitope comprised within or overlapping with the sequence LCRATNFIGVAEKAFW (SEQ ID NO: 251); g. an epitope comprised within or overlapping with the sequence GQQEQLVFGSGDAVE (SEQ ID NO: 252) and/or an epitope comprised within or overlapping with the sequence VLVGPQRL (SEQ ID NO: 253); and h. an epitope comprised within or overlapping with the sequence VLERSPHRPILQAG (SEQ ID NO: 254) and/or an epitope comprised within or overlapping with the sequence HCKVYSDAQP (SEQ ID NO: 255) and/or an epitope comprised within or overlapping with the sequence YVTVLKSWISESVEADVRLR (SEQ ID NO: 256) and/or an epitope comprised within or overlapping with the sequence LCRATNFIGVAEKAF (SEQ ID NO: 257). In an embodiment of the invention, the antibody or antigen-binding fragment thereof binds to one or more epitopes of FGFR3 selected from: a. an epitope consisting of the sequence SCPPPGGGPMGPTVWVKDGTGLVPSER (SEQ ID NO: 245) and/or an epitope consisting of the sequence YSCRQRLTQRVL (SEQ ID NO: 246); b. an epitope consisting of the sequence LLAVPAAN (SEQ ID NO: 247), and/or an epitope consisting of the sequence VLERSPHRPILQAG (SEQ ID NO: 248) and/or an epitope consisting of the sequence YVTVLKSWISE (SEQ ID NO: 249) and/or or an epitope consisting of the sequence ADVRLR (SEQ ID NO: 250) and/or an epitope consisting of the sequence LCRATNFIGVAEKAFW (SEQ ID NO: 251); c. an epitope consisting of the sequence GQQEQLVFGSGDAVE (SEQ ID NO: 252) and/or an epitope consisting of the sequence VLVGPQRL (SEQ ID NO: 253); and d. an epitope consisting of the sequence VLERSPHRPILQAG (SEQ ID NO: 254) and/or an epitope consisting of the sequence HCKVYSDAQP (SEQ ID NO: 255) and/or an epitope consisting of the sequence YVTVLKSWISESVEADVRLR (SEQ ID NO: 256) and/or an epitope consisting of the sequence LCRATNFIGVAEKAF (SEQ ID NO: 257).

Also provided herein is an isolated antibody or antigen-binding fragment thereof that specifically binds to FGFR3 or an antigenic fragment thereof that binds to the same FGFR3 epitope as or competes for binding to FGFR3 with an antibody or antigen-binding fragment described herein.

For example, the present invention provides an antibody or antigen-binding fragment thereof that specifically binds to FGFR3 or an antigenic fragment thereof that is characterized by one or more of the following: Binds (e.g., in a surface plasmon resonance assay) to monomeric human FGFR3b (e.g., tagged, for example, at the C-terminus, with myc-myc-His₆) at 25° C. with an affinity (K_{D}) of about 16 nM or greater affinity (e.g., about 16 nM, 12 nM, 10 nM, 7 nM, 5 nM, 4 nM, 3 nM, 2 nM, 1 nM, 0.5 nM, 0.22 nM, 0.2 nM, 0.19 nM, 0.14 nM, 0.1 nM); Binds (e.g., in a surface plasmon resonance assay) to monomeric Cynomolgous monkey FGFR3b (e.g., tagged, for example, at the C-terminus, with myc-myc-His₆) at 25° C. with an affinity (K_{D}) of about 20 nM or greater affinity (e.g., about 20 nM, 16 nM, 15 nM, 10 nM, 8 nM, 5 nM, 4 nM, 3 nM, 2 nM, 1 nM, 0.9 nM, 0.65 nM, 0.3 nM, 0.28 nM, 0.2 nM, 0.15 nM, 0.1 nM); Binds (e.g., in a surface plasmon resonance assay) monomeric murine FGFR3b (e.g., tagged, for example, at the C-terminus, with myc-myc-His₆) at 25° C. with an affinity (K_{D}) of about 70 nM or greater affinity (e.g., about 70 nM, 20 nM, 17 nM, 12 nM, 10 nM, 9 nM, 8 nM, 0.1 nM); Does not bind significantly to monomeric human FGFR3c (e.g., tagged, for example, at the C-terminus, with myc-myc-His₆) at 25° C. (e.g., in a surface plasmon resonance assay); Binds (e.g., in a surface plasmon resonance assay) dimeric human FGFR3b (e.g., tagged, for example, at the C-terminus, with a mouse Fc (mFc)) 25° C. with an affinity of about 0.6 nM of greater affinity (e.g., about 0.58 nM, 0.17 nM, 0.11 nM, 0.04 nM, 0.03 nM, 0.02 nM, 0.01 nM, 0.023 nM, 0.061 nM, 0.031 nM, 0.016 nM, 0.034 nM, 0.027 nM); Blocks about 68% or more binding of FGF1 acidic to human FGFR3b-mFc at 200 nM antibody (3.g., 90% or 05%); Binds to monomeric Cynomolgous and monomeric mouse FGFR3b with a K_{D} that is within about 0.1 nM of the K_{D} for binding to monomeric or dimeric human FGFR3b; Blocks binding of 4 nM human FGFR3b-mFc to human FGF1 acidic protein with an IC₅₀ of about 15 nM or a lower concentration (e.g., IC₅₀ of about 1, 2 or 3 nM); Competes for binding to hFGFR3b.mmH with another anti-FGFR3 antibody as set forth in Table 3-1 herein; Blocks intracellular signaling in an engineered IL-3-dependent Ba/F3 murine hematopoietic cell line genetically modified to stably express wild-type or S249C mutant human fibroblast growth factor receptor 3b that has been stimulated with human heparin, e.g., at about 5 micrograms/ml, and human FGF1 ligand, e.g., at about 1 nM, with an IC₅₀ of about 18 nM or a lower concentration (e.g., about 0.51 nM, 0.5 nM, 0.61 nM, 0.6 nM, or 0.012 nM); Blocks intracellular signaling in an engineered IL-3-dependent Ba/F3 murine hematopoietic cell line genetically modified to stably express S249C mutant human fibroblast growth factor receptor 3b that has been stimulated with human heparin, e.g., at about 5 micrograms/ml, and human FGF1 ligand, e.g., at about 1 nM, with an IC₅₀ that is lower than for blocking intracellular signaling in an engineered IL-3-dependent Ba/F3 murine hematopoietic cell line genetically modified to stably express wild-type human fibroblast growth factor receptor 3b that has been stimulated with human FGF1 ligand, e.g., at about 1 nM, and human heparin, e.g., at about 5 micrograms/ml; Blocks dimerization of FGFR3 (e.g., wild-type or S249C mutant), e.g., with stronger inhibition than that of REGN6331, for example, as measured in a non-reducing SDS-PAGE assay; Reduces tumor size (e.g., bladder cancer tumor expressing FGFR3 (e.g., S249C mutant)) in a subject administered the antibody or fragment; Reduces CD73 expression in a tumor (e.g., bladder cancer tumor expressing FGFR3 (e.g., S249C mutant)) in a subject administered the antibody or fragment; Inhibits FGF1/heparin stimulation induced phosphorylation of MAPK, e.g., in a BaF3 cell expressing wild-type FGFR3 (e.g., S249C mutant); Inhibits proliferation of UMUC14 bladder cancer cells expressing endogenous FGFR3 S249C mutation (e.g., in a cancer cell spheroid proliferation assay); Inhibits tumor growth in a mouse (e.g., SCID mouse) xenograft model of bladder cancer cell line UMUC14 expressing FGFR3 (e.g., S249C mutation); inhibits CD73-dependent, adenosine-mediated inhibition of immune cell activation; increases the ratio of CD8/CD4 and/or CD8+/T_{reg} in tumor tissue having tumor cells expressing FGFR3 (e.g., S249C mutant); Inhibits FGF3-mediated activation of CD73 expression and/or enzymatic activity (e.g., adenosine production) (e.g., on a tumor cell expressing FGFR3 (e.g., S249C mutant)); Inhibits FGFR3-dependent, adenosine-mediated immune cell suppression; Inhibits growth of BaF3 cells expressing the FGFR3 double mutant S249C and V557M, or S249C and V557L, wherein the antibody or antigen-binding fragment comprises immunoglobulin chains including any of the amino acid sequences as set forth herein.

The present invention also provides a complex comprising such an antibody or antigen-binding fragment thereof bound to FGFR3 or an antigenic fragment thereof is also part of the present invention.

A pharmaceutical formulation comprising an anti-FGFR3 antibody or fragment as set forth herein and a pharmaceutically acceptable carrier is also within the scope of the present invention.

The present further provides an isolated polypeptide comprising an amino acid sequence selected from SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229 and 231; as well as an isolated polynucleotide that encodes any one or more of said polypeptides, e.g., comprising a nucleotide sequence selected from that set forth in SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228 and 230. The present invention also includes a vector that comprises a polynucleotide as set forth herein. The present invention also provides host cell (e.g., a eukaryotic or mammalian cell, such as a CHO cell) that comprises a polynucleotide set forth herein.

The present invention also provides a method for making an anti-FGFR3 antibody or fragment as set forth herein including the steps of introducing a polynucleotide that encodes the chains of the antibody or fragment into a host cell (e.g., CHO cell) and incubating the host cell that comprises the polynucleotide in a culture medium under conditions favorable to expression of the chains and, optionally, isolation of the antibody or fragment from the host cell and/or culture medium.

The present invention further provides a method for administering an anti-FGFR3 antibody or fragment as set forth herein to a subject comprising introducing (e.g., by injection, for example, intramuscular, intravenous or subcutaneous injection) the antibody or fragment into the body of the subject.

The present invention also provides a method for treating or preventing an FGFR3-mediated condition (e.g., cancer (e.g., mediated by cancer cells expressing an FGFR3 S249C mutation), bladder cancer, brain cancer, breast cancer, cervical cancer, colorectal cancer, endometrial cancer, gastric cancer, head & neck cancer, kidney cancer, lung cancer, multiple myeloma, ovarian cancer, pancreatic cancer, urothelial cancer, achondroplasia, crouzon syndrome with acanthosis nigricans, epidermal nevus, hypochondroplasia; lacrimo-auriculo-dento-digital (LADD) syndrome, muenke syndrome, severe achondroplasia with developmental delay and acanthosis nigricans (SADDAN) and/or thanatophoric dysplasia), in a subject in need thereof, comprising administering a therapeutically effective amount of an anti-FGFR3 antibody or fragment to the subject.

The present invention provides a method for reducing metastasis (e.g., CD73-dependent or adenosine-dependent) of a tumor cell expressing FGFR3 (e.g., S249C mutant), reducing the concentration of adenosine in a tumor expressing FGFR3 (e.g., S249C mutant), reducing CD73-dependent catalysis of AMP to adenosine by a tumor expressing FGFR3 (e.g., S249C mutant), and/or inhibiting adenosine-mediated suppression of T-cell function in a tumor expressing FGFR3 (e.g., S249C mutant), in the body of a subject in need thereof (e.g., a subject with cancer such as bladder cancer) comprising administering a therapeutically effective amount of the anti-FGFR3 antibody or fragment to the subject.

### BRIEF DESCRIPTION OF THE FIGURES

FIG. 1. BaF3/FGFR3 WT (top, left and right panels) and BaF3/FGFR3_S249C (bottom, left and right panels) cells were treated with a titration of H4H30063P (left, top and bottom panels) or H4H30102P2 (right, top and bottom panels), alongside a comparator antibody (REGN6331) and isotype matched negative controls (REGN1945, REGN1932) in the presence of 1 nM FGF1 and 5 μg/ml heparin. CellTiter-Glo, which measures ATP levels, was used to determine cell growth.

FIG. 2. Non-reducing and reducing SDS-PAGE analysis of FGFR3 dimerization in the presence of various concentrations of H4H30063P or COMP6331 (REGN6331). The mobility of the FGFR3 dimer and monomer are indicated.

FIG. 3. Western blot analysis of phosphorylated MAPK, from in vitro BaF3/hFGFR3b WT (wild-type), in the presence of no antibody (-), 100 nM control antibody (Con Ig; REGN6331) or 100 nM H4H30063P either with or without FGF1 & Heparin stimulation.

FIG. 4. Proliferation of UMUC14 bladder cancer cells treated with a range of concentrations (Log₁₀ scale) of control antibody (IgG control), H4H30063P, H4H30071P or REGN6331 relative to that of untreated cells.

FIG. 5. UMUC14 tumor size (mm³) in xenograft SCID mice treated with control antibody (10 mg/kg), H4H30063P (3 mg/kg or 10 mg/kg) or REGN6331 (3 mg/kg or 10 mg/kg) over time.

FIG. 6. Western blot analysis of CD73 protein expression in in vitro UMUC14 cells treated with control antibody (Con) or H4H30063P (FGFR3 Ab) or AZD4547 (FGFR TKI) at 1 nM, 10 nM and 100 nM concentrations.

FIG. 7. Western blot analysis of CD73 protein expression in in vivo UMUC14 cells from xenograft mice treated with control antibody (Con) or H4H30063P (FGFR3 Ab).

FIG. 8. Quantitation of Western blot band intensity of CD73/Actin from the blot in FIG. 7.

FIG. 9. Western blot analysis of expression of CD73 protein in cell line expressing empty vector (EV), Wild-type FGFR3 (FGFR3), FGFR3-TACC3 mutant, FGFR3 S249C mutant or FGFR3 Y375C mutant.

FIGS. 10A-10C. Western blot analysis of expression of CD73 protein in tumor cells from xenograft SCID mice expressing (FIG. 10A) empty vector (EV) or (FIG. 10B) FGFR3 S249C mutant or (FIG. 10C) FGFR3-TACC3 mutant.

FIGS. 11A-11B. Western blot analysis of expression of CD73 protein level in Fadu S249C tumor cells in xenograft mice treated with control antibody, H4H30063P antibody or AZD4547 (FIG. 11A) and graphical quantitation of band intensity (FIG. 11B).

FIGS. 12A-12B. FGFR3 antibody inhibits proliferation of BaF3 cell expressing TKI resistant mutations V557L/M. The pan-FGFR TKIs AZD4547 and erdafitinib potently inhibited the growth of BaF3 cells expressing FGFR3 S249C (IC50 9.9 nM and 1.2 nM, respectively) (FIG. 12A, left), but were ineffective in cells expressing FGFR3 that contains V557M/L mutation alone or both the S249C and V557M/L mutations (FIG. 12A, right, S249C+V557L; FIG. 12B, S249C+V557M). In contrast, FGFR3 antibody H4H30063P was able to strongly inhibit the growth of BaF3 cells expressing the double mutant FGFR3 (S249C plus V557M or L; see, e.g., FIG. 12B and FIG. 12A, right, respectively). The IC50 values for H4H30063P against S249C, S249C/V557M, and S249CN557L were 1.36 nM, 3.9 nM, and 4.3 nM, respectively. See FIGS. 12A-12B.

FIG. 13. HDX epitope mapping results for anti-FGFR3b H4H30117P2, H4H30063P.

FIG. 14. HDX epitope mapping results for anti-FGFR3b H4H30045P and H4H30108P2.

FIG. 15. HDX protection by FGFR3 antibodies. Regions with above 20% and 25% HDX protection are shown.

FIGS. 16A-16B. HDX epitope mapping results of FGFR3 antibodies. Depiction of HDX epitope mapping structures (FIG. 16A). Table showing FGFR3b residues with significant changes in deuterium percent (%) (FIG. 16B).

FIG. 17. HDX epitope mapping results for H4H30063P. Figure discloses SEQ ID NO: 238.

FIG. 18. HDX epitope mapping results for H4H30108P2. Figure discloses SEQ ID NO: 238.

FIG. 19. HDX epitope mapping results for H4H30117P2. Figure discloses SEQ ID NO: 238.

FIG. 20. HDX epitope mapping results for H4H30045P. Figure discloses SEQ ID NO: 238.

FIG. 21. Exemplary Hydrogen-Deuterium Exchange Mass Spectrometry experimental process.

### DETAILED DESCRIPTION OF THE INVENTION

The present invention provides anti-FGFR3 antibodies that exhibit in vitro and in vivo properties that are superior. In some embodiments, antibodies of the present invention exhibit greater binding to human monomeric FGFR3b while not exhibiting such binding to FGFR3c. This property facilitates a more targeted approach to treating cancer, i.e., the ability to inhibit the b-isoform but not the c-isoform. In contrast, the Bioclin Therapeutics anti-FGFR3 antibody, B-701, was observed to exhibit binding to FGFR3c. The anti-FGFR3 antibodies herein also exhibited superior biological activities, relative to B-701, including for example, greater inhibition of FGFR3b dimerization, in vitro cancer cell proliferation and tumor growth in a mouse xenograft model.

### FGFR3

The term “FGFR3” refers to monomeric or dimeric human FGFR3b to which the antibodies and antigen-binding fragments thereof of the present invention bind specifically.

The fibroblast growth factor receptor 3 (FGFR3) belongs to a family of structurally related tyrosine kinase receptors including four different genes (FGFR1-4). These receptors have three glycosylated extracellular immunoglobulin-like domains (Ig-like), a transmembrane domain and a split intracellular tyrosine-kinase domain. Ligand binding induces FGFR dimerization, resulting in autophosphorylation of the kinase domain and interaction with and phosphorylation of effector signaling proteins. Alternative mRNA splicing mechanisms generate many different receptor isoforms, which differ in ligand specificity. The isoforms FGFR3b and FGFR3c result from a mutually exclusive splicing event, in which the second half of the third Ig-like domain is encoded by either the 151 nucleotides of exon 8 or the 145 nucleotides of exon 9. These two isoforms have different tissue distributions: for example, FGFR3b is the main form in epithelial cells whereas FGFR3c is the predominant form in chondrocytes.

Mutations of FGFR3 are associated with autosomal dominant dwarfism and craniosynostosis syndromes such as hypochondroplasia, achondroplasia, severe achondroplasia with developmental delay and acanthosis nigricans (SADDAN), thanatophoric dysplasia, Crouzon syndrome with acanthosis nigricans and Muenke coronal craniosynostosis. Reports have demonstrated that these mutations lead to constitutive activation of the receptor.

In addition, there is an oncogenic role for FGFR3 in human cancer. Indeed, somatic activating mutations in FGFR3 have been reported in multiple myeloma and, more recently, in two epithelial malignancies, i.e., bladder- and cervix carcinomas. FGFR3 is a driver in muscle invasive bladder cancer (MIBC). See Cappellen et al., Frequent activating mutations of FGFR3 in human bladder and cervix carcinomas Nat Genet 1999 23: 18-20; Chesi et al., Frequent translocation t(4;14)(p16.3;q32.3) in multiple myeloma is associated with increased expression and activating mutations of fibroblast growth factor receptor 3 Nat Genet 1997 16: 260-264; and Richelda et al. A novel translocation t(4;14)(p16.3;q32.3) in multiple myeloma involves the fibroblast growth-factor receptor 3 gene Blood 1997 90: 4062-4070. FGFR3 mutations are rare in multiple myeloma and cervix carcinomas, whereas their high incidence in bladder carcinomas (74% of non-invasive papillary tumors) suggests that the constitutive activation of FGFR3 is an important event for bladder tumorigenesis. See Fracchiolla et al., FGFR3 gene mutations associated with human skeletal disorders occur rarely in multiple myeloma Blood 1998 92: 2987-2989; Wu et al., Somatic mutations of fibroblast growth factor receptor 3 (FGFR3) are uncommon in carcinomas of the uterine cervix Oncogene 2000 19: 5543-5546; Billerey et al. Frequent FGFR3 mutations in papillary non-invasive bladder (pTa) tumors Am J Pathol 2001 158: 1955-1959; and Van Rhijn et al., The fibroblast growth factor receptor 3 (FGFR3) mutation is a strong indicator of superficial bladder cancer with low recurrence rate Cancer Res 2001 61: 1265-1268. Many mutations identified in bladder tumours are identical to the activating mutations responsible for thanatophoric dysplasia, a lethal form of dwarfism. See Cappellen et al., Frequent activating mutations of FGFR3 in human bladder and cervix carcinomas Nat Genet 1999 23: 18-20; Billerey et al., Frequent FGFR3 mutations in papillary non-invasive bladder (pTa) tumors Am J Pathol 2001 158: 1955-1959; Van Rhijn et al., The fibroblast growth factor receptor 3 (FGFR3) mutation is a strong indicator of superficial bladder cancer with low recurrence rate Cancer Res 2001 61: 1265-1268; and Sibley et al., Loss of heterozygosity at 4p16.3 and mutation of FGFR3 in transitional cell carcinoma Oncogene 2001 20: 686-691. Two of the 117 FGFR3 mutations identified by these groups, the A393E and K652Q mutations, do not correspond to thanatophoric dysplasia mutations. The A393E mutation is identical to a mutation associated with a craniosynostosis syndrome (Crouzon syndrome with acanthosis nigricans) and the K652Q mutation is identical to a mutation associated with hypochondroplasia.

In an embodiment of the invention, the human FGFR3c isoform comprises the amino acid sequence:

(SEQ ID NO: 232)
ESLGTEQRVVGRAAEVPGPEPGQQEQLVFGSGDAVELSCPPPGGGPMGPT
VWVKDGTGLVPSERVLVGPQRLQVLNASHEDSGAYSCRQRLTQRVLCHFS
VRVTDAPSSGDDEDGEDEAEDTGVDTGAPYWTRPERMDKKLLAVPAANTV
RFRCPAAGNPTPSISWLKNGREFRGEHRIGGIKLRHQQWSLVMESVVPSD
RGNYTCVVENKFGSIRQTYTLDVLERSPHRPILQAGLPANQTAVLGSDVE
FHCKVYSDAQPHIQWLKHVEVNGSKVGPDGTPYVTVLKTAGANTTDKELE
VLSLHNVTFEDAGEYTCLAGNSIGFSHHSAWLVVLPAEEELVEADEAGSV
YAG

In an embodiment of the invention, the human FGFR3b isoform comprises the amino acid sequence:

(SEQ ID NO: 233)
ESLGTEQRVVGRAAEVPGPEPGQQEQLVFGSGDAVELSCPPPGGGPMGPT
VWVKDGTGLVPSERVLVGPQRLQVLNASHEDSGAYSCRQRLTQRVLCHFS
VRVTDAPSSGDDEDGEDEAEDTGVDTGAPYWTRPERMDKKLLAVPAANTV
RFRCPAAGNPTPSISWLKNGREFRGEHRIGGIKLRHQQWSLVMESVVPSD
RGNYTCVVENKFGSIRQTYTLDVLERSPHRPILQAGLPANQTAVLGSDVE
FHCKVYSDAQPHIQWLKHVEVNGSKVGPDGTPYVTVLKSWISESVEADVR
LRLANVSERDGGEYLCRATNFIGVAEKAFWLSVHGPRAAEEELVEADEAG
SVYAG

In an embodiment of the invention, an FGFR3 referred to herein comprises one or more of the following mutations: S249C, R248C, G372C, Y375C, K650E, or FGFR3-TACC3. See Singh et al., Transforming fusions of FGFR and TACC genes in human glioblastoma. Science (New York, NY) 2012; 337:1231-1235. Tomlinson et al., Knockdown by shRNA identifies S249C mutant FGFR3 as a potential therapeutic target in bladder cancer, Oncogene 2007 Aug. 30 26(40):5889-99; Otsuka et al., Constitutively Active FGFR3 with Lys650Glu Mutation Enhances Bortezomib Sensitivity in Plasma Cell Malignancy, Anticancer Research January 2011, 31 (1) 113-122.

### Antigen-Binding Proteins

The present invention provides antigen-binding proteins, such as antibodies (e.g., human antibodies, monoclonal antibodies and recombinant antibodies) and antigen-binding fragments thereof, that specifically bind to FGFR3 protein (e.g., monomeric or dimeric FGFR3b) or an antigenic fragment thereof (e.g., the extracellular domain of FGFR3). In an embodiment of the invention, the FGFR3 is an activating mutant (e.g., as discussed herein). Antigen-binding proteins that bind to the same epitope on FGFR3 as, or compete for binding to FGFR3 with, any of the antigen-binding proteins set forth herein (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2), are also part of the present invention.

The term “antibody”, as used herein, refers to immunoglobulin molecules comprising four polypeptide chains, two heavy chains (HCs) and two light chains (LCs), inter-connected by disulfide bonds (e.g., IgG). In an embodiment of the invention, each antibody heavy chain (HC) comprises a heavy chain variable region (“HCVR” or “V_{H}”) (e.g., SEQ ID NO: 2 or a variant thereof) and a heavy chain constant region; and each antibody light chain (LC) comprises a light chain variable region (“LCVR or “V_{L}”) (e.g., SEQ ID NO: 10 or a variant thereof) and a light chain constant region (CL). The V_{H} and V_{L} regions can be further subdivided into regions of hypervariability, termed complementarity determining regions (CDR), interspersed with regions that are more conserved, termed framework regions (FR).

In an embodiment of the invention, an anti-FGFR3 antigen-binding protein, e.g., antibody or antigen-binding fragment, comprises a heavy chain constant domain, e.g., of the type IgA (e.g., IgA1 or IgA2), IgD, IgE, IgG (e.g., IgG1, IgG2, IgG3 and IgG4 (e.g., comprising a S228P and/or S108P mutation)) or IgM. In an embodiment of the invention, an antigen-binding protein, e.g., antibody or antigen-binding fragment, comprises a light chain constant domain, e.g., of the type kappa or lambda. In an embodiment of the invention, a V_{H} as set forth herein is linked to a human heavy chain constant domain (e.g., IgG) and a V_{L} as set forth herein is linked to a human light chain constant domain (e.g., kappa). The present invention includes antigen-binding proteins comprising the variable domains set forth herein (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2), which are linked to a heavy and/or light chain constant domain, e.g., as set forth herein.

In an embodiment of the invention, the assignment of amino acids to each framework or CDR domain is in accordance with the definitions of Sequences of Proteins of Immunological Interest, Kabat et al.; National Institutes of Health, Bethesda, Md.; 5^{th} ed.; NIH Publ. No. 91-3242 (1991); Kabat (1978) Adv. Prot. Chem. 32:1-75; Kabat et al., (1977) J. Biol. Chem. 252:6609-6616; Chothia et al., (1987) J Mol. Biol. 196:901-917 or Chothia et al., (1989) Nature 342:878-883. Thus, the present invention includes antibodies and antigen-binding fragments including the CDRs of a V_{H} and the CDRs of a V_{L}, which V_{H} and V_{L} comprise amino acid sequences as set forth herein (or a variant thereof), wherein the CDRs are as defined, for example, according to Kabat and/or Chothia.

An FGFR3 binding protein described herein may be an antigen-binding fragment of an antibody. The terms “antigen-binding portion” or “antigen-binding fragment” of an antibody, as used herein, refers to an immunoglobulin molecule that binds antigen but that does not include all of the sequences of a full antibody (preferably, the full antibody is an IgG). Non-limiting examples of antigen-binding fragments include: (i) Fab fragments; (ii) F(ab′)2 fragments; (iii) Fd fragments; (iv) Fv fragments; (v) single-chain Fv (scFv) molecules; and (vi) dAb fragments; consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, one-armed antibodies, domain-deleted antibodies, chimeric antibodies, CDR-grafted antibodies, diabodies, triabodies, tetrabodies, minibodies and small modular immunopharmaceuticals (SMIPs), are also encompassed within the expression “antigen-binding fragment,” as used herein.

“Isolated” antigen-binding proteins (e.g., antibodies or antigen-binding fragments thereof), polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules (e.g., minor or insignificant amounts of impurity may remain) or to an absence of water, buffers, or salts or to components of a pharmaceutical formulation that includes the antigen-binding proteins (e.g., antibodies or antigen-binding fragments).

The amino acid sequences of polypeptide of the present invention and the nucleotide sequences of polynucleotides of the present invention are set forth below in Tables A and B, respectively.

TABLE A
Amino Acid Sequences of Exemplary Immunoglobulin Chains of the Present Invention

| | HCVR | HCDR1 | HCDR2 | HCDR3 | LCVR | LCDR1 | LCDR2 | LCDR3 | HC* | LC |
|---|---|---|---|---|---|---|---|---|---|---|
| H4H30063P | 2 | 4 | 6 | 8 | 10 | 12 | 14 | 16 | 18 | 20 |
| (REGN15684) | | | | | | | | | | |
| H4H30066P | 22 | 24 | 26 | 28 | 30 | 32 | 34 | 36 | 38 | 40 |
| H4H30071P | 42 | 44 | 46 | 48 | 50 | 52 | 54 | 56 | 58 | 60 |
| H4H30089P2 | 62 | 64 | 66 | 68 | 70 | 72 | 74 | 76 | 78 | 80 |
| H4H30093P2 | 82 | 84 | 86 | 88 | 90 | 92 | 94 | 96 | 98 | 100 |
| H4H30102P2 | 102 | 104 | 106 | 108 | 110 | 112 | 114 | 116 | 118 | 120 |
| H4H30076P | 122 | 124 | 126 | 128 | 130 | 132 | 34 | 134 | 136 | 138 |
| H4H30105P2 | 140 | 142 | 144 | 146 | 148 | 150 | 14 | 153 | 155 | 157 |
| H4H30108P2 | 159 | 161 | 163 | 165 | 148 | 150 | 14 | 153 | 167 | 157 |
| H4H30117P2 | 169 | 171 | 173 | 175 | 148 | 150 | 14 | 153 | 177 | 157 |
| H4H30045P | 179 | 181 | 183 | 185 | 187 | 189 | 191 | 193 | 195 | 197 |
| H4H30061P | 199 | 201 | 203 | 205 | 207 | 209 | 211 | 213 | 215 | 217 |
| H4H30095P2 | 219 | 221 | 223 | 225 | 227 | 32 | 34 | 76 | 229 | 231 |

*In an embodiment of the invention, the heavy chain lacks the C-terminal Lysine.

TABLE B
Nucleotide Sequences of Exemplary Polynucleotides Encoding
Immunoglobulin Chains of the Present Invention

| | HCVR | HCDR1 | HCDR2 | HCDR3 | LCVR | LCDR1 | LCDR2 | LCDR3 | HC* | LC |
|---|---|---|---|---|---|---|---|---|---|---|
| H4H30063P | 1 | 3 | 5 | 7 | 9 | 11 | 13 | 15 | 17 | 19 |
| (REGN15684) | | | | | | | | | | |
| H4H30066P | 21 | 23 | 25 | 27 | 29 | 31 | 33 | 35 | 37 | 39 |
| H4H30071P | 41 | 43 | 45 | 47 | 49 | 51 | 53 | 55 | 57 | 59 |
| H4H30089P2 | 61 | 63 | 65 | 67 | 69 | 71 | 73 | 75 | 77 | 79 |
| H4H30093P2 | 81 | 83 | 85 | 87 | 89 | 91 | 93 | 95 | 97 | 99 |
| H4H30102P2 | 101 | 103 | 105 | 107 | 109 | 111 | 113 | 115 | 117 | 119 |
| H4H30076P | 121 | 123 | 125 | 127 | 129 | 131 | 33 | 133 | 135 | 137 |
| H4H30105P2 | 139 | 141 | 143 | 145 | 147 | 149 | 151 | 152 | 154 | 156 |
| H4H30108P2 | 158 | 160 | 162 | 164 | 147 | 149 | 151 | 152 | 166 | 156 |
| H4H30117P2 | 168 | 170 | 172 | 174 | 147 | 149 | 151 | 152 | 176 | 156 |
| H4H30045P | 178 | 180 | 182 | 184 | 186 | 188 | 190 | 192 | 194 | 196 |
| H4H30061P | 198 | 200 | 202 | 204 | 206 | 208 | 210 | 212 | 214 | 216 |
| H4H30095P2 | 218 | 220 | 222 | 224 | 226 | 31 | 33 | 75 | 228 | 230 |

*In an embodiment of the invention, the heavy chain lacks a codon encoding the C-terminal Lysine.

Sequences of immunoglobulin chains of anti-FGFR3 antibodies and antigen-binding fragments of the present invention are set forth below. Thus, the present invention includes any antibody or antigen-binding fragment thereof that includes a HCVR and LCVR having amino acid sequences as set forth below or an HCVR and LCVR having the HCDRs and LCDRs thereof, respectively.

H4H30063P
HCVR Nucleotide Sequence
(SEQ ID NO: 1)
CAGGTGCAGCTGCAGGAGTCGGGCCCAGGACTGGTGAAGCCTTCGGAGACCCTGTCCCTCACCTGCACTGTCTCTGGTGA
CTCCATTAATAGTTACTTCTGGAGCTGGATCCGGCAGTTGCCAGGGAAGGAACTGGAGTGGATTGGCCATATCTATTCTA
GTGGGAGTACCAGATACAACCCCTCCCTCCAGAGTCGAGTCACCATATCAATAGACACGTCCAAGAACCAGTTCTCCCTG
AAGCTGAGTTCTGTGACCGCTGCGGACACGGCCGTATATTACTGTGCGAGGGGCGCCAGCGCAGTTGACTACTGGGGCCA
GGGAACCCTGGTCACCGTCTCCTCA
HCVR Amino Acid Sequence
(SEQ ID NO: 2)
QVQLQESGPGLVKPSETLSLTCTVSGDSINSYFWSWIRQLPGKELEWIGHIYSSGSTRYNPSLQSRVTISIDTSKNQFSL
KLSSVTAADTAVYYCARGASAVDYWGQGTLVTVSS
HCDR1 Nucleotide Sequence
(SEQ ID NO: 3)
GGT GAC TCC ATT AAT AGT TAC TTC
HCDR1 Amino Acid Sequence
(SEQ ID NO: 4)
G D S I N S Y F
HCDR2 Nucleotide Sequence
(SEQ ID NO: 5)
ATC TAT TCT AGT GGG AGT ACC
HCDR2 Amino Acid Sequence
(SEQ ID NO: 6)
I Y S S G S T
HCDR3 Nucleotide Sequence
(SEQ ID NO: 7)
GCG AGG GGC GCC AGC GCA GTT GAC TAC
HCDR3 Amino Acid Sequence
(SEQ ID NO: 8)
A R G A S A V D Y
LCVR Nucleotide Sequence
(SEQ ID NO: 9)
GAAATTGTGTTGACGCAGTCTCCAGGCACCCTGTCTTTGTCTCCAGGGGAAAGAGCCACCCTCTCCTGCAGGACCAGTCA
GAGTATTAGCAGCGGCTATTTAGCCTGGTACCAGCAGAAACCTGGCCAGGCTCCCAGGCTCCTCATCTATGGTGCATCCA
GAAGGGCCACTGGCATCCCAGACAGGTTCAGTGGCAGTGGGTCTGGGACAGACTTCACTCTCACCATCAGCAGACTGGAG
CCTGAAGACTTTGTAGTGTATTACTGTCAACAATATGGTAGCTCACCATACACTTTTGGCCAGGGGACCAAGCTGGAGAT
CAAA
LCVR Amino Acid Sequence
(SEQ ID NO: 10)
EIVLTQSPGTLSLSPGERATLSCRTSQSISSGYLAWYQQKPGQAPRLLIYGASRRATGIPDRFSGSGSGTDFTLTISRLE
PEDFVVYYCQQYGSSPYTFGQGTKLEIK
LCDR1 Nucleotide Sequence
(SEQ ID NO: 11)
CAG AGT ATT AGC AGC GGC TAT
LCDR1 Amino Acid Sequence
(SEQ ID NO: 12)
Q S I S S G Y
LCDR2 Nucleotide Sequence
(SEQ ID NO: 13)
GGT GCA TCC
LCDR2 Amino Acid Sequence
(SEQ ID NO: 14)
G A S
LCDR3 Nucleotide Sequence
(SEQ ID NO: 15)
CAA CAA TAT GGT AGC TCA CCA TAC ACT
LCDR3 Amino Acid Sequence
(SEQ ID NO: 16)
Q Q Y G S S P Y T
Heavy Chain Nucleotide Sequence
(SEQ ID NO: 17)
CAGGTGCAGCTGCAGGAGTCGGGCCCAGGACTGGTGAAGCCTTCGGAGACCCTGTCCCTCACCTGCACTGTCTCTGGTGA
CTCCATTAATAGTTACTTCTGGAGCTGGATCCGGCAGTTGCCAGGGAAGGAACTGGAGTGGATTGGCCATATCTATTCTA
GTGGGAGTACCAGATACAACCCCTCCCTCCAGAGTCGAGTCACCATATCAATAGACACGTCCAAGAACCAGTTCTCCCTG
AAGCTGAGTTCTGTGACCGCTGCGGACACGGCCGTATATTACTGTGCGAGGGGCGCCAGCGCAGTTGACTACTGGGGCCA
GGGAACCCTGGTCACCGTCTCCTCAGCCTCCACCAAGGGCCCATCGGTCTTCCCCCTGGCGCCCTGCTCCAGGAGCACCT
CCGAGAGCACAGCCGCCCTGGGCTGCCTGGTCAAGGACTACTTCCCCGAACCGGTGACGGTGTCGTGGAACTCAGGCGCC
CTGACCAGCGGCGTGCACACCTTCCCGGCTGTCCTACAGTCCTCAGGACTCTACTCCCTCAGCAGCGTGGTGACCGTGCC
CTCCAGCAGCTTGGGCACGAAGACCTACACCTGCAACGTAGATCACAAGCCCAGCAACACCAAGGTGGACAAGAGAGTTG
AGTCCAAATATGGTCCCCCATGCCCACCCTGCCCAGCACCTGAGTTCCTGGGGGGACCATCAGTCTTCCTGTTCCCCCCA
AAACCCAAGGACACTCTCATGATCTCCCGGACCCCTGAGGTCACGTGCGTGGTGGTGGACGTGAGCCAGGAAGACCCCGA
GGTCCAGTTCAACTGGTACGTGGATGGCGTGGAGGTGCATAATGCCAAGACAAAGCCGCGGGAGGAGCAGTTCAACAGCA
CGTACCGTGTGGTCAGCGTCCTCACCGTCCTGCACCAGGACTGGCTGAACGGCAAGGAGTACAAGTGCAAGGTCTCCAAC
AAAGGCCTCCCGTCCTCCATCGAGAAAACCATCTCCAAAGCCAAAGGGCAGCCCCGAGAGCCACAGGTGTACACCCTGCC
CCCATCCCAGGAGGAGATGACCAAGAACCAGGTCAGCCTGACCTGCCTGGTCAAAGGCTTCTACCCCAGCGACATCGCCG
TGGAGTGGGAGAGCAATGGGCAGCCGGAGAACAACTACAAGACCACGCCTCCCGTGCTGGACTCCGACGGCTCCTTCTTC
CTCTACAGCAGGCTCACCGTGGACAAGAGCAGGTGGCAGGAGGGGAATGTCTTCTCATGCTCCGTGATGCATGAGGCTCT
GCACAACCACTACACACAGAAGTCCCTCTCCCTGTCTCTGGGTAAATGA
Heavy Chain Amino Acid Sequence
(SEQ ID NO: 18)
QVQLQESGPGLVKPSETLSLTCTVSGDSINSYFWSWIRQLPGKELEWIGHIYSSGSTRYNPSLQSRVTISIDTSKNQFSL
KLSSVTAADTAVYYCARGASAVDYWGQGTLVTVSSASTKGPSVFPLAPCSRSTSESTAALGCLVKDYFPEPVTVSWNSGA
LTSGVHTFPAVLQSSGLYSLSSVVTVPSSSLGTKTYTCNVDHKPSNTKVDKRVESKYGPPCPPCPAPEFLGGPSVFLFPP
KPKDTLMISRTPEVTCVVVDVSQEDPEVQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEWESNGQPENNYKTTPPVLDSDGSFF
LYSRLTVDKSRWQEGNVFSCSVMHEALHNHYTQKSLSLSLGK
Light Chain Nucleotide Sequence
(SEQ ID NO: 19)
GAAATTGTGTTGACGCAGTCTCCAGGCACCCTGTCTTTGTCTCCAGGGGAAAGAGCCACCCTCTCCTGCAGGACCAGTCA
GAGTATTAGCAGCGGCTATTTAGCCTGGTACCAGCAGAAACCTGGCCAGGCTCCCAGGCTCCTCATCTATGGTGCATCCA
GAAGGGCCACTGGCATCCCAGACAGGTTCAGTGGCAGTGGGTCTGGGACAGACTTCACTCTCACCATCAGCAGACTGGAG
CCTGAAGACTTTGTAGTGTATTACTGTCAACAATATGGTAGCTCACCATACACTTTTGGCCAGGGGACCAAGCTGGAGAT
CAAACGAACTGTGGCTGCACCATCTGTCTTCATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTGACGCTGAGCAAAGCAGACTA
CGAGAAACACAAAGTCTACGCCTGCGAAGTCACCCATCAGGGCCTGAGCTCGCCCGTCACAAAGAGCTTCAACAGGGGAG
AGTGTTAG
Light Chain Amino Acid Sequence
(SEQ ID NO: 20)
EIVLTQSPGTLSLSPGERATLSCRTSQSISSGYLAWYQQKPGQAPRLLIYGASRRATGIPDRFSGSGSGTDFTLTISRLE
PEDFVVYYCQQYGSSPYTFGQGTKLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
H4H30066P
HCVR Nucleotide Sequence
(SEQ ID NO: 21)
CAGGTGCAGCTGGTACAGTCTGGGGCTGAGGTGAAGAAGCCTGGGGCCTCAGTGAAGGTCTCCTGCAAGGTTTCCGGATA
CACCCTCACTGAATTATCCATGCACTGGGTGCGACAAGCTCCTGGAAAAGGGCTTGAGTGGATGGGAGGTTTTGATCCTG
AAGATGGTGAAATAATCTACGCACAGAAGTTCCAGGGCAGAGTCACCATGACCGAGGACACATCTACAGACACAGCCTAC
ATGGACCTGAGCAGTCTGACATCTGAAGACACGGCCGTGTATTACTGTGCAACGGAGAAGCAGCAACTGGTACGAAAATA
CTACTTCTACTACGGTTTGGCCGTCTGGGGCCAAGGGACCACGGTCACCGTCTCCTCA
HCVR Amino Acid Sequence
(SEQ ID NO: 22)
QVQLVQSGAEVKKPGASVKVSCKVSGYTLTELSMHWVRQAPGKGLEWMGGFDPEDGEIIYAQKFQGRVTMTEDTSTDTAY
MDLSSLTSEDTAVYYCATEKQQLVRKYYFYYGLAVWGQGTTVTVSS
HCDR1 Nucleotide Sequence
(SEQ ID NO: 23)
GGA TAC ACC CTC ACT GAA TTA TCC
HCDR1 Amino Acid Sequence
(SEQ ID NO: 24)
G Y T L T E L S
HCDR2 Nucleotide Sequence
(SEQ ID NO: 25)
TTT GAT CCT GAA GAT GGT GAA ATA
HCDR2 Amino Acid Sequence
(SEQ ID NO: 26)
F D P E D G E I
HCDR3 Nucleotide Sequence
(SEQ ID NO: 27)
GCA ACG GAG AAG CAG CAA CTG GTA CGA AAA TAC TAC TIC TAC TAC GGT TTG GCC GTC
HCDR3 Amino Acid Sequence
(SEQ ID NO: 28)
A T E K Q Q L V R K Y Y F Y Y G L A V
LCVR Nucleotide Sequence
(SEQ ID NO: 29)
GACATCCAGATGACCCAGTCTCCATCCTCCCTGTCTGCATCTGTAGGAGACAGAGTCACCATCACTTGCCGGGCAAGTCA
GAGCATTAGCAGTTATTTAAATTGGTATCAGCAGAAACCAGGGAAAGTCCCTAAGCTCCTGATCTATGCTGCATCCAGTT
TGCAAAGTGGGGTCCCATCAAGGTTCAGTGGCAGTGGATCTGGGACAGATTTCACTCTCACCATCAGCAGTCTGCAACCT
GAAGATTTTGCAACTTACTACTGTCAACAGAGTTACAGTCCCCCATTCACTTTCGGCCCTGGGACCAAAGTGGATATCAA
A
LCVR Amino Acid Sequence
(SEQ ID NO: 30)
DIQMTQSPSSLSASVGDRVTITCRASQSISSYLNWYQQKPGKVPKLLIYAASSLQSGVPSRFSGSGSGTDFTLTISSLQP
EDFATYYCQQSYSPPFTFGPGTKVDIK
LCDR1 Nucleotide Sequence
(SEQ ID NO: 31)
CAG AGC ATT AGC AGT TAT
LCDR1 Amino Acid Sequence
(SEQ ID NO: 32)
Q S I S S Y
LCDR2 Nucleotide Sequence
(SEQ ID NO: 33)
GCT GCA TCC
LCDR2 Amino Acid Sequence
(SEQ ID NO: 34)
A A S
LCDR3 Nucleotide Sequence
(SEQ ID NO: 35)
CAA CAG AGT TAC AGT CCC CCA TTC ACT
LCDR3 Amino Acid Sequence
(SEQ ID NO: 36)
Q Q S Y S P P F T
Heavy Chain Nucleotide Sequence
(SEQ ID NO: 37)
CAGGTGCAGCTGGTACAGTCTGGGGCTGAGGTGAAGAAGCCTGGGGCCTCAGTGAAGGTCTCCTGCAAGGTTTCCGGATA
CACCCTCACTGAATTATCCATGCACTGGGTGCGACAAGCTCCTGGAAAAGGGCTTGAGTGGATGGGAGGTTTTGATCCTG
AAGATGGTGAAATAATCTACGCACAGAAGTTCCAGGGCAGAGTCACCATGACCGAGGACACATCTACAGACACAGCCTAC
ATGGACCTGAGCAGTCTGACATCTGAAGACACGGCCGTGTATTACTGTGCAACGGAGAAGCAGCAACTGGTACGAAAATA
CTACTTCTACTACGGTTTGGCCGTCTGGGGCCAAGGGACCACGGTCACCGTCTCCTCAGCCTCCACCAAGGGCCCATCGG
TCTTCCCCCTGGCGCCCTGCTCCAGGAGCACCTCCGAGAGCACAGCCGCCCTGGGCTGCCTGGTCAAGGACTACTTCCCC
GAACCGGTGACGGTGTCGTGGAACTCAGGCGCCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGCACGAAGACCTACACCTGCAACGTAGATCACA
AGCCCAGCAACACCAAGGTGGACAAGAGAGTTGAGTCCAAATATGGTCCCCCATGCCCACCCTGCCCAGCACCTGAGTTC
CTGGGGGGACCATCAGTCTTCCTGTTCCCCCCAAAACCCAAGGACACTCTCATGATCTCCCGGACCCCTGAGGTCACGTG
CGTGGTGGTGGACGTGAGCCAGGAAGACCCCGAGGTCCAGTTCAACTGGTACGTGGATGGCGTGGAGGTGCATAATGCCA
AGACAAAGCCGCGGGAGGAGCAGTTCAACAGCACGTACCGTGTGGTCAGCGTCCTCACCGTCCTGCACCAGGACTGGCTG
AACGGCAAGGAGTACAAGTGCAAGGTCTCCAACAAAGGCCTCCCGTCCTCCATCGAGAAAACCATCTCCAAAGCCAAAGG
GCAGCCCCGAGAGCCACAGGTGTACACCCTGCCCCCATCCCAGGAGGAGATGACCAAGAACCAGGTCAGCCTGACCTGCC
TGGTCAAAGGCTTCTACCCCAGCGACATCGCCGTGGAGTGGGAGAGCAATGGGCAGCCGGAGAACAACTACAAGACCACG
CCTCCCGTGCTGGACTCCGACGGCTCCTTCTTCCTCTACAGCAGGCTCACCGTGGACAAGAGCAGGTGGCAGGAGGGGAA
TGTCTTCTCATGCTCCGTGATGCATGAGGCTCTGCACAACCACTACACACAGAAGTCCCTCTCCCTGTCTCTGGGTAAAT
GA
Heavy Chain Amino Acid
(SEQ ID NO: 38)
QVQLVQSGAEVKKPGASVKVSCKVSGYTLTELSMHWVRQAPGKGLEWMGGFDPEDGEIIYAQKFQGRVTMTEDTSTDTAY
MDLSSLTSEDTAVYYCATEKQQLVRKYYFYYGLAVWGQGTTVTVSSASTKGPSVFPLAPCSRSTSESTAALGCLVKDYFP
EPVTVSWNSGALTSGVHTFPAVLQSSGLYSLSSVVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSLSLGK
Light Chain Nucleotide Sequence
(SEQ ID NO: 39)
GACATCCAGATGACCCAGTCTCCATCCTCCCTGTCTGCATCTGTAGGAGACAGAGTCACCATCACTTGCCGGGCAAGTCA
GAGCATTAGCAGTTATTTAAATTGGTATCAGCAGAAACCAGGGAAAGTCCCTAAGCTCCTGATCTATGCTGCATCCAGTT
TGCAAAGTGGGGTCCCATCAAGGTTCAGTGGCAGTGGATCTGGGACAGATTTCACTCTCACCATCAGCAGTCTGCAACCT
GAAGATTTTGCAACTTACTACTGTCAACAGAGTTACAGTCCCCCATTCACTTTCGGCCCTGGGACCAAAGTGGATATCAA
ACGAACTGTGGCTGCACCATCTGTCTTCATCTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTGGATAACGCCCTCCAATCGGGTAACTCCCAG
GAGAGTGTCACAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAACAGGGGAGAGT
GTTAG
Light Chain Amino Acid
(SEQ ID NO: 40)
DIQMTQSPSSLSASVGDRVTITCRASQSISSYLNWYQQKPGKVPKLLIYAASSLQSGVPSRFSGSGSGTDFTLTISSLQP
EDFATYYCQQSYSPPFTFGPGTKVDIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
H4H30071P
HCVR Nucleotide Sequence
(SEQ ID NO: 41)
CAGGTACAGCTGCAGCAGTCAGGTCCAGGACTGGTGAAGCCCTCGCAGACCCTCTCACTCACCTGTGCCATCTCCGGGGA
CAGTGTCTCTAGGAACAGTGCTGCTTGGAACTGGATCAGGCAGTCCCCATCGAGTGGCCTTGAGTGGCTGGGAAGGACAT
ACTACAGGTCCAAGTGGTTTTATGATTATGCAATATCTGTGAAAAGTCGAATAACCGTCAACCCAGACACATCCAAGAAC
CAATTCTCCCTTCACCTGAACTCTGTGACTCCCGAAGACACGGCTGTCTATTACTGTGCGAGAGGCTACGGTGGCTACGA
GGACTACTTTGACTACTGGGGCCAGGGAACCCTGGTCACCGTCTCCTCA
HCVR Amino Acid Sequence
(SEQ ID NO: 42)
QVQLQQSGPGLVKPSQTLSLTCAISGDSVSRNSAAWNWIRQSPSSGLEWLGRTYYRSKWFYDYAISVKSRITVNPDTSKN
QFSLHLNSVTPEDTAVYYCARGYGGYEDYFDYWGQGTLVTVSS
HCDR1 Nucleotide Sequence
(SEQ ID NO: 43)
GGG GAC AGT GTC TCT AGG AAC AGT GCT GCT
HCDR1 Amino Acid Sequence
(SEQ ID NO: 44)
G D S V S R N S A A
HCDR2 Nucleotide Sequence
(SEQ ID NO: 45)
ACA TAC TAC AGG TCC AAG TGG TTT TAT
HCDR2 Amino Acid Sequence
(SEQ ID NO: 46)
T Y Y R S K W F Y
HCDR3 Nucleotide Sequence
(SEQ ID NO: 47)
GCG AGA GGC TAC GGT GGC TAC GAG GAC TAC TTT GAC TAC
HCDR3 Amino Acid Sequence
(SEQ ID NO: 48)
A R G Y G G Y E D Y F D Y
LCVR Nucleotide Sequence
(SEQ ID NO: 49)
GACATCCAGATGACCCAGTCTCCATCTTCCGTGTCTGCATCTGTAGGAGACAGAGTCACCATCACTTGTCGGGCGAGTCA
GGGTATTAGCAGCTGGTTAGCCTGGTATCAGCAGAAACCAGGGAAAGCCCCTAAGCTCCTGATCTATGGTGCATCCAGTT
TGCAAAGTGGGGTCCCATCAAGGTTCAGCGGCAGTGGATCTGGGACAGATTTCACTCTCACCATCAGCAGCCTGCAGCCT
GAAGATTTTGCAACTTACTTTTGTCAACAGGGTAGCAGTTTCCCGTACACTTTTGGCCAGGGGACCAAGCTGGAGATCAA
A
LCVR Amino Acid Sequence
(SEQ ID NO: 50)
DIQMTQSPSSVSASVGDRVTITCRASQGISSWLAWYQQKPGKAPKLLIYGASSLQSGVPSRFSGSGSGTDFTLTISSLQP
EDFATYFCQQGSSFPYTFGQGTKLEIK
LCDR1 Nucleotide Sequence
(SEQ ID NO: 51)
CAG GGT ATT AGC AGC TGG
LCDR1 Amino Acid Sequence
(SEQ ID NO: 52)
Q G I S S W
LCDR2 Nucleotide Sequence
(SEQ ID NO: 53)
GGT GCA TCC
LCDR2 Amino Acid Sequence
(SEQ ID NO: 54)
G A S
LCDR3 Nucleotide Sequence
(SEQ ID NO: 55)
CAA CAG GGT AGC AGT TTC CCG TAC ACT
LCDR3 Amino Acid Sequence
(SEQ ID NO: 56)
Q Q G S S F P Y T
Heavy Chain Nucleotide Sequence
(SEQ ID NO: 57)
CAGGTACAGCTGCAGCAGTCAGGTCCAGGACTGGTGAAGCCCTCGCAGACCCTCTCACTCACCTGTGCCATCTCCGGGGA
CAGTGTCTCTAGGAACAGTGCTGCTTGGAACTGGATCAGGCAGTCCCCATCGAGTGGCCTTGAGTGGCTGGGAAGGACAT
ACTACAGGTCCAAGTGGTTTTATGATTATGCAATATCTGTGAAAAGTCGAATAACCGTCAACCCAGACACATCCAAGAAC
CAATTCTCCCTTCACCTGAACTCTGTGACTCCCGAAGACACGGCTGTCTATTACTGTGCGAGAGGCTACGGTGGCTACGA
GGACTACTTTGACTACTGGGGCCAGGGAACCCTGGTCACCGTCTCCTCAGCCTCCACCAAGGGCCCATCGGTCTTCCCCC
TGGCGCCCTGCTCCAGGAGCACCTCCGAGAGCACAGCCGCCCTGGGCTGCCTGGTCAAGGACTACTTCCCCGAACCGGTG
ACGGTGTCGTGGAACTCAGGCGCCCTGACCAGCGGCGTGCACACCTTCCCGGCTGTCCTACAGTCCTCAGGACTCTACTC
CCTCAGCAGCGTGGTGACCGTGCCCTCCAGCAGCTTGGGCACGAAGACCTACACCTGCAACGTAGATCACAAGCCCAGCA
ACACCAAGGTGGACAAGAGAGTTGAGTCCAAATATGGTCCCCCATGCCCACCCTGCCCAGCACCTGAGTTCCTGGGGGGA
CCATCAGTCTTCCTGTTCCCCCCAAAACCCAAGGACACTCTCATGATCTCCCGGACCCCTGAGGTCACGTGCGTGGTGGT
GGACGTGAGCCAGGAAGACCCCGAGGTCCAGTTCAACTGGTACGTGGATGGCGTGGAGGTGCATAATGCCAAGACAAAGC
CGCGGGAGGAGCAGTTCAACAGCACGTACCGTGTGGTCAGCGTCCTCACCGTCCTGCACCAGGACTGGCTGAACGGCAAG
GAGTACAAGTGCAAGGTCTCCAACAAAGGCCTCCCGTCCTCCATCGAGAAAACCATCTCCAAAGCCAAAGGGCAGCCCCG
AGAGCCACAGGTGTACACCCTGCCCCCATCCCAGGAGGAGATGACCAAGAACCAGGTCAGCCTGACCTGCCTGGTCAAAG
GCTTCTACCCCAGCGACATCGCCGTGGAGTGGGAGAGCAATGGGCAGCCGGAGAACAACTACAAGACCACGCCTCCCGTG
CTGGACTCCGACGGCTCCTTCTTCCTCTACAGCAGGCTCACCGTGGACAAGAGCAGGTGGCAGGAGGGGAATGTCTTCTC
ATGCTCCGTGATGCATGAGGCTCTGCACAACCACTACACACAGAAGTCCCTCTCCCTGTCTCTGGGTAAATGA
Heavy Chain Amino Acid
(SEQ ID NO: 58)
QVQLQQSGPGLVKPSQTLSLTCAISGDSVSRNSAAWNWIRQSPSSGLEWLGRTYYRSKWFYDYAISVKSRITVNPDTSKN
QFSLHLNSVTPEDTAVYYCARGYGGYEDYFDYWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDHKPSNTKVDKRVESKYGPPCPPCPAPEFLGG
PSVFLFPPKPKDTLMISRTPEVTCVVVDVSQEDPEVQFNWYVDGVEVHNAKTKPREEQFNSTYRVVSVLTVLHQDWLNGK
EYKCKVSNKGLPSSIEKTISKAKGQPREPQVYTLPPSQEEMTKNQVSLTCLVKGFYPSDIAVEWESNGQPENNYKTTPPV
LDSDGSFFLYSRLTVDKSRWQEGNVFSCSVMHEALHNHYTQKSLSLSLGK
Light Chain Nucleotide Sequence
(SEQ ID NO: 59)
GACATCCAGATGACCCAGTCTCCATCTTCCGTGTCTGCATCTGTAGGAGACAGAGTCACCATCACTTGTCGGGCGAGTCA
GGGTATTAGCAGCTGGTTAGCCTGGTATCAGCAGAAACCAGGGAAAGCCCCTAAGCTCCTGATCTATGGTGCATCCAGTT
TGCAAAGTGGGGTCCCATCAAGGTTCAGCGGCAGTGGATCTGGGACAGATTTCACTCTCACCATCAGCAGCCTGCAGCCT
GAAGATTTTGCAACTTACTTTTGTCAACAGGGTAGCAGTTTCCCGTACACTTTTGGCCAGGGGACCAAGCTGGAGATCAA
ACGAACTGTGGCTGCACCATCTGTCTTCATCTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTGGATAACGCCCTCCAATCGGGTAACTCCCAG
GAGAGTGTCACAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAACAGGGGAGAGT
GTTAG
Light Chain Amino Acid
(SEQ ID NO: 60)
DIQMTQSPSSVSASVGDRVTITCRASQGISSWLAWYQQKPGKAPKLLIYGASSLQSGVPSRFSGSGSGTDFTLTISSLQP
EDFATYFCQQGSSFPYTFGQGTKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
H4H30089P2
HCVR Nucleotide Sequence
(SEQ ID NO: 61)
GAGGTGCAGCTGGTGGAGTCTGGGGGAGGCTTGGTACAGCCTGGAGGGTCCCTGAGACTCTCCTGTATAGTCTCTGGATT
CATCTTCAGTAGTTATGAAATGAGCTGGCTCCGCCAGGCTCCAGGGAAGGGCCTGGAGTGGATTTCATATATTAGTAGTA
GTGGTCGTGTCATATACTATGCAGACTCTGTGAAGGGCCGATTCACCATCTCCAGAGACAGCGCCAAGAATTCACTGTAT
CTGGAAATGAATAGTCTGAGAGCCGAAGACACGGCTATATATTATTGTACGAGAAAGTGGGATAGTAGTGGCCCATTTGA
CTTCTGGGGCCAGGGAACCCTGGTCACCGTCTCCTCA
HCVR Amino Acid Sequence
(SEQ ID NO: 62)
EVQLVESGGGLVQPGGSLRLSCIVSGFIFSSYEMSWLRQAPGKGLEWISYISSSGRVIYYADSVKGRFTISRDSAKNSLY
LEMNSLRAEDTAIYYCTRKWDSSGPFDFWGQGTLVTVSS
HCDR1 Nucleotide Sequence
(SEQ ID NO: 63)
GGA TTC ATC TTC AGT AGT TAT GAA
HCDR1 Amino Acid Sequence
(SEQ ID NO: 64)
G F I F S S Y E
HCDR2 Nucleotide Sequence
(SEQ ID NO: 65)
ATT AGT AGT AGT GGT CGT GTC ATA
HCDR2 Amino Acid Sequence
(SEQ ID NO: 66)
I S S S G R V I
HCDR3 Nucleotide Sequence
(SEQ ID NO: 67)
ACG AGA AAG TGG GAT AGT AGT GGC CCA TTT GAC TTC
HCDR3 Amino Acid Sequence
(SEQ ID NO: 68)
T R K W D S S G P F D F
LCVR Nucleotide Sequence
(SEQ ID NO: 69)
GACATCCAGATGACCCAGTCTCCATCCTCCCTGTCTGCATCTGTAGGAGACAGAGTCACCATCACTTGCCGGGCAAGTCA
GAGCATTAGCAGCTATTTAAATTGGTATCAGCAGAAACCAGGGAAAGCCCCTAAGCTCCTGATCTATGCTGCATCCAGTT
TGCAAAGTGGGGTCCCGTCAAGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGCCAAGGGACACGACTGGAGAT
TAAA
LCVR Amino Acid Sequence
(SEQ ID NO: 70)
DIQMTQSPSSLSASVGDRVTITCRASQSISSYLNWYQQKPGKAPKLLIYAASSLQSGVPSRFSGSGSGTDFTLTISSLQP
EDFATYYCQQSYSTPPITFGQGTRLEIK
LCDR1 Nucleotide Sequence
(SEQ ID NO: 71)
CAG AGC ATT AGC AGC TAT
LCDR1 Amino Acid Sequence
(SEQ ID NO: 72)
Q S I S S Y
LCDR2 Nucleotide Sequence
(SEQ ID NO: 73)
GCT GCA TCC
LCDR2 Amino Acid Sequence
(SEQ ID NO: 74)
A A S
LCDR3 Nucleotide Sequence
(SEQ ID NO: 75)
CAA CAG AGT TAC AGT ACC CCT CCG ATC ACC
LCDR3 Amino Acid Sequence
(SEQ ID NO: 76)
Q Q S Y S T P P I T
Heavy Chain Nucleotide Sequence
(SEQ ID NO: 77)
GAGGTGCAGCTGGTGGAGTCTGGGGGAGGCTTGGTACAGCCTGGAGGGTCCCTGAGACTCTCCTGTATAGTCTCTGGATT
CATCTTCAGTAGTTATGAAATGAGCTGGCTCCGCCAGGCTCCAGGGAAGGGCCTGGAGTGGATTTCATATATTAGTAGTA
GTGGTCGTGTCATATACTATGCAGACTCTGTGAAGGGCCGATTCACCATCTCCAGAGACAGCGCCAAGAATTCACTGTAT
CTGGAAATGAATAGTCTGAGAGCCGAAGACACGGCTATATATTATTGTACGAGAAAGTGGGATAGTAGTGGCCCATTTGA
CTTCTGGGGCCAGGGAACCCTGGTCACCGTCTCCTCAGCCTCCACCAAGGGCCCATCGGTCTTCCCCCTGGCGCCCTGCT
CCAGGAGCACCTCCGAGAGCACAGCCGCCCTGGGCTGCCTGGTCAAGGACTACTTCCCCGAACCGGTGACGGTGTCGTGG
AACTCAGGCGCCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACGAAGACCTACACCTGCAACGTAGATCACAAGCCCAGCAACACCAAGGTGG
ACAAGAGAGTTGAGTCCAAATATGGTCCCCCATGCCCACCCTGCCCAGCACCTGAGTTCCTGGGGGGACCATCAGTCTTC
CTGTTCCCCCCAAAACCCAAGGACACTCTCATGATCTCCCGGACCCCTGAGGTCACGTGCGTGGTGGTGGACGTGAGCCA
GGAAGACCCCGAGGTCCAGTTCAACTGGTACGTGGATGGCGTGGAGGTGCATAATGCCAAGACAAAGCCGCGGGAGGAGC
AGTTCAACAGCACGTACCGTGTGGTCAGCGTCCTCACCGTCCTGCACCAGGACTGGCTGAACGGCAAGGAGTACAAGTGC
AAGGTCTCCAACAAAGGCCTCCCGTCCTCCATCGAGAAAACCATCTCCAAAGCCAAAGGGCAGCCCCGAGAGCCACAGGT
GTACACCCTGCCCCCATCCCAGGAGGAGATGACCAAGAACCAGGTCAGCCTGACCTGCCTGGTCAAAGGCTTCTACCCCA
GCGACATCGCCGTGGAGTGGGAGAGCAATGGGCAGCCGGAGAACAACTACAAGACCACGCCTCCCGTGCTGGACTCCGAC
GGCTCCTTCTTCCTCTACAGCAGGCTCACCGTGGACAAGAGCAGGTGGCAGGAGGGGAATGTCTTCTCATGCTCCGTGAT
GCATGAGGCTCTGCACAACCACTACACACAGAAGTCCCTCTCCCTGTCTCTGGGTAAATGA
Heavy Chain Amino Acid
(SEQ ID NO: 78)
EVQLVESGGGLVQPGGSLRLSCIVSGFIFSSYEMSWLRQAPGKGLEWISYISSSGRVIYYADSVKGRFTISRDSAKNSLY
LEMNSLRAEDTAIYYCTRKWDSSGPFDFWGQGTLVTVSSASTKGPSVFPLAPCSRSTSESTAALGCLVKDYFPEPVTVSW
NSGALTSGVHTFPAVLQSSGLYSLSSVVTVPSSSLGTKTYTCNVDHKPSNTKVDKRVESKYGPPCPPCPAPEFLGGPSVF
LFPPKPKDTLMISRTPEVTCVVVDVSQEDPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK
Light Chain Nucleotide Sequence
(SEQ ID NO: 79)
GACATCCAGATGACCCAGTCTCCATCCTCCCTGTCTGCATCTGTAGGAGACAGAGTCACCATCACTTGCCGGGCAAGTCA
GAGCATTAGCAGCTATTTAAATTGGTATCAGCAGAAACCAGGGAAAGCCCCTAAGCTCCTGATCTATGCTGCATCCAGTT
TGCAAAGTGGGGTCCCGTCAAGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGCCAAGGGACACGACTGGAGAT
TAAACGAACTGTGGCTGCACCATCTGTCTTCATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTGACGCTGAGCAAAGCAGACTA
CGAGAAACACAAAGTCTACGCCTGCGAAGTCACCCATCAGGGCCTGAGCTCGCCCGTCACAAAGAGCTTCAACAGGGGAG
AGTGTTAG
Light Chain Amino Acid
(SEQ ID NO: 80)
DIQMTQSPSSLSASVGDRVTITCRASQSISSYLNWYQQKPGKAPKLLIYAASSLQSGVPSRFSGSGSGTDFTLTISSLQP
EDFATYYCQQSYSTPPITFGQGTRLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
H4H30093P2
HCVR Nucleotide Sequence
(SEQ ID NO: 81)
CAGGTGCAGCTGGTGGAGTCTGGGGGAGGCTTGGTCAAGCCTGGAGGGTCCCTAAGACTCTCCTGTGCAGCCTCTGGATT
CACCTTCAATGACTACCCAATGAGCTGGATCCGCCAGGCTCCAGGGAAGGGACTGGAGTGGGTTTCATACATTACTAGCA
GTAGTGGTAGTACCATATACTACGCAGACTCTGTGAAGGGCCGATTCACCATCTCCAGGGACAACGCCAAGAACTCACTG
TATCTGCAAATGAACAGCCTGAGAGCCGAGGACACGGCCGTGTATTACTGTGCGAGAGAGGTTGTAGTGGCTACGATTGG
GGGCTACTACGGTATGGACGTCTGGGGCCAAGGGACCACGGTCACCGTCTCCTCA
HCVR Amino Acid Sequence
(SEQ ID NO: 82)
QVQLVESGGGLVKPGGSLRLSCAASGFTFNDYPMSWIRQAPGKGLEWVSYITSSSGSTIYYADSVKGRFTISRDNAKNSL
YLQMNSLRAEDTAVYYCAREVVVATIGGYYGMDVWGQGTTVTVSS
HCDR1 Nucleotide Sequence
(SEQ ID NO: 83)
GGA TTC ACC TTC AAT GAC TAC CCA
HCDR1 Amino Acid Sequence
(SEQ ID NO: 84)
G F T F N D Y P
HCDR2 Nucleotide Sequence
(SEQ ID NO: 85)
ATT ACT AGC AGT AGT GGT AGT ACC ATA
HCDR2 Amino Acid Sequence
(SEQ ID NO: 86)
I T S S S G S T I
HCDR3 Nucleotide Sequence
(SEQ ID NO: 87)
GCG AGA GAG GTT GTA GTG GCT ACG ATT GGG GGC TAC TAC GGT ATG GAC GTC
HCDR3 Amino Acid Sequence
(SEQ ID NO: 88)
A R E V V V A T I G G Y Y G M D V
LCVR Nucleotide Sequence
(SEQ ID NO: 89)
GACATCCAGATGACCCAGTCTCCATCCTCCCTGTCTGCATCTGTAGGAGACAGAGTCACCATCACTTGCCGGGCAAGTCA
GAGCATTAGCAGCTATTTAAATTGGTATCAGCAGAAACCAGGGAAAGCCCCTAAGCTCCTGATCTATGCTGCATCCAGTT
TGCAAAGTGGGGTCCCGTCAAGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGCCAAGGGACACGACTGGAGAT
TAAA
LCVR Amino Acid Sequence
(SEQ ID NO: 90)
DIQMTQSPSSLSASVGDRVTITCRASQSISSYLNWYQQKPGKAPKLLIYAASSLQSGVPSRFSGSGSGTDFTLTISSLQP
EDFATYYCQQSYSTPPITFGQGTRLEIK
LCDR1 Nucleotide Sequence
(SEQ ID NO: 91)
CAG AGC ATT AGC AGC TAT
LCDR1 Amino Acid Sequence
(SEQ ID NO: 92)
Q S I S S Y
LCDR2 Nucleotide Sequence
(SEQ ID NO: 93)
GCT GCA TCC
LCDR2 Amino Acid Sequence
(SEQ ID NO: 94)
A A S
LCDR3 Nucleotide Sequence
(SEQ ID NO: 95)
CAA CAG AGT TAC AGT ACC CCT CCG ATC ACC
LCDR3 Amino Acid Sequence
(SEQ ID NO: 96)
Q Q S Y S T P P I T
Heavy Chain Nucleotide Sequence
(SEQ ID NO: 97)
CAGGTGCAGCTGGTGGAGTCTGGGGGAGGCTTGGTCAAGCCTGGAGGGTCCCTAAGACTCTCCTGTGCAGCCTCTGGATT
CACCTTCAATGACTACCCAATGAGCTGGATCCGCCAGGCTCCAGGGAAGGGACTGGAGTGGGTTTCATACATTACTAGCA
GTAGTGGTAGTACCATATACTACGCAGACTCTGTGAAGGGCCGATTCACCATCTCCAGGGACAACGCCAAGAACTCACTG
TATCTGCAAATGAACAGCCTGAGAGCCGAGGACACGGCCGTGTATTACTGTGCGAGAGAGGTTGTAGTGGCTACGATTGG
GGGCTACTACGGTATGGACGTCTGGGGCCAAGGGACCACGGTCACCGTCTCCTCAGCCTCCACCAAGGGCCCATCGGTCT
TCCCCCTGGCGCCCTGCTCCAGGAGCACCTCCGAGAGCACAGCCGCCCTGGGCTGCCTGGTCAAGGACTACTTCCCCGAA
CCGGTGACGGTGTCGTGGAACTCAGGCGCCCTGACCAGCGGCGTGCACACCTTCCCGGCTGTCCTACAGTCCTCAGGACT
CTACTCCCTCAGCAGCGTGGTGACCGTGCCCTCCAGCAGCTTGGGCACGAAGACCTACACCTGCAACGTAGATCACAAGC
CCAGCAACACCAAGGTGGACAAGAGAGTTGAGTCCAAATATGGTCCCCCATGCCCACCCTGCCCAGCACCTGAGTTCCTG
GGGGGACCATCAGTCTTCCTGTTCCCCCCAAAACCCAAGGACACTCTCATGATCTCCCGGACCCCTGAGGTCACGTGCGT
GGTGGTGGACGTGAGCCAGGAAGACCCCGAGGTCCAGTTCAACTGGTACGTGGATGGCGTGGAGGTGCATAATGCCAAGA
CAAAGCCGCGGGAGGAGCAGTTCAACAGCACGTACCGTGTGGTCAGCGTCCTCACCGTCCTGCACCAGGACTGGCTGAAC
GGCAAGGAGTACAAGTGCAAGGTCTCCAACAAAGGCCTCCCGTCCTCCATCGAGAAAACCATCTCCAAAGCCAAAGGGCA
GCCCCGAGAGCCACAGGTGTACACCCTGCCCCCATCCCAGGAGGAGATGACCAAGAACCAGGTCAGCCTGACCTGCCTGG
TCAAAGGCTTCTACCCCAGCGACATCGCCGTGGAGTGGGAGAGCAATGGGCAGCCGGAGAACAACTACAAGACCACGCCT
CCCGTGCTGGACTCCGACGGCTCCTTCTTCCTCTACAGCAGGCTCACCGTGGACAAGAGCAGGTGGCAGGAGGGGAATGT
CTTCTCATGCTCCGTGATGCATGAGGCTCTGCACAACCACTACACACAGAAGTCCCTCTCCCTGTCTCTGGGTAAATGA
Heavy Chain Amino Acid
(SEQ ID NO: 98)
QVQLVESGGGLVKPGGSLRLSCAASGFTFNDYPMSWIRQAPGKGLEWVSYITSSSGSTIYYADSVKGRFTISRDNAKNSL
YLQMNSLRAEDTAVYYCAREVVVATIGGYYGMDVWGQGTTVTVSSASTKGPSVFPLAPCSRSTSESTAALGCLVKDYFPE
PVTVSWNSGALTSGVHTFPAVLQSSGLYSLSSVVTVPSSSLGTKTYTCNVDHKPSNTKVDKRVESKYGPPCPPCPAPEFL
GGPSVFLFPPKPKDTLMISRTPEVTCVVVDVSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSLGK
Light Chain Nucleotide Sequence
(SEQ ID NO: 99)
GACATCCAGATGACCCAGTCTCCATCCTCCCTGTCTGCATCTGTAGGAGACAGAGTCACCATCACTTGCCGGGCAAGTCA
GAGCATTAGCAGCTATTTAAATTGGTATCAGCAGAAACCAGGGAAAGCCCCTAAGCTCCTGATCTATGCTGCATCCAGTT
TGCAAAGTGGGGTCCCGTCAAGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGCCAAGGGACACGACTGGAGAT
TAAACGAACTGTGGCTGCACCATCTGTCTTCATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTGACGCTGAGCAAAGCAGACTA
CGAGAAACACAAAGTCTACGCCTGCGAAGTCACCCATCAGGGCCTGAGCTCGCCCGTCACAAAGAGCTTCAACAGGGGAG
AGTGTTAG
Light Chain Amino Acid
(SEQ ID NO: 100)
DIQMTQSPSSLSASVGDRVTITCRASQSISSYLNWYQQKPGKAPKLLIYAASSLQSGVPSRFSGSGSGTDFTLTISSLQP
EDFATYYCQQSYSTPPITFGQGTRLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
H4H30102P2
HCVR Nucleotide Sequence
(SEQ ID NO: 101)
GAGGTGCAGCTGGTGGAGTCTGGGGGAGACTTGGTACAGCCTGGAGGGTCCCTGAGACTCTCCTGTGCAGCCTCTGGATT
CACCTTCAGTAGTTATGAAATGAACTGGGTCCGCCAGGCTCCAGGGAAGGGGCTGGAGTGGGTTTCATACATTAGTAATA
GTGGTTCTACCATATACTACGCAGACTCTGTGAAGGGCCGATTCACCATCTCCAGAGACAACGCCGAGACCTCACTGTAT
CTGCAAATGAACAGCCTGAGAGCCGAGGACACGGCTGTTTATTACTGTGCGAGAGAGGGATGGGGCGCATATTGTGCTGG
TGATTGCTATTCTGGTTTTGATATTTGGGGCCAAGGGACAATGGTCACCGTCTCTTCA
HCVR Amino Acid Sequence
(SEQ ID NO: 102)
EVQLVESGGDLVQPGGSLRLSCAASGFTFSSYEMNWVRQAPGKGLEWVSYISNSGSTIYYADSVKGRFTISRDNAETSLY
LQMNSLRAEDTAVYYCAREGWGAYCAGDCYSGFDIWGQGTMVTVSS
HCDR1 Nucleotide Sequence
(SEQ ID NO: 103)
GGA TTC ACC TTC AGT AGT TAT GAA
HCDR1 Amino Acid Sequence
(SEQ ID NO: 104)
G F T F S S Y E
HCDR2 Nucleotide Sequence
(SEQ ID NO: 105)
ATT AGT AAT AGT GGT TCT ACC ATA
HCDR2 Amino Acid Sequence
(SEQ ID NO: 106)
I S N S G S T I
HCDR3 Nucleotide Sequence
(SEQ ID NO: 107)
GCG AGA GAG GGA TGG GGC GCA TAT TGT GCT GGT GAT TGC TAT TCT GGT TTT GAT ATT
HCDR3 Amino Acid Sequence
(SEQ ID NO: 108)
A R E G W G A Y C A G D C Y S G F D I
LCVR Nucleotide Sequence
(SEQ ID NO: 109)
GACATCCAGATGACCCAGTCTCCATCCTCCCTGTCTGCATCTGTAGGAGACAGAGTCACCATCACTTGCCGGGCAAGTCA
GAGCATTAGCAGCTATTTAAATTGGTATCAGCAGAAACCAGGGAAAGCCCCTAAGCTCCTGATCTATGCTGCATCCAGTT
TGCAAAGTGGGGTCCCGTCAAGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGCCAAGGGACACGACTGGAGAT
TAAA
LCVR Amino Acid Sequence
(SEQ ID NO: 110)
DIQMTQSPSSLSASVGDRVTITCRASQSISSYLNWYQQKPGKAPKLLIYAASSLQSGVPSRFSGSGSGTDFTLTISSLQP
EDFATYYCQQSYSTPPITFGQGTRLEIK
LCDR1 Nucleotide Sequence
(SEQ ID NO: 111)
CAG AGC ATT AGC AGC TAT
LCDR1 Amino Acid Sequence
(SEQ ID NO: 112)
Q S I S S Y
LCDR2 Nucleotide Sequence
(SEQ ID NO: 113)
GCT GCA TCC
LCDR2 Amino Acid Sequence
(SEQ ID NO: 114)
A A S
LCDR3 Nucleotide Sequence
(SEQ ID NO: 115)
CAA CAG AGT TAC AGT ACC CCT CCG ATC ACC
LCDR3 Amino Acid Sequence
(SEQ ID NO: 116)
Q Q S Y S T P P I T
Heavy Chain Nucleotide Sequence
(SEQ ID NO: 117)
GAGGTGCAGCTGGTGGAGTCTGGGGGAGACTTGGTACAGCCTGGAGGGTCCCTGAGACTCTCCTGTGCAGCCTCTGGATT
CACCTTCAGTAGTTATGAAATGAACTGGGTCCGCCAGGCTCCAGGGAAGGGGCTGGAGTGGGTTTCATACATTAGTAATA
GTGGTTCTACCATATACTACGCAGACTCTGTGAAGGGCCGATTCACCATCTCCAGAGACAACGCCGAGACCTCACTGTAT
CTGCAAATGAACAGCCTGAGAGCCGAGGACACGGCTGTTTATTACTGTGCGAGAGAGGGATGGGGCGCATATTGTGCTGG
TGATTGCTATTCTGGTTTTGATATTTGGGGCCAAGGGACAATGGTCACCGTCTCTTCAGCCTCCACCAAGGGCCCATCGG
TCTTCCCCCTGGCGCCCTGCTCCAGGAGCACCTCCGAGAGCACAGCCGCCCTGGGCTGCCTGGTCAAGGACTACTTCCCC
GAACCGGTGACGGTGTCGTGGAACTCAGGCGCCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGCACGAAGACCTACACCTGCAACGTAGATCACA
AGCCCAGCAACACCAAGGTGGACAAGAGAGTTGAGTCCAAATATGGTCCCCCATGCCCACCCTGCCCAGCACCTGAGTTC
CTGGGGGGACCATCAGTCTTCCTGTTCCCCCCAAAACCCAAGGACACTCTCATGATCTCCCGGACCCCTGAGGTCACGTG
CGTGGTGGTGGACGTGAGCCAGGAAGACCCCGAGGTCCAGTTCAACTGGTACGTGGATGGCGTGGAGGTGCATAATGCCA
AGACAAAGCCGCGGGAGGAGCAGTTCAACAGCACGTACCGTGTGGTCAGCGTCCTCACCGTCCTGCACCAGGACTGGCTG
AACGGCAAGGAGTACAAGTGCAAGGTCTCCAACAAAGGCCTCCCGTCCTCCATCGAGAAAACCATCTCCAAAGCCAAAGG
GCAGCCCCGAGAGCCACAGGTGTACACCCTGCCCCCATCCCAGGAGGAGATGACCAAGAACCAGGTCAGCCTGACCTGCC
TGGTCAAAGGCTTCTACCCCAGCGACATCGCCGTGGAGTGGGAGAGCAATGGGCAGCCGGAGAACAACTACAAGACCACG
CCTCCCGTGCTGGACTCCGACGGCTCCTTCTTCCTCTACAGCAGGCTCACCGTGGACAAGAGCAGGTGGCAGGAGGGGAA
TGTCTTCTCATGCTCCGTGATGCATGAGGCTCTGCACAACCACTACACACAGAAGTCCCTCTCCCTGTCTCTGGGTAAAT
GA
Heavy Chain Amino Acid
(SEQ ID NO: 118)
EVQLVESGGDLVQPGGSLRLSCAASGFTFSSYEMNWVRQAPGKGLEWVSYISNSGSTIYYADSVKGRFTISRDNAETSLY
LQMNSLRAEDTAVYYCAREGWGAYCAGDCYSGFDIWGQGTMVTVSSASTKGPSVFPLAPCSRSTSESTAALGCLVKDYFP
EPVTVSWNSGALTSGVHTFPAVLQSSGLYSLSSVVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSLSLGK
Light Chain Nucleotide Sequence
(SEQ ID NO: 119)
GACATCCAGATGACCCAGTCTCCATCCTCCCTGTCTGCATCTGTAGGAGACAGAGTCACCATCACTTGCCGGGCAAGTCA
GAGCATTAGCAGCTATTTAAATTGGTATCAGCAGAAACCAGGGAAAGCCCCTAAGCTCCTGATCTATGCTGCATCCAGTT
TGCAAAGTGGGGTCCCGTCAAGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGCCAAGGGACACGACTGGAGAT
TAAACGAACTGTGGCTGCACCATCTGTCTTCATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTGACGCTGAGCAAAGCAGACTA
CGAGAAACACAAAGTCTACGCCTGCGAAGTCACCCATCAGGGCCTGAGCTCGCCCGTCACAAAGAGCTTCAACAGGGGAG
AGTGTTAG
Light Chain Amino Acid
(SEQ ID NO: 120)
DIQMTQSPSSLSASVGDRVTITCRASQSISSYLNWYQQKPGKAPKLLIYAASSLQSGVPSRFSGSGSGTDFTLTISSLQP
EDFATYYCQQSYSTPPITFGQGTRLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
H4H30076P
HCVR Nucleotide Sequence
(SEQ ID NO: 121)
GAGGTGCAGCTGGTGGAGTCTGGAGGAGGCTTGGTCCAGCCTGGGGGGTCCCTGAGACTCTCCTGTGCAGCCTCTGGGTT
CACCGTCAGTAGCAACTACATGAGCTGGGTCCGCCAGGCTCCAGGGAAGGGGCTGGAGTGGGTCTCAATTATTTATAGCG
GTGGTCACACATACTACTCAGACTCCGTGAAGGGCCGATTCACCATCTCCAGACACAATTCCAAGAACACTCTGTATCTT
CAAATGAACAGCCTGAGAGGTGGGGACACGGCCGTGTATTACTGTGCGAGAGGGTATACCAGTGGCTGGTACGGATTTGA
CTTCTGGGGCCAGGGAACCCTGGTCACCGTCTCCTCA
HCVR Amino Acid Sequence
(SEQ ID NO: 122)
EVQLVESGGGLVQPGGSLRLSCAASGFTVSSNYMSWVRQAPGKGLEWVSIIYSGGHTYYSDSVKGRFTISRHNSKNTLYL
QMNSLRGGDTAVYYCARGYTSGWYGFDFWGQGTLVTVSS
HCDR1 Nucleotide Sequence
(SEQ ID NO: 123)
GGG TTC ACC GTC AGT AGC AAC TAC
HCDR1 Amino Acid Sequence
(SEQ ID NO: 124)
G F T V S S N Y
HCDR2 Nucleotide Sequence
(SEQ ID NO: 125)
ATT TAT AGC GGT GGT CAC ACA
HCDR2 Amino Acid Sequence
(SEQ ID NO: 126)
I Y S G G H T
HCDR3 Nucleotide Sequence
(SEQ ID NO: 127)
GCG AGA GGG TAT ACC AGT GGC TGG TAC GGA TTT GAC TTC
HCDR3 Amino Acid Sequence
(SEQ ID NO: 128)
A R G Y T S G W Y G F D F
LCVR Nucleotide Sequence
(SEQ ID NO: 129)
GACATCCAGATGACCCAGTCTCCATCTTCCGTGTCTGCATCTGTAGGAGACAGAGTCACCATCACTTGTCGGGCGAGTCA
GGGTATTAGCACCTGGTTAGCCTGGTATCAGCAGAAACCAGGGAAAGCCCCTAAGCTCCTGATCTATGCTGCATCCAGTT
TGCAAAGTGGGGTCCCGTCAAGATTCAGCGGCACTGGATCTGGGACAGATTTCACTCTCACCATCAGCAGCCTGCAGCCT
GAAGATTTTGCAACTTATTATTGTCAGCAGACTAACAGTTTCCCGTGGACGTTCGGCCAAGGGACCAAGGTGGAAATCAA
A
LCVR Amino Acid Sequence
(SEQ ID NO: 130)
DIQMTQSPSSVSASVGDRVTITCRASQGISTWLAWYQQKPGKAPKLLIYAASSLQSGVPSRFSGTGSGTDFTLTISSLQP
EDFATYYCQQTNSFPWTFGQGTKVEIK
LCDR1 Nucleotide Sequence
(SEQ ID NO: 131)
CAG GGT ATT AGC ACC TGG
LCDR1 Amino Acid Sequence
(SEQ ID NO: 132)
Q G I S T W
LCDR2 Nucleotide Sequence
(SEQ ID NO: 33)
GCT GCA TCC
LCDR2 Amino Acid Sequence
(SEQ ID NO: 34)
A A S
LCDR3 Nucleotide Sequence
(SEQ ID NO: 133)
CAG CAG ACT AAC AGT TTC CCG TGG ACG
LCDR3 Amino Acid Sequence
(SEQ ID NO: 134)
Q Q T N S F P W T
Heavy Chain Nucleotide Sequence
(SEQ ID NO: 135)
GAGGTGCAGCTGGTGGAGTCTGGAGGAGGCTTGGTCCAGCCTGGGGGGTCCCTGAGACTCTCCTGTGCAGCCTCTGGGTT
CACCGTCAGTAGCAACTACATGAGCTGGGTCCGCCAGGCTCCAGGGAAGGGGCTGGAGTGGGTCTCAATTATTTATAGCG
GTGGTCACACATACTACTCAGACTCCGTGAAGGGCCGATTCACCATCTCCAGACACAATTCCAAGAACACTCTGTATCTT
CAAATGAACAGCCTGAGAGGTGGGGACACGGCCGTGTATTACTGTGCGAGAGGGTATACCAGTGGCTGGTACGGATTTGA
CTTCTGGGGCCAGGGAACCCTGGTCACCGTCTCCTCAGCCTCCACCAAGGGCCCATCGGTCTTCCCCCTGGCGCCCTGCT
CCAGGAGCACCTCCGAGAGCACAGCCGCCCTGGGCTGCCTGGTCAAGGACTACTTCCCCGAACCGGTGACGGTGTCGTGG
AACTCAGGCGCCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACGAAGACCTACACCTGCAACGTAGATCACAAGCCCAGCAACACCAAGGTGG
ACAAGAGAGTTGAGTCCAAATATGGTCCCCCATGCCCACCCTGCCCAGCACCTGAGTTCCTGGGGGGACCATCAGTCTTC
CTGTTCCCCCCAAAACCCAAGGACACTCTCATGATCTCCCGGACCCCTGAGGTCACGTGCGTGGTGGTGGACGTGAGCCA
GGAAGACCCCGAGGTCCAGTTCAACTGGTACGTGGATGGCGTGGAGGTGCATAATGCCAAGACAAAGCCGCGGGAGGAGC
AGTTCAACAGCACGTACCGTGTGGTCAGCGTCCTCACCGTCCTGCACCAGGACTGGCTGAACGGCAAGGAGTACAAGTGC
AAGGTCTCCAACAAAGGCCTCCCGTCCTCCATCGAGAAAACCATCTCCAAAGCCAAAGGGCAGCCCCGAGAGCCACAGGT
GTACACCCTGCCCCCATCCCAGGAGGAGATGACCAAGAACCAGGTCAGCCTGACCTGCCTGGTCAAAGGCTTCTACCCCA
GCGACATCGCCGTGGAGTGGGAGAGCAATGGGCAGCCGGAGAACAACTACAAGACCACGCCTCCCGTGCTGGACTCCGAC
GGCTCCTTCTTCCTCTACAGCAGGCTCACCGTGGACAAGAGCAGGTGGCAGGAGGGGAATGTCTTCTCATGCTCCGTGAT
GCATGAGGCTCTGCACAACCACTACACACAGAAGTCCCTCTCCCTGTCTCTGGGTAAATGA
Heavy Chain Amino Acid Sequence
(SEQ ID NO: 136)
EVQLVESGGGLVQPGGSLRLSCAASGFTVSSNYMSWVRQAPGKGLEWVSIIYSGGHTYYSDSVKGRFTISRHNSKNTLYL
QMNSLRGGDTAVYYCARGYTSGWYGFDFWGQGTLVTVSSASTKGPSVFPLAPCSRSTSESTAALGCLVKDYFPEPVTVSW
NSGALTSGVHTFPAVLQSSGLYSLSSVVTVPSSSLGTKTYTCNVDHKPSNTKVDKRVESKYGPPCPPCPAPEFLGGPSVF
LFPPKPKDTLMISRTPEVTCVVVDVSQEDPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK*
Light Chain Nucleotide Sequence
(SEQ ID NO: 137)
GACATCCAGATGACCCAGTCTCCATCTTCCGTGTCTGCATCTGTAGGAGACAGAGTCACCATCACTTGTCGGGCGAGTCA
GGGTATTAGCACCTGGTTAGCCTGGTATCAGCAGAAACCAGGGAAAGCCCCTAAGCTCCTGATCTATGCTGCATCCAGTT
TGCAAAGTGGGGTCCCGTCAAGATTCAGCGGCACTGGATCTGGGACAGATTTCACTCTCACCATCAGCAGCCTGCAGCCT
GAAGATTTTGCAACTTATTATTGTCAGCAGACTAACAGTTTCCCGTGGACGTTCGGCCAAGGGACCAAGGTGGAAATCAA
ACGAACTGTGGCTGCACCATCTGTCTTCATCTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTGGATAACGCCCTCCAATCGGGTAACTCCCAG
GAGAGTGTCACAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAACAGGGGAGAGT
GTTAG
Light Chain Amino Acid Sequence
(SEQ ID NO: 138)
DIQMTQSPSSVSASVGDRVTITCRASQGISTWLAWYQQKPGKAPKLLIYAASSLQSGVPSRFSGTGSGTDFTLTISSLQP
EDFATYYCQQTNSFPWTFGQGTKVEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC*
H4H30105P2
HCVR Nucleotide Sequence
(SEQ ID NO: 139)
GAGGTGCAGCTGGTGCAGTCTGGAGCAGAGGTGAAAAAGCCCGGGGAGTCTCTGAAGATCTCCTGTAAGGGTTCTGGATA
CAGCTTTACCAGCTACTGGATCGCCTGGGTGCGCCAGATGCCCGGGAAAGGCCTGGAGTGGATGGGGATCATCTATCCTG
GTGACTCTGATACCAGATACAGCCCGTCCTTCCAAGGCCAGGTCACCATGTCAGCCGACAAGTCCATCAGGATCGCCTAC
CTGCAGTGGAGCAGCCTGAAGGCCTCGGACACCGCCATGTATTATTGTGCGAGACTTGATTATAGCGGCAGCTGGTTTGA
CTACTGGGGCCAGGGAACCCTGGTCACCGTCTCCTCA
HCVR Amino Acid Sequence
(SEQ ID NO: 140)
EVQLVQSGAEVKKPGESLKISCKGSGYSFTSYWIAWVRQMPGKGLEWMGIIYPGDSDTRYSPSFQGQVTMSADKSIRIAY
LQWSSLKASDTAMYYCARLDYSGSWFDYWGQGTLVTVSS
HCDR1 Nucleotide Sequence
(SEQ ID NO: 141)
GGA TAC AGC TTT ACC AGC TAC TGG
HCDR1 Amino Acid Sequence
(SEQ ID NO: 142)
G Y S F T S Y W
HCDR2 Nucleotide Sequence
(SEQ ID NO: 143)
ATC TAT CCT GGT GAC TCT GAT ACC
HCDR2 Amino Acid Sequence
(SEQ ID NO: 144)
I Y P G D S D T
HCDR3 Nucleotide Sequence
(SEQ ID NO: 145)
GCG AGA CTT GAT TAT AGC GGC AGC TGG TTT GAC TAC
HCDR3 Amino Acid Sequence
(SEQ ID NO: 146)
A R L D Y S G S W F D Y
LCVR Nucleotide Sequence
(SEQ ID NO: 147)
GAAATAGTTTTGACACAGAGTCCCGGCACACTGTCACTCTCTCCCGGGGAAAGAGCCACCTTGTCATGTAGAGCAAGTCA
GTCAGTCTCTAGCTCTTATCTCGCCTGGTACCAGCAGAAGCCGGGACAGGCCCCTAGACTGCTGATCTACGGGGCAAGTT
CCAGGGCCACCGGAATCCCCGACCGGTTCAGTGGAAGCGGAAGCGGAACCGATTTTACTTTGACGATTTCTAGACTGGAG
CCAGAGGATTTCGCCGTTTACTATTGTCAACAGTACGGAAGCAGCCCGTGGACGTTTGGCCAGGGCACGAAGGTAGAAAT
CAAG
LCVR Amino Acid Sequence
(SEQ ID NO: 148)
EIVLTQSPGTLSLSPGERATLSCRASQSVSSSYLAWYQQKPGQAPRLLIYGASSRATGIPDRFSGSGSGTDFTLTISRLE
PEDFAVYYCQQYGSSPWTFGQGTKVEIK
LCDR1 Nucleotide Sequence
(SEQ ID NO: 149)
CAG TCA GTC TCT AGC TCT TAT
LCDR1 Amino Acid Sequence
(SEQ ID NO: 150)
Q S V S S S Y
LCDR2 Nucleotide Sequence
(SEQ ID NO: 151)
GGG GCA AGT
LCDR2 Amino Acid Sequence
(SEQ ID NO: 14)
G A S
LCDR3 Nucleotide Sequence
(SEQ ID NO: 152)
CAA CAG TAC GGA AGC AGC CCG TGG ACG
LCDR3 Amino Acid Sequence
(SEQ ID NO: 153)
Q Q Y G S S P W T
Heavy Chain Nucleotide Sequence
(SEQ ID NO: 154)
GAGGTGCAGCTGGTGCAGTCTGGAGCAGAGGTGAAAAAGCCCGGGGAGTCTCTGAAGATCTCCTGTAAGGGTTCTGGATA
CAGCTTTACCAGCTACTGGATCGCCTGGGTGCGCCAGATGCCCGGGAAAGGCCTGGAGTGGATGGGGATCATCTATCCTG
GTGACTCTGATACCAGATACAGCCCGTCCTTCCAAGGCCAGGTCACCATGTCAGCCGACAAGTCCATCAGGATCGCCTAC
CTGCAGTGGAGCAGCCTGAAGGCCTCGGACACCGCCATGTATTATTGTGCGAGACTTGATTATAGCGGCAGCTGGTTTGA
CTACTGGGGCCAGGGAACCCTGGTCACCGTCTCCTCAGCCTCCACCAAGGGCCCATCGGTCTTCCCCCTGGCGCCCTGCT
CCAGGAGCACCTCCGAGAGCACAGCCGCCCTGGGCTGCCTGGTCAAGGACTACTTCCCCGAACCGGTGACGGTGTCGTGG
AACTCAGGCGCCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACGAAGACCTACACCTGCAACGTAGATCACAAGCCCAGCAACACCAAGGTGG
ACAAGAGAGTTGAGTCCAAATATGGTCCCCCATGCCCACCCTGCCCAGCACCTGAGTTCCTGGGGGGACCATCAGTCTTC
CTGTTCCCCCCAAAACCCAAGGACACTCTCATGATCTCCCGGACCCCTGAGGTCACGTGCGTGGTGGTGGACGTGAGCCA
GGAAGACCCCGAGGTCCAGTTCAACTGGTACGTGGATGGCGTGGAGGTGCATAATGCCAAGACAAAGCCGCGGGAGGAGC
AGTTCAACAGCACGTACCGTGTGGTCAGCGTCCTCACCGTCCTGCACCAGGACTGGCTGAACGGCAAGGAGTACAAGTGC
AAGGTCTCCAACAAAGGCCTCCCGTCCTCCATCGAGAAAACCATCTCCAAAGCCAAAGGGCAGCCCCGAGAGCCACAGGT
GTACACCCTGCCCCCATCCCAGGAGGAGATGACCAAGAACCAGGTCAGCCTGACCTGCCTGGTCAAAGGCTTCTACCCCA
GCGACATCGCCGTGGAGTGGGAGAGCAATGGGCAGCCGGAGAACAACTACAAGACCACGCCTCCCGTGCTGGACTCCGAC
GGCTCCTTCTTCCTCTACAGCAGGCTCACCGTGGACAAGAGCAGGTGGCAGGAGGGGAATGTCTTCTCATGCTCCGTGAT
GCATGAGGCTCTGCACAACCACTACACACAGAAGTCCCTCTCCCTGTCTCTGGGTAAATGA
Heavy Chain Amino Acid Sequence
(SEQ ID NO: 155)
EVQLVQSGAEVKKPGESLKISCKGSGYSFTSYWIAWVRQMPGKGLEWMGIIYPGDSDTRYSPSFQGQVTMSADKSIRIAY
LOWSSLKASDTAMYYCARLDYSGSWFDYWGQGTLVTVSSASTKGPSVFPLAPCSRSTSESTAALGCLVKDYFPEPVTVSW
NSGALTSGVHTFPAVLQSSGLYSLSSVVTVPSSSLGTKTYTCNVDHKPSNTKVDKRVESKYGPPCPPCPAPEFLGGPSVF
LFPPKPKDTLMISRTPEVTCVVVDVSQEDPEVQFNWYVDGVEVHNAKTKPREEQFNSTYRVVSVLTVLHQDWINGKEYKC
KVSNKGLPSSIEKTISKAKGQPREPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK*
Light Chain Nucleotide Sequence
(SEQ ID NO: 156)
GAAATAGTTTTGACACAGAGTCCCGGCACACTGTCACTCTCTCCCGGGGAAAGAGCCACCTTGTCATGTAGAGCAAGTCA
GTCAGTCTCTAGCTCTTATCTCGCCTGGTACCAGCAGAAGCCGGGACAGGCCCCTAGACTGCTGATCTACGGGGCAAGTT
CCAGGGCCACCGGAATCCCCGACCGGTTCAGTGGAAGCGGAAGCGGAACCGATTTTACTTTGACGATTTCTAGACTGGAG
CCAGAGGATTTCGCCGTTTACTATTGTCAACAGTACGGAAGCAGCCCGTGGACGTTTGGCCAGGGCACGAAGGTAGAAAT
CAAGCGAACTGTGGCTGCACCATCTGTCTTCATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTGACGCTGAGCAAAGCAGACTA
CGAGAAACACAAAGTCTACGCCTGCGAAGTCACCCATCAGGGCCTGAGCTCGCCCGTCACAAAGAGCTTCAACAGGGGAG
AGTGTTAG
Light Chain Amino Acid Sequence
(SEQ ID NO: 157)
EIVLTQSPGTLSLSPGERATLSCRASQSVSSSYLAWYQQKPGQAPRLLIYGASSRATGIPDRFSGSGSGTDFTLTISRLE
PEDFAVYYCQQYGSSPWTFGQGTKVEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC*
H4H30108P2
HCVR Nucleotide Sequence
(SEQ ID NO: 158)
CAGGTCCAGCTGGTGCAGTCTGGGGCTGAGGTGAAGAAGCCTGGGGCCTCAGTGAAGGTCTCCTGCAAGGCTTCTGGATA
CACCTTCAACAGTTATGATGTCAACTGGGTGCGACAGGCCACTGGACAAGGGCTTGAGTGGATGGGATGGATGAACCCTC
ACAGTGGTAACACAGGCTACGCACAGAAGTTCCAGGGCAGAGTCACTATGACCAGGGACACCTCCACAAGCACAAGCTAT
ATGGAGTTGAGCAGCCTGACATCTGAGGACACGGCCGTATATTACTGTGCGAGAGGCCCTTTTTCTCTACTTTCAGCTGA
ATTCTTCCAGCACTGGGGCCAGGGCACCCTGGTCACCGTCTCCTCA
HCVR Amino Acid Sequence
(SEQ ID NO: 159)
QVQLVQSGAEVKKPGASVKVSCKASGYTFNSYDVNWVRQATGQGLEWMGWMNPHSGNTGYAQKFQGRVTMTRDTSTSTSY
MELSSLTSEDTAVYYCARGPFSLLSAEFFQHWGQGTLVTVSS
HCDR1 Nucleotide Sequence
(SEQ ID NO: 160)
GGA TAC ACC TTC AAC AGT TAT GAT
HCDR1 Amino Acid Sequence
(SEQ ID NO: 161)
G Y T F N S Y D
HCDR2 Nucleotide Sequence
(SEQ ID NO: 162)
ATG AAC CCT CAC AGT GGT AAC ACA
HCDR2 Amino Acid Sequence
(SEQ ID NO: 163)
M N P H S G N T
HCDR3 Nucleotide Sequence
(SEQ ID NO: 164)
GCG AGA GGC CCT TTT TCT CTA CTT TCA GCT GAA TTC TTC CAG CAC
HCDR3 Amino Acid Sequence
(SEQ ID NO: 165)
A R G P F S L L S A E F F Q H
LCVR Nucleotide Sequence
(SEQ ID NO: 147)
GAAATAGTTTTGACACAGAGTCCCGGCACACTGTCACTCTCTCCCGGGGAAAGAGCCACCTTGTCATGTAGAGCAAGTCA
GTCAGTCTCTAGCTCTTATCTCGCCTGGTACCAGCAGAAGCCGGGACAGGCCCCTAGACTGCTGATCTACGGGGCAAGTT
CCAGGGCCACCGGAATCCCCGACCGGTTCAGTGGAAGCGGAAGCGGAACCGATTTTACTTTGACGATTTCTAGACTGGAG
CCAGAGGATTTCGCCGTTTACTATTGTCAACAGTACGGAAGCAGCCCGTGGACGTTTGGCCAGGGCACGAAGGTAGAAAT
CAAG
LCVR Amino Acid Sequence
(SEQ ID NO: 148)
EIVLTQSPGTLSLSPGERATLSCRASQSVSSSYLAWYQQKPGQAPRLLIYGASSRATGIPDRFSGSGSGTDFTLTISRLE
PEDFAVYYCQQYGSSPWTFGQGTKVEIK
LCDR1 Nucleotide Sequence
(SEQ ID NO: 149)
CAG TCA GTC TCT AGC TCT TAT
LCDR1 Amino Acid Sequence
(SEQ ID NO: 150)
Q S V S S S Y
LCDR2 Nucleotide Sequence
(SEQ ID NO: 151)
GGG GCA AGT
LCDR2 Amino Acid Sequence
(SEQ ID NO: 14)
G A S
LCDR3 Nucleotide Sequence
(SEQ ID NO: 152)
CAA CAG TAC GGA AGC AGC CCG TGG ACG
LCDR3 Amino Acid Sequence
(SEQ ID NO: 153)
Q Q Y G S S P W T
Heavy Chain Nucleotide Sequence
(SEQ ID NO: 166)
CAGGTCCAGCTGGTGCAGTCTGGGGCTGAGGTGAAGAAGCCTGGGGCCTCAGTGAAGGTCTCCTGCAAGGCTTCTGGATA
CACCTTCAACAGTTATGATGTCAACTGGGTGCGACAGGCCACTGGACAAGGGCTTGAGTGGATGGGATGGATGAACCCTC
ACAGTGGTAACACAGGCTACGCACAGAAGTTCCAGGGCAGAGTCACTATGACCAGGGACACCTCCACAAGCACAAGCTAT
ATGGAGTTGAGCAGCCTGACATCTGAGGACACGGCCGTATATTACTGTGCGAGAGGCCCTTTTTCTCTACTTTCAGCTGA
ATTCTTCCAGCACTGGGGCCAGGGCACCCTGGTCACCGTCTCCTCAGCCTCCACCAAGGGCCCATCGGTCTTCCCCCTGG
CGCCCTGCTCCAGGAGCACCTCCGAGAGCACAGCCGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTACAGTCCTCAGGACTCTACTCCCT
CAGCAGCGTGGTGACCGTGCCCTCCAGCAGCTTGGGCACGAAGACCTACACCTGCAACGTAGATCACAAGCCCAGCAACA
CCAAGGTGGACAAGAGAGTTGAGTCCAAATATGGTCCCCCATGCCCACCCTGCCCAGCACCTGAGTTCCTGGGGGGACCA
TCAGTCTTCCTGTTCCCCCCAAAACCCAAGGACACTCTCATGATCTCCCGGACCCCTGAGGTCACGTGCGTGGTGGTGGA
CGTGAGCCAGGAAGACCCCGAGGTCCAGTTCAACTGGTACGTGGATGGCGTGGAGGTGCATAATGCCAAGACAAAGCCGC
GGGAGGAGCAGTTCAACAGCACGTACCGTGTGGTCAGCGTCCTCACCGTCCTGCACCAGGACTGGCTGAACGGCAAGGAG
TACAAGTGCAAGGTCTCCAACAAAGGCCTCCCGTCCTCCATCGAGAAAACCATCTCCAAAGCCAAAGGGCAGCCCCGAGA
GCCACAGGTGTACACCCTGCCCCCATCCCAGGAGGAGATGACCAAGAACCAGGTCAGCCTGACCTGCCTGGTCAAAGGCT
TCTACCCCAGCGACATCGCCGTGGAGTGGGAGAGCAATGGGCAGCCGGAGAACAACTACAAGACCACGCCTCCCGTGCTG
GACTCCGACGGCTCCTTCTTCCTCTACAGCAGGCTCACCGTGGACAAGAGCAGGTGGCAGGAGGGGAATGTCTTCTCATG
CTCCGTGATGCATGAGGCTCTGCACAACCACTACACACAGAAGTCCCTCTCCCTGTCTCTGGGTAAATGA
Heavy Chain Amino Acid Sequence
(SEQ ID NO: 167)
QVQLVQSGAEVKKPGASVKVSCKASGYTFNSYDVNWVRQATGQGLEWMGWMNPHSGNTGYAQKFQGRVTMTRDTSTSTSY
MELSSLTSEDTAVYYCARGPFSLLSAEFFQHWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTKVDKRVESKYGPPCPPCPAPEFLGGP
SVFLFPPKPKDTLMISRTPEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWESNGQPENNYKTTPPVL
DSDGSFFLYSRLTVDKSRWQEGNVFSCSVMHEALHNHYTQKSLSLSLGK*
Light Chain Nucleotide Sequence
(SEQ ID NO: 156)
GAAATAGTTTTGACACAGAGTCCCGGCACACTGTCACTCTCTCCCGGGGAAAGAGCCACCTTGTCATGTAGAGCAAGTCA
GTCAGTCTCTAGCTCTTATCTCGCCTGGTACCAGCAGAAGCCGGGACAGGCCCCTAGACTGCTGATCTACGGGGCAAGTT
CCAGGGCCACCGGAATCCCCGACCGGTTCAGTGGAAGCGGAAGCGGAACCGATTTTACTTTGACGATTTCTAGACTGGAG
CCAGAGGATTTCGCCGTTTACTATTGTCAACAGTACGGAAGCAGCCCGTGGACGTTTGGCCAGGGCACGAAGGTAGAAAT
CAAGCGAACTGTGGCTGCACCATCTGTCTTCATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTGACGCTGAGCAAAGCAGACTA
CGAGAAACACAAAGTCTACGCCTGCGAAGTCACCCATCAGGGCCTGAGCTCGCCCGTCACAAAGAGCTTCAACAGGGGAG
AGTGTTAG
Light Chain Amino Acid Sequence
(SEQ ID NO: 157)
EIVLTQSPGTLSLSPGERATLSCRASQSVSSSYLAWYQQKPGQAPRLLIYGASSRATGIPDRFSGSGSGTDFTLTISRLE
PEDFAVYYCQQYGSSPWTFGQGTKVEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC*
H4H30117P2
HCVR Nucleotide Sequence
(SEQ ID NO: 168)
CAGGTCCAGCTGGTACAGTCTGGGGCTGAGGTGAAGAAGCCTGGGTCCTCGGTGAAGGTCTCCTGCAAGGCTTCTGGAGA
CACCTTCAGTAACTATGTTATCGGCTGGGTGCGACAGGCCCCTGGACAAGGGCTTGAGTGGATGGGAGGGATCATCCCTA
TCTTTGGTACAACAAACTACGCACAGCAGTTCCAGGGCAGAGTCACGATTACCACGGACGAATCCACGAGCACGGCCTAC
ATGGAGCTGAGCAGCCTGAGATCTGAGGACACGGCCGTGTATTACTGTGCGAGAGATGGGAACTACGGTGACTACTTTGA
CTACTGGGGCCAGGGAACCCTGGTCACCGTCTCCTCA
HCVR Amino Acid Sequence
(SEQ ID NO: 169)
QVQLVQSGAEVKKPGSSVKVSCKASGDTFSNYVIGWVRQAPGQGLEWMGGIIPIFGTTNYAQQFQGRVTITTDESTSTAY
MELSSLRSEDTAVYYCARDGNYGDYFDYWGQGTLVTVSS
HCDR1 Nucleotide Sequence
(SEQ ID NO: 170)
GGA GAC ACC TTC AGT AAC TAT GTT
HCDR1 Amino Acid Sequence
(SEQ ID NO: 171)
G D T F S N Y V
HCDR2 Nucleotide Sequence
(SEQ ID NO: 172)
ATC ATC CCT ATC TTT GGT ACA ACA
HCDR2 Amino Acid Sequence
(SEQ ID NO: 173)
I I P I F G T T
HCDR3 Nucleotide Sequence
(SEQ ID NO: 174)
GCG AGA GAT GGG AAC TAC GGT GAC TAC TTT GAC TAC
HCDR3 Amino Acid Sequence
(SEQ ID NO: 175)
A R D G N Y G D Y F D Y
LCVR Nucleotide Sequence
(SEQ ID NO: 147)
GAAATAGTTTTGACACAGAGTCCCGGCACACTGTCACTCTCTCCCGGGGAAAGAGCCACCTTGTCATGTAGAGCAAGTCA
GTCAGTCTCTAGCTCTTATCTCGCCTGGTACCAGCAGAAGCCGGGACAGGCCCCTAGACTGCTGATCTACGGGGCAAGTT
CCAGGGCCACCGGAATCCCCGACCGGTTCAGTGGAAGCGGAAGCGGAACCGATTTTACTTTGACGATTTCTAGACTGGAG
CCAGAGGATTTCGCCGTTTACTATTGTCAACAGTACGGAAGCAGCCCGTGGACGTTTGGCCAGGGCACGAAGGTAGAAAT
CAAG
LCVR Amino Acid Sequence
(SEQ ID NO: 148)
EIVLTQSPGTLSLSPGERATLSCRASQSVSSSYLAWYQQKPGQAPRLLIYGASSRATGIPDRFSGSGSGTDFTLTISRLE
PEDFAVYYCQQYGSSPWTFGQGTKVEIK
LCDR1 Nucleotide Sequence
(SEQ ID NO: 149)
CAG TCA GTC TCT AGC TCT TAT
LCDR1 Amino Acid Sequence
(SEQ ID NO: 150)
Q S V S S S Y
LCDR2 Nucleotide Sequence
(SEQ ID NO: 151)
GGG GCA AGT
LCDR2 Amino Acid Sequence
(SEQ ID NO: 14)
G A S
LCDR3 Nucleotide Sequence
(SEQ ID NO: 152)
CAA CAG TAC GGA AGC AGC CCG TGG ACG
LCDR3 Amino Acid Sequence
(SEQ ID NO: 153)
Q Q Y G S S P W T
Heavy Chain Nucleotide Sequence
(SEQ ID NO: 176)
CAGGTCCAGCTGGTACAGTCTGGGGCTGAGGTGAAGAAGCCTGGGTCCTCGGTGAAGGTCTCCTGCAAGGCTTCTGGAGA
CACCTTCAGTAACTATGTTATCGGCTGGGTGCGACAGGCCCCTGGACAAGGGCTTGAGTGGATGGGAGGGATCATCCCTA
TCTTTGGTACAACAAACTACGCACAGCAGTTCCAGGGCAGAGTCACGATTACCACGGACGAATCCACGAGCACGGCCTAC
ATGGAGCTGAGCAGCCTGAGATCTGAGGACACGGCCGTGTATTACTGTGCGAGAGATGGGAACTACGGTGACTACTTTGA
CTACTGGGGCCAGGGAACCCTGGTCACCGTCTCCTCAGCCTCCACCAAGGGCCCATCGGTCTTCCCCCTGGCGCCCTGCT
CCAGGAGCACCTCCGAGAGCACAGCCGCCCTGGGCTGCCTGGTCAAGGACTACTTCCCCGAACCGGTGACGGTGTCGTGG
AACTCAGGCGCCCTGACCAGCGGCGTGCACACCTTCCCGGCTGTCCTACAGTCCTCAGGACTCTACTCCCTCAGCAGCGT
GGTGACCGTGCCCTCCAGCAGCTTGGGCACGAAGACCTACACCTGCAACGTAGATCACAAGCCCAGCAACACCAAGGTGG
ACAAGAGAGTTGAGTCCAAATATGGTCCCCCATGCCCACCCTGCCCAGCACCTGAGTTCCTGGGGGGACCATCAGTCTTC
CTGTTCCCCCCAAAACCCAAGGACACTCTCATGATCTCCCGGACCCCTGAGGTCACGTGCGTGGTGGTGGACGTGAGCCA
GGAAGACCCCGAGGTCCAGTTCAACTGGTACGTGGATGGCGTGGAGGTGCATAATGCCAAGACAAAGCCGCGGGAGGAGC
AGTTCAACAGCACGTACCGTGTGGTCAGCGTCCTCACCGTCCTGCACCAGGACTGGCTGAACGGCAAGGAGTACAAGTGC
AAGGTCTCCAACAAAGGCCTCCCGTCCTCCATCGAGAAAACCATCTCCAAAGCCAAAGGGCAGCCCCGAGAGCCACAGGT
GTACACCCTGCCCCCATCCCAGGAGGAGATGACCAAGAACCAGGTCAGCCTGACCTGCCTGGTCAAAGGCTTCTACCCCA
GCGACATCGCCGTGGAGTGGGAGAGCAATGGGCAGCCGGAGAACAACTACAAGACCACGCCTCCCGTGCTGGACTCCGAC
GGCTCCTTCTTCCTCTACAGCAGGCTCACCGTGGACAAGAGCAGGTGGCAGGAGGGGAATGTCTTCTCATGCTCCGTGAT
GCATGAGGCTCTGCACAACCACTACACACAGAAGTCCCTCTCCCTGTCTCTGGGTAAATGA
Heavy Chain Amino Acid Sequence
(SEQ ID NO: 177)
QVQLVQSGAEVKKPGSSVKVSCKASGDTFSNYVIGWVRQAPGQGLEWMGGIIPIFGTTNYAQQFQGRVTITTDESTSTAY
MELSSLRSEDTAVYYCARDGNYGDYFDYWGQGTLVTVSSASTKGPSVFPLAPCSRSTSESTAALGCLVKDYFPEPVTVSW
NSGALTSGVHTFPAVLQSSGLYSLSSVVTVPSSSLGTKTYTCNVDHKPSNTKVDKRVESKYGPPCPPCPAPEFLGGPSVF
LFPPKPKDTLMISRTPEVTCVVVDVSQEDPEVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK*
Light Chain Nucleotide Sequence
(SEQ ID NO: 156)
GAAATAGTTTTGACACAGAGTCCCGGCACACTGTCACTCTCTCCCGGGGAAAGAGCCACCTTGTCATGTAGAGCAAGTCA
GTCAGTCTCTAGCTCTTATCTCGCCTGGTACCAGCAGAAGCCGGGACAGGCCCCTAGACTGCTGATCTACGGGGCAAGTT
CCAGGGCCACCGGAATCCCCGACCGGTTCAGTGGAAGCGGAAGCGGAACCGATTTTACTTTGACGATTTCTAGACTGGAG
CCAGAGGATTTCGCCGTTTACTATTGTCAACAGTACGGAAGCAGCCCGTGGACGTTTGGCCAGGGCACGAAGGTAGAAAT
CAAGCGAACTGTGGCTGCACCATCTGTCTTCATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTGACGCTGAGCAAAGCAGACTA
CGAGAAACACAAAGTCTACGCCTGCGAAGTCACCCATCAGGGCCTGAGCTCGCCCGTCACAAAGAGCTTCAACAGGGGAG
AGTGTTAG
Light Chain Amino Acid Sequence
(SEQ ID NO: 157)
EIVLTQSPGTLSLSPGERATLSCRASQSVSSSYLAWYQQKPGQAPRLLIYGASSRATGIPDRFSGSGSGTDFTLTISRLE
PEDFAVYYCQQYGSSPWTFGQGTKVEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC*
H4H30045P
HCVR Nucleotide Sequence
(SEQ ID NO: 178)
CAGGTTCAGCTGGTGCAGTCTGGAGCTGAGGTGAAGAAGCCTGGGGCCTCAGTGAAGGTCTCCTGCAAGACTTCTGGTTA
CAGATTCGTCAACTATGGTTTCAGCTGGGTGCGCCAGGCCCCTGGACAAGGCCTTGAATGGATGGGATGGATCAGCCCTT
ATAATGGTAACACAAACTATATACAGAATCTCCAGGACAGAATCACCATGACCACAGACACCTCTACGAACACAGCCTAC
ATGGAACTGACGAACCTGAGATCTGACGACACGGCCGTATATTACTGTGCGACCTTAACTGGGGTTCACTTTGACTACTG
GGGCCAGGGAACCCTGGTCACCGTCTCCTCA
HCVR Amino Acid Sequence
(SEQ ID NO: 179)
QVQLVQSGAEVKKPGASVKVSCKTSGYRFVNYGFSWVRQAPGQGLEWMGWISPYNGNTNYIQNLQDRITMTTDTSTNTAY
MELTNLRSDDTAVYYCATLTGVHFDYWGQGTLVTVSS
HCDR1 Nucleotide Sequence
(SEQ ID NO: 180)
GGT TAC AGA TTC GTC AAC TAT GGT
HCDR1 Amino Acid Sequence
(SEQ ID NO: 181)
G Y R F V N Y G
HCDR2 Nucleotide Sequence
(SEQ ID NO: 182)
ATC AGC CCT TAT AAT GGT AAC ACA
HCDR2 Amino Acid Sequence
(SEQ ID NO: 183)
I S P Y N G N T
HCDR3 Nucleotide Sequence
(SEQ ID NO: 184)
GCG ACC TTA ACT GGG GTT CAC TTT GAC TAC
HCDR3 Amino Acid Sequence
(SEQ ID NO: 185)
A T L T G V H F D Y
LCVR Nucleotide Sequence
(SEQ ID NO: 186)
GCCATCCAGATGACCCAGTCTCCATCCTCCCTGTCTGCATCTGTAGGAGACAGAGTCACCATCACTTGCCGGGCAAGTCA
GGGCATTAGAAATGATTTAGGCTGGTATCAGCAGAAACCAGGGAAAGCCCCTAATCTCCTGATCTTTGAAACATCTCGTT
TACAAAGTGGGGTCCCTTCGAGGTTCAGCGGCAGTGGTTCTGGCACAGATTTCACTCTCACCATCAACAGCCTTCAGCCT
GAAGATTTTGCAACTTATTACTGTCTACAAGATTACAATTACCCGTGGACGTTCGGCCAAGGGACCAAGGTGGAAATCAA
A
LCVR Amino Acid Sequence
(SEQ ID NO: 187)
AIQMTQSPSSLSASVGDRVTITCRASQGIRNDLGWYQQKPGKAPNLLIFETSRLQSGVPSRFSGSGSGTDFTLTINSLQP
EDFATYYCLQDYNYPWTFGQGTKVEIK
LCDR1 Nucleotide Sequence
(SEQ ID NO: 188)
CAG GGC ATT AGA AAT GAT
LCDR1 Amino Acid Sequence
(SEQ ID NO: 189)
Q G I R N D
LCDR2 Nucleotide Sequence
(SEQ ID NO: 190)
GAA ACA TCT
LCDR2 Amino Acid Sequence
(SEQ ID NO: 191)
E T S
LCDR3 Nucleotide Sequence
(SEQ ID NO: 192)
CTA CAA GAT TAC AAT TAC CCG TGG ACG
LCDR3 Amino Acid Sequence
(SEQ ID NO: 193)
L Q D Y N Y P W T
Heavy Chain Nucleotide Sequence
(SEQ ID NO: 194)
CAGGTTCAGCTGGTGCAGTCTGGAGCTGAGGTGAAGAAGCCTGGGGCCTCAGTGAAGGTCTCCTGCAAGACTTCTGGTTA
CAGATTCGTCAACTATGGTTTCAGCTGGGTGCGCCAGGCCCCTGGACAAGGCCTTGAATGGATGGGATGGATCAGCCCTT
ATAATGGTAACACAAACTATATACAGAATCTCCAGGACAGAATCACCATGACCACAGACACCTCTACGAACACAGCCTAC
ATGGAACTGACGAACCTGAGATCTGACGACACGGCCGTATATTACTGTGCGACCTTAACTGGGGTTCACTTTGACTACTG
GGGCCAGGGAACCCTGGTCACCGTCTCCTCAGCCTCCACCAAGGGCCCATCGGTCTTCCCCCTGGCGCCCTGCTCCAGGA
GCACCTCCGAGAGCACAGCCGCCCTGGGCTGCCTGGTCAAGGACTACTTCCCCGAACCGGTGACGGTGTCGTGGAACTCA
GGCGCCCTGACCAGCGGCGTGCACACCTTCCCGGCTGTCCTACAGTCCTCAGGACTCTACTCCCTCAGCAGCGTGGTGAC
CGTGCCCTCCAGCAGCTTGGGCACGAAGACCTACACCTGCAACGTAGATCACAAGCCCAGCAACACCAAGGTGGACAAGA
GAGTTGAGTCCAAATATGGTCCCCCATGCCCACCCTGCCCAGCACCTGAGTTCCTGGGGGGACCATCAGTCTTCCTGTTC
CCCCCAAAACCCAAGGACACTCTCATGATCTCCCGGACCCCTGAGGTCACGTGCGTGGTGGTGGACGTGAGCCAGGAAGA
CCCCGAGGTCCAGTTCAACTGGTACGTGGATGGCGTGGAGGTGCATAATGCCAAGACAAAGCCGCGGGAGGAGCAGTTCA
ACAGCACGTACCGTGTGGTCAGCGTCCTCACCGTCCTGCACCAGGACTGGCTGAACGGCAAGGAGTACAAGTGCAAGGTC
TCCAACAAAGGCCTCCCGTCCTCCATCGAGAAAACCATCTCCAAAGCCAAAGGGCAGCCCCGAGAGCCACAGGTGTACAC
CCTGCCCCCATCCCAGGAGGAGATGACCAAGAACCAGGTCAGCCTGACCTGCCTGGTCAAAGGCTTCTACCCCAGCGACA
TCGCCGTGGAGTGGGAGAGCAATGGGCAGCCGGAGAACAACTACAAGACCACGCCTCCCGTGCTGGACTCCGACGGCTCC
TTCTTCCTCTACAGCAGGCTCACCGTGGACAAGAGCAGGTGGCAGGAGGGGAATGTCTTCTCATGCTCCGTGATGCATGA
GGCTCTGCACAACCACTACACACAGAAGTCCCTCTCCCTGTCTCTGGGTAAATGA
Heavy Chain Amino Acid Sequence
(SEQ ID NO: 195)
QVQLVQSGAEVKKPGASVKVSCKTSGYRFVNYGFSWVRQAPGQGLEWMGWISPYNGNTNYIQNLQDRITMTTDTSTNTAY
MELTNLRSDDTAVYYCATLTGVHFDYWGQGTLVTVSSASTKGPSVFPLAPCSRSTSESTAALGCLVKDYFPEPVTVSWNS
GALTSGVHTFPAVLQSSGLYSLSSVVTVPSSSLGTKTYTCNVDHKPSNTKVDKRVESKYGPPCPPCPAPEFLGGPSVFLF
PPKPKDTLMISRTPEVTCVVVDVSQEDPEVQFNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK*
Light Chain Nucleotide Sequence
(SEQ ID NO: 196)
GCCATCCAGATGACCCAGTCTCCATCCTCCCTGTCTGCATCTGTAGGAGACAGAGTCACCATCACTTGCCGGGCAAGTCA
GGGCATTAGAAATGATTTAGGCTGGTATCAGCAGAAACCAGGGAAAGCCCCTAATCTCCTGATCTTTGAAACATCTCGTT
TACAAAGTGGGGTCCCTTCGAGGTTCAGCGGCAGTGGTTCTGGCACAGATTTCACTCTCACCATCAACAGCCTTCAGCCT
GAAGATTTTGCAACTTATTACTGTCTACAAGATTACAATTACCCGTGGACGTTCGGCCAAGGGACCAAGGTGGAAATCAA
ACGAACTGTGGCTGCACCATCTGTCTTCATCTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTGGATAACGCCCTCCAATCGGGTAACTCCCAG
GAGAGTGTCACAGAGCAGGACAGCAAGGACAGCACCTACAGCCTCAGCAGCACCCTGACGCTGAGCAAAGCAGACTACGA
GAAACACAAAGTCTACGCCTGCGAAGTCACCCATCAGGGCCTGAGCTCGCCCGTCACAAAGAGCTTCAACAGGGGAGAGT
GTTAG
Light Chain Amino Acid Sequence
(SEQ ID NO: 197)
AIQMTQSPSSLSASVGDRVTITCRASQGIRNDLGWYQQKPGKAPNLLIFETSRLQSGVPSRFSGSGSGTDFTLTINSLQP
EDFATYYCLQDYNYPWTFGQGTKVEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSENRGEC*
H4H30061P
HCVR Nucleotide Sequence
(SEQ ID NO: 198)
GAGGTGCAGCTGGTGGAGTCTGGGGGAGGCTTGGCCCAGCCTGGGGGGTCCCTGAAACTCTCTTGTGAAGCCTCTGGATT
CACGTTCAGTGATTCTGCAATGCACTGGGTCCGCCAGGCTTCCGGAAAAGGGCTGGAGTGGGTTGGTCGTATTAGAAGCA
AAGCTAATAGTTACGCGACAGGATATGCTGCGTCGGTGAAAGGCAGGTTCACCATCTCCAGAGATGATTCAAAGAACATG
GCGTTTCTGGAAATGAACAGCCTGAAAACCGAAGACACGGCCGTATATTACTGTCTCCGACAAACTTACGGTGACCCCTG
GGGCCAGGGAACCCTGGTCACCGTCTCCTCA
HCVR Amino Acid Sequence
(SEQ ID NO: 199)
EVQLVESGGGLAQPGGSLKLSCEASGFTFSDSAMHWVRQASGKGLEWVGRIRSKANSYATGYAASVKGRFTISRDDSKNM
AFLEMNSLKTEDTAVYYCLRQTYGDPWGQGTLVTVSS
HCDR1 Nucleotide Sequence
(SEQ ID NO: 200)
GGA TTC ACG TTC AGT GAT TCT GCA
HCDR1 Amino Acid Sequence
(SEQ ID NO: 201)
G F T F S D S A
HCDR2 Nucleotide Sequence
(SEQ ID NO: 202)
ATT AGA AGC AAA GCT AAT AGT TAC GCG ACA
HCDR2 Amino Acid Sequence
(SEQ ID NO: 203)
I R S K A N S Y A T
HCDR3 Nucleotide Sequence
(SEQ ID NO: 204)
CTC CGA CAA ACT TAC GGT GAC CCC
HCDR3 Amino Acid Sequence
(SEQ ID NO: 205)
L R Q T Y G D P
LCVR Nucleotide Sequence
(SEQ ID NO: 206)
GATGTTGTGATGACTCAGTCTCCACTCTCCCTGTCCGTCACCCTTGGACAGCCGGCCTCCATCTCCTGCAGGTCTAGTCT
AAGCCTCGTATACAGTGATGGAAACAACTACTTGAATTGGTTTCAGCAGAGGCCAGGCCAATCTCCAAGGCGCCTACTTT
ATAAAGTTTTTAACCGGGACTCTGGGGTCCCAGACAGATTCAGCGGCAGTGGGTCAGGCACTGATTTCACACTGAAAATC
AGCAGGGTGGAGGCTGAGGATGTTGGGGTTTATTACTGCATGCAAGGAACACACTGGCCGTGGACGTTCGGCCAAGGGAC
CAAGGTGGAAATCAAA
LCVR Amino Acid Sequence
(SEQ ID NO: 207)
DVVMTQSPLSLSVTLGQPASISCRSSLSLVYSDGNNYLNWFQQRPGQSPRRLLYKVFNRDSGVPDRFSGSGSGTDFTLKI
SRVEAEDVGVYYCMQGTHWPWTFGQGTKVEIK
LCDR1 Nucleotide Sequence
(SEQ ID NO: 208)
CTA AGC CTC GTA TAC AGT GAT GGA AAC AAC TAC
LCDR1 Amino Acid Sequence
(SEQ ID NO: 209)
L S L V Y S D G N N Y
LCDR2 Nucleotide Sequence
(SEQ ID NO: 210)
AAA GTT TTT
LCDR2 Amino Acid Sequence
(SEQ ID NO: 211)
K V F
LCDR3 Nucleotide Sequence
(SEQ ID NO: 212)
ATG CAA GGA ACA CAC TGG CCG TGG ACG
LCDR3 Amino Acid Sequence
(SEQ ID NO: 213)
M Q G T H W P W T
Heavy Chain Nucleotide Sequence
(SEQ ID NO: 214)
GAGGTGCAGCTGGTGGAGTCTGGGGGAGGCTTGGCCCAGCCTGGGGGGTCCCTGAAACTCTCTTGTGAAGCCTCTGGATT
CACGTTCAGTGATTCTGCAATGCACTGGGTCCGCCAGGCTTCCGGAAAAGGGCTGGAGTGGGTTGGTCGTATTAGAAGCA
AAGCTAATAGTTACGCGACAGGATATGCTGCGTCGGTGAAAGGCAGGTTCACCATCTCCAGAGATGATTCAAAGAACATG
GCGTTTCTGGAAATGAACAGCCTGAAAACCGAAGACACGGCCGTATATTACTGTCTCCGACAAACTTACGGTGACCCCTG
GGGCCAGGGAACCCTGGTCACCGTCTCCTCAGCCTCCACCAAGGGCCCATCGGTCTTCCCCCTGGCGCCCTGCTCCAGGA
GCACCTCCGAGAGCACAGCCGCCCTGGGCTGCCTGGTCAAGGACTACTTCCCCGAACCGGTGACGGTGTCGTGGAACTCA
GGCGCCCTGACCAGCGGCGTGCACACCTTCCCGGCTGTCCTACAGTCCTCAGGACTCTACTCCCTCAGCAGCGTGGTGAC
CGTGCCCTCCAGCAGCTTGGGCACGAAGACCTACACCTGCAACGTAGATCACAAGCCCAGCAACACCAAGGTGGACAAGA
GAGTTGAGTCCAAATATGGTCCCCCATGCCCACCCTGCCCAGCACCTGAGTTCCTGGGGGGACCATCAGTCTTCCTGTTC
CCCCCAAAACCCAAGGACACTCTCATGATCTCCCGGACCCCTGAGGTCACGTGCGTGGTGGTGGACGTGAGCCAGGAAGA
CCCCGAGGTCCAGTTCAACTGGTACGTGGATGGCGTGGAGGTGCATAATGCCAAGACAAAGCCGCGGGAGGAGCAGTTCA
ACAGCACGTACCGTGTGGTCAGCGTCCTCACCGTCCTGCACCAGGACTGGCTGAACGGCAAGGAGTACAAGTGCAAGGTC
TCCAACAAAGGCCTCCCGTCCTCCATCGAGAAAACCATCTCCAAAGCCAAAGGGCAGCCCCGAGAGCCACAGGTGTACAC
CCTGCCCCCATCCCAGGAGGAGATGACCAAGAACCAGGTCAGCCTGACCTGCCTGGTCAAAGGCTTCTACCCCAGCGACA
TCGCCGTGGAGTGGGAGAGCAATGGGCAGCCGGAGAACAACTACAAGACCACGCCTCCCGTGCTGGACTCCGACGGCTCC
TTCTTCCTCTACAGCAGGCTCACCGTGGACAAGAGCAGGTGGCAGGAGGGGAATGTCTTCTCATGCTCCGTGATGCATGA
GGCTCTGCACAACCACTACACACAGAAGTCCCTCTCCCTGTCTCTGGGTAAATGA
Heavy Chain Amino Acid Sequence
(SEQ ID NO: 215)
EVQLVESGGGLAQPGGSLKLSCEASGFTFSDSAMHWVRQASGKGLEWVGRIRSKANSYATGYAASVKGRFTISRDDSKNM
AFLEMNSLKTEDTAVYYCLRQTYGDPWGQGTLVTVSSASTKGPSVFPLAPCSRSTSESTAALGCLVKDYFPEPVTVSWNS
GALTSGVHTFPAVLQSSGLYSLSSVVTVPSSSLGTKTYTCNVDHKPSNTKVDKRVESKYGPPCPPCPAPEFLGGPSVFLF
PPKPKDTLMISRTPEVTCVVVDVSQEDPEVQFNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK*
Light Chain Nucleotide Sequence
(SEQ ID NO: 216)
GATGTTGTGATGACTCAGTCTCCACTCTCCCTGTCCGTCACCCTTGGACAGCCGGCCTCCATCTCCTGCAGGTCTAGTCT
AAGCCTCGTATACAGTGATGGAAACAACTACTTGAATTGGTTTCAGCAGAGGCCAGGCCAATCTCCAAGGCGCCTACTTT
ATAAAGTTTTTAACCGGGACTCTGGGGTCCCAGACAGATTCAGCGGCAGTGGGTCAGGCACTGATTTCACACTGAAAATC
AGCAGGGTGGAGGCTGAGGATGTTGGGGTTTATTACTGCATGCAAGGAACACACTGGCCGTGGACGTTCGGCCAAGGGAC
CAAGGTGGAAATCAAACGAACTGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGGTGGATAACGCCCTCCAA
TCGGGTAACTCCCAGGAGAGTGTCACAGAGCAGGACAGCAAGGACAGCACCTACAGCCTCAGCAGCACCCTGACGCTGAG
CAAAGCAGACTACGAGAAACACAAAGTCTACGCCTGCGAAGTCACCCATCAGGGCCTGAGCTCGCCCGTCACAAAGAGCT
TCAACAGGGGAGAGTGTTAG
Light Chain Amino Acid Sequence
(SEQ ID NO: 217)
DVVMTQSPLSLSVTLGQPASISCRSSLSLVYSDGNNYLNWFQQRPGQSPRRLLYKVFNRDSGVPDRFSGSGSGTDFTLKI
SRVEAEDVGVYYCMQGTHWPWTFGQGTKVEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC*
H4H30095P2
HCVR Nucleotide Sequence
(SEQ ID NO: 218)
CAGGTTCAGCTGGTGCAGTCTGGAGTTGAGGTGAAGAAGCCTGGGGCCTCAGTGAAGGTCTCCTGCAAGGCTTCTGGTTA
CACCTTTAAGTTTTATGGTATCAGTTGGGTGCGACAGGCCCCTGGACAAGGGCTTGAGTGGATGGGCTGGATCAGTGTTT
ACAATGGTAAAACAAAGTATGCACAGAAGCTCCAGGGCAGAGTCACCATGACAACAGACACATCCACGAGCACAGCCTAC
ATGGAGCTGAGGAGCCTGAGATCTGACGACACGGCCGTGTATTTTTGTGCGAGAGATGGGGACTATGAGAGTAGTGGTTA
TCCGTTTGACTACTGGGGCCAGGGAACCCTGGTCACCGTCTCCTCA
HCVR Amino Acid Sequence
(SEQ ID NO: 219)
QVQLVQSGVEVKKPGASVKVSCKASGYTFKFYGISWVRQAPGQGLEWMGWISVYNGKTKYAQKLQGRVTMTTDTSTSTAY
MELRSLRSDDTAVYFCARDGDYESSGYPFDYWGQGTLVTVSS
HCDR1 Nucleotide Sequence
(SEQ ID NO: 220)
GGT TAC ACC TTT AAG TTT TAT GGT
HCDR1 Amino Acid Sequence
(SEQ ID NO: 221)
G Y T F K F Y G
HCDR2 Nucleotide Sequence
(SEQ ID NO: 222)
ATC AGT GTT TAC AAT GGT AAA ACA
HCDR2 Amino Acid Sequence
(SEQ ID NO: 223)
I S V Y N G K T
HCDR3 Nucleotide Sequence
(SEQ ID NO: 224)
GCG AGA GAT GGG GAC TAT GAG AGT AGT GGT TAT CCG TTT GAC TAC
HCDR3 Amino Acid Sequence
(SEQ ID NO: 225)
A R D G D Y E S S G Y P F D Y
LCVR Nucleotide Sequence
(SEQ ID NO: 226)
GACATCCAGATGACCCAGTCTCCATCCTCCCTGTCTGCATCTGTAGGAGACAGAGTCACCATCACTTGCCGGGCAAGTCA
GAGCATTAGCAGCTATTTAAATTGGTATCAGCAGAAACCAGGGAAAGCCCCTAAGCTCCTGATCTATGCTGCATCCAGTT
TGCAAAGTGGGGTCCCGTCAAGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGCCAAGGGACACGACTGGAGAT
TAAA
LCVR Amino Acid Sequence
(SEQ ID NO: 227)
DIQMTQSPSSLSASVGDRVTITCRASQSISSYLNWYQQKPGKAPKLLIYAASSLQSGVPSRESGSGSGTDFTLTISSLQP
EDFATYYCQQSYSTPPITFGQGTRLEIK
LCDR1 Nucleotide Sequence
(SEQ ID NO: 31)
CAG AGC ATT AGC AGT TAT
LCDR1 Amino Acid Sequence
(SEQ ID NO: 32)
Q S I S S Y
LCDR2 Nucleotide Sequence
(SEQ ID NO: 33)
GCT GCA TCC
LCDR2 Amino Acid Sequence
(SEQ ID NO: 34)
A A S
LCDR3 Nucleotide Sequence
(SEQ ID NO: 75)
CAA CAG AGT TAC AGT ACC CCT CCG ATC ACC
LCDR3 Amino Acid Sequence
(SEQ ID NO: 76)
Q Q S Y S T P P I T
Heavy Chain Nucleotide Sequence
(SEQ ID NO: 228)
CAGGTTCAGCTGGTGCAGTCTGGAGTTGAGGTGAAGAAGCCTGGGGCCTCAGTGAAGGTCTCCTGCAAGGCTTCTGGTTA
CACCTTTAAGTTTTATGGTATCAGTTGGGTGCGACAGGCCCCTGGACAAGGGCTTGAGTGGATGGGCTGGATCAGTGTTT
ACAATGGTAAAACAAAGTATGCACAGAAGCTCCAGGGCAGAGTCACCATGACAACAGACACATCCACGAGCACAGCCTAC
ATGGAGCTGAGGAGCCTGAGATCTGACGACACGGCCGTGTATTTTTGTGCGAGAGATGGGGACTATGAGAGTAGTGGTTA
TCCGTTTGACTACTGGGGCCAGGGAACCCTGGTCACCGTCTCCTCAGCCTCCACCAAGGGCCCATCGGTCTTCCCCCTGG
CGCCCTGCTCCAGGAGCACCTCCGAGAGCACAGCCGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTACAGTCCTCAGGACTCTACTCCCT
CAGCAGCGTGGTGACCGTGCCCTCCAGCAGCTTGGGCACGAAGACCTACACCTGCAACGTAGATCACAAGCCCAGCAACA
CCAAGGTGGACAAGAGAGTTGAGTCCAAATATGGTCCCCCATGCCCACCCTGCCCAGCACCTGAGTTCCTGGGGGGACCA
TCAGTCTTCCTGTTCCCCCCAAAACCCAAGGACACTCTCATGATCTCCCGGACCCCTGAGGTCACGTGCGTGGTGGTGGA
CGTGAGCCAGGAAGACCCCGAGGTCCAGTTCAACTGGTACGTGGATGGCGTGGAGGTGCATAATGCCAAGACAAAGCCGC
GGGAGGAGCAGTTCAACAGCACGTACCGTGTGGTCAGCGTCCTCACCGTCCTGCACCAGGACTGGCTGAACGGCAAGGAG
TACAAGTGCAAGGTCTCCAACAAAGGCCTCCCGTCCTCCATCGAGAAAACCATCTCCAAAGCCAAAGGGCAGCCCCGAGA
GCCACAGGTGTACACCCTGCCCCCATCCCAGGAGGAGATGACCAAGAACCAGGTCAGCCTGACCTGCCTGGTCAAAGGCT
TCTACCCCAGCGACATCGCCGTGGAGTGGGAGAGCAATGGGCAGCCGGAGAACAACTACAAGACCACGCCTCCCGTGCTG
GACTCCGACGGCTCCTTCTTCCTCTACAGCAGGCTCACCGTGGACAAGAGCAGGTGGCAGGAGGGGAATGTCTTCTCATG
CTCCGTGATGCATGAGGCTCTGCACAACCACTACACACAGAAGTCCCTCTCCCTGTCTCTGGGTAAATGA
Heavy Chain Amino Acid Sequence
(SEQ ID NO: 229)
QVQLVQSGVEVKKPGASVKVSCKASGYTFKFYGISWVRQAPGOGLEWMGWISVYNGKTKYAQKLQGRVTMTTDTSTSTAY
MELRSLRSDDTAVYFCARDGDYESSGYPFDYWGQGTLVTVSSASTKGPSVFPLAPCSRSTSESTAALGCLVKDYFPEPVT
VSWNSGALTSGVHTFPAVLQSSGLYSLSSVVTVPSSSLGTKTYTCNVDHKPSNTKVDKRVESKYGPPCPPCPAPEFLGGP
SVFLFPPKPKDTLMISRTPEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWESNGQPENNYKTTPPVL
DSDGSFFLYSRLTVDKSRWQEGNVFSCSVMHEALHNHYTQKSLSLSLGK*
Light Chain Nucleotide Sequence
(SEQ ID NO: 230)
GACATCCAGATGACCCAGTCTCCATCCTCCCTGTCTGCATCTGTAGGAGACAGAGTCACCATCACTTGCCGGGCAAGTCA
GAGCATTAGCAGCTATTTAAATTGGTATCAGCAGAAACCAGGGAAAGCCCCTAAGCTCCTGATCTATGCTGCATCCAGTT
TGCAAAGTGGGGTCCCGTCAAGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGCCAAGGGACACGACTGGAGAT
TAAACGAACTGTGGCTGCACCATCTGTCTTCATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAGGTGGATAACGCCCTCCAATCGGGTAACTCC
CAGGAGAGTGTCACAGAGCAGGACAGCAAGGACAGCACCTACAGCCTCAGCAGCACCCTGACGCTGAGCAAAGCAGACTA
CGAGAAACACAAAGTCTACGCCTGCGAAGTCACCCATCAGGGCCTGAGCTCGCCCGTCACAAAGAGCTTCAACAGGGGAG
AGTGTTAG
Light Chain Amino Acid Sequence
(SEQ ID NO: 231)
DIQMTQSPSSLSASVGDRVTITCRASQSISSYLNWYQQKPGKAPKLLIYAASSLQSGVPSRFSGSGSGTDFTLTISSLQP
EDFATYYCQQSYSTPPITFGQGTRLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC*

The present invention provides an isolated antibody or antigen-binding fragment thereof that specifically binds to FGFR3 (e.g., monomeric or dimeric human FGFR3b) or an antigenic fragment thereof comprising: a heavy chain variable region (HCVR) that comprises the HCDR1, HCDR2 and HCDR3 of a HCVR that comprises the amino acid sequence set forth in SEQ ID NO: 2, 22, 42, 62, 82, 102, 122, 140, 159, 169, 179, 199 or 219 (e.g., fused to an IgG4 Fc having a S108P mutation), and a light chain variable region (LCVR) that comprises the LCDR1, LCDR2 and LCDR3 of a LCVR that comprises the amino acid sequence set forth in SEQ ID NO: 10, 30, 50, 70, 90, 110, 130, 148, 187, 207, or 227.

The present invention also provides an isolated antibody or antigen-binding fragment thereof that specifically binds to FGFR3 (e.g., monomeric or dimeric human FGFR3b) or an antigenic fragment thereof comprising: (a) a heavy chain variable region (HCVR) that comprises the HCDR1, HCDR2 and HCDR3 of a HCVR that comprises the amino acid sequence set forth in SEQ ID NO: 2, and a light chain variable region (LCVR) that comprises the LCDR1, LCDR2 and LCDR3 of a LCVR that comprises the amino acid sequence set forth in SEQ ID NO: 10, (b) a heavy chain variable region (HCVR) that comprises the HCDR1, HCDR2 and HCDR3 of a HCVR that comprises the amino acid sequence set forth in SEQ ID NO: 22, and a light chain variable region (LCVR) that comprises the LCDR1, LCDR2 and LCDR3 of a LCVR that comprises the amino acid sequence set forth in SEQ ID NO: 30, (c) a heavy chain variable region (HCVR) that comprises the HCDR1, HCDR2 and HCDR3 of a HCVR that comprises the amino acid sequence set forth in SEQ ID NO: 42, and a light chain variable region (LCVR) that comprises the LCDR1, LCDR2 and LCDR3 of a LCVR that comprises the amino acid sequence set forth in SEQ ID NO: 50, (d) a heavy chain variable region (HCVR) that comprises the HCDR1, HCDR2 and HCDR3 of a HCVR that comprises the amino acid sequence set forth in SEQ ID NO: 62, and a light chain variable region (LCVR) that comprises the LCDR1, LCDR2 and LCDR3 of a LCVR that comprises the amino acid sequence set forth in SEQ ID NO: 70, (e) a heavy chain variable region (HCVR) that comprises the HCDR1, HCDR2 and HCDR3 of a HCVR that comprises the amino acid sequence set forth in SEQ ID NO: 82, and a light chain variable region (LCVR) that comprises the LCDR1, LCDR2 and LCDR3 of a LCVR that comprises the amino acid sequence set forth in SEQ ID NO: 90, (f) a heavy chain variable region (HCVR) that comprises the HCDR1, HCDR2 and HCDR3 of a HCVR that comprises the amino acid sequence set forth in SEQ ID NO: 102 (e.g., fused to an IgG4 Fc having a S108P mutation), and a light chain variable region (LCVR) that comprises the LCDR1, LCDR2 and LCDR3 of a LCVR that comprises the amino acid sequence set forth in SEQ ID NO: 110, and/or (g) a heavy chain variable region (HCVR) that comprises the HCDR1, HCDR2 and HCDR3 of a HCVR that comprises the amino acid sequence set forth in SEQ ID NO: 122, and a light chain variable region (LCVR) that comprises the LCDR1, LCDR2 and LCDR3 of a LCVR that comprises the amino acid sequence set forth in SEQ ID NO: 130, (h) a heavy chain variable region (HCVR) that comprises the HCDR1, HCDR2 and HCDR3 of a HCVR that comprises the amino acid sequence set forth in SEQ ID NO: 140, and a light chain variable region (LCVR) that comprises the LCDR1, LCDR2 and LCDR3 of a LCVR that comprises the amino acid sequence set forth in SEQ ID NO: 148, (i) a heavy chain variable region (HCVR) that comprises the HCDR1, HCDR2 and HCDR3 of a HCVR that comprises the amino acid sequence set forth in SEQ ID NO: 159, and a light chain variable region (LCVR) that comprises the LCDR1, LCDR2 and LCDR3 of a LCVR that comprises the amino acid sequence set forth in SEQ ID NO: 148, (j) a heavy chain variable region (HCVR) that comprises the HCDR1, HCDR2 and HCDR3 of a HCVR that comprises the amino acid sequence set forth in SEQ ID NO: 169, and a light chain variable region (LCVR) that comprises the LCDR1, LCDR2 and LCDR3 of a LCVR that comprises the amino acid sequence set forth in SEQ ID NO: 148, (k) a heavy chain variable region (HCVR) that comprises the HCDR1, HCDR2 and HCDR3 of a HCVR that comprises the amino acid sequence set forth in SEQ ID NO: 179, and a light chain variable region (LCVR) that comprises the LCDR1, LCDR2 and LCDR3 of a LCVR that comprises the amino acid sequence set forth in SEQ ID NO: 187, (l) a heavy chain variable region (HCVR) that comprises the HCDR1, HCDR2 and HCDR3 of a HCVR that comprises the amino acid sequence set forth in SEQ ID NO: 199, and a light chain variable region (LCVR) that comprises the LCDR1, LCDR2 and LCDR3 of a LCVR that comprises the amino acid sequence set forth in SEQ ID NO: 207, and/or (m) a heavy chain variable region (HCVR) that comprises the HCDR1, HCDR2 and HCDR3 of a HCVR that comprises the amino acid sequence set forth in SEQ ID NO: 219, and a light chain variable region (LCVR) that comprises the LCDR1, LCDR2 and LCDR3 of a LCVR that comprises the amino acid sequence set forth in SEQ ID NO: 227.

The present invention also provides an isolated antibody or antigen-binding fragment thereof that specifically binds to FGFR3 (e.g., monomeric or dimeric human FGFR3b) or an antigenic fragment thereof comprising: (a) a heavy chain variable region that comprises an HCDR1 that comprises the amino acid sequence set forth in SEQ ID NO: 4, an HCDR2 that comprises the amino acid sequence set forth in SEQ ID NO: 6, and an HCDR3 that comprises the amino acid sequence set forth in SEQ ID NO: 8, and a light chain variable region that comprises an LCDR1 that comprises the amino acid sequence set forth in SEQ ID NO: 12, an LCDR2 that comprises the amino acid sequence set forth in SEQ ID NO: 14, and an LCDR3 that comprises the amino acid sequence set forth in SEQ ID NO: 16; (b) a heavy chain variable region that comprises an HCDR1 that comprises the amino acid sequence set forth in SEQ ID NO: 24, an HCDR2 that comprises the amino acid sequence set forth in SEQ ID NO: 26, and an HCDR3 that comprises the amino acid sequence set forth in SEQ ID NO: 28, and a light chain variable region that comprises an LCDR1 that comprises the amino acid sequence set forth in SEQ ID NO: 32, an LCDR2 that comprises the amino acid sequence set forth in SEQ ID NO: 34, and an LCDR3 that comprises the amino acid sequence set forth in SEQ ID NO: 36; (c) a heavy chain variable region that comprises an HCDR1 that comprises the amino acid sequence set forth in SEQ ID NO: 44, an HCDR2 that comprises the amino acid sequence set forth in SEQ ID NO: 46, and an HCDR3 that comprises the amino acid sequence set forth in SEQ ID NO: 48, and a light chain variable region that comprises an LCDR1 that comprises the amino acid sequence set forth in SEQ ID NO: 52, an LCDR2 that comprises the amino acid sequence set forth in SEQ ID NO: 54, and an LCDR3 that comprises the amino acid sequence set forth in SEQ ID NO: 56; (d) a heavy chain variable region that comprises an HCDR1 that comprises the amino acid sequence set forth in SEQ ID NO: 64, an HCDR2 that comprises the amino acid sequence set forth in SEQ ID NO: 66, and an HCDR3 that comprises the amino acid sequence set forth in SEQ ID NO: 68, and a light chain variable region that comprises an LCDR1 that comprises the amino acid sequence set forth in SEQ ID NO: 72, an LCDR2 that comprises the amino acid sequence set forth in SEQ ID NO: 74, and an LCDR3 that comprises the amino acid sequence set forth in SEQ ID NO: 76; (e) a heavy chain variable region that comprises an HCDR1 that comprises the amino acid sequence set forth in SEQ ID NO: 84, an HCDR2 that comprises the amino acid sequence set forth in SEQ ID NO: 86, and an HCDR3 that comprises the amino acid sequence set forth in SEQ ID NO: 88, and a light chain variable region that comprises an LCDR1 that comprises the amino acid sequence set forth in SEQ ID NO: 92, an LCDR2 that comprises the amino acid sequence set forth in SEQ ID NO: 94, and an LCDR3 that comprises the amino acid sequence set forth in SEQ ID NO: 96; (f) a heavy chain variable region that comprises an HCDR1 that comprises the amino acid sequence set forth in SEQ ID NO: 104, an HCDR2 that comprises the amino acid sequence set forth in SEQ ID NO: 106, and an HCDR3 that comprises the amino acid sequence set forth in SEQ ID NO: 108 (e.g., fused to an IgG4 Fc having a S108P mutation), and a light chain variable region that comprises an LCDR1 that comprises the amino acid sequence set forth in SEQ ID NO: 112, an LCDR2 that comprises the amino acid sequence set forth in SEQ ID NO: 114, and an LCDR3 that comprises the amino acid sequence set forth in SEQ ID NO: 116; and/or (g) a heavy chain variable region that comprises an HCDR1 that comprises the amino acid sequence set forth in SEQ ID NO: 124, an HCDR2 that comprises the amino acid sequence set forth in SEQ ID NO: 126, and an HCDR3 that comprises the amino acid sequence set forth in SEQ ID NO: 128, and a light chain variable region that comprises an LCDR1 that comprises the amino acid sequence set forth in SEQ ID NO: 132, an LCDR2 that comprises the amino acid sequence set forth in SEQ ID NO: 34, and an LCDR3 that comprises the amino acid sequence set forth in SEQ ID NO: 134; (h) a heavy chain variable region that comprises an HCDR1 that comprises the amino acid sequence set forth in SEQ ID NO: 142, an HCDR2 that comprises the amino acid sequence set forth in SEQ ID NO: 144, and an HCDR3 that comprises the amino acid sequence set forth in SEQ ID NO: 146, and a light chain variable region that comprises an LCDR1 that comprises the amino acid sequence set forth in SEQ ID NO: 150, an LCDR2 that comprises the amino acid sequence set forth in SEQ ID NO: 14, and an LCDR3 that comprises the amino acid sequence set forth in SEQ ID NO: 153; (i) a heavy chain variable region that comprises an HCDR1 that comprises the amino acid sequence set forth in SEQ ID NO: 161, an HCDR2 that comprises the amino acid sequence set forth in SEQ ID NO: 163, and an HCDR3 that comprises the amino acid sequence set forth in SEQ ID NO: 165, and a light chain variable region that comprises an LCDR1 that comprises the amino acid sequence set forth in SEQ ID NO: 150, an LCDR2 that comprises the amino acid sequence set forth in SEQ ID NO: 14, and an LCDR3 that comprises the amino acid sequence set forth in SEQ ID NO: 153; (j) a heavy chain variable region that comprises an HCDR1 that comprises the amino acid sequence set forth in SEQ ID NO: 171, an HCDR2 that comprises the amino acid sequence set forth in SEQ ID NO: 173, and an HCDR3 that comprises the amino acid sequence set forth in SEQ ID NO: 175, and a light chain variable region that comprises an LCDR1 that comprises the amino acid sequence set forth in SEQ ID NO: 150, an LCDR2 that comprises the amino acid sequence set forth in SEQ ID NO: 14, and an LCDR3 that comprises the amino acid sequence set forth in SEQ ID NO: 153; (k) a heavy chain variable region that comprises an HCDR1 that comprises the amino acid sequence set forth in SEQ ID NO: 181, an HCDR2 that comprises the amino acid sequence set forth in SEQ ID NO: 183, and an HCDR3 that comprises the amino acid sequence set forth in SEQ ID NO: 185, and a light chain variable region that comprises an LCDR1 that comprises the amino acid sequence set forth in SEQ ID NO: 189, an LCDR2 that comprises the amino acid sequence set forth in SEQ ID NO: 191, and an LCDR3 that comprises the amino acid sequence set forth in SEQ ID NO: 193; (l) a heavy chain variable region that comprises an HCDR1 that comprises the amino acid sequence set forth in SEQ ID NO: 201, an HCDR2 that comprises the amino acid sequence set forth in SEQ ID NO: 203, and an HCDR3 that comprises the amino acid sequence set forth in SEQ ID NO: 205, and a light chain variable region that comprises an LCDR1 that comprises the amino acid sequence set forth in SEQ ID NO: 209, an LCDR2 that comprises the amino acid sequence set forth in SEQ ID NO: 211, and an LCDR3 that comprises the amino acid sequence set forth in SEQ ID NO: 213; and/or (m) a heavy chain variable region that comprises an HCDR1 that comprises the amino acid sequence set forth in SEQ ID NO: 221, an HCDR2 that comprises the amino acid sequence set forth in SEQ ID NO: 223, and an HCDR3 that comprises the amino acid sequence set forth in SEQ ID NO: 225, and a light chain variable region that comprises an LCDR1 that comprises the amino acid sequence set forth in SEQ ID NO: 32, an LCDR2 that comprises the amino acid sequence set forth in SEQ ID NO: 34, and an LCDR3 that comprises the amino acid sequence set forth in SEQ ID NO: 76.

The present invention further provides an isolated antibody or antigen-binding fragment thereof that specifically binds to FGFR3 or an antigenic fragment thereof comprising a heavy chain variable region that comprises the amino acid sequence set forth in SEQ ID NO: 2, 22, 42, 62, 82, 102 (e.g., fused to an IgG4 Fc having a S108P mutation), 122, 140, 159, 169, 179, 199 or 219, and a light chain variable region that comprises the amino acid sequence set forth in SEQ ID NO: 10, 30, 50, 70, 90, 110, 130, 148, 187, 207, or 227.

In addition, the present invention provides an isolated antibody or antigen-binding fragment thereof that specifically binds to FGFR3 (e.g., monomeric or dimeric human FGFR3b) or an antigenic fragment thereof comprising: (a) a heavy chain variable region that comprises the amino acid sequence set forth in SEQ ID NO: 2, and a light chain variable region that comprises the amino acid sequence set forth in SEQ ID NO: 10; (b) a heavy chain variable region that comprises the amino acid sequence set forth in SEQ ID NO: 22, and a light chain variable region that comprises the amino acid sequence set forth in SEQ ID NO: 30; (c) a heavy chain variable region that comprises the amino acid sequence set forth in SEQ ID NO: 42, and a light chain variable region that comprises the amino acid sequence set forth in SEQ ID NO: 50; (d) a heavy chain variable region that comprises the amino acid sequence set forth in SEQ ID NO: 62, and a light chain variable region that comprises the amino acid sequence set forth in SEQ ID NO: 70; (e) a heavy chain variable region that comprises the amino acid sequence set forth in SEQ ID NO: 82, and a light chain variable region that comprises the amino acid sequence set forth in SEQ ID NO: 90; (f) a heavy chain variable region that comprises the amino acid sequence set forth in SEQ ID NO: 102, and a light chain variable region that comprises the amino acid sequence set forth in SEQ ID NO: 110; e.g., wherein the heavy chain variable region is fused to an IgG4 Fc having a S108P mutation; (g) a heavy chain variable region that comprises the amino acid sequence set forth in SEQ ID NO: 122, and a light chain variable region that comprises the amino acid sequence set forth in SEQ ID NO: 130; (h) a heavy chain variable region that comprises the amino acid sequence set forth in SEQ ID NO: 140, and a light chain variable region that comprises the amino acid sequence set forth in SEQ ID NO: 148; (i) a heavy chain variable region that comprises the amino acid sequence set forth in SEQ ID NO: 159, and a light chain variable region that comprises the amino acid sequence set forth in SEQ ID NO: 148; (j) a heavy chain variable region that comprises the amino acid sequence set forth in SEQ ID NO: 169, and a light chain variable region that comprises the amino acid sequence set forth in SEQ ID NO: 148; (k) a heavy chain variable region that comprises the amino acid sequence set forth in SEQ ID NO: 179, and a light chain variable region that comprises the amino acid sequence set forth in SEQ ID NO: 187; (I) a heavy chain variable region that comprises the amino acid sequence set forth in SEQ ID NO: 199, and a light chain variable region that comprises the amino acid sequence set forth in SEQ ID NO: 207; and/or (m) a heavy chain variable region that comprises the amino acid sequence set forth in SEQ ID NO: 219, and a light chain variable region that comprises the amino acid sequence set forth in SEQ ID NO: 227.

The present invention provides an isolated antibody or antigen-binding fragment thereof that specifically binds to FGFR3 (e.g., monomeric or dimeric human FGFR3b) or an antigenic fragment thereof comprising (a) a heavy chain that comprises the amino acid sequence set forth in SEQ ID NO: 18, 38, 58, 78, 98, 118, 136, 155, 167, 177, 195, 215 or 229, and a light chain that comprises the amino acid sequence set forth in SEQ ID NO: 20, 40, 60, 80, 100, 120,138, 157, 197, 217, or 231.

The present invention also provides an isolated antibody or antigen-binding fragment thereof that specifically binds to FGFR3 or an antigenic fragment thereof comprising: (a) a heavy chain that comprises the amino acid sequence set forth in SEQ ID NO: 18, and a light chain that comprises the amino acid sequence set forth in SEQ ID NO: 20; (b) a heavy chain variable region that comprises the amino acid sequence set forth in SEQ ID NO: 38, and a light chain variable region that comprises the amino acid sequence set forth in SEQ ID NO: 40; (c) a heavy chain variable region that comprises the amino acid sequence set forth in SEQ ID NO: 58, and a light chain variable region that comprises the amino acid sequence set forth in SEQ ID NO: 60; (d) a heavy chain variable region that comprises the amino acid sequence set forth in SEQ ID NO: 78, and a light chain variable region that comprises the amino acid sequence set forth in SEQ ID NO: 80; (e) a heavy chain variable region that comprises the amino acid sequence set forth in SEQ ID NO: 98, and a light chain variable region that comprises the amino acid sequence set forth in SEQ ID NO: 100; (f) a heavy chain variable region that comprises the amino acid sequence set forth in SEQ ID NO: 118, and a light chain variable region that comprises the amino acid sequence set forth in SEQ ID NO: 120; and/or (g) a heavy chain that comprises the amino acid sequence set forth in SEQ ID NO: 136, and a light chain that comprises the amino acid sequence set forth in SEQ ID NO: 138; (h) a heavy chain that comprises the amino acid sequence set forth in SEQ ID NO: 155, and a light chain that comprises the amino acid sequence set forth in SEQ ID NO: 157; (i) a heavy chain that comprises the amino acid sequence set forth in SEQ ID NO: 167, and a light chain that comprises the amino acid sequence set forth in SEQ ID NO: 157; (j) a heavy chain that comprises the amino acid sequence set forth in SEQ ID NO: 177, and a light chain that comprises the amino acid sequence set forth in SEQ ID NO: 157; (k) a heavy chain that comprises the amino acid sequence set forth in SEQ ID NO: 195, and a light chain that comprises the amino acid sequence set forth in SEQ ID NO: 197; (l) a heavy chain that comprises the amino acid sequence set forth in SEQ ID NO: 215, and a light chain that comprises the amino acid sequence set forth in SEQ ID NO: 217; and/or (m) a heavy chain that comprises the amino acid sequence set forth in SEQ ID NO: 229, and a light chain that comprises the amino acid sequence set forth in SEQ ID NO: 231.

In an embodiment of the invention an anti-FGFR3 antigen-binding protein, such as an antibody or antigen-binding fragment thereof, has one or more of the following characteristics:
Binds (e.g., in a surface plasmon resonance assay) to monomeric human FGFR3b (e.g., tagged, for example, at the C-terminus, with myc-myc-His₆) at 25° C. with an affinity (K_{D}) of about 16 nM or greater affinity (e.g., about 16 nM, 12 nM, 10 nM, 7 nM, 5 nM, 4 nM, 3 nM, 2 nM, 1 nM, 0.5 nM, 0.22 nM, 0.2 nM, 0.19 nM, 0.14 nM, 0.1 nM);
Binds (e.g., in a surface plasmon resonance assay) to monomeric Cynomolgous monkey FGFR3b (e.g., tagged, for example, at the C-terminus, with myc-myc-His₆) at 25° C. with an affinity (K_{D}) of about 20 nM or greater affinity (e.g., about 20 nM, 16 nM, 15 nM, 10 nM, 8 nM, 5 nM, 4 nM, 3 nM, 2 nM, 1 nM, 0.9 nM, 0.65 nM, 0.3 nM, 0.28 nM, 0.2 nM, 0.15 nM, 0.1 nM);
Binds (e.g., in a surface plasmon resonance assay) monomeric murine FGFR3b (e.g., tagged, for example, at the C-terminus, with myc-myc-His₆) at 25° C. with an affinity (K_{D}) of about 70 nM or greater affinity (e.g., about 70 nM, 20 nM, 17 nM, 12 nM, 10 nM, 9 nM, 8 nM, 0.1 nM);
Does not bind significantly to monomeric human FGFR3c (e.g., tagged, for example, at the C-terminus, with myc-myc-His₆) at 25° C. (e.g., in a surface plasmon resonance assay);
Binds (e.g., in a surface plasmon resonance assay) dimeric human FGFR3b (e.g., tagged, for example, at the C-terminus, with a mouse Fc (mFc)) at 25° C. with an affinity of about 0.6 nM of greater affinity (e.g., about 0.58 nM, 0.17 nM, 0.11 nM, 0.04 nM, 0.03 nM, 0.02 nM, 0.01 nM, 0.023 nM, 0.061 nM, 0.031 nM, 0.016 nM, 0.034 nM, 0.027 nM);
Blocks about 68% or more binding of FGF1 acidic to human FGFR3b-mFc at 200 nM antibody (e.g., 90% or 05%);
Binds to monomeric Cynomolgous and monomeric mouse FGFR3b with a K_{D} that is within about 0.1 nM of the K_{D} for binding to monomeric or dimeric human FGFR3b;
Blocks binding of 4 nM human FGFR3b-mFc to human FGF1 acidic protein with an IC₅₀ of about 15 nM or a lower concentration (e.g., IC₅₀ of about 1, 2 or 3 nM);
Competes for binding to hFGFR3b.mmH with another anti-FGFR3 antibody as set forth in Table 3-1 herein;
Blocks intracellular signaling in an engineered IL-3-dependent Ba/F3 murine hematopoietic cell line genetically modified to stably express wild-type or S249C mutant human fibroblast growth factor receptor 3b that has been stimulated with human heparin, e.g., at about 5 micrograms/ml, and human FGF1 ligand, e.g., at about 1 nM, with an IC₅₀ of about 18 nM or a lower concentration (e.g., about 0.51 nM, 0.5 nM, 0.61 nM, 0.6 nM, or 0.012 nM);
Blocks intracellular signaling in an engineered IL-3-dependent Ba/F3 murine hematopoietic cell line genetically modified to stably express S249C mutant human fibroblast growth factor receptor 3b that has been stimulated with human heparin, e.g., at about 5 micrograms/ml, and human FGF1 ligand, e.g., at about 1 nM, with an IC₅₀ that is lower than for blocking intracellular signaling in an engineered IL-3-dependent Ba/F3 murine hematopoietic cell line genetically modified to stably express wild-type human fibroblast growth factor receptor 3b that has been stimulated with human FGF1 ligand, e.g., at about 1 nM, and human heparin, e.g., at about 5 micrograms/ml;
Blocks dimerization of FGFR3 (e.g., wild-type or S249C mutant), e.g., with stronger inhibition than that of REGN6331, for example, as measured in a non-reducing SDS-PAGE assay;
Reduces tumor size (e.g., bladder cancer tumor expressing FGFR3 (e.g., S249C mutant)) in a subject administered the antibody or fragment;
Reduces CD73 expression in a tumor (e.g., bladder cancer tumor expressing FGFR3 (e.g., S249C mutant)) in a subject administered the antibody or fragment;
Inhibits FGF1/heparin stimulation induced phosphorylation of MAPK, e.g., in a BaF3 cell expressing wild-type FGFR3 (e.g., S249C mutant);
Inhibits proliferation of UMUC14 bladder cancer cells expressing endogenous FGFR3 S249C mutation (e.g., in a cancer cell spheroid proliferation assay);
Inhibits tumor growth in a mouse (e.g., SCID mouse) xenograft model of bladder cancer cell line UMUC14 expressing FGFR3 (e.g., S249C mutation);
Inhibits CD73-dependent, adenosine-mediated inhibition of immune cell activation;
Increases the ratio of CD8/CD4 and/or CD8+/Tᵣₑg in tumor tissue having tumor cells expressing FGFR3 (e.g., S249C mutant);
Inhibits FGF3-mediated activation of CD73 expression and/or enzymatic activity (e.g., adenosine production) (e.g., on a tumor cell expressing FGFR3 (e.g., S249C mutant));
Inhibits FGFR3-dependent, adenosine-mediated immune cell suppression;
Inhibits growth of BaF3 cells expressing the FGFR3 double mutant S249C and V557M, or S249C and V557L, wherein the antibody or antigen-binding fragment comprises immunoglobulin chains including any of the amino acid sequences as set forth herein.

The present invention includes monoclonal anti-FGFR3 antigen-binding proteins, e.g., antibodies and antigen-binding fragments thereof (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2), as well as monoclonal compositions comprising a plurality of isolated monoclonal antigen-binding proteins. The term “monoclonal antibody” or “mAb”, as used herein, refers to a member of a population of substantially homogeneous antibodies, i.e., the antibody molecules comprising the population are identical in amino acid sequence except for possible naturally occurring mutations that may be present in minor amounts. A “plurality” of such monoclonal antibodies and fragments in a composition refers to a concentration of identical (i.e., as discussed above, in amino acid sequence except for possible naturally occurring mutations that may be present in minor amounts) antibodies and fragments which is above that which would normally occur in nature, e.g., in the blood of a host organism such as a mouse or a human.

In an embodiment of the invention, an anti-FGFR3 antigen-binding protein, e.g., antibody or antigen-binding fragment comprises a heavy chain constant domain, e.g., of the type IgA (e.g., IgA1 or IgA2), IgD, IgE, IgG (e.g., IgG1, IgG2, IgG3 and IgG4 (e.g., comprising a S228P and/or S108P mutation)) or IgM. In an embodiment of the invention, an antigen-binding protein, e.g., antibody or antigen-binding fragment, comprises a light chain constant domain, e.g., of the type kappa or lambda. The present invention includes antigen-binding proteins comprising the V_{H} and V_{L} variable domains set forth herein (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2) which are linked to a heavy and/or light chain constant domain, e.g., as set forth above.

The term “human” antigen-binding protein, such as an antibody or antigen-binding fragment, as used herein, includes antibodies and fragments having human amino acid sequence; for example, variable and constant regions derived from human germline immunoglobulin sequences whether in a human cell or grafted into a non-human cell, e.g., a mouse cell. See e.g., U.S. Pat. No. 8,502,018, 6,596,541 or 5,789,215. The human antibodies and antigen-binding fragments of the invention may, in an embodiment of the invention, include amino acid residues not encoded by human germline immunoglobulin sequences (e.g., having mutations introduced by random or site-specific mutagenesis in vitro or by somatic mutation in vivo), for example in the CDRs and, in particular, CDR3. However, the term “human antibody”, as used herein, is not intended to include mAbs in which CDR sequences derived from the germline of another mammalian species (e.g., mouse) have been grafted onto human FR sequences. The term includes antibodies recombinantly produced in a non-human mammal or in cells of a non-human mammal. The term is not intended to include antibodies isolated from or generated in a human subject. The present invention includes human antigen-binding proteins (e.g., antibodies or antigen-binding fragments thereof such as H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2).

The present invention includes anti-FGFR3 chimeric antigen-binding proteins, e.g., antibodies and antigen-binding fragments thereof, and methods of use thereof. As used herein, a “chimeric antibody” is an antibody having the variable domain from a first antibody and the constant domain from a second antibody, where the first and second antibodies are from different species. (see e.g., U.S. Pat. No. 4,816,567; and Morrison et al., (1984) Proc. Natl. Acad. Sci. USA 81: 6851-6855). The present invention includes chimeric antibodies comprising the variable domains which are set forth herein (e.g., from H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2) and a non-human constant domain.

The term “recombinant” antigen-binding proteins, such as antibodies or antigen-binding fragments thereof, refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a host cell (e.g., Chinese hamster ovary (CHO) cell) or cellular expression system or isolated from a recombinant combinatorial human antibody library. The present invention includes recombinant antigen-binding proteins, such as antibodies and antigen-binding fragments as set forth herein (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2).

An antigen-binding fragment of an antibody will, in an embodiment of the invention, comprise less than a full antibody but still binds specifically to antigen, e.g., FGFR3, e.g., including at least one variable domain. The variable domain may be of any size or amino acid composition and will generally comprise at least one (e.g., 3) CDR(s), which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} and/or V_{L} domain which are bound non-covalently.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-CH1; (ii) V_{H}-CH2; (iii) V_{H}-CH3; (iv) V_{H}-CH1-CH2; (v) V_{H}-CH1-CH2-CH3; (vi) V_{H}-CH2-CH3; (vii) V_{H}-CL; (viii) V_{L}-CH1; (ix) V_{L}-CH2; (x) V_{L}-CH3; (xi) V_{L}-CH1-CH2; (xii) V_{L}-CH1-CH2-CH3; (xiii) V_{L}-CH2-CH3; and (xiv) V_{L}-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)). The present invention includes an antigen-binding fragment of an antigen-binding protein such as an antibody set forth herein, for example, H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2.

Antigen-binding proteins (e.g., antibodies and antigen-binding fragments) may be monospecific or multi-specific (e.g., bispecific). Multispecific antigen-binding proteins are discussed further herein. The present invention includes monospecific as well as multispecific (e.g., bispecific) antigen-binding fragments comprising one or more variable domains from an antigen-binding protein that is specifically set forth herein (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2).

In some embodiments, an antigen-binding fragment described herein may be an scFv. An scFv (single chain fragment variable) has variable regions of heavy (V_{H}) and light (V_{L}) domains (in either order), which, preferably, are joined together by a flexible linker (e.g., peptide linker). The length of the flexible linker used to link both of the V regions may be important for yielding the correct folding of the polypeptide chain. Previously, it has been estimated that the peptide linker must span 3.5 nm (35 Å) between the carboxy terminus of the variable domain and the amino terminus of the other domain without affecting the ability of the domains to fold and form an intact antigen-binding site (Huston et al., Protein engineering of single-chain Fv analogs and fusion proteins. Methods in Enzymology. 1991; 203:46-88). In an embodiment, the linker comprises an amino acid sequence of such length to separate the variable domains by about 3.5 nm. In an embodiment, an anti-FGFR3 scFv comprises the arrangement of variable regions as follows LCVR-HCVR or HCVR-LCVR, wherein the HCVR and LCVR are optionally connected by a linker.

In some embodiments, an antigen-binding fragment described herein may be a Fab.

In some embodiments, an antigen-binding fragment described herein may be a bivalent antibody.

In some embodiments, an anti-FGFR3 antibody described herein comprises a monovalent or “one-armed” antibody. The monovalent or “one-armed” antibodies as used herein refer to immunoglobulin proteins comprising a single variable domain. For example, the one-armed antibody may comprise a single variable domain within a Fab wherein the Fab is linked to at least one Fc fragment. In certain embodiments, the one-armed antibody comprises: (i) a heavy chain comprising a heavy chain constant region and a heavy chain variable region, (ii) a light chain comprising a light chain constant region and a light chain variable region, and (iii) a polypeptide comprising a Fc fragment or a truncated heavy chain. In certain embodiments, the Fc fragment or a truncated heavy chain comprised in the separate polypeptide is a “dummy Fc,” which refers to an Fc fragment that is not linked to an antigen binding domain. The one-armed antibodies described herein may comprise any of the HCVR/LCVR pairs or CDR amino acid sequences as set forth in Table 1-1 herein. One-armed antibodies comprising a full-length heavy chain, a full-length light chain and an additional Fc domain polypeptide can be constructed using standard methodologies (see e.g., WO2010151792, which is incorporated herein by reference in its entirety), wherein the heavy chain constant region differs from the Fc domain polypeptide by at least two amino acids (e.g., H95R and Y96F according to the IMGT exon numbering system, or H435R and Y436F according to the EU numbering system). Such modifications are useful in purification of the monovalent antibodies (see WO2010151792).

The term “specifically binds” or “binds specifically” refers to those antigen-binding proteins (e.g., antibodies or antigen-binding fragments thereof) having a binding affinity to an antigen, such as human FGFR3 protein (e.g., FGFR3b isoform), mouse FGFR3 protein (e.g., FGFR3b isoform) or cynomolgous monkey FGFR3 protein (e.g., FGFR3b isoform), expressed as K_{D}, of at least about 10⁻⁹ M (e.g., 0.01, 0.1, 0.2, 0.3, 0.4, 0.5, 0.6, 0.7, 0.8, 0.9 or 1.0 nM), as measured by real-time, label free bio-layer interferometry assay, for example, at 25° C. or 37° C., e.g., an Octet® HTX biosensor, or by surface plasmon resonance, e.g., BIACORE™, or by solution-affinity ELISA. The present invention includes antigen-binding proteins that specifically bind to FGFR3 protein (e.g., FGFR3b isoform). “Anti-FGFR3” refers to an antigen-binding protein (or other molecule), for example an antibody or antigen-binding fragment thereof, that binds specifically to FGFR3 (e.g., FGFR3b isoform).

The present invention includes antigen-binding proteins, e.g., antibodies or antigen-binding fragments, that bind to the same epitope as an antigen-binding protein of the present invention (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2).

An antigen is a molecule, such as a peptide (e.g., FGFR3 or a fragment thereof (an antigenic fragment)), to which, for example, an antibody or antigen-binding fragment thereof binds. The specific region on an antigen that an antibody recognizes and binds to is called the epitope. Antigen-binding proteins (e.g., antibodies) of the present invention that specifically bind to such antigens are part of the present invention.

The term “epitope” refers to an antigenic determinant (e.g., on FGFR3b) that interacts with a specific antigen-binding site of an antigen-binding protein, e.g., a variable region of an antibody, known as a paratope. A single antigen may have more than one epitope. Thus, different antibodies may bind to different areas on an antigen and may have different biological effects. The term “epitope” may also refer to a site on an antigen to which B and/or T cells respond and/or to a region of an antigen that is bound by an antibody. Epitopes may be defined as structural or functional. Functional epitopes are generally a subset of the structural epitopes and have those residues that directly contribute to the affinity of the interaction. Epitopes may be linear or conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes to which antigen-binding proteins of the present invention bind may be included in fragments of FGFR3, e.g., human FGFR3b, for example the extracellular domain thereof. Antigen-binding proteins (e.g., antibodies) of the present invention that bind to such epitopes are part of the present invention.

Methods for determining the epitope of an antigen-binding protein, e.g., antibody or fragment or polypeptide, include alanine scanning mutational analysis, peptide blot analysis (Reineke (2004) Methods Mol. Biol. 248: 443-63), peptide cleavage analysis, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antigen-binding protein (e.g., antibody or fragment or polypeptide) interacts is hydrogen/deuterium exchange detected by mass spectrometry. See, e.g., Ehring (1999) Analytical Biochemistry 267: 252-259; Engen and Smith (2001) Anal. Chem. 73: 256A-265A.

The present invention includes antigen-binding proteins that compete for binding to FGFR3, e.g., an FGFR3b epitope as discussed herein, with an antigen-binding protein of the present invention, e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2. The term “competes” as used herein, refers to an antigen-binding protein (e.g., antibody or antigen-binding fragment thereof) that binds to an antigen (e.g., FGFR3) and inhibits or blocks the binding of another antigen-binding protein (e.g., antibody or antigen-binding fragment thereof) to the antigen. Unless otherwise stated, the term also includes competition between two antigen-binding proteins e.g., antibodies, in both orientations, i.e., a first antibody that binds antigen and blocks binding by a second antibody and vice versa. Thus, in an embodiment of the invention, competition occurs in one such orientation. In certain embodiments, the first antigen-binding protein (e.g., antibody) and second antigen-binding protein (e.g., antibody) may bind to the same epitope. Alternatively, the first and second antigen-binding proteins (e.g., antibodies) may bind to different, but, for example, overlapping or non-overlapping epitopes, wherein binding of one inhibits or blocks the binding of the second antibody, e.g., via steric hindrance. Competition between antigen-binding proteins (e.g., antibodies) may be measured by methods known in the art, for example, by a real-time, label-free bio-layer interferometry assay. Also, binding competition between anti-FGFR3 antigen-binding proteins (e.g., monoclonal antibodies (mAbs)) can be determined using a real time, label-free bio-layer interferometry assay on an Octet RED384 biosensor (Pall ForteBio Corp.).

Typically, an antibody or antigen-binding fragment of the invention which is modified in some way retains the ability to specifically bind to FGFR3 (e.g., FGFR3b), e.g., retains at least 10% of its FGFR3 binding activity (when compared to the parental antibody) when that activity is expressed on a molar basis. Preferably, an antibody or antigen-binding fragment of the invention retains at least 20%, 50%, 70%, 80%, 90%, 95% or 100% or more of the FGFR3 binding affinity as the parental antibody. It is also intended that an antibody or antigen-binding fragment of the invention may include conservative or non-conservative amino acid substitutions (referred to as “conservative variants” or “function conserved variants” of the antibody) that do not substantially alter its biologic activity.

A “variant” of a polypeptide, such as an immunoglobulin chain (e.g., an H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2 V_{H}, V_{L}, HC or LC or CDR thereof comprising the amino acid sequence specifically set forth herein), refers to a polypeptide comprising an amino acid sequence that is at least about 70-99.9% (e.g., at least 70, 72, 74, 75, 76, 79, 80, 81, 82, 83, 84, 85, 86, 87, 88, 89, 90, 91, 92, 93, 94, 95, 96, 97, 98, 99, 99.5 or 99.9%) identical or similar to a referenced amino acid sequence that is set forth herein (e.g., any of SEQ ID NOs: 2, 10, 18, 20, 22, 30, 38, 40, 42, 50, 58, 60, 62, 70, 78, 80, 82, 90, 98, 100, 102, 110, 118, 120, 122, 130, 136, 138, 140, 148, 155, 157, 159, 167, 169, 177, 179, 187, 195, 197, 199, 207, 215, 217, 219, 227, 229 or 231); when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment).

Moreover, a variant of a polypeptide may include a polypeptide such as an immunoglobulin chain (e.g., an H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2 V_{H}, V_{L}, HC or LC or CDR thereof) which may include the amino acid sequence of the reference polypeptide whose amino acid sequence is specifically set forth herein but for one or more (e.g., 1, 2, 3, 4, 5, 6, 7, 8, 9 or 10) mutations, e.g., one or more missense mutations (e.g., conservative substitutions), non-sense mutations, deletions, or insertions. See Table A. For example, the present invention includes anti-FGFR3 antigen-binding proteins which include an immunoglobulin light chain (or V_{L}) variant comprising the amino acid sequence set forth in SEQ ID NO: 10 but having one or more of such mutations and/or an immunoglobulin heavy chain (or V_{H}) variant comprising the amino acid sequence set forth in SEQ ID NO: 2 but having one or more of such mutations. In an embodiment of the invention, an anti-FGFR3 antigen-binding protein includes an immunoglobulin light chain variant comprising CDR-L1, CDR-L2 and CDR-L3 wherein one or more (e.g., 1 or 2 or 3) of such CDRs has one or more of such mutations (e.g., conservative substitutions) and/or an immunoglobulin heavy chain variant comprising CDR-H1, CDR-H2 and CDR-H3 wherein one or more (e.g., 1 or 2 or 3) of such CDRs has one or more of such mutations (e.g., conservative substitutions).

The following references relate to BLAST algorithms often used for sequence analysis: BLAST ALGORITHMS: Altschul et al. (2005) FEBS J. 272(20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

A “conservatively modified variant” or a “conservative substitution”, e.g., of an immunoglobulin chain set forth herein, refers to a variant wherein there is one or more substitutions of amino acids in a polypeptide with other amino acids having similar characteristics (e.g., charge, side-chain size, hydrophobicity/hydrophilicity, backbone conformation and rigidity, etc.). Such changes can frequently be made without significantly disrupting the biological activity of the antibody or fragment. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4^{th} Ed.)). In addition, substitutions of structurally or functionally similar amino acids are less likely to significantly disrupt biological activity. The present invention includes anti-FGFR3 antigen-binding proteins comprising such conservatively modified variant immunoglobulin chains.

Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Alternatively, a conservative replacement is any change having a positive value in the PAM250 log-likelihood matrix disclosed in Gonnet et al. (1992) Science 256: 1443-45.

“H4H30063P” (“REGN15684”); “H4H30066P”; “H4H30071P”; “H4H30089P2”; “H4H30093P2”; “H4H30102P2”; “H4H30076P”; “H4H30105P2”; “H4H30108P2”; “H4H30117P2”; “H4H30045P”; “H4H30061P”; and “H4H30095P2” unless otherwise stated, refer to anti-FGFR3 antigen-binding proteins, e.g., antibodies and antigen-binding fragments thereof (including multispecific antigen-binding proteins), comprising an immunoglobulin heavy chain variable region (V_{H}) and an immunoglobulin light chain variable region (V_{L}) comprising the amino acid sequence pair specifically set forth in SEQ ID NOs: 2 & 10; 22 & 30; 42 & 50; 62 & 70; 82 & 90; 102 & 110; 122 & 130; 140 & 148; 159 & 148; 169 & 148; 179 & 187; 199 & 207; and 219 & 227 (or a variant of any of said sequences), as set forth in Table A; or comprising an immunoglobulin heavy chain (HC) and an immunoglobulin light chain (LC) comprising the amino acid sequence pair specifically set forth in SEQ ID NOs: 18 & 20; 38 & 40;58&60;78&80;98&100;118&120;136&138;155&157;167&157;177&157;195 & 197; 215 & 217; or 229 & 231 (or a variant of any of said sequences), as set forth in Table A; or that comprise a heavy chain or V_{H} that comprises the CDRs thereof (CDR-H1 (or a variant thereof), CDR-H2 (or a variant thereof) and CDR-H3 (or a variant thereof)) and/or a light chain or V_{L} that comprises the CDRs thereof (CDR-L1 (or a variant thereof), CDR-L2 (or a variant thereof) and CDR-L3 (or a variant thereof)). In an embodiment of the invention, the V_{H} is linked to an IgG constant heavy chain domain, for example, human IgG constant heavy chain domain (e.g., IgG1 or IgG4 (e.g., comprising the S228P and/or S108P mutation)) and/or the V_{L} is linked to a light chain constant domain, for example a human light chain constant domain (e.g., lambda or kappa constant light chain domain). Polynucleotides encoding one or more of any such immunoglobulin chains (e.g., V_{H}, V_{L}, HC and/or LC) forms part of the present invention.

Antibodies and antigen-binding fragments of the present invention (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2) comprise immunoglobulin chains including the amino acid sequences specifically set forth herein (and variants thereof) as well as cellular and in vitro post-translational modifications to the antibody or fragment. For example, the present invention includes antibodies and antigen-binding fragments thereof that specifically bind to FGFR3 comprising heavy and/or light chain amino acid sequences set forth herein as well as antibodies and fragments wherein one or more asparagine, serine and/or threonine residues is glycosylated, one or more asparagine residues is deamidated, one or more residues (e.g., Met, Trp and/or His) is oxidized, the N-terminal glutamine is pyroglutamate (pyroE) and/or the C-terminal lysine or other amino acid is missing.

The present invention provides a vessel (e.g., a plastic or glass vial, e.g., with a cap or a chromatography column, hollow bore needle or a syringe cylinder) comprising an anti-FGFR3 antigen-binding protein of the present invention, e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2.

The present invention also provides an injection device comprising one or more antigen-binding proteins (e.g., antibody or antigen-binding fragment) that bind specifically to FGFR3, e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2, or a pharmaceutical formulation thereof. The injection device may be packaged into a kit. An injection device is a device that introduces a substance into the body of a subject via a parenteral route, e.g., intraocular, intravitreal, intramuscular, subcutaneous or intravenous. For example, an injection device may be a syringe or an auto-injector (e.g., pre-filled with the pharmaceutical formulation) which, for example, includes a cylinder or barrel for holding fluid to be injected (e.g., comprising the antibody or fragment or a pharmaceutical formulation thereof), a needle for piecing skin, blood vessels or other tissue for injection of the fluid; and a plunger for pushing the fluid out of the cylinder and through the needle bore and into the body of the subject.

### Post-Translational Modifications

The anti-FGFR3 antibodies and antigen-binding fragments of the present invention may be modified after translation, e.g., glycosylated.

For example, antibodies and antigen-binding fragments of the present invention may be glycosylated (e.g., N-glycosylated and/or O-glycosylated) or aglycosylated. Typically, antibodies and antigen-binding fragments are glycosylated at the conserved residue N297 of the IgG Fc domain. Some antibodies and fragments include one or more additional glycyosylation sites in a variable region. In an embodiment of the invention, the glycosylation site is in the following context: FN₂₉₇S or YN₂₉₇S.

In an embodiment of the invention, said glycosylation is any one or more of three different N-glycan types: high mannose, complex and/or hybrid that are found on IgGs with their respective linkage. Complex and hybrid types exist with core fucosylation, addition of a fucose residue to the innermost N-acetylglucosamine, and without core fucosylation.

In an embodiment of the invention, an antibody or fragment of the present invention is afucosylated. Some IgG1 antibodies rely on the Fc-mediated immune effector function, antibody-dependent cellular cytotoxicity (ADCC), as the major mode of action to deplete tumor cells. This effector function is modulated by the N-linked glycosylation in the Fc region of the antibody. In particular, absence of core fucose on the Fc N-glycan has been shown to increase IgG1 Fc binding affinity to the FcγRIIIa present on immune effector cells such as natural killer cells and lead to enhanced ADCC activity.

The antibodies and antigen-binding fragments of the present invention may also be post-translationally modified in other ways including, for example: Glu or Gln cyclization at N-terminus; Loss of positive N-terminal charge; Lys variants at C-terminus; Deamidation (Asn to Asp); Isomerization (Asp to isoAsp); Deamidation (Gln to Glu); Oxidation (Cys, His, Met, Tyr, Trp); and/or Disulfide bond heterogeneity (Shuffling, thioether and trisulfide formation).

### Polynucleotides and Methods of Making

A polynucleotide includes DNA and RNA. The present invention includes any polynucleotide of the present invention, for example, encoding an immunoglobulin V_{H}, V_{L}, CDR-H, CDR-L, HC or LC of H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; and/or H4H30093P2, optionally, which is operably linked to a promoter or other expression control sequence. For example, the present invention provides any polynucleotide (e.g., DNA) that includes a nucleotide sequence set forth in SEQ ID NO: 1; 3; 5; 7; 9; 11; 13; 15; 17; 19; 21; 23; 25; 27; 29; 31; 33; 35; 37; 39; 41; 43; 45; 47; 49; 51; 53; 55; 57; 59; 61; 63; 65; 67; 69; 71; 73; 75; 77; 79; 81; 83; 85; 87; 89; 91; 93; 95; 97; 99; 101; 103; 105; 107; 109; 111; 113; 115; 117; 119; 121; 123; 125; 127; 129; 131; 133; 135; 137; 139; 141; 143; 145; 147; 149; 151; 152; 154; 156; 158; 160; 162; 164; 166; 168; 170; 172; 174; 176; 178; 180; 182; 184; 186; 188; 190; 192; 194; 196; 198; 200; 202; 204; 206; 208; 210; 212; 214; 216; 218; 220; 222; 224; 226; 228; or 230. In an embodiment of the invention, a polynucleotide of the present invention is fused to a secretion signal sequence. Polypeptides encoded by such polynucleotides are also within the scope of the present invention.

In general, a “promoter” or “promoter sequence” is a DNA regulatory region capable of binding an RNA polymerase in a cell (e.g., directly or through other promoter-bound proteins or substances) and initiating transcription of a coding sequence. A promoter may be operably linked to other expression control sequences, including enhancer and repressor sequences and/or with a polynucleotide of the invention. Promoters which may be used to control gene expression include, but are not limited to, cytomegalovirus (CMV) promoter (U.S. Pat. Nos. 5,385,839 and 5,168,062), the SV40 early promoter region (Benoist et al., (1981) Nature 290:304-310), the promoter contained in the 3′ long terminal repeat of Rous sarcoma virus (Yamamoto et al., (1980) Cell 22:787-797), the herpes thymidine kinase promoter (Wagner et al., (1981) Proc. Natl. Acad. Sci. USA 78:1441-1445), the regulatory sequences of the metallothionein gene (Brinster et al., (1982) Nature 296:39-42); prokaryotic expression vectors such as the beta-lactamase promoter (VIIIa-Komaroff et al., (1978) Proc. Natl. Acad. Sci. USA 75:3727-3731), or the tac promoter (DeBoer et al., (1983) Proc. Natl. Acad. Sci. USA 80:21-25); see also “Useful proteins from recombinant bacteria” in Scientific American (1980) 242:74-94; and promoter elements from yeast or other fungi such as the Gal4 promoter, the ADC (alcohol dehydrogenase) promoter, PGK (phosphoglycerol kinase) promoter or the alkaline phosphatase promoter.

A polynucleotide encoding a polypeptide is “operably linked” to a promoter or other expression control sequence when, in a cell or other expression system, the sequence directs RNA polymerase mediated transcription of the coding sequence into RNA, preferably mRNA, which then may be RNA spliced (if it contains introns) and, optionally, translated into a protein encoded by the coding sequence.

The present invention includes a polynucleotide comprising the following polynucleotide pairs encoding a V_{H} and V_{L}.
SEQ ID NO: 1 and SEQ ID NO: 9;
SEQ ID NO: 21 and SEQ ID NO: 29;
SEQ ID NO: 41 and SEQ ID NO: 49;
SEQ ID NO: 61 and SEQ ID NO: 69;
SEQ ID NO: 81 and SEQ ID NO: 89;
SEQ ID NO: 101 and SEQ ID NO: 109;
SEQ ID NO: 121 and SEQ ID NO: 129;
SEQ ID NO: 139 and SEQ ID NO:147;
SEQ ID NO: 158 and SEQ ID NO:147;
SEQ ID NO: 168 and SEQ ID NO: 147;
SEQ ID NO: 178 and SEQ ID NO: 186;
SEQ ID NO: 198 and SEQ ID NO: 206; and/or
SEQ ID NO: 218 and SEQ ID NO: 226;
   or two separate polynucleotides, each including one of the sequences
SEQ ID NO: 1 and SEQ ID NO: 9;
SEQ ID NO: 21 and SEQ ID NO: 29;
SEQ ID NO: 41 and SEQ ID NO: 49;
SEQ ID NO: 61 and SEQ ID NO: 69;
SEQ ID NO: 81 and SEQ ID NO: 89;
SEQ ID NO: 101 and SEQ ID NO: 109
SEQ ID NO: 121 and SEQ ID NO: 129;
SEQ ID NO: 139 and SEQ ID NO:147;
SEQ ID NO: 158 and SEQ ID NO:147;
SEQ ID NO: 168 and SEQ ID NO: 147;
SEQ ID NO: 178 and SEQ ID NO: 186;
SEQ ID NO: 198 and SEQ ID NO: 206; or
SEQ ID NO: 218 and SEQ ID NO: 226.

The present invention includes a polynucleotide comprising the following polynucleotide sets which encode a CDR-H1, CDR-H2, CDR-H3, CDR-L1, CDR-L2 and CDR-L3:
SEQ ID NOs: 3, 5, 7, 11, 13 and 15;
SEQ ID NOs: 23, 25, 27, 31, 33 and 35;
SEQ ID NOs: 43, 45, 47, 51, 53 and 55;
SEQ ID NOs: 63, 65, 67, 71, 73 and 75;
SEQ ID NOs: 83, 85, 87, 91, 93 and 95;
SEQ ID NOs: 103, 105, 107, 111, 113 and 115;
SEQ ID NOs: 123, 125, 127, 131, 33 and 133;
SEQ ID NOs: 141, 143, 415, 149, 151 and 152;
SEQ ID NOs: 160, 162, 164, 149, 151 and 152;
SEQ ID NOs: 170, 172, 174, 149, 151 and 152;
SEQ ID NOs: 180, 182, 184, 188, 190 and 192;
SEQ ID NOs: 200, 202, 204, 208, 210 and 212; and/or
SEQ ID NOs: 220, 222, 224, 31, 33 and 75;
   or two separate polynucleotides, each including one of the sequences
SEQ ID NOs: 3, 5 and 7; and 11, 13 and 15;
SEQ ID NOs: 23, 25 and 27; and 31, 33 and 35;
SEQ ID NOs: 43, 45 and 47; and 51, 53 and 55;
SEQ ID NOs: 63, 65 and 67; and 71, 73 and 75;
SEQ ID NOs: 83, 85 and 87; and 91, 93 and 95;
SEQ ID NOs: 103, 105 and 107; and 111, 113 and 115;
SEQ ID NOs: 123, 125, and 127; 131, 33 and 133;
SEQ ID NOs: 141, 143, and 415; 149, 151 and 152;
SEQ ID NOs: 160, 162, and 164; 149, 151 and 152;
SEQ ID NOs: 170, 172, and 174; 149, 151 and 152;
SEQ ID NOs: 180, 182, and 184; 188, 190 and 192;
SEQ ID NOs: 200, 202, and 204; 208, 210 and 212; or
SEQ ID NOs: 220, 222, and 224; 31, 33 and 75.

The present invention includes a polynucleotide comprising the following polynucleotide pairs encoding a HC and LC:
SEQ ID NO: 17 and SEQ ID NO: 19;
SEQ ID NO: 37 and SEQ ID NO: 39;
SEQ ID NO: 57 and SEQ ID NO: 59;
SEQ ID NO: 77 and SEQ ID NO: 79;
SEQ ID NO: 97 and SEQ ID NO: 99;
SEQ ID NO: 117 and SEQ ID NO: 119;
SEQ ID NO: 135 and SEQ ID NO: 137;
SEQ ID NO: 154 and SEQ ID NO: 156;
SEQ ID NO: 166 and SEQ ID NO: 156;
SEQ ID NO: 176 and SEQ ID NO: 156;
SEQ ID NO: 194 and SEQ ID NO: 196;
SEQ ID NO: 214 and SEQ ID NO: 216; and/or
SEQ ID NO: 228 and SEQ ID NO: 230;
   or two separate polynucleotides, each including one of the sequences
SEQ ID NO: 17 and SEQ ID NO: 19;
SEQ ID NO: 37 and SEQ ID NO: 39;
SEQ ID NO: 57 and SEQ ID NO: 59;
SEQ ID NO: 77 and SEQ ID NO: 79;
SEQ ID NO: 97 and SEQ ID NO: 99;
SEQ ID NO: 117 and SEQ ID NO: 119;
SEQ ID NO: 135 and SEQ ID NO: 137;
SEQ ID NO: 154 and SEQ ID NO: 156;
SEQ ID NO: 166 and SEQ ID NO: 156;
SEQ ID NO: 176 and SEQ ID NO: 156;
SEQ ID NO: 194 and SEQ ID NO: 196;
SEQ ID NO: 214 and SEQ ID NO: 216; and/or
SEQ ID NO: 228 and SEQ ID NO: 230.

Host cells including the two separate polynucleotides as discussed above, each integrated into chromosomal DNA of the host cell at different loci or ectopic, wherein such polynucleotide are maintained in separate genetic elements, are within the scope of the present invention.

The present invention includes polynucleotides encoding immunoglobulin polypeptide chains which are variants of those whose nucleotide sequence is specifically set forth herein. A “variant” of a polynucleotide refers to a polynucleotide comprising a nucleotide sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical to a referenced nucleotide sequence that is set forth herein; when the comparison is performed by a BLAST algorithm wherein the parameters of the algorithm are selected to give the largest match between the respective sequences over the entire length of the respective reference sequences (e.g., expect threshold: 10; word size: 28; max matches in a query range: 0; match/mismatch scores: 1, −2; gap costs: linear). In an embodiment of the invention, a variant of a nucleotide sequence specifically set forth herein comprises one or more (e.g., 1, 2, 3, 4, 5, 6, 7, 8, 9, 10, 11 or 12) point mutations, insertions (e.g., in frame insertions) or deletions (e.g., in frame deletions) of one or more nucleotides. Such mutations may, in an embodiment of the invention, be missense or nonsense mutations. In an embodiment of the invention, such a variant polynucleotide encodes an immunoglobulin polypeptide chain which can be incorporated into an anti-FGFR3 antigen-binding protein, i.e., such that the protein retains specific binding to FGFR3.

Eukaryotic and prokaryotic host cells, including mammalian cells, may be used as hosts for expression of an anti-FGFR3 antigen-binding protein (e.g., antibody or antigen-binding fragment thereof). Such host cells are well known in the art and many are available from the American Type Culture Collection (ATCC). These host cells include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. The present invention includes an isolated host cell (e.g., a CHO cell or any type of host cell set forth above) comprising an antigen-binding protein, a V_{H}, V_{L}, HC, LC or CDRs thereof (or variant thereof), such as H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2; and/or a polynucleotide encoding one or more immunoglobulin chains thereof (e.g., as discussed herein). In an embodiment of the invention, a host cell includes two separate polynucleotides, one encoding a V_{H} and the other encoding a V_{L}; or one encoding a HC and the other encoding a LC.

The present invention also includes a cell which is expressing FGFR3 or an antigenic fragment or fusion thereof (e.g., His₆ (SEQ ID NO: 235), Fc (e.g., mouse Fc (mFc)), myc, or mycmycHis6 (mmh)) which is bound by an antigen-binding protein of the present invention (e.g., an antibody or antigen-binding fragment thereof), for example, H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2, for example, wherein the cell is in the body of a subject or is in vitro. In addition, the present invention also provides a complex comprising an anti-FGFR3 antigen-binding protein, e.g., antibody or antigen-binding fragment thereof, as discussed herein complexed with FGFR3 polypeptide or an antigenic fragment thereof or fusion thereof and/or with a secondary antibody or antigen-binding fragment thereof (e.g., detectably labeled secondary antibody) that binds specifically to the anti-FGFR3 antibody or fragment. In an embodiment of the invention, the complex is in vitro (e.g., is immobilized to a solid substrate) or is in the body of a subject.

In an embodiment of the invention, a myc tag has the amino acid sequence EQKLISEEDLGG (SEQ ID NO: 234), a His₆ (SEQ ID NO: 235) or hexahis (SEQ ID NO: 235) or hexahistidine (SEQ ID NO: 235) tag has the amino acid sequence HHHHHH (SEQ ID NO: 235), an mmh tag has the amino acid sequence EQKLISEEDLGGEQKLISEEDLHHHHHH (SEQ ID NO: 236) and a mouse Fc tag has the amino acid sequence

(SEQ ID NO: 237)
EPRGPTIKPCPPCKCPAPNLLGGPSVFIFPPKIKDVLMISLSPIVTCVV
VDVSEDDPDVQISWFVNNVEVHTAQTQTHREDYNSTLRVVSALPIQHQD
WMSGKEFKCKVNNKDLPAPIERTISKPKGSVRAPQVYVLPPPEEEMTKK
QVTLTCMVTDEMPEDIYVEWTNNGKTELNYKNTPVLDSDGSYFMYSKLR
VEKKNWVERNSYSCSVVHEGLHNHHTTKSFSRTPGK.

Recombinant anti-FGFR3 antigen-binding proteins, e.g., antibodies and antigen-binding fragments, disclosed herein may also be produced in an E. coli/T7 expression system. In this embodiment, polynucleotides encoding the anti-FGFR3 antibody immunoglobulin molecules of the invention (e.g., HC, LC, V_{H} and/or V_{L} or CDRs thereof of (H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2)) may be inserted into a pET-based plasmid and expressed in the E. coli/T7 system. For example, the present invention includes methods for expressing an antibody or antigen-binding fragment thereof or immunoglobulin chain thereof in a host cell (e.g., bacterial host cell such as E. coli such as BL21 or BL21 DE3) comprising expressing T7 RNA polymerase in the cell which also includes a polynucleotide encoding an immunoglobulin chain (e.g., including the nucleotide sequence in any one or more of SEQ ID NOs: 1, 3, 5, 7, 9, 11, 13, 15, 17 or 19; or a variant thereof) that is operably linked to a T7 promoter. For example, in an embodiment of the invention, a bacterial host cell, such as an E. coli, includes a polynucleotide encoding the T7 RNA polymerase gene operably linked to a lac promoter and expression of the polymerase and the chain is induced by incubation of the host cell with IPTG (isopropyl-beta-D-thiogalactopyranoside). See U.S. Pat. Nos. 4,952,496 and 5,693,489 or Studier & Moffatt, Use of bacteriophage T7 RNA polymerase to direct selective high-level expression of cloned genes, J. Mol. Biol. 1986 May 5;189(1): 113-30.

There are several methods by which to produce recombinant antibodies which are known in the art. One example of a method for recombinant production of antibodies is disclosed in U.S. Pat. No. 4,816,567.

Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art. See, for example, U.S. Pat. Nos. 4,399,216; 4,912,040; 4,740,461 and 4,959,455. Thus, the present invention includes recombinant methods for making an anti-FGFR3 (e.g., monomeric or dimeric FGFR3b) antigen-binding protein, such as an antibody or antigen-binding fragment thereof of the present invention, or an immunoglobulin chain thereof, comprising (i) introducing, into a host cell, one or more polynucleotides (e.g., including the nucleotide sequence in any one or more of SEQ ID NOs: 1, 9, 17 and/or 19; or a variant thereof) encoding light and/or heavy immunoglobulin chains of the antigen-binding protein, e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2, for example, wherein the polynucleotide is in a vector; and/or integrates into the host cell chromosome and/or is operably linked to a promoter; (ii) culturing the host cell (e.g., CHO or Pichia or Pichia pastoris) under conditions favorable to expression of the polynucleotide and, (iii) optionally, isolating the antigen-binding protein (e.g., antibody or antigen-binding fragment) or chain from the host cell and/or medium in which the host cell is grown. When making an antigen-binding protein (e.g., antibody or antigen-binding fragment) comprising more than one immunoglobulin chain, e.g., an antibody that comprises two heavy immunoglobulin chains and two light immunoglobulin chains, co-expression of the chains in a single host cell leads to association of the chains, e.g., in the cell or on the cell surface or outside the cell if such chains are secreted, so as to form the antigen-binding protein (e.g., antibody or antigen-binding fragment). The methods of the present invention include those wherein only a heavy immunoglobulin chain or only a light immunoglobulin chain or both (e.g., any of those discussed herein including mature fragments and/or variable domains thereof) are expressed in a cell. Such single chains are useful, for example, as intermediates in the expression of an antibody or antigen-binding fragment that includes such a chain. For example, the present invention also includes anti-FGFR3 antigen-binding proteins, such as antibodies and antigen-binding fragments thereof which are the product of the production methods set forth herein, and, optionally, the purification methods set forth herein.

In an embodiment of the invention, a method for making an anti-FGFR3 (e.g., monomeric or dimeric FGFR3b) antigen-binding protein, e.g., antibody or antigen-binding fragment thereof, includes a method of purifying the antigen-binding protein, e.g., by column chromatography, precipitation and/or filtration. As discussed, the product of such a method also forms part of the present invention.

### Preparation of Human Antibodies

The anti-FGFR3 (e.g., monomeric or dimeric FGFR3b) antibodies and antigen-binding fragments of the present invention (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2) can be fully human antibodies and fragments. Methods for generating monoclonal antibodies, including fully human monoclonal antibodies are known in the art. Any such known methods can be used in the context of the present invention to make human antibodies that specifically bind to human FGFR3.

Using VELOCIMMUNE™ technology, for example, or any other similar known method for generating fully human monoclonal antibodies, high affinity chimeric antibodies to FGFR3 are initially isolated having a human variable region and a mouse constant region. As in the experimental section below, the antibodies are characterized and selected for desirable characteristics, including affinity, ligand blocking activity, selectivity, epitope, etc. If necessary, mouse constant regions are replaced with a desired human constant region, for example wild-type or modified IgG1 or IgG4, to generate a fully human anti-FGFR3 antibody. While the constant region selected may vary according to specific use, high affinity antigen-binding and target specificity characteristics reside in the variable region. In certain instances, fully human anti-FGFR3 antibodies are isolated directly from antigen-positive B cells. See, for example, U.S. Pat. No. 6,596,541, Regeneron Pharmaceuticals, VELOCIMMUNE®.

### Anti-FGFR3 Antibodies Comprising Fc Variants

According to certain embodiments of the present invention, anti-FGFR3 (e.g., monomeric or dimeric FGFR3b) antibodies and antigen-binding fragments (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2) are provided comprising an Fc domain comprising one or more mutations which enhance or diminish antibody binding to the FcRn receptor, e.g., at acidic pH as compared to neutral pH (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2). For example, the present invention includes anti-FGFR3 antibodies comprising a mutation in the CH2 or a CH3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal.

Non-limiting examples of such Fc modifications include, e.g., a modification at position:
250 (e.g., E or Q);
250 and 428 (e.g., L or F);
252 (e.g., L/Y/F/W or T),
254 (e.g., S or T), and/or
256 (e.g., S/R/Q/E/D or T);
   and/or a modification at position:
428 and/or 433 (e.g., H/L/R/S/P/Q or K), and/or
434 (e.g., A, W, H/F or Y);
   and/or a modification at position:
250 and/or 428;
   and/or a modification at position:
307 or 308 (e.g., 308F, V308F), and/or
434.

In an embodiment of the invention, the modification comprises:
a 428L (e.g., M428L) and 434S (e.g., N434S) modification;
a 428L, 2591 (e.g., V2591), and 308F (e.g., V308F) modification;
a 433K (e.g., H433K) and a 434 (e.g., 434Y) modification;
a 252, 254, and 256 (e.g., 252Y, 254T, and 256E) modification;
a 250Q and 428L modification (e.g., T250Q and M428L); and/or
a 307 and/or 308 modification (e.g., 308F or 308P).

For example, the present invention includes anti-FGFR3 antibodies comprising an Fc domain comprising one or more pairs or groups of mutations selected from the group consisting of:
250Q and 248L (e.g., T250Q and M248L);
252Y, 254T and 256E (e.g., M252Y, S254T and T256E);
257I and 311I (e.g., P257I and Q311I);
257I and 434H (e.g., P257I and N434H);
376V and 434H (e.g., D376V and N434H);
307A, 380A and 434A (e.g., T307A, E380A and N434A);
428L and 434S (e.g., M428L and N434S); and
433K and 434F (e.g., H433K and N434F).

In yet another embodiment, the modification comprises a 265A (e.g., D265A) and/or a 297A (e.g., N297A) modification.

In an embodiment of the invention, the heavy chain constant domain is gamma-4 comprising an S228P and/or S108P mutation. See Angal et al., A single amino acid substitution abolishes the heterogeneity of chimeric mouse/human (IgG4) antibody, Mol Immunol. 1993 January; 30(1):105-108.

All possible combinations of the foregoing Fc domain mutations, and other mutations within the antibody variable domains disclosed herein, are contemplated within the scope of the present invention.

The anti-FGFR3 antibodies of the present invention may comprise a modified Fc domain having reduced effector function. As used herein, a “modified Fc domain having reduced effector function” means any Fc portion of an immunoglobulin that has been modified, mutated, truncated, etc., relative to a wild-type, naturally occurring Fc domain such that a molecule comprising the modified Fc exhibits a reduction in the severity or extent of at least one effect selected from the group consisting of cell killing (e.g., ADCC and/or CDC), complement activation, phagocytosis and opsonization, relative to a comparator molecule comprising the wild-type, naturally occurring version of the Fc portion. In certain embodiments, a “modified Fc domain having reduced effector function” is an Fc domain with reduced or attenuated binding to an Fc receptor (e.g., FcγR).

In certain embodiments of the present invention, the modified Fc domain is a variant IgG1 Fc or a variant IgG4 Fc comprising a substitution in the hinge region. For example, a modified Fc for use in the context of the present invention may comprise a variant IgG1 Fc wherein at least one amino acid of the IgG1 Fc hinge region is replaced with the corresponding amino acid from the IgG2 Fc hinge region. Alternatively, a modified Fc for use in the context of the present invention may comprise a variant IgG4 Fc wherein at least one amino acid of the IgG4 Fc hinge region is replaced with the corresponding amino acid from the IgG2 Fc hinge region. Non-limiting, exemplary modified Fc regions that can be used in the context of the present invention are set forth in US Patent Application Publication No. 2014/0243504, the disclosure of which is hereby incorporated by reference in its entirety, as well as any functionally equivalent variants of the modified Fc regions set forth therein.

The present disclosure also includes antigen-binding proteins, antibodies or antigen-binding fragments, comprising a HCVR set forth herein and a chimeric heavy chain constant (CH) region, wherein the chimeric CH region comprises segments derived from the CH regions of more than one immunoglobulin isotype. For example, the antibodies of the disclosure may comprise a chimeric CH region comprising part or all of a CH2 domain derived from a human IgG1, human IgG2 or human IgG4 molecule, combined with part or all of a CH3 domain derived from a human IgG1, human IgG2 or human IgG4 molecule. According to certain embodiments, the antibodies of the disclosure comprise a chimeric CH region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric CH region as described herein may, in certain embodiments, exhibit modified Fc effector functions without adversely affecting the therapeutic or pharmacokinetic properties of the antibody. (See, e.g., WO2014/022540).

Other modified Fc domains and Fc modifications that can be used in the context of the present invention include any of the modifications as set forth in US2014/0171623; U.S. Pat. No. 8,697,396; US2014/0134162; WO2014/043361, the disclosures of which are hereby incorporated by reference in their entireties. Methods of constructing antibodies or other antigen-binding fusion proteins comprising a modified Fc domain as described herein are known in the art.

In some cases, the anti-FGFR3 antibodies may comprise one or more mutations in a framework region, e.g., in the CH1 domain, CH2 domain, CH3 domain, hinge region, or a combination thereof. In some embodiments, the one or more mutations are to stabilize the antibody and/or to increase half-life. In some embodiments, the one or more mutations are to modulate Fc receptor interactions, to reduce or eliminate Fc effector functions such as FcyR, antibody-dependent cell-mediated cytotoxicity (ADCC), or complement-dependent cytotoxicity (CDC). In additional embodiments, the one or more mutations are to modulate glycosylation.

In some embodiments, one, two or more mutations (e.g., amino acid substitutions) are introduced into the Fc region of an antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody, such as serum half-life, complement fixation, Fc receptor binding and/or antigen-dependent cellular cytotoxicity. In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., PCT Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo. In some embodiments, the Fc region comprises a mutation at residue position L234, L235, or a combination thereof. In some embodiments, the mutations comprise L234 and L235. In some embodiments, the mutations comprise L234A and L235A.

### Immunoconjugates

The invention encompasses anti-FGFR3 (e.g., monomeric or dimeric FGFR3b) antigen-binding proteins, e.g., antibodies or antigen-binding fragments, conjugated to another moiety, e.g., a therapeutic moiety (an “immunoconjugate”) (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2). In an embodiment of the invention, an anti-FGFR3 antigen-binding protein, e.g., antibody or antigen-binding fragment, is conjugated to any of the further therapeutic agents set forth herein. As used herein, the term “immunoconjugate” refers to an antigen-binding protein, e.g., an antibody or antigen-binding fragment, which is chemically or biologically linked to another antigen-binding protein, a drug, a radioactive agent, a reporter moiety, an enzyme, a peptide, a protein or a therapeutic agent.

### Treatment and Administration

The present invention provides methods for treating or preventing an FGFR3-mediated condition, in a subject, comprising administering a therapeutically effective amount of anti-FGFR3 (e.g., monomeric or dimeric FGFR3b) antigen-binding protein (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2) to the subject; optionally, in association with a further therapeutic agent.

An FGFR3-mediated condition is any condition that is mediated at least in part by the activity of FGFR3, for example, tyrosine kinase activity of FGFR3 or activity of molecules downstream of FGFR3 (e.g., CD73 or the MEK pathway in a tumor cell expressing FGFR3).

An FGFR3-mediated condition can also include T-cell suppression mediated by a tumor cell expressing FGFR3, e.g., adenosine-mediated suppression of T-cells via the A2A receptor, for example, wherein the CD73 catalyzes conversion of AMP to adenosine. CD73 (NT5E, ecto-5′-nucleotidase) is a glycosylphosphatidylinositol- (GPI-) anchored cell-surface enzyme that plays a crucial role in the purinergic signaling pathway by dephosphorylating AMP (adenosine monophosphate) into adenosine. Extracellular adenosine itself is involved in tumor immunoescape and invasion of tumor cells, while nonenzymatic functions of CD73 are related to cell adhesion and migration of tumor cells.

FGFR3-mediated conditions include, for example,
cancer,
bladder cancer,
brain cancer,
breast cancer,
cervical cancer,
colorectal cancer,
endometrial cancer,
gastric cancer,
head & neck cancer
kidney cancer,
lung cancer,
multiple myeloma,
ovarian cancer,
pancreatic cancer,
urothelial cancer,
achondroplasia,
crouzon syndrome with acanthosis nigricans,
epidermal nevus,
hypochondroplasia;
lacrimo-auriculo-dento-digital (LADD) syndrome,
muenke syndrome,
severe achondroplasia with developmental delay and acanthosis nigricans (SADDAN), and/or
thanatophoric dysplasia.

Achondroplasia is a form of short-limbed dwarfism. Crouzon syndrome with acanthosis nigricans is a condition that causes premature joining of the bones of the skull (craniosynostosis), leading to a misshapen head and distinctive facial features, and a skin abnormality called acanthosis nigricans that is characterized by thick, dark, velvety skin in body folds and creases. Epidermal nevus is abnormal skin growths that are composed of skin cells called keratinocytes. Hypochondroplasia is a form of short-limbed dwarfism that is milder than achondroplasia. Lacrimo-auriculo-dento-digital (LADD) syndrome is an extremely rare genetic disorder characterized by abnormalities affecting the lacrimal and salivary glands and ducts, ears, teeth and fingers and toes. The most common findings involve malformations in the network of structures of the eye that secrete tears and drain them from the eyes (lacrimal apparatus) and abnormalities of the forearms and fingers. Specific symptoms may vary greatly from person to person. LADD syndrome may occur sporadically or be inherited in an autosomal dominant pattern. Muenke syndrome is a condition that causes craniosynostosis, leading to a misshapen head and distinctive facial features. Additional signs and symptoms can include hearing loss, subtle hand and foot abnormalities, and developmental delay. SADDAN (severe achondroplasia with developmental delay and acanthosis nigricans) is characterized by short-limb dwarfism (achondroplasia); profound developmental delay; and thick, dark, velvety skin. Thanatophoric dysplasia is a severe skeletal disorder characterized by extremely short limbs and folds of extra (redundant) skin on the arms and legs. Other features of this condition include a narrow chest, short ribs, underdeveloped lungs, and an enlarged head with a large forehead and prominent, wide-spaced eyes. Bladder cancer includes non-muscle invasive bladder cancer (NMIBC) and muscle-invasive bladder cancer (MIBC).

An effective or therapeutically effective amount of anti-FGFR3 antigen-binding protein, e.g., antibody or antigen-binding fragment, for treating or preventing an FGFR3-mediated condition refers to the amount of the antigen-binding protein sufficient to alleviate one or more signs and/or symptoms of the disease or condition in the treated subject, whether by inducing the regression or elimination of such signs and/or symptoms or by inhibiting the progression of such signs and/or symptoms. In an embodiment of the invention, an effective or therapeutically effective amount of anti-FGFR3 antigen-binding protein is about 2-30 mg/kg. This dose may be administered, for example, about once a month. The dose amount may vary depending upon the age and the size of a subject to be administered, target disease, conditions, route of administration, and the like. In certain embodiments, the initial dose may be followed by administration of a second or a plurality of subsequent doses of antigen-binding protein in an amount that can be approximately the same or less or more than that of the initial dose, wherein the subsequent doses are separated by days or weeks or months.

The present invention provides methods for administering an anti-FGFR3 (e.g., monomeric or dimeric FGFR3b) antigen-binding protein, e.g., antibody or antigen-binding fragment thereof (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2) to a subject, comprising introducing the protein or a pharmaceutical formulation thereof into the body of the subject. For example, in an embodiment of the invention, the method comprises piercing the body of the subject, e.g., with a needle of a syringe, and injecting the antigen-binding protein or a pharmaceutical formulation thereof into the body of the subject, e.g., into the eye, vein, artery, muscular tissue or subcutis of the subject.

As used herein, the term “subject” refers to a mammal (e.g., rat, mouse, cat, dog, cow, sheep, horse, goat, rabbit), preferably a human, for example, in need of prevention and/or treatment of an FGFR3-mediated condition. The subject may have an FGFR3-mediated condition or be predisposed to developing such a condition. In an embodiment of the invention, the subject has an FGFR3 genotype selected from: S249C, R248C, G372C, Y375C, K650E and/or FGFR3-TACC3 (e.g., heterozygous or homozygous).

### Combinations and Pharmaceutical Formulation

The present invention provides compositions that include anti-FGFR3 (e.g., monomeric or dimeric FGFR3b) antigen-binding proteins, such as antibodies or antigen-binding fragments (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2), in association with one or more ingredients; as well as methods of use thereof and methods of making such compositions. Pharmaceutic formulations comprising an anti-FGFR3 antigen-binding protein and a pharmaceutically acceptable carrier or excipient are part of the present invention.

To prepare pharmaceutical formulations of the anti-FGFR3 antigen-binding proteins, e.g., antibodies and antigen-binding fragments thereof (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2), antigen-binding protein is admixed with a pharmaceutically acceptable carrier or excipient. See, e.g., Remington's Pharmaceutical Sciences and U.S. Pharmacopeia: National Formulary, Mack Publishing Company, Easton, Pa. (1984); Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y. In an embodiment of the invention, the pharmaceutical formulation is sterile. Such compositions are part of the present invention.

Pharmaceutical formulations of the present invention include an anti-FGFR3 antigen-binding protein (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2) and a pharmaceutically acceptable carrier including, for example, water and buffering agents.

The scope of the present invention includes desiccated, e.g., freeze-dried, compositions comprising an anti-FGFR3 antigen-binding protein, e.g., antibody or antigen-binding fragment thereof (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2), or a pharmaceutical formulation thereof that includes a pharmaceutically acceptable carrier but substantially lacks water.

In a further embodiment of the invention, a further therapeutic agent that is administered to a subject in association with an anti-FGFR3 antigen-binding protein, e.g., antibody or antigen-binding fragment thereof (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2), disclosed herein is administered to the subject in accordance with the Physicians' Desk Reference 2003 (Thomson Healthcare; 57^{th} edition (Nov. 1, 2002)).

The mode of administration of an anti-FGFR3 antigen-binding protein or composition thereof can vary. Routes of administration include parenteral, non-parenteral, oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, intraocular, intravitreal, transdermal or intra-arterial.

The present invention provides a vessel (e.g., a plastic or glass vial, e.g., with a cap or a chromatography column, hollow bore needle or a syringe cylinder) comprising any of the anti-FGFR3 antigen-binding proteins, e.g., antibodies or antigen-binding fragments thereof (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2), or a pharmaceutical formulation comprising a pharmaceutically acceptable carrier thereof.

The present invention includes combinations including an anti-FGFR3 antigen-binding protein, e.g., antibody or antigen-binding fragment thereof of the present invention (e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2), in association with one or more further therapeutic agents. The anti-FGFR3 antigen-binding protein and the further therapeutic agent can be in a single composition or in separate compositions. For example, in an embodiment of the invention, the further therapeutic agent is cancer therapeutic agent. In an embodiment of the invention, the further therapeutic agent is
an FGFR inhibitor (e.g., erdafitinib)
pemigatinib,
infigratinib,
rogaratinib,
dexamethasone
alkylating drug (e.g., altretamine, trabectedin or busulfan),
a nitrosourea (e.g., carmustine or lomustine),
a cytotoxic antibiotic (e.g., an anthracycline, doxorubicin, valrubicin, bleomycin or dactinomycin),
an antimetabolite drug (e.g., methotrexate, floxuridine, clofarabine or pralatrexate),
a vinca alkaloid (e.g., vinblastine, vinorelbine, vincristine or vindesine),
a photodynamic drug (e.g., porfimer sodium or aminolevulinic acid),
a platinum drug (cisplatin or phenanthriplatin),
a taxane (e.g., paclitaxel or docetaxel),
a topoisomerase inhibitor (e.g., irinotecan, topotecan, etoposide or teniposide),
ziv-aflibercept
an anti-cancer antibody (e.g., rituximab, trastuzumab, cetuximab, cemiplimab, pembrolizumab, panitumumab or bevacizumab).

As discussed herein, the present invention includes methods for treating or preventing an FGFR3-mediated condition in a subject in need of said treatment or prevention by administering an anti-FGFR3 antigen-binding protein, e.g., H4H30063P; H4H30089P2; H4H30071P; H4H30066P; H4H30102P2; H4H30076P; H4H30105P2; H4H30108P2; H4H30117P2; H4H30045P; H4H30061P; H4H30095P2; or H4H30093P2, which may be in association with a further therapeutic agent.

The term “in association with” indicates that components, an anti-FGFR3 antigen-binding protein, e.g., antibody or antigen-binding fragment thereof of the present invention, along with another agent such as methotrexate, can be formulated into a single composition, e.g., for simultaneous delivery, or formulated separately into two or more compositions (e.g., a kit including each component). Components administered in association with each another can be administered to a subject at a different time than when the other component is administered; for example, each administration may be given non-simultaneously (e.g., separately or sequentially) at intervals over a given period of time. Separate components administered in association with each another may also be administered sequentially, though essentially simultaneously, during the same administration session. Moreover, the separate components administered in association with each another may be administered to a subject by the same or by a different route.

### EXAMPLES

### Example 1: Binding Kinetics

Biacore binding kinetics of anti-FGFR3 antibodies, in an antibody capture format, to monomeric human FGFR3b, cynomolgus FGFR3b, murine FGFR3b, human FGFR3c, and dimeric human FGFR3b ecto domain recombinant proteins at 25° C. were analyzed.

Equilibrium dissociation constants (K_{D} values) for human FGFR3b expressed with a C-terminal myc-myc-hexahistidine tag (hFGFR3b.mmH, REGN3152) or cynomolgus FGFR3b expressed with a C-terminal myc-myc-hexahistidine tag (mfFGFR3b.mmH, REGN3521), or murine FGFR3b expressed with a C-terminal myc-myc-hexahistidine tag (mFGFR3b.mmH, REGN3215) or human FGFR3c expressed with a C-terminal myc-myc-hexahistidine tag (hFGFR3c.mmH, REGN3155) or human FGFR3b expressed with an C-terminal murine Fc tag (hFGFR3b.mFc, REGN3153) binding to purified anti-FGFR3 antibodies were determined using a real-time surface plasmon resonance biosensor technology using a Biacore 3000 or Biacore 4000 instrument. The CM5 Biacore sensor surface was derivatized by amine coupling with a monoclonal mouse anti-human Fc monoclonal antibody (REGN2567). All Biacore binding studies were performed in a buffer composed of 0.01M HEPES pH 7.4, 0.15 M NaCl, 3 mM EDTA, 0.05% v/v Surfactant P20 (HBS-EP running buffer). Different concentrations of monomeric proteins prepared in HBS-EP running buffer (ranging from 90 nM to 3.33 nM in 3-fold serial dilutions) or dimeric protein (hFGFR3b.mFc, REGN3153) prepared in HBS-EP running buffer (ranging from 30 to 10 nM in 3-fold serial dilution) were injected over the captured anti-FGFR3 antibodies at a flow rate of 30 μL/minute. Antibody-reagent association was monitored for 5 minutes while dissociation in HBS-EP running buffer was monitored for 10 minutes. At the end of each cycle, the anti-FGFR3 antibody capture surface was regenerated using a 10 sec injection of 20 mM phosphoric acid. All binding kinetics experiments were performed at 25° C.

The specific SPR-Biacore sensorgrams were obtained by a double referencing procedure. The double referencing was performed by first subtracting the signal of each injection over a reference surface (anti-hFc) from the signal over the experimental surface (anti-hFc-captured anti-FGFR3 antibodies) thereby removing contributions from refractive index changes. In addition, running buffer injections were performed to allow subtraction of the signal changes resulting from the dissociation of captured antibodies from the coupled anti-hFc surface. Kinetic association (kₐ) and dissociation (k_{d}) rate constants were determined by fitting the real-time sensorgrams to a 1:1 binding model using Scrubber v2.0c curve fitting software. Binding dissociation equilibrium constants (K_{D}) and dissociative half-lives (t½) were calculated from the kinetic rate constants as:

$${K}_{D} ( M ) = \frac{kd}{ka} , and t 1 / 2 ( min ) = \frac{ln ( 2 )}{60 * kd}$$

Monomeric kinetics results are presented in Tables 1-1 through 1-4. Dimeric kinetic results are presented in Table 1-5.

REGN3152 (human FGFR3b expressed with a C-terminal myc-myc-hexahistidine tag
(hFGFR3b.mmH))
(SEQ ID NO: 238)
ESLGTEQRVVGRAAEVPGPEPGQQEQLVFGSGDAVELSCPPPGGGPMGPTVWVKDGTGLVPSERVLVGPQRLQVLNASHE
DSGAYSCRQRLTQRVLCHFSVRVTDAPSSGDDEDGEDEAEDTGVDTGAPYWTRPERMDKKLLAVPAANTVRERCPAAGNP
TPSISWLKNGREFRGEHRIGGIKLRHQQWSLVMESVVPSDRGNYTCVVENKFGSIRQTYTLDVLERSPHRPILQAGLPAN
QTAVLGSDVEFHCKVYSDAQPHIQWLKHVEVNGSKVGPDGTPYVTVLKSWISESVEADVRLRLANVSERDGGEYLCRATN
FIGVAEKAFWLSVHGPRAAEEELVEADEAGSVYAGEQKLISEEDLGGEQKLISEEDLHHHHHH
REGN3153 (human FGFR3b expressed with a C-terminal murine Fc tag
(hFGFR3b.mFc))
(SEQ ID NO: 239)
ESLGTEQRVVGRAAEVPGPEPGQQEQLVFGSGDAVELSCPPPGGGPMGPTVWVKDGTGLVPSERVLVGPQRLQVLNASHE
DSGAYSCRQRLTQRVLCHFSVRVTDAPSSGDDEDGEDEAEDTGVDTGAPYWTRPERMDKKLLAVPAANTVRFRCPAAGNP
TPSISWLKNGREFRGEHRIGGIKLRHQQWSLVMESVVPSDRGNYTCVVENKFGSIRQTYTLDVLERSPHRPILQAGLPAN
QTAVLGSDVEFHCKVYSDAQPHIQWLKHVEVNGSKVGPDGTPYVTVLKSWISESVEADVRLRLANVSERDGGEYLCRATN
FIGVAEKAFWLSVHGPRAAEEELVEADEAGSVYAGEPRGPTIKPCPPCKCPAPNLLGGPSVFIFPPKIKDVLMISLSPIV
TCVVVDVSEDDPDVQISWFVNNVEVHTAQTQTHREDYNSTLRVVSALPIQHQDWMSGKEFKCKVNNKDLPAPIERTISKP
KGSVRAPQVYVLPPPEEEMTKKQVTLTCMVTDEMPEDIYVEWTNNGKTELNYKNTEPVLDSDGSYFMYSKLRVEKKNWVE
RNSYSCSVVHEGLHNHHTTKSESRTPGK
REGN3215 (murine FGFR3b expressed with a C-terminal myc-myc-hexahistidine tag
(mFGFR3b.mmH))
(SEQ ID NO: 240)
EPPGPEQRVVRRAAEVPGPEPSQQEQVAFGSGDTVELSCHPPGGAPTGPTVWAKDGTGLVASHRILVGPQRLQVLNASHE
DAGVYSCQHRLTRRVLCHFSVRVTDAPSSGDDEDGEDVAEDTGAPYWTRPERMDKKLLAVPAANTVRFRCPAAGNPTPSI
SWLKNGKEFRGEHRIGGIKLRHQQWSLVMESVVPSDRGNYTCVVENKFGSIRQTYTLDVLERSPHRPILQAGLPANQTAI
LGSDVEFHCKVYSDAQPHIQWLKHVEVNGSKVGPDGTPYVTVLKSWISENVEADARLRLANVSERDGGEYLCRATNFIGV
AEKAFWLRVHGPQAAEEELMETDEAGSVYAGEQKLISEEDLGGEQKLISEEDLHHHHHH
REGN3155 (human FGFR3c expressed with a C-terminal myc-myc-hexahistidine tag
(hFGFR3c.mmH))
(SEQ ID NO: 241)
ESLGTEQRVVGRAAEVPGPEPGQQEQLVFGSGDAVELSCPPPGGGPMGPTVWVKDGTGLVPSERVLVGPQRLQVLNASHE
DSGAYSCRQRLTQRVLCHFSVRVTDAPSSGDDEDGEDEAEDTGVDTGAPYWTRPERMDKKLLAVPAANTVRFRCPAAGNP
TPSISWLKNGREFRGEHRIGGIKLRHQQWSLVMESVVPSDRGNYTCVVENKEGSIRQTYTLDVLERSPHRPILQAGLPAN
QTAVLGSDVEFHCKVYSDAQPHIQWLKHVEVNGSKVGPDGTPYVTVLKTAGANTTDKELEVLSLHNVTFEDAGEYTCLAG
NSIGESHHSAWLVVLPAEEELVEADEAGSVYAGEQKLISEEDLGGEQKLISEEDLHHHHHH
REGN3521 (mf FGFR3b ecto (R357G)(E23-G377;R357G).mmH)
(SEQ ID NO: 242)
ESLGTEQRVVGRVAEVSGPEPSQQEQLVFGSGDAVELSCPPPGGGPMGPTVWVKDGAGLVPSERVLVGPQ
RLQVLNASHEDSGAYSCRQRLTQLVLCHFSVRVTDAPSSGDDEDGEDEAEDTGVDTGAPYWTRPERMDKKL
LAVPAANTVRFRCPAAGNPTPSISWLKNGKEFRGEHRIGGIKLRHQQWSLVMESVVPSDRGNYTCVVENKFG
SIRQTYTLDVLERSPHRPILQAGLPANQTAVLGSDVEFHCKVYSDAQPHIQWLKHVEVNGSKVGPDGTPYVTVL
KSWISESVEADVRLRLANVSERDGGEYLCRATNFIGVAEKAFWLSVHGPRAAEEELVEADEAGSVYAGE
QKLISEEDLGGEQKLISEEDLHHHHHH

TABLE 1-1
Kinetic and Equilibrium Binding Parameters of Monomeric Human
FGFR3b to Surface-captured anti-FGFR3 Antibodies at 25° C.

| | mAb | 90 nM | | | | |
|---|---|---|---|---|---|---|
| | Capture | hFGFR3b.mmH | | | | t½ |
| | (RU) | Bind (RU) | ka (1/Ms) | kd (1/s) | KD (M) | (min) |
| H4H30045P | 584.3 ± 7.5 | 124.1 | 3.85E+05 | 6.24E−03 | 1.62E−08 | 1.9 |
| H4H30061P | 561.3 ± 8.2 | 249.2 | 1.73E+05 | 8.74E−05 | 5.06E−10 | 132.2 |
| H4H30063P | 577.0 ± 5.6 | 240.5 | 1.72E+05 | 3.83E−05 | 2.22E−10 | 301.9 |
| H4H30066P | 486.8 ± 2.9 | 224.5 | 4.96E+05 | 6.35E−04 | 1.28E−09 | 18.2 |
| H4H30071P | 515.2 ± 7.9 | 253.0 | 2.16E+05 | 2.96E−05 | 1.37E−10 | 390.5 |
| H4H30076P | 436.8 ± 13.8 | 198.0 | 2.36E+05 | 4.39E−05 | 1.86E−10 | 263.3 |
| H4H30089P2 | 479.7 ± 3.5 | 152.1 | 2.76E+05 | 2.82E−03 | 1.02E−08 | 4.1 |
| H4H30093P2 | 496.4 ± 5.2 | 217.0 | 4.06E+05 | 2.09E−03 | 5.14E−09 | 5.5 |
| H4H30095P2 | 506.5 ± 1.1 | 239.4 | 6.98E+05 | 5.00E−03 | 7.16E−09 | 2.3 |
| H4H30102P2 | 438.3 ± 2.7 | 134.8 | 1.68E+05 | 2.43E−04 | 1.44E−09 | 47.5 |
| H4H30105P2 | 467.9 ± 0.3 | 157.7 | 4.38E+05 | 5.11E−03 | 1.17E−08 | 2.3 |
| H4H30108P2 | 544.3 ± 3.1 | 227.2 | 1.76E+05 | 1.59E−04 | 9.05E−10 | 72.6 |
| H4H30117P2 | 412.2 ± 0.8 | 177.5 | 6.56E+05 | 6.30E−03 | 9.60E−09 | 1.8 |
| COMP6331 | 145.4 ± 3.4 | 58.38 | 8.83E+05 | 6.16E−04 | 6.97E−10 | 18.76 |

* See V_{H} and V_{L} set forth in WO2016/134234

REGN6331 Heavy Chain
(SEQ ID NO: 244)
EVQLVESGGGLVQPGGSLRLSCAASGFTFTSTGISWVRQAPGKGLEWVGRIYPTSGSTNYADSVKGRFTISADTSKNTAY
LQMNSLRAEDTAVYYCARTYGIYDLYVDYTEYVMDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTHTCPPCP
APELLGGPSVFLFPPKPKDTLMISRTPEVTCVVVDVSHEDPEVKENWYVDGVEVHNAKTKPREEQYNSTYRVVSVLTVLH
QDWLNGKEYKCKVSNKALPAPIEKTISKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLSPGK
REGN6331 Light Chain
(SEQ ID NO: 243)
DIQMTQSPSSLSASVGDRVTITCRASQDVDTSLAWYKQKPGKAPKLLIYSASFLYSGVPSRFSGSGSGTDFTLTISSLQP
EDFATYYCQQSTGHPQTFGQGTKVEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC

TABLE 1-2
Kinetic and Equilibrium Binding Parameters of Monomeric Cynomolgus
FGFR3b to Surface-captured anti-FGFR3 Antibodies at 25° C.

| | mAb | 90 nM | | | | |
|---|---|---|---|---|---|---|
| | Capture | mfFGFR3b.mmH | | | | t½ |
| Ab PID # | (RU) | Bind (RU) | ka (1/Ms) | kd (1/s) | KD (M) | (min) |
| H4H30045P | 587.4 ± 1.6 | 71.9 | 4.29E+05 | 8.82E−03 | 2.06E−08 | 1.3 |
| H4H30061P | 555.8 ± 4.2 | 197.3 | 1.51E+05 | 9.83E−05 | 6.50E−10 | 117.5 |
| H4H30063P | 574.1 ± 4.8 | 212.5 | 1.53E+05 | 4.21E−05 | 2.75E−10 | 274.7 |
| H4H30066P | 487.2 ± 3.8 | 203.2 | 3.91E+05 | 7.79E−04 | 1.99E−09 | 14.8 |
| H4H30071P | 507.6 ± 6.8 | 233.1 | 1.80E+05 | 2.22E−05 | 1.23E−10 | 520.3 |
| H4H30076P | 437.5 ± 6.9 | 179.7 | 2.00E+05 | 3.30E−05 | 1.65E−10 | 350.2 |
| H4H30089P2 | 486.8 ± 4.6 | 147.3 | 2.16E+05 | 2.21E−03 | 1.03E−08 | 5.2 |
| H4H30093P2 | 499.6 ± 3.1 | 3.4 | NB* | NB* | NB* | NB* |
| H4H30095P2 | 509.2 ± 1.7 | 205.9 | 5.27E+05 | 4.37E−03 | 8.29E−09 | 2.6 |
| H4H30102P2 | 442.5 ± 2.0 | 125.9 | 1.31E+05 | 2.30E−04 | 1.75E−09 | 50.2 |
| H4H30105P2 | 468.6 ± 2.4 | 67.8 | 5.47E+05 | 8.02E−03 | 1.47E−08 | 1.4 |
| H4H30108P2 | 543.7 ± 3.1 | 211.5 | 1.49E+05 | 1.36E−04 | 9.09E−10 | 85.2 |
| H4H30117P2 | 413.8 ± 1.4 | 147.6 | 5.50E+05 | 8.70E−03 | 1.58E−08 | 1.3 |
| COMP6331 | NT** | NT** | NT** | NT** | NT** | NT** |

*NB = Non-Binding
**NT = Not Tested

TABLE 1-3
Kinetic and Equilibrium Binding Parameters of Monomeric Murine
FGFR3b to Surface-captured anti-FGFR3 Antibodies at 25° C.

| | mAb | 90 nM | | | | |
|---|---|---|---|---|---|---|
| | Capture | mFGFR3b.mmH | | | | t½ |
| Ab PID # | (RU) | Bind (RU) | ka (1/Ms) | kd (1/s) | KD (M) | (min) |
| H4H30045P | 586.0 ± 2.6 | 2.4 | NB** | NB** | NB** | NB** |
| H4H30061P | 561.0 ± 1.5 | −8.5 | NB** | NB** | NB** | NB** |
| H4H30063P | 570.0 ± 6.5 | 259.2 | 2.04E+05 | 2.78E−05 | 1.37E−10 | 415.5 |
| H4H30066P | 486.0 ± 2.9 | 64.6 | 2.88E+05 | 4.87E−03 | 1.69E−08 | 2.4 |
| H4H30071P | 511.0 ± 3.2 | 174.3 | 2.20E+05 | 1.69E−03 | 7.65E−09 | 6.9 |
| H4H30076P | 438.1 ± 14.9 | 201.1 | 2.56E+05 | 3.61E−05 | 1.41E−10 | 319.7 |
| H4H30089P2 | 484.0 ± 7.0 | 60.0 | 2.81E+05 | 2.38E−03 | 8.45E−09 | 4.9 |
| H4H30093P2 | 503.8 ± 4.9 | 11.2 | 1.36E+05 | 9.67E−03 | 7.09E−08 | 1.2 |
| H4H30095P2 | NA* | NA* | NA* | NA* | NA* | NA* |
| H4H30102P2 | NA* | NA* | NA* | NA* | NA* | NA* |
| H4H30105P2 | 469.9 ± 0.9 | 8.3 | 1.49E+05 | 1.22E−03 | 8.19E−09 | 9.5 |
| H4H30108P2 | 541.4 ± 3.4 | 121.1 | 1.62E+05 | 1.99E−03 | 1.23E−08 | 5.8 |
| H4H30117P2 | 414.0 ± 1.0 | 0.9 | NB** | NB** | NB** | NB** |
| COMP6331 | NT*** | NT*** | NT*** | NT*** | NT** | NT*** |

*NA: Not Acquired. Technical issue prevented data acquisition and analysis.
**NB = Non-Binding
***NT: Not Tested

TABLE 1-4
Kinetic and Equilibrium Binding Parameters of Monomeric Human
FGFR3c to Surface-captured anti-FGFR3 Antibodies at 25° C.

| | mAb | 90 nM | | | | |
|---|---|---|---|---|---|---|
| | Capture | hFGFR3c.mmH | | | | t½ |
| Ab PID # | (RU) | Bind (RU) | ka (1/Ms) | kd (1/s) | KD (M) | (min) |
| H4H30045P | 583.4 ± 1.4 | 77.1 | 3.81E+05 | 7.46E−03 | 1.96E−08 | 1.5 |
| H4H30061P | 555.4 ± 4.1 | 203.8 | 1.51E+05 | 7.92E−05 | 5.26E−10 | 145.8 |
| H4H30063P | 577.8 ± 1.9 | −19.6 | NB* | NB* | NB* | NB* |
| H4H30066P | 485.8 ± 2.4 | 0.5 | NB* | NB* | NB* | NB* |
| H4H30071P | 519.0 ± 0.5 | −4.8 | NB* | NB* | NB* | NB* |
| H4H30076P | 435.7 ± 9.9 | −9.7 | NB* | NB* | NB* | NB* |
| H4H30089P2 | 484.6 ± 5.0 | −6.7 | NB* | NB* | NB* | NB* |
| H4H30093P2 | 503.2 ± 4.1 | 182.1 | 3.63E+05 | 3.34E−03 | 9.20E−09 | 3.5 |
| H4H30095P2 | 508.5 ± 0.9 | 221.8 | 5.75E+05 | 4.54E−03 | 7.88E−09 | 2.5 |
| H4H30102P2 | 448.0 ± 0.7 | −11.7 | NB* | NB* | NB* | NB* |
| H4H30105P2 | 469.7 ± 0.8 | 130.6 | 3.87E+05 | 6.52E−03 | 1.68E−08 | 1.8 |
| H4H30108P2 | 541.2 ± 1.9 | −2.5 | NB* | NB* | NB* | NB* |
| H4H30117P2 | 413.7 ± 0.6 | 165.5 | 5.95E+05 | 7.32E−03 | 1.23E−08 | 1.6 |
| COMP6331 | 141.5 ± 3.2 | 39.56 | 5.44E+05 | 1.32E−03 | 2.42E−09 | 8.77 |

*NB = Non-Binding

TABLE 1-5
Kinetic and Equilibrium Binding Parameters of Dimeric Human
FGFR3b to Surface-captured anti-FGFR3 Antibodies at 25° C.

| | mAb | 30 nM | | | | |
|---|---|---|---|---|---|---|
| | Capture | hFGFR3b.mFc | | | | t½ |
| Ab PID # | (RU) | Bind (RU) | ka (1/Ms) | kd (1/s) | KD (M) | (min) |
| H4H30045P | 573.0 ± 4.6 | 265.5 | 2.77E+05 | 1.61E−04 | 5.81E−10 | 71.8 |
| H4H30061P | 544.3 ± 4.8 | 259.7 | 2.31E+05 | ≤1E−5 | 4.33E−11 | ≥1155 |
| H4H30063P | 567.0 ± 7.1 | 296.1 | 4.42E+05 | ≤1E−5 | 2.26E−11 | ≥1155 |
| H4H30066P | 484.6 ± 2.2 | 282.3 | 6.32E+05 | ≤1E−5 | 1.58E−11 | ≥1155 |
| H4H30071P | 502.2 ± 2.5 | 235.8 | 3.22E+05 | ≤1E−5 | 3.10E−11 | ≥1155 |
| H4H30076P | 435.0 ± 2.6 | 204.1 | 4.07E+05 | ≤1E−5 | 2.46E−11 | ≥1155 |
| H4H30089P2 | 485.6 ± 4.1 | 213.9 | 3.12E+05 | 1.90E−05 | 6.10E−11 | 607.9 |
| H4H30093P2 | 497.8 ± 8.2 | 316.9 | 5.85E+05 | 1.55E−05 | 2.65E−11 | 744.7 |
| H4H30095P2 | 504.5 ± 2.0 | 404.9 | 8.67E+05 | 9.88E−05 | 1.14E−10 | 117.0 |
| H4H30102P2 | 443.7 ± 1.7 | 131.4 | 2.97E+05 | ≤1E−5 | 3.37E−11 | ≥1155 |
| H4H30105P2 | NA* | NA* | NA* | NA* | NA* | NA* |
| H4H30108P2 | 532.1 ± 3.5 | 213.0 | 3.11E+05 | ≤1E−5 | 3.22E−11 | ≥1155 |
| H4H30117P2 | 411.4 ± 1.5 | 337.0 | 7.87E+05 | 1.33E−04 | 1.69E−10 | 87.0 |
| COMP6331 | NT** | NT** | NT** | NT** | NT** | NT** |

*NA = Not Acquired. Technical issue prevented data acquisition and analysis
**NT = Not Tested

### Example 2: Blocking Assays

Anti-FGFR3 antibodies blocking dimeric human FGFR3b or FGFR3c binding to human FGF acidic or FGF basic by ELISA was analyzed.

TABLE 2-1
Reagents used

| Reagent | Source | Identification |
|---|---|---|
| hFGF acidic | R&D Systems | 231-BC-025/CF |
| hFGF basic | Biolegend | 571508 |
| hFGFR3c-hFc | R&D Systems | 766-FR-050 |
| hFGFR3b-mFc | | REGN3153 |
| Human IgG1 | | REGN1932 |
| negative control | | |
| antibody (specific for | | |
| cat allergy antigen - | | |
| Fel d1) | | |
| Human IgG4 | | REGN1945 |
| negative control | | |
| antibody (specific for | | |
| cat allergy antigen - | | |
| Fel d1) | | |
| anti-FGFR3-hIgG1 | | REGN6331 |
| comparator | | |
| Peroxidase | Jackson | 109-035-098 |
| AffiniPure Goat Anti- | ImmunoResearch | |
| Human IgG, Fcγ | | |
| fragment specific | | |
| Peroxidase | Jackson | 115-035-164 |
| AffiniPure Goat Anti- | ImmunoResearch | |
| Mouse IgG | | |
| (subclasses | | |
| 1 + 2a + 2b + 3), Fcγ | | |
| Fragment Specific | | |
| 1X PBS | Irvine Scientific | 9236 |
| 1X PBST (0.05% | Sigma | P3563 |
| tween-20 in PBS) | | |
| BSA: albumin | Sigma | A8577-1L |
| solution from bovine | | |
| serum, 30% | | |
| Heparin | Sigma | H4784-1G |
| 3-3′, 5-5′- | BD Biosciences | 51-2606KC |
| tetramethylbenzidine | | |
| (TMB) Substrate | | |
| Reagent Set - | | |
| Substrate Reagent A | | |
| 3-3′, 5-5′- | BD Biosciences | 51-2607KC |
| tetramethylbenzidine | | |
| (TMB) Substrate | | |
| Reagent Set - | | |
| Substrate Reagent B | | |
| 2N Sulfuric Acid | VWR | BDH7500-1 |
| Reacti-Bind 96-well | ThermoFisher | P15041 |
| plates | Scientific | |
| 96-well deep well | VWR | 82006-448, |
| plates | | 82007-292 |
| 96-Well | Fisher Scientific | Fisher 12-565-369 |
| Polypropylene | | |
| storage microplates | | |
| Aquamax Plate | Molecular Devices | Aquamax 2000 |
| Washer 2000 | | |
| VICTOR ™ X5 | PerkinElmer | Model #2030 |
| Multilabel Plate | | |
| Reader | | |
| GraphPad Prism | GraphPad Software | Version 9 |

An ELISA-based blocking assay was developed to determine the ability of anti-FGFR3 antibodies to block the binding of human fibroblast growth factor receptor 3 isoform b (hFGFR3b) or human fibroblast growth factor receptor 3 isoform c (hFGFR3c) to human fibroblast growth factor acidic (hFGF acidic) or basic (hFGF basic) ligands.

The human FGFR3b recombinant protein used in the experiments had the hFGFR3b extracellular domain (amino acids E23-G377) expressed with the Fc portion of the mouse IgG2a at the C-terminus (amino acids E98-K330) (hFGFR3b-mFc, accession #NM_001163213.1). The hFGFR3c and hFGF acidic proteins were purchased commercially. The human FGFR3c protein had the hFGFR3c extracellular domain (amino acids Glu23-Gly375) expressed with Fc portion of the human IgG1 at the C-terminus (amino acids Pro100-Lys330) (hFGFR3c-hFc, accession #P22607) and the hFGF acidic protein was expressed with amino acids Ala2-Asp155 (accession #P05230.1). The hFGF basic protein was purchased commercially. It was expressed with amino acids Ala144-Ser288 (accession #NM_002006).

In the blocking assay, a 96-well microtiter plate was coated with either hFGF acidic or hFGF basic proteins at 2 mg/ml in PBS+10 mg/ml heparin overnight at 4° C. Nonspecific binding sites were subsequently blocked using a 0.5% (w/v) solution of BSA+10 μg/ml heparin in PBS. In other 96-well microtiter plates, a fixed amount of 4 nM hFGFR3b-mFc, 0.4 nM or 7 nM hFGFR3c-hFc was bound for one hour with anti-FGFR3, anti-FGFR3 comparator, or irrelevant human IgG1 or IgG4 isotype antibody at dilutions from 3.4 pM to 200 nM in PBS+0.5% BSA+10 μg/ml heparin. The fixed concentration of hFGFR3b or hFGFR3c proteins was selected to be near the concentration that generated 50% of the maximal binding (EC₅₀ value) to plate-adhered hFGF acidic or hFGF basic protein. The antibody complexes with 4 nM hFGFR3b-mFc or 0.4 nM hFGFR3c-hFc were transferred to microtiter plates coated with hFGF acidic protein. In parallel, antibody complexes with 7 nM hFGFR3c-hFc were added to the microtiter plates coated with hFGF basic protein. After a 1 hour incubation at room temperature, plates were washed, and plate-bound hFGFR3b-mFc or hFGFR3c-hFc proteins were detected with horseradish peroxidase (HRP) conjugated goat anti-mouse or goat anti-human Fcγ fragment specific antibodies. The plates were then developed using TMB substrate solution (BD Biosciences) according to the manufacturer's recommended procedure and absorbance at 450 nm was measured on a Victor X5 plate reader.

Binding data were analyzed using a sigmoidal (four-parameter logistic) dose-response model using GraphPad Prism software. The calculated IC₅₀ value, defined as the concentration of antibody required to block 50% of hFGFR3b-mFc or hFGFR3c-hFc binding to plate-coated hFGF acidic or hFGF basic protein, was used as an indicator of blocking potency. Percent blocking of FGFR3 antibody at a given concentration was calculated based on the formula shown below.

$$\% Blocking = 100 - ( \frac{\begin{matrix}Experimental {Signal}_{( highest Ab conc )} - \\ Background {Signal}_{( buffer )}\end{matrix}}{\begin{matrix}Maximum {Signal}_{( hFGFR3b - mFc or hFGF3c - hFc )} - \\ Background {Signal}_{( buffer )}\end{matrix}} ) \times 100$$

Antibodies that blocked binding more than 50% at the highest concentration tested were classified as blockers and IC₅₀ values were reported.

The ability of anti-FGFR3 antibodies to block human FGFR3b binding to hFGF acidic protein or hFGFR3c binding to hFGF acidic or hFGF basic proteins was evaluated using a sandwich ELISA-based blocking assay. In this assay, a fixed concentration of the hFGFR3b-mFc or hFGFR3c-hFc was pre-incubated with a wide concentration range of anti-FGFR3 antibodies before binding to plate immobilized hFGF acidic or basic proteins, and the plate-bound hFGFR3b-mFc or hFGFR3c-hFc was detected with HRP-conjugated goat anti-mouse or goat anti-human Fcγ fragment specific antibodies, respectively. Six anti-FGFR3 antibodies were evaluated for inhibition of hFGFR3b-mFc binding to hFGF acidic protein. Antibody H4H30093P2, which also binds hFGFR3c, was additionally tested for inhibition of hFGFR3c-hFc binding to hFGF acidic or hFGF basic proteins. The IC₅₀ values and maximum blocking at the highest tested concentrations of the FGFR3 antibodies are summarized in Table 2-2.

All six anti-FGFR3 antibodies (H4H30063P, H4H30066P, H4H30071P, H4H30089P2, H4H30093P2 and H4H30102P2) displayed concentration-dependent blocking of hFGFR3b-mFc binding to hFGF acidic protein with the extent of block ranging from 68% to 95% at the highest antibody concentration tested (200 nM). The IC₅₀ values for these blocking antibodies ranged from 2 nM to 15 nM. Antibody H4H30093P2 displayed less than 50% blocking of the binding of hFGFR3c to hFGF acidic or hFGF basic at the highest antibody concentration tested and was classified as a non-blocker for hFGFR3c. The anti-FGFR3 comparator antibody blocked the binding of hFGFR3b to hFGF acidic with an IC₅₀ of 1.5 nM, and binding of hFGFR3c binding to hFGF acidic or hFGF basic with an IC₅₀ of 0.1 nM and 8.9 nM, respectively. The human IgG1 or IgG4 isotype control antibodies did not block in any assays.

TABLE 2-2
IC₅₀ values and maximum blocking at the highest
tested concentrations of the FGFR3 antibodies

| | Antibody blocking | | Antibody blocking | | Antibody blocking | |
|---|---|---|---|---|---|---|
| | 4 nM hFGFR3b-mFc | | 0.4 nM hFGFR3c-hFc | | 7 nM hFGFR3c-hFc | |
| | binding to hFGF | | binding to hFGF | | binding to hFGF | |
| | acidic protein | | acidic protein | | basic protein | |
| | | % Blocking | | % Blocking | | % Blocking |
| | | at 200 nM | | at 200 nM | | at 200 nM |
| Antibody | IC₅₀, M | mAb | IC₅₀, M | mAb | IC₅₀, M | mAb |
| H4H30063P | 2.0E−09 | 95 | ND | ND | ND | ND |
| H4H30066P | 3.2E−09 | 90 | ND | ND | ND | ND |
| H4H30071P | 3.6E−09 | 88 | ND | ND | ND | ND |
| H4H30089P2 | 1.5E−08 | 68 | ND | ND | ND | ND |
| H4H30093P2 | 2.5E−09 | 91 | Nbl | −7 | Nbl | −15 |
| H4H30102P2 | 2.5E−09 | 92 | ND | ND | ND | ND |
| hlgG4 isotype | Nbl | 14 | Nbl | 16 | Nbl | −5 |
| control | | | | | | |
| anti-FGFR3- | 1.5E−09 | 99 | 1.0E−10 | 86 | 8.9E−09 | 87 |
| hlgG1 | | | | | | |
| comparator (*) | | | | | | |
| hlgG1 isotype | Nbl | −7 | Nbl | 14 | Nbl | −20 |
| control (*) | | | | | | |

Nbl: Non-blocking, % blocking is less than or equal to 50
ND: Not determined
(*) anti-FGFR3-hlgG1 comparator and hlgG1 isotype control were tested in a separate experiment for blocking of hFGFR3b binding to hFGF acidic protein or hFGFR3c binding to hFGF basic protein

### Example 3: Octet Cross-Competition Analysis of Anti-FGFR3 Antibodies

Binding competition between anti-FGFR3 monoclonal antibodies that had been previously determined to bind to hFGFR3b.mmH was determined using a real time, label-free bio-layer interferometry (BLI) assay on an Octet HTX biosensor (ForteBio Corp., A Division of Pall Life Sciences). The entire experiment was performed at 25° C. in buffer comprised of 0.01 M HEPES pH7.4, 0.15 M NaCl, 3 mM EDTA, 0.05% v/v Surfactant P20, 0.1 mg/mL BSA (Octet HBS-EP buffer) with the plate shaking at a speed of 1000 rpm. To assess whether two antibodies were able to compete with one another for binding to hFGFR3b ecto domain expressed with a C-terminal myc-myc-histidine tag (hFGFR3b.mmH, REGN3152) approximately 0.2 nm of hFGFR3b.mmH was first captured onto anti-penta-His antibody coated Octet biosensors (Fortebio Inc, #18-5079) by submerging the biosensors for 23 seconds into wells containing a 20 μg/mL solution of hFGFR3b.mmH. The antigen-captured biosensors were then saturated with the first anti-FGFR3 monoclonal antibody (subsequently referred to as mAb-1) by immersion into wells containing a 50 μg/mL solution of mAb-1 for 5 minutes. The biosensors were then subsequently submerged into wells containing a 50 μg/mL solution of a second anti-FGFR3 monoclonal antibody (subsequently referred to as mAb-2) for 3 minutes. The real-time binding response was monitored during the course of the experiment and the binding response at the end of every step was recorded. The response of mAb-2 binding to hFGFR3b.mmH pre-complexed with mAb-1 was compared and the competitive or non-competitive property of an anti-FGFR3 monoclonal antibody toward another antibody was determined using a 50% inhibition threshold. Table 3-1 summarizes cross-competing antibodies which competed binding to hFGFR3b.mmH independent of the order of the sequential binding of mAb-1 and mAb-2.

TABLE 3-1
Cross-competition of anti-FGFR3 antibodies
for binding to hFGFR3b.mmH.

| First mAb (mAb-1) | mAb-2 Antibodies |
|---|---|
| captured using AHC | Shown to Compete |
| Octet Biosensors | with mAb-1 |
| H4H30045P | H4H30045P |
| | H4H30095P2 |
| H4H30061P | H4H30093P2 |
| | H4H30061P |
| H4H30063P | H4H30063P |
| | H4H30076P |
| H4H30066P | H4H30089P2 |
| | H4H30108P2 |
| | H4H30071P |
| | H4H30102P2 |
| | H4H30066P |
| H4H30071P | H4H30089P2 |
| | H4H30108P2 |
| | H4H30071P |
| | H4H30102P2 |
| | H4H30066P |
| H4H30076P | H4H30063P |
| | H4H30076P |
| | H4H30089P2 |
| H4H30089P2 | H4H30076P |
| | H4H30089P2 |
| | H4H30108P2 |
| | H4H30071P |
| | H4H30102P2 |
| | H4H30066P |
| H4H30093P2 | H4H30093P2 |
| | H4H30061P |
| H4H30095P2 | H4H30045P |
| | H4H30095P2 |
| | H4H30105P2 |
| | H4H30117P2 |
| H4H30102P2 | H4H30089P2 |
| | H4H30108P2 |
| | H4H30071P |
| | H4H30102P2 |
| | H4H30066P |
| H4H30105P2 | H4H30095P2 |
| | H4H30105P2 |
| | H4H30117P2 |
| H4H30108P2 | H4H30089P2 |
| | H4H30108P2 |
| | H4H30071P |
| | H4H30102P2 |
| | H4H30066P |
| H4H30117P2 | H4H30095P2 |
| | H4H30105P2 |
| | H4H30117P2 |

### Example 4: Characterization of FGFR3 Antibodies in Cell Growth Assays Using Engineered BaF3 Cells Overexpressing Human FGFR3 WT or FGFR3-S249C Mutant Receptor

The ability of anti-FGFR3 antibodies to block FGFR3 signaling was evaluated using a proliferation assay using an engineered IL-3-dependent Ba/F3 murine hematopoietic cell line genetically modified to stably express human fibroblast growth factor receptor 3b wild-type or mutant S249C.

FGFR3 antigen binding molecules and the controls tested in this experiment are as shown in Table 4-1.

TABLE 4-1
FGFR3b Binding Molecules and Controls

| mAb Identifier | Description |
|---|---|
| H4H30063P | FGFR3b mAb, hIgG4^{P} |
| H4H30102P2 | FGFR3b mAb, hIgG4^{P} |
| REGN6331 | Relates to FGFR3b mAb, hIgG1 |
| | (mAb 184.6.1 N54S, Genentech) |
| REGN1932 | Anti-FelD1, Isotype control, hIgG1 |
| REGN1945 | Anti-FelD1, Isotype control, hIgG4^{P} |

### Cell Lines:

BAF3/FGFR3b (ACL11991): IL-3 dependent murine pro B cell line, engineered to stably express full length human FGFR3b, (accession number NP_001156685.1, amino acids M1-T808). Cells are maintained in RPMI 1640+10% FBS+P/S/G+1 ng/ml mouse IL-3+500 ug/ml Neomycin; 37° C. 5% CO₂.
BAF3/FGFR3b_S249C (ACL11986): IL-3 dependent murine pro B cell line, engineered to stably express full length human FGFR3b, (accession number NP_001156685.1, amino acids M1-T808, S249C). Cells are maintained in RPMI 1640+10% FBS+P/S/G+1 ng/ml mouse IL-3+500 ug/ml Neomycin; 37° C. 5% CO₂.

The FGFR3 receptor is a member of the receptor tyrosine kinase (RTK) family, which regulates cell proliferation, survival, differentiation and migration of multicellular organisms. The activation of the wild-type (WT) receptor occurs via binding of its soluble ligand, e.g., FGF1, which drives the homo-dimerization and autophosphorylation of FGFR3, leading eventually to the activation of a plethora of intracellular signaling cascades such as Ras/MAPK, PLCγ1/PKC, PI3-kinase/Akt, and STAT pathways (Xie et al., FGF/FGFR signaling in health and disease, Signal Transduction and Targeted Therapy (2020) 5:181). Mutations in FGFR3, leading to aberrant FGFR3 activation, have been associated with numerous types of human malignancies. The mutation, S249C, leads to constitutive, ligand independent, FGFR3 activation (Tomlinson et al., Knockdown by shRNA identifies S249C mutant FGFR3 as a potential therapeutic target in bladder cancer, Oncogene. 2007 Aug. 30; 26(40): 5889-5899).

To study the blocking effects of anti-FGFR3b antibodies on signaling, a proliferation assay was deployed using an engineered IL-3-dependent Ba/F3 murine hematopoietic cell line genetically modified to stably express human fibroblast growth factor receptor 3b wild-type or mutant S249C (FGFR3b—accession number NP_001156685.1, amino acids M1-T808) (Kong et al., Ba/F3 transformation assays, Oncotarget, 2017, Vol. 8, (No. 22), pp: 35488-35489)). Engineered BaF3/FGFR3b cells were stimulated with ligand (human FGF1) in the presence of titrated antibodies and cell growth was evaluated using CellTiter-Glo which measures ATP, a product of viable, proliferating cells.

Engineered BaF3/hFGFR (WT or S249C) cells grown in culture medium (RPM11640+10% FBS+Penicillin/Streptomycin/L-Glutamine+1 ng/mL mouse IL-3+500 ug/mL Neomycin) were washed and plated in IL-3-free culture media containing 5 ug/ml heparin and 1 nM human FGF1. Cells were plated out at 10⁵ cells/well into 96-well white tissue culture plates, followed by the addition of 1:4 serially diluted antibodies, ranging from 1.5 pM to 100 nM including a no antibody containing control (plotted at 0.4 pM). After addition of antibodies, the 96-well white microtiter plates were incubated at 37° C./5% CO₂ for 72 h followed by the addition of an equal volume of CellTiter-Glo™ (Promega) reagent to lyse cells and detect luciferase activity. The emitted light was captured in Relative Light Units (RLU) on a multi-label plate reader Envision (PerkinElmer). EC₅₀ values of the antibodies were determined from a 4-parameter logistic equation over a 10-point dose response curve (the 10^{th} point containing no antibody) using GraphPad Prism software.

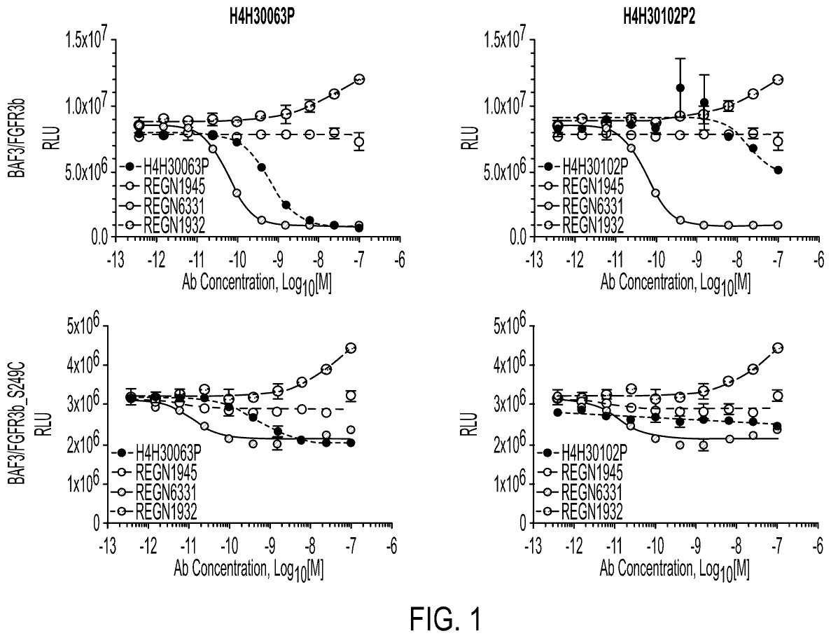

Anti-FGFR3b antibodies, H4H30063P and H4H30102P2, were tested alongside comparator antibody, REGN6331, and isotype matched negative controls in the presence of 1 nM FGF1 and 5 ug/ml heparin (see Table 4-2 and FIG. 1).

Anti-FGFR3b antibodies, H4H30063P and H4H30102P2, and their corresponding matched isotype control, REGN1945, were tested alongside comparator antibody REGN6331 and isotype matched control, REGN1932, in the presence of 1 nM FGF1+5 ug/ml heparin. It was observed that, in experiments using BaF3/hFGFR3b WT cell lines, antibodies H4H30063P and REGN6331 exhibited a similar level of maximal inhibition of proliferation, whereas antibody H4H30102P2 did not reach the same level of maximum inhibition. The degree of ligand mediated growth in BaF3/FGFR3_S249C cells was minimal due to the S249C mutation leading to constitutive FGFR3b activity. Nonetheless, ligand addition led to slight enhancement of proliferation, which can be inhibited to similar maximum levels by antibodies H4H30063P and REGN6331, while H4H30102P2 led to minimal inhibition with non-calculatable IC50 values.

TABLE 4-2
Maximum Inhibition and Potency values of Antibodies

| MABM | Max Blocking (%)^{a} | | IC50 [M] | |
|---|---|---|---|---|
| Identifier | BAF3/FGFR3b | BAF3/FGFR3b_S249C | BAF3/FGFR3b | BAF3/FGFR3b_S249C |
| H4H30063P | 98.01 | 93.36 | 6.06E−10 | 5.08E−10 |
| H4H30102P2 | 41.07 | 34.55 | 1.86E−08 | NC |
| REGN6331 | 97.60 | 109.56 | 5.79E−11 | 1.22E−11 |
| REGN1945 | ND | ND | ND | ND |
| REGN1932 | NC | NC | NC | NC |

^{a}Maximal Blocking is given as the mean maximum blocking detected within the tested dose range. Abbreviations: ND: Not Determined; NC: Not Calculated; a potency value could not be determined by PRISM

$$Blocking = 100 - ( \begin{matrix}Mean RLU at 100 nm mAb - RLU No Ligand & \times 100 \\ Mean RLU at 0 nm mAb - RLU No Ligand & \end{matrix} )$$

### Example 5: Characterization of Mechanism of Action of FGFR3 Antibodies

Assays evaluating the following were performed: (1) Inhibition of FGFR3 S249C dimerization using bladder cancer cell line UMUC14 with endogenous FGFR3 S249C mutation; and (2) Inhibition of FGF1/heparin induced downstream signaling in BaF3 cell expressing wild-type FGFR3.

### Cell Lines:

UMUC14 (Sigma-Aldrich): Cells are maintained in MEM+1% Non-Essential Amino Acids (NEAA) 10% FBS+P/S; 37° C. 5% CO₂.
BAF3/FGFR3b (ACL11991): IL-3 dependent murine pro B cell line, engineered to stably express full length human FGFR3b, (accession number NP_001156685.1, amino acids M1-T808). Cells are maintained in RPMI 1640+10% FBS+P/S/G+1 ng/ml mouse IL-3+500 ug/ml Neomycin; 37° C. 5% CO₂

### Reagents:

rhFGF Acidic, R&D Systems, cat 232-FA, reconstituted at 100 mg/ml in PBS/0.1% BSA
Heparin sodium salt from porcine intestinal mucosa, Sigma Aldrich, cat H3393-100KU, reconstituted at 50 mg/ml in H₂O
FGFR3 (B-9), Santa Cruz Biotechnology, cat sc-13121
ECL Anti-Mouse IgG Horseradish Peroxidase Linked Whole Antibody (from sheep) GPR, cat NXA934V,
SuperSignal West Pico PLUS Chemiluminescent Substrate, Thermo Scientific, cat 34578,
Cell Lysis Buffer(10×), Cell Signaling Technology, cat 9803.
Phospho-p44/42 MAPK(Erk1/2). Cell Signaling Technology, cat 4370.
p44/42 MAPK(Erk1/2). Cell Signaling Technology, cat 4695.
Goat anti-rabbit IgG (H+L), HRP-linked Antibody. Seracare, Cat no 5450-0010

For the receptor dimerization assay, UMUC14 bladder cancer cells were seeded in 6 well tissue culture plate (Corning) in complete medium (MEM medium with 10% FBS, 1% Non Essential Amino Acids, Pen/Strep) and cultured overnight at 37° C. 5% CO₂. Cells were then serum starved overnight in starvation medium (MEM medium with 0.5% FBS, Pen/Strep) followed by antibody treatment for 3 hrs at indicated dose. Cells were washed with pre-chilled PBS and collected in lysis buffer containing protease inhibitors. Equal amount of cell lysate were analyzed by either reducing or non-reducing SDS-PAGE. Blots were blocked in 0.5% Tween 20 in Tris buffered saline (TBS) containing 5% non-fat dry milk followed by overnight incubation with anti-FGFR3 primary antibody (Santa Cruz). After washing 3× with TBST and incubation with secondary antibody, membranes were developed with SuperSignal West Pico or Femto substrate and luminescence images were captured with a C300 imager (Azure Biosystems).

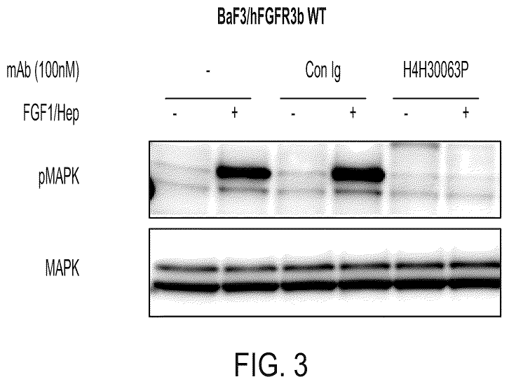

To test the effects of anti-FGFR3 antibody on ligand-induced signaling, an engineered IL-3-dependent Ba/F3 murine hematopoietic cell line genetically modified to stably express wild-type human fibroblast growth factor receptor 3b was used. Engineered BaF3/FGFR3b cells were incubated overnight in starvation medium (RPM11640+Penicillin/Streptomycin/L-Glutamine+1 ng/mL mouse IL-3). After starvation, cells were either untreated or pretreated with 100 nM antibodies for 3 hrs followed by ligand (human 100 ng/ml FGF1 and 10 mg/ml heparin) stimulation for 10 min at 37° C. See FIG. 3. Cells were lysed in lysis buffer and equal amount of cell lysates were analyzed by SDS-PAGE. Blots were incubated with pMAPK or MAPK antibody overnight followed by incubation with anti-rabbit HRP 2^{nd} antibody. Membranes were developed with SuperSignal West Pico or Femto substrate and luminescence images were captured with a C300 imager (Azure Biosystems).

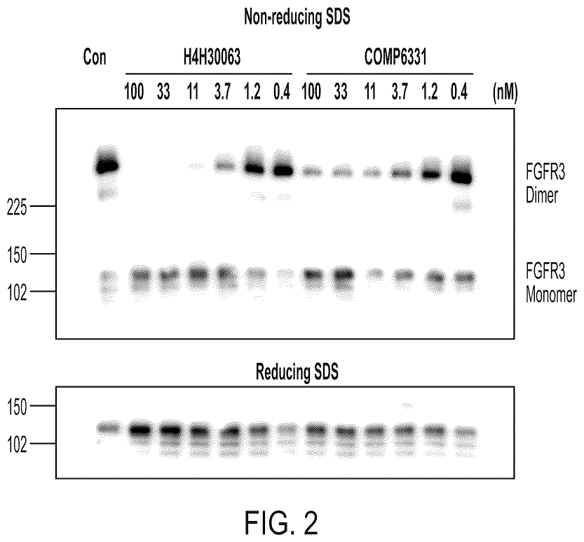

Antibody H4H30063P exhibited dose dependent inhibition of FGFR3 S249C dimerization and stronger inhibition of receptor dimerization than comparator antibody REGN6331. See FIG. 2. In addition, H4H30063P decreased FGF1/Hep stimulation-dependent phosphorylation of MAPK. See FIG. 3.

### Example 6: Characterization of FGFR3 Antibodies in Cancer Cell Spheroid Proliferation Assays Using Bladder Cancer Cell Line UMUC14 with Endogenous FGFR3 S249C Mutation

Anti-FGFR3 antibody-mediated inhibition of cancer cell proliferation was evaluated.

### Cell Lines

UMUC14 (Sigma-Aldrich): Cells are maintained in MEM+1% Non-Essential Amino Acids (NEAA) 10% FBS+P/S; 37° C. 5% CO₂.

### Reagents

Corning Spheroid Microplate 96 Well, Corning, cat #4520
CellTiter-Glo 3D Cell Viability Assay, Promega, cat #G9682

7500 UMUC14 bladder cancer cells were seeded in U-bottom low attachment 96-well spheroid plate (Corning) in culture medium (MEM medium with 10% FBS, 1% Non-Essential Amino Acids, Pen/Strep). Cells were cultured for 48 hours at 37° C. 5% CO₂ to allow tumor spheroid formation. Tumor spheroids were treated with antibodies at concentration ranging from 100 nM to 15.2 pM with 1:3 serial dilution as indicated in the figure. Cells were cultured for 5-6 days, and then subjected to CellTiter-Glo 3D viability assays (Promega) following the manufacturer's protocol. Luminescence signal was read on SpectraMax M3 plate reader. Cell proliferation was expressed as percentages of untreated controls. Data were analyzed using GrasphPad Prism software using three parameter nonlinear curve fit.

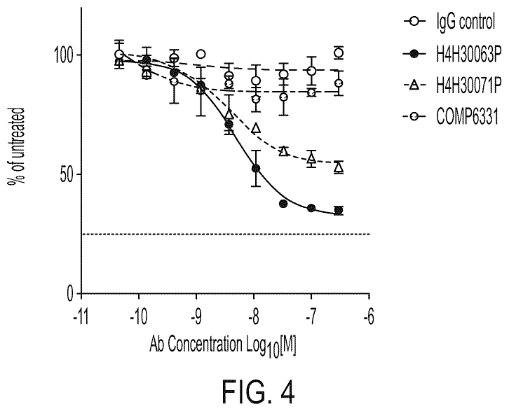

H4H30063P exhibited stronger inhibition of proliferation than antibody H4H30071P and comparator antibody REGN6331. See FIG. 4.

### Example 7: In Vivo Characterization of FGFR3 Antibodies in Tumor Growth Inhibition Assays Using Xenograft Model of Bladder Cancer Cell Line UMUC14 with Endogenous FGFR3 S249C Mutation

Inhibition of bladder cancer cell line growth in a mouse xenograft model exposed to anti-FGFR3 antibody was evaluated in this example.

### Cell Lines

UMUC14 (Sigma-Aldrich): Cells are maintained in MEM+1% Non-Essential Amino Acids (NEAA)+10% FBS+P/S; 37° C. 5% CO₂.

### Reagents

Matrigel Basement Membrane Matrix. Corning, cat #354234, lot: 8085009

Tumor cells (5×10⁶ UMUC14 in 50% Matrigel, 100 ul) were implanted subcutaneously into the right flank of 6 to 8-week-old female SCID mice (Jackson Laboratory). Once tumors were established (˜200 mm³ in volume), mice were randomized into treatment groups (n=10 mice per group) and injected intraperitoneally twice per week with anti-FGFR3 antibodies or isotype control at 3 or 10 mg/kg. Tumor volume was expressed in mm³ using the formula: V=0.5×a×b² where a and b were the long and short diameters of the tumor, respectively. All data were analyzed using GraphPad Prism and tumor sizes were graph as mean+SEM.

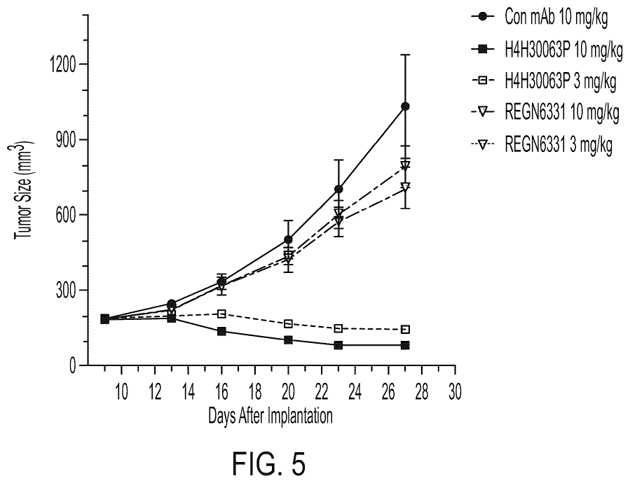

H4H30063P exhibited stronger tumor growth inhibition than comparator antibody REGN6331 in UMUC14 xenograft model with endogenous expression of FGFR3 S249C mutation. See FIG. 5.

### Example 8: Effect of FGFR3 Oncogenic Signaling on CD73 Expression

The effects of FGFR3 mutations and inhibitors of FGFR3 on the expression of CD73 was evaluated.

### Cell Lines:

UMUC14 (Sigma-Aldrich): Cells are maintained in MEM+1% Non-Essential Amino Acids (NEAA) 10% FBS+P/S; 37° C. 5% CO₂
Fadu EV and S249C Cells are maintained in MEM+10% FBS+Pen/Strep+400 ug/ml Neomycin; 37° C. 5% CO₂

### Reagents:

FGFR-3 (B-9), Santa Cruz Biotechnology, Cat #sc-13121
NTSE/CD73(D7F9A) Rabbit mAb; Cell Signaling Technology. Cat #13160
Goat anti-rabbit IgG (H+L), HRP-conjugated, Seracare, Cat #5450-0010
Peroxidase Anti-Mouse IgG(H+L), Vector Laboratories, Cat #PI-2000
SuperSignal West Pico PLUS chemiluminescent substrate, Thermo Scientific, Cat #34578
SuperSignal West Femto Maximum Substrate, Thermo Scientific, Cat #34096
Cell Lysis Buffer(10×), Cell Signaling Technology, Cat #9803
RIPA buffer, Sigma Aldrich, Cat #R0278
Matrigel, Corning, Cat #354234

To investigate the effect of anti-FGFR3 antibody or tyrosine kinase inhibitor (AZD4547; commercially available) on CD73 expression, UMUC14 bladder cancer cells were seeded in 6 well tissue culture plate (Corning) in complete medium (MEM medium with 10% FBS, 1% Non-Essential Amino Acids, Pen/Strep) and cultured overnight at 37° C. 5% CO₂. Cells were then treated with antibodies or inhibitors at indicated concentration (100 nM, 10 nM or 1 nM) for 48 hrs. Cells were washed with pre-chilled PBS and collected in lysis buffer containing protease and phosphatase inhibitors. Equal amount of cell lysates were analyzed by SDS-PAGE. Blots were blocked in 0.5% Tween 20 in Tris buffered saline (TBS) containing 5% non-fat dry milk followed by overnight incubation with anti-CD73 primary antibody (Cell Signaling Technology). After washing 3× with TBST and incubation with secondary antibody, membranes were developed with SuperSignal West Pico or Femto substrate and luminescence images were captured with a C300 imager (Azure Biosystems).

To test the effect of FGFR3 oncogenic signaling on CD73 expression in vivo, 3×10⁶ Fadu cells expressing FGFR3 oncogenic mutation (S249C or FGFR3-TACC3) or empty vector control (EV) were implanted subcutaneously into the right flank of 6 to 8-week-old female SCID mice (Jackson Laboratory). ˜16 day post implantation, tumors were collected, snap frozen and lysed in RIPA buffer. Equal amount of tumor lysates were analyzed by SDS-PAGE and blotted with anti-CD73 and anti-FGFR3 antibodies as described above.

To test the effect of anti-FGFR3 inhibitory antibody or tyrosine kinase inhibitor (AZD4547) on CD73 expression in vivo, tumor cells (5×10⁶ UMUC14, 3×10⁶ Fadu EV or S249C, in 50% Matrigel) were implanted subcutaneously into the right flank of 6 to 8-week-old female SCID mice (Jackson Laboratory). Once tumors were established (˜200 mm³ in volume), mice were randomized into treatment groups (n=5-6 mice per group) and injected intraperitoneally once with anti-FGFR3 antibodies (15 mg/kg) or isotype control. Tyrosine kinase inhibitor AZD4547 (25 mg/kg) was administered once daily by oral gavage for 3 days. Tumors were collected 4 days post treatment initiation. Tumors were collected, snap frozen and lysed in RIPA buffer. Equal amount of tumor lysates were analyzed by SDS-PAGE and blotted with anti-CD73 and anti-FGFR3 antibodies as described above. Membranes were developed with SuperSignal West Pico or Femto substrate and luminescence images were captured with a C300 imager (Azure Biosystems). Intensity of each CD73 and actin band was quantitated using Image J (NIH), and the ratio of CD73/actin for each sample were graphed using Prism Graphpad.

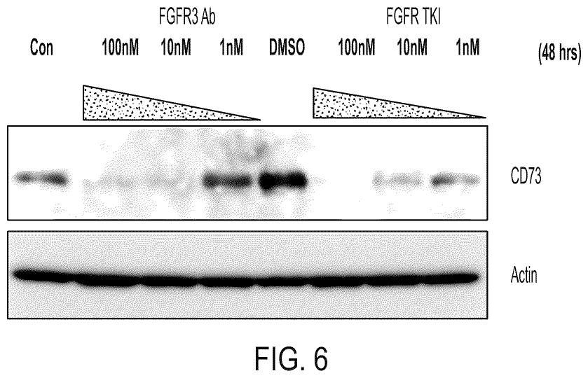

Downregulation of CD73 protein level in response to FGFR3 inhibition in UMUC14 cells in vitro. As shown in FIG. 6, both anti-FGFR3 inhibitory antibody and the pan-FGFR tyrosine kinase inhibitor (AZD4547) demonstrated dose dependent downregulation of CD73 expression. CD73 band intensity on the blot decreased as molar concentration of each agent increased from 1 nM to 100 nM.

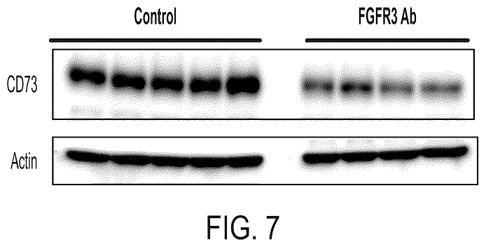

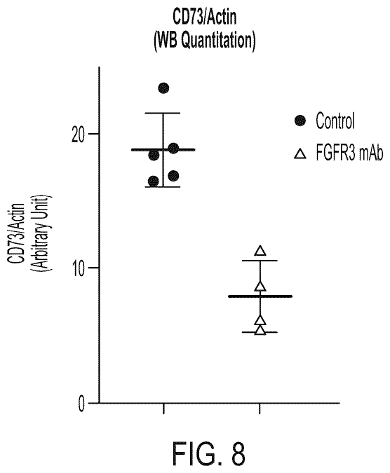

Downregulation of CD73 protein level in response to FGFR3 inhibition in UMUC14 cells in vivo. CD73 band intensity decreased with increasing anti-FGFR3 antibody concentration in the blot. See FIG. 7. The band intensity of CD73/actin is graphically summarized in FIG. 8.

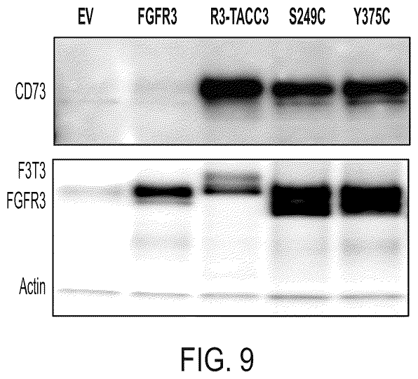

FGFR3 activation is sufficient to induce CD73 expression. As demonstrated in the blot of FIG. 9, in vitro cells expressing constitutively active FGFR3 mutants without ligand-induced activation exhibited increased expression of CD73 relative to control and wild-type FGFR3.

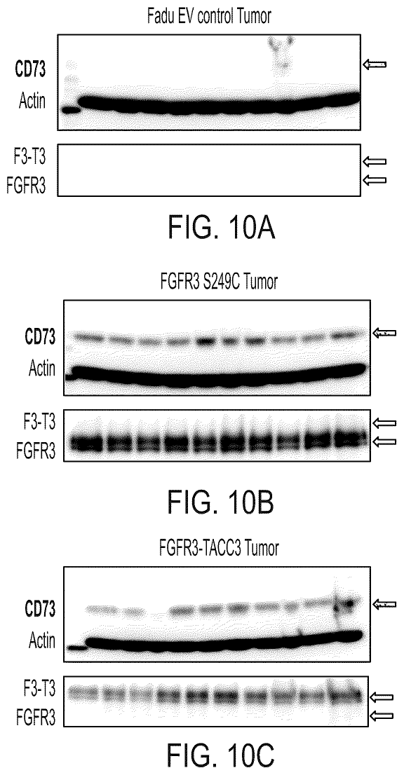

Upregulation of CD73 expression in Fadu tumors engineered to express FGFR3 S249C mutant or FGFR3-TACC3 fusion. Expression of CD73 in Fadu tumors expressing empty vector (EV; FIG. 10A) exhibited little or no activation of CD73 expression, whereas tumors expressing S249C (FIG. 10B) or FGFR-TACC3 (FIG. 10C) FGFR3 mutants exhibited increased CD73 expression.

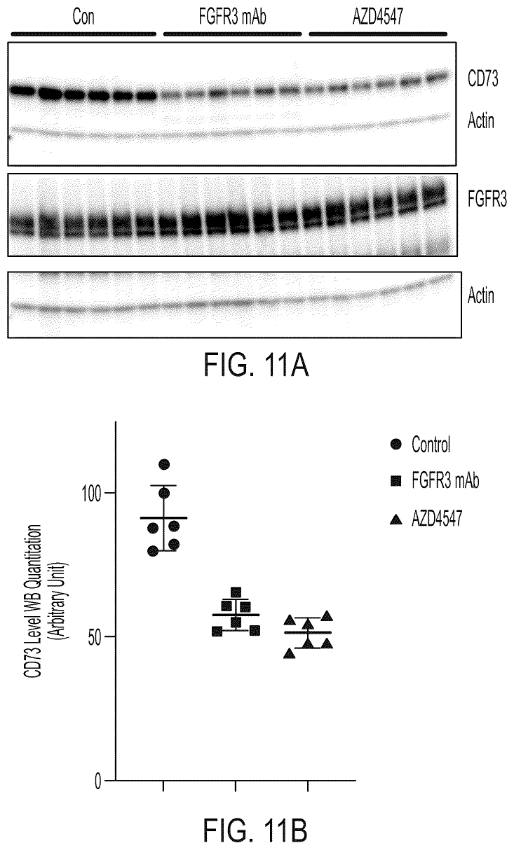

Downregulation of CD73 protein level in response to FGFR3 inhibition in Fadu S249C tumors in vivo. Mice having FADU tumors, expressing FGFR3 S249C mutants, treated with one dose intraperitoneal injection of FGFR3 mAb (15 mg/kg) or daily gavage of AZD4547 (25 mg/kg) exhibited decreased expression of CD73 relative to mice treated with control. The CD73 band intensity in blot shown in FIG. 11A was quantitated in the graph of FIG. 11B.

### Example 9: Antibody Inhibition of Proliferation of BaF3 Cell Expressing TKI Resistant Mutations

IL-3 dependent murine pro B cell line BaF3 cells were engineered to stably express full length human FGFR3b S249C, V557L, V557M, S249C/V557L, S249C/V557M (accession number for WT FGFR3b NP_001156685.1, amino acids M1-T808). Cells were maintained in RPMI 1640+10% FBS+P/S/G+1 ng/ml mouse IL-3+500 ug/ml Neomycin; 37° C. 5% CO₂. For proliferation assays, cells were washed with IL-3-free culture media and plated at 10⁵ cells/well into 96-well black wall tissue culture plates. Cells were treated with 1:3 serially diluted antibody as indicated in the presence or absence of 5 μg/ml heparin and 1 nM human FGF1. After addition of antibodies, cells were incubated at 37° C./5% CO₂ for 72 h followed by the addition of an equal volume of CellTiter-Glo™ (Promega) reagent to lyse cells and detect luciferase activity. Luminescence signal was quantitated with Envision plate reader (PerkinElmer). EC50 values of the antibodies were determined using GraphPad Prism software.

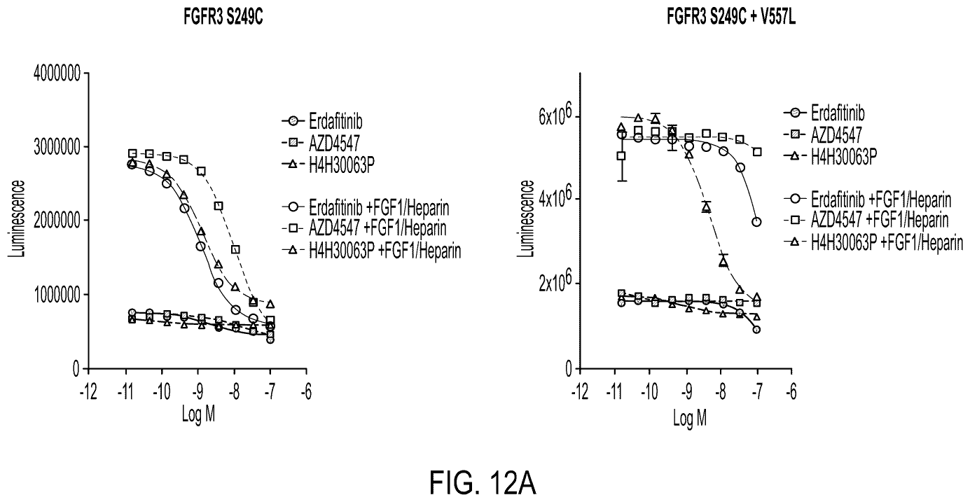

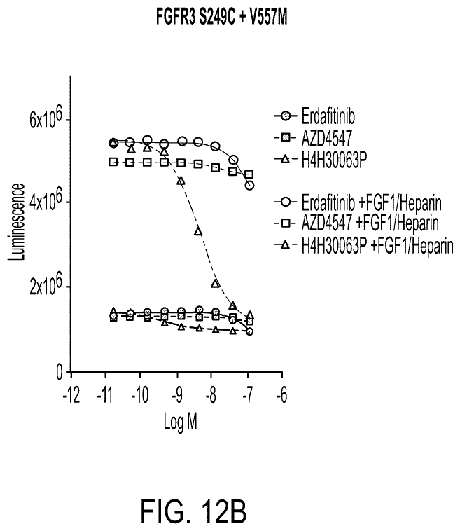

Acquired resistance to FGFR TKIs has been reported and it is frequently associated with secondary mutations in the kinase domain (Facchinetti F et al AACR 2023, abstract 3458, Chell V et al Oncogene 2013). One of the resistance mutation hotspots that appears to drive disease progression is the gatekeeper mutation V555M/L. To test the efficacy of the FGFR3 antibodies in the context of TKI resistance mutations, BaF3 cell lines were generated expressing FGFR3 S249C with or without the gatekeeper mutation V557M or V557L. The pan-FGFR TKIs AZD4547 and erdafitinib potently inhibited the growth of BaF3 cells expressing FGFR3 S249C (IC50 9.9 nM and 1.2 nM, respectively) (FIG. 12A, left), but were ineffective in cells expressing FGFR3 that contains V557M/L mutation alone or both the S249C and V557M/L mutations (FIG. 12A, right, S249C+V557L; FIG. 12B, S249C+V557M). In contrast, FGFR3 antibody H4H30063P was able to strongly inhibit the growth of BaF3 cells expressing the double mutant FGFR3 (S249C plus V557M or L; see, e.g., FIG. 12B and FIG. 12A, right, respectively). The IC50 values for H4H30063P against S249C, S249C/V557M, and S249CN557L were 1.36 nM, 3.9 nM, and 4.3 nM, respectively. See FIGS. 12A-12B.

### Example 10: Hydrogen/Deuterium Exchange (HDX) Epitope Mapping

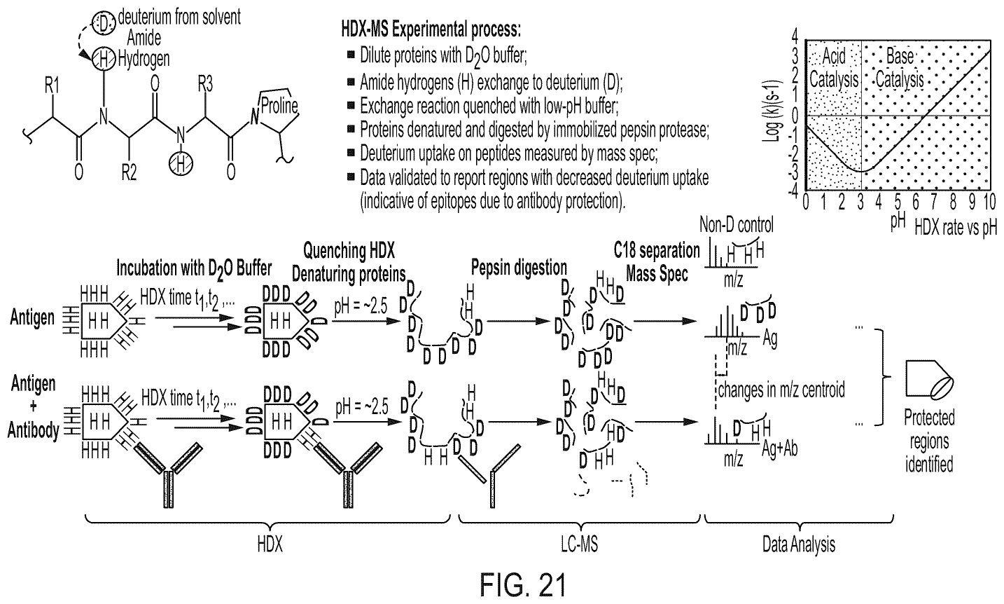

HDX experiment was performed using a customized HDX automation system (NovaBioAssays, MA) coupled to a Q Exactive HF mass spectrometer (Thermo Fisher Scientific, MA). FIG. 21 shows an exemplary HDX-Mass Spectrometry experimental process.

To initiate deuterium exchange, 10 μL of protein sample (hFGFR3b.mmh alone or hFGFR3b.mmh in mixture with H4H30117P2, H4H30063P, H4H30045P, or H4H30108P2 at 2:1 ratio) was diluted with 90 μL PBS-D20 buffer (10 mM, pH 7.4 at 25° C.). After 5 or 10 min, deuterium exchange was quenched by adding 100 μL quenching buffer (0.5 M TCEP, 4 M guanidine hydrochloride, pH 2.08) followed by 90 seconds incubation at 20° C. The quenched samples were digested by online pepsin/protease XIII column at room temperature (NovaBioAssays, MA) with 100 μL/min 0.1% formic acid in water. Peptic peptides were trapped by an ACQUITY UPLC Peptide BEH C18 VanGuard Pre-column (2.0×5 mm, Waters, MA) and further separated by an ACQUITY UPLC Peptide BEH C18 column (2.0×50 mm, Waters, MA) at −5° C., using a 15-min gradient with 0.1% formic acid in water and 0.1% formic acid in acetonitrile as mobile phases at 200 μL/min. Eluted peptides were analyzed by the Q Exactive HF mass spectrometry in LC-MS/MS or LC-MS mode.

Deuterium uptake percentage (D %) of individual peptides was calculated. Differences in deuterium uptake were calculated as AD %=D % of hFGFR3b-antibody−D % of hFGFR3b. Differences were considered significant if |ΔD|>5% (averaged from 2 replicates). Mass spectra of peptides showing significant differences were confirmed manually.

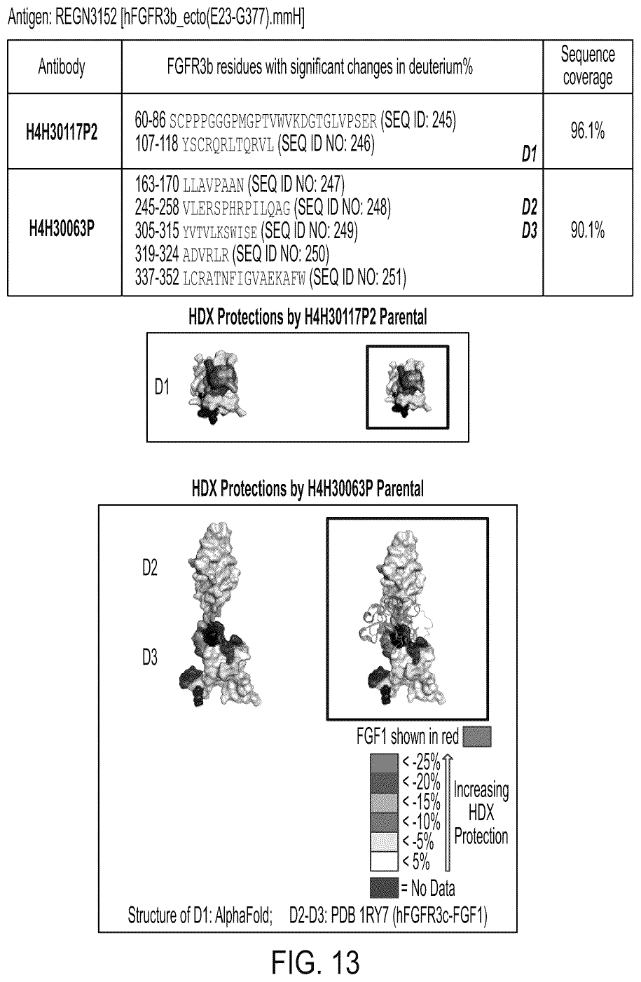

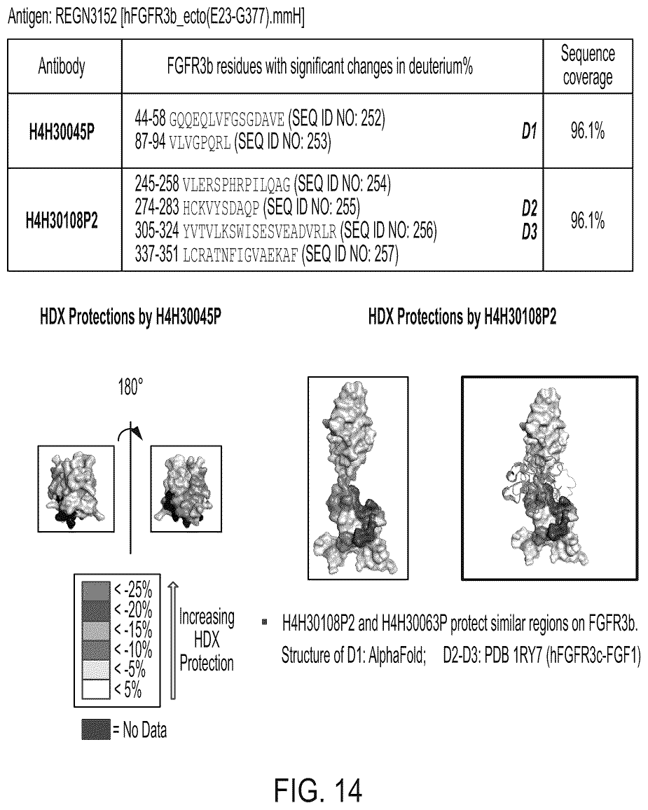

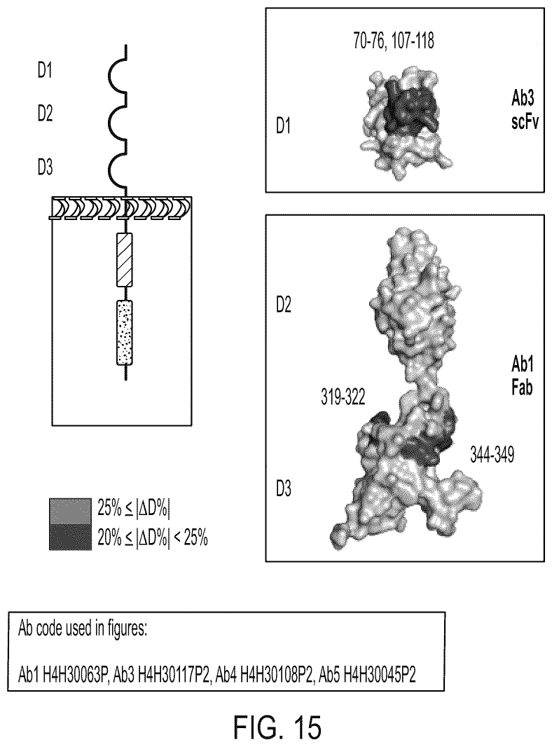

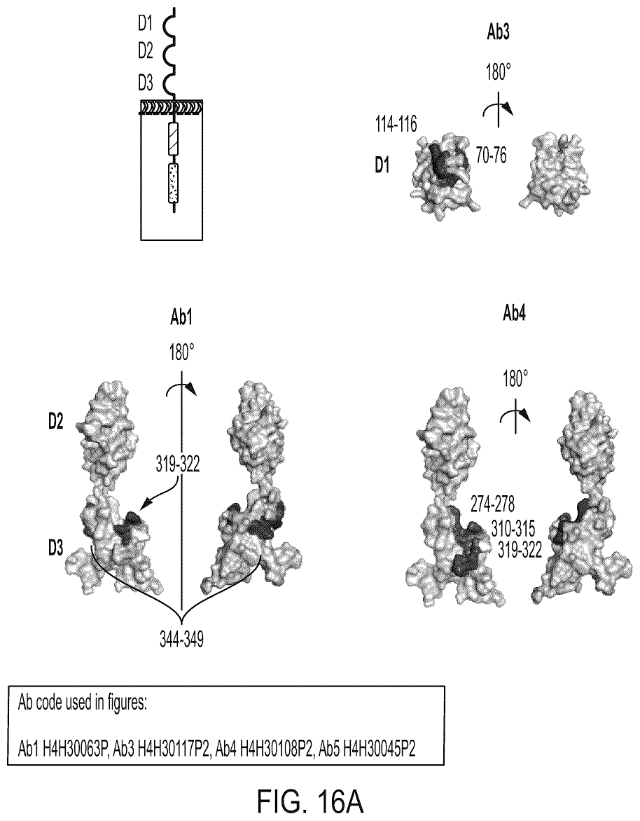

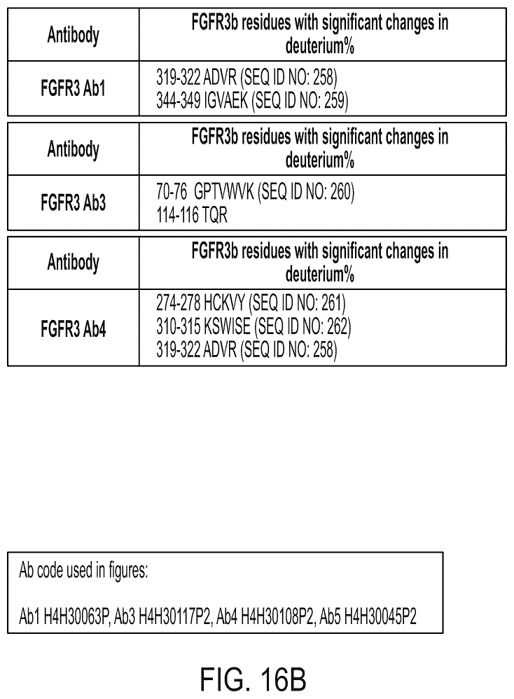

HDX epitope mapping results for anti-FGFR3b antibodies H4H30117P2, H4H30063P are shown in FIG. 13. HDX epitope mapping results for anti-FGFR3b antibodies H4H30045P and H4H30108P2 are shown in FIG. 14. HDX protection of FGFR3 by anti-FGFR3b antibodies H4H30063P Fab and H4H30117P2 scFv are shown in FIG. 15. HDX epitope mapping of FGFR3 antibodies is shown in FIGS. 16A-16B. As shown in the structures, the epitopes comprise segments of peptides, forming a continuous surface patch for interaction. The epitopes are likely conformational epitopes.

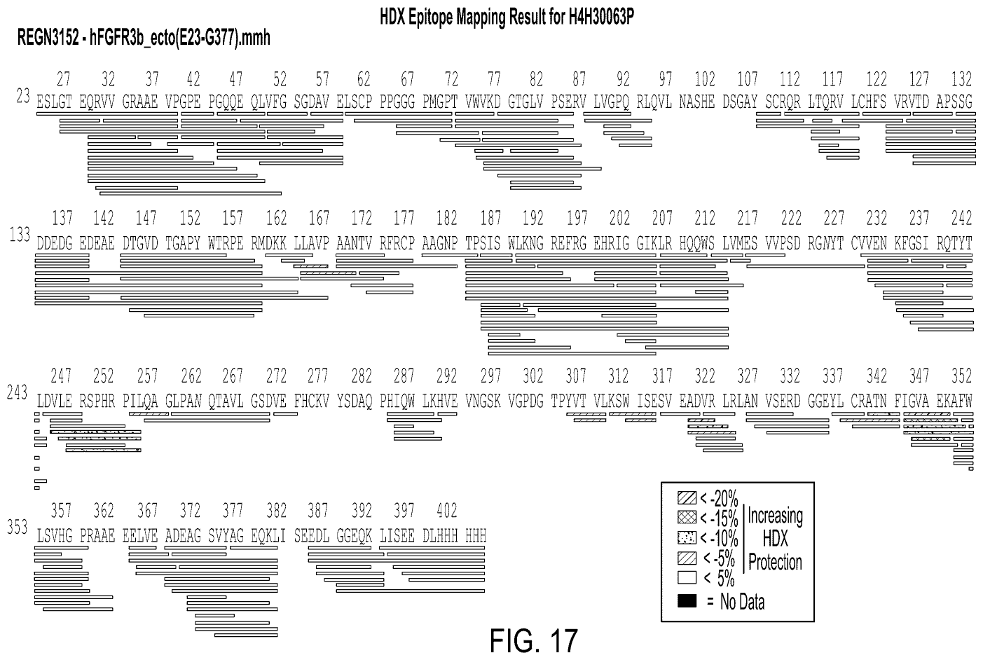

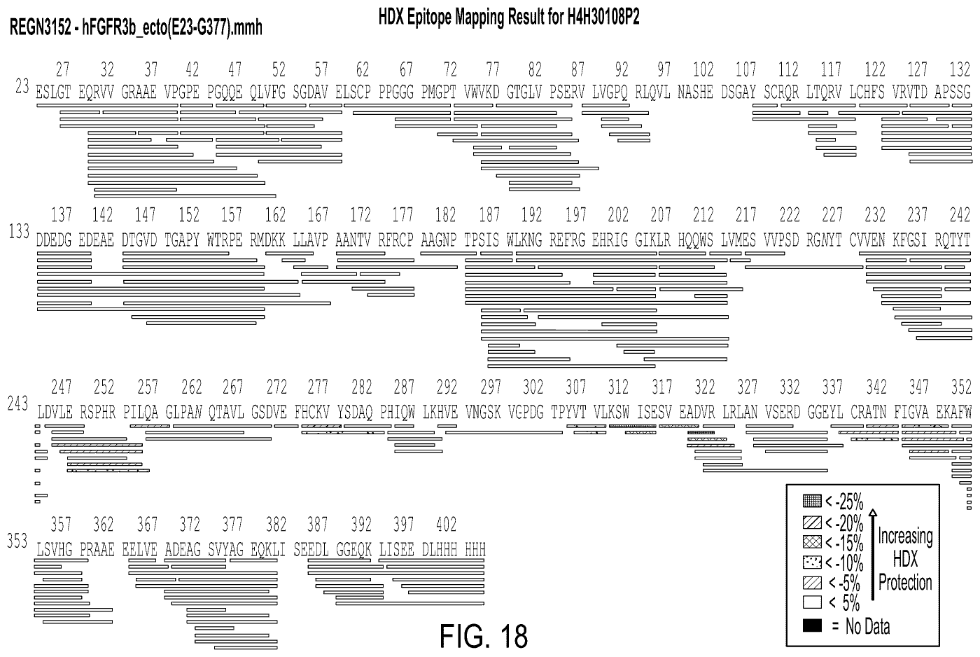

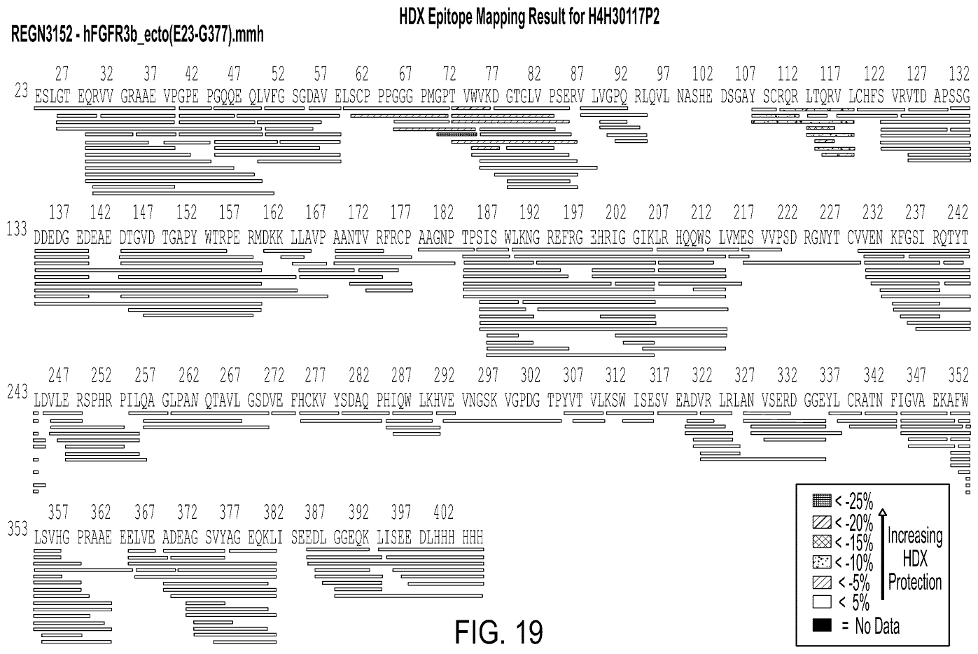

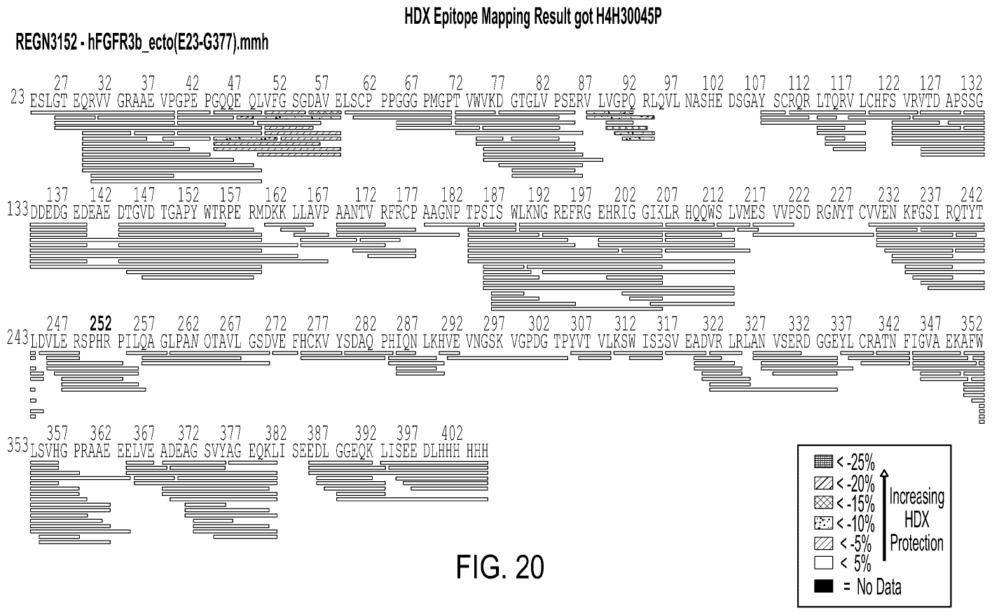

HDX epitope mapping results for H4H30063P are shown in FIG. 17. HDX epitope mapping results for H4H30108P2 are shown in FIG. 18. HDX epitope mapping results for H4H30117P2 are shown in FIG. 19. HDX epitope mapping results for H4H30045P are shown in FIG. 20.

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants to relate to each and every individual publication, database entry (e.g., Genbank sequences or GeneID entries), patent application, or patent, each of which is clearly identified in even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

1. An isolated antibody or antigen-binding fragment thereof that specifically binds to FGFR3 or an antigenic fragment thereof comprising: a heavy chain variable region (HCVR) that comprises the HCDR1, HCDR2 and HCDR3 of a HCVR that comprises the amino acid sequence set forth in SEQ ID NO: 2, 22, 42, 62, 82, 102, 122, 140, 159, 169, 179, 199 or 219 and a light chain variable region (LCVR) that comprises the LCDR1, LCDR2 and LCDR3 of a LCVR that comprises the amino acid sequence set forth in SEQ ID NO: 10, 30, 50, 70, 90, 110, 130, 148, 187, 207, or 227.

2. The isolated antibody or antigen-binding fragment thereof of claim 1, comprising:
(a) a heavy chain variable region (HCVR) that comprises the HCDR1, HCDR2 and HCDR3 of a HCVR that comprises the amino acid sequence set forth in SEQ ID NO: 2, and a light chain variable region (LCVR) that comprises the LCDR1, LCDR2 and LCDR3 of a LCVR that comprises the amino acid sequence set forth in SEQ ID NO: 10,
(b) a heavy chain variable region (HCVR) that comprises the HCDR1, HCDR2 and HCDR3 of a HCVR that comprises the amino acid sequence set forth in SEQ ID NO: 22, and a light chain variable region (LCVR) that comprises the LCDR1, LCDR2 and LCDR3 of a LCVR that comprises the amino acid sequence set forth in SEQ ID NO: 30,
(c) a heavy chain variable region (HCVR) that comprises the HCDR1, HCDR2 and HCDR3 of a HCVR that comprises the amino acid sequence set forth in SEQ ID NO: 42, and a light chain variable region (LCVR) that comprises the LCDR1, LCDR2 and LCDR3 of a LCVR that comprises the amino acid sequence set forth in SEQ ID NO: 50,
(d) a heavy chain variable region (HCVR) that comprises the HCDR1, HCDR2 and HCDR3 of a HCVR that comprises the amino acid sequence set forth in SEQ ID NO: 62, and a light chain variable region (LCVR) that comprises the LCDR1, LCDR2 and LCDR3 of a LCVR that comprises the amino acid sequence set forth in SEQ ID NO: 70,
(e) a heavy chain variable region (HCVR) that comprises the HCDR1, HCDR2 and HCDR3 of a HCVR that comprises the amino acid sequence set forth in SEQ ID NO: 82, and a light chain variable region (LCVR) that comprises the LCDR1, LCDR2 and LCDR3 of a LCVR that comprises the amino acid sequence set forth in SEQ ID NO: 90,
(f) a heavy chain variable region (HCVR) that comprises the HCDR1, HCDR2 and HCDR3 of a HCVR that comprises the amino acid sequence set forth in SEQ ID NO: 102, and a light chain variable region (LCVR) that comprises the LCDR1, LCDR2 and LCDR3 of a LCVR that comprises the amino acid sequence set forth in SEQ ID NO: 110,
(g) a heavy chain variable region (HCVR) that comprises the HCDR1, HCDR2 and HCDR3 of a HCVR that comprises the amino acid sequence set forth in SEQ ID NO: 122, and a light chain variable region (LCVR) that comprises the LCDR1, LCDR2 and LCDR3 of a LCVR that comprises the amino acid sequence set forth in SEQ ID NO: 130,
(h) a heavy chain variable region (HCVR) that comprises the HCDR1, HCDR2 and HCDR3 of a HCVR that comprises the amino acid sequence set forth in SEQ ID NO: 140, and a light chain variable region (LCVR) that comprises the LCDR1, LCDR2 and LCDR3 of a LCVR that comprises the amino acid sequence set forth in SEQ ID NO: 148,
(i) a heavy chain variable region (HCVR) that comprises the HCDR1, HCDR2 and HCDR3 of a HCVR that comprises the amino acid sequence set forth in SEQ ID NO: 159, and a light chain variable region (LCVR) that comprises the LCDR1, LCDR2 and LCDR3 of a LCVR that comprises the amino acid sequence set forth in SEQ ID NO: 148,
(j) a heavy chain variable region (HCVR) that comprises the HCDR1, HCDR2 and HCDR3 of a HCVR that comprises the amino acid sequence set forth in SEQ ID NO: 169, and a light chain variable region (LCVR) that comprises the LCDR1, LCDR2 and LCDR3 of a LCVR that comprises the amino acid sequence set forth in SEQ ID NO: 148,
(k) a heavy chain variable region (HCVR) that comprises the HCDR1, HCDR2 and HCDR3 of a HCVR that comprises the amino acid sequence set forth in SEQ ID NO: 179, and a light chain variable region (LCVR) that comprises the LCDR1, LCDR2 and LCDR3 of a LCVR that comprises the amino acid sequence set forth in SEQ ID NO: 187,
(l) a heavy chain variable region (HCVR) that comprises the HCDR1, HCDR2 and HCDR3 of a HCVR that comprises the amino acid sequence set forth in SEQ ID NO: 199, and a light chain variable region (LCVR) that comprises the LCDR1, LCDR2 and LCDR3 of a LCVR that comprises the amino acid sequence set forth in SEQ ID NO: 207,
and/or
(m) a heavy chain variable region (HCVR) that comprises the HCDR1, HCDR2 and HCDR3 of a HCVR that comprises the amino acid sequence set forth in SEQ ID NO: 219, and a light chain variable region (LCVR) that comprises the LCDR1, LCDR2 and LCDR3 of a LCVR that comprises the amino acid sequence set forth in SEQ ID NO: 227.

3. The isolated antibody or antigen-binding fragment thereof of claim 1, comprising:
(a) a heavy chain variable region that comprises an HCDR1 that comprises the amino acid sequence set forth in SEQ ID NO: 4, an HCDR2 that comprises the amino acid sequence set forth in SEQ ID NO: 6, and an HCDR3 that comprises the amino acid sequence set forth in SEQ ID NO: 8, and a light chain variable region that comprises an LCDR1 that comprises the amino acid sequence set forth in SEQ ID NO: 12, an LCDR2 that comprises the amino acid sequence set forth in SEQ ID NO: 14, and an LCDR3 that comprises the amino acid sequence set forth in SEQ ID NO: 16;
(b) a heavy chain variable region that comprises an HCDR1 that comprises the amino acid sequence set forth in SEQ ID NO: 24, an HCDR2 that comprises the amino acid sequence set forth in SEQ ID NO: 26, and an HCDR3 that comprises the amino acid sequence set forth in SEQ ID NO: 28, and a light chain variable region that comprises an LCDR1 that comprises the amino acid sequence set forth in SEQ ID NO: 32, an LCDR2 that comprises the amino acid sequence set forth in SEQ ID NO: 34, and an LCDR3 that comprises the amino acid sequence set forth in SEQ ID NO: 36;
(c) a heavy chain variable region that comprises an HCDR1 that comprises the amino acid sequence set forth in SEQ ID NO: 44, an HCDR2 that comprises the amino acid sequence set forth in SEQ ID NO: 46, and an HCDR3 that comprises the amino acid sequence set forth in SEQ ID NO: 48, and a light chain variable region that comprises an LCDR1 that comprises the amino acid sequence set forth in SEQ ID NO: 52, an LCDR2 that comprises the amino acid sequence set forth in SEQ ID NO: 54, and an LCDR3 that comprises the amino acid sequence set forth in SEQ ID NO: 56;
(d) a heavy chain variable region that comprises an HCDR1 that comprises the amino acid sequence set forth in SEQ ID NO: 64, an HCDR2 that comprises the amino acid sequence set forth in SEQ ID NO: 66, and an HCDR3 that comprises the amino acid sequence set forth in SEQ ID NO: 68, and a light chain variable region that comprises an LCDR1 that comprises the amino acid sequence set forth in SEQ ID NO: 72, an LCDR2 that comprises the amino acid sequence set forth in SEQ ID NO: 74, and an LCDR3 that comprises the amino acid sequence set forth in SEQ ID NO: 76;
(e) a heavy chain variable region that comprises an HCDR1 that comprises the amino acid sequence set forth in SEQ ID NO: 84, an HCDR2 that comprises the amino acid sequence set forth in SEQ ID NO: 86, and an HCDR3 that comprises the amino acid sequence set forth in SEQ ID NO: 88, and a light chain variable region that comprises an LCDR1 that comprises the amino acid sequence set forth in SEQ ID NO: 92, an LCDR2 that comprises the amino acid sequence set forth in SEQ ID NO: 94, and an LCDR3 that comprises the amino acid sequence set forth in SEQ ID NO: 96;
(f) a heavy chain variable region that comprises an HCDR1 that comprises the amino acid sequence set forth in SEQ ID NO: 104, an HCDR2 that comprises the amino acid sequence set forth in SEQ ID NO: 106, and an HCDR3 that comprises the amino acid sequence set forth in SEQ ID NO: 108, and a light chain variable region that comprises an LCDR1 that comprises the amino acid sequence set forth in SEQ ID NO: 112, an LCDR2 that comprises the amino acid sequence set forth in SEQ ID NO: 114, and an LCDR3 that comprises the amino acid sequence set forth in SEQ ID NO: 116,
(g) a heavy chain variable region that comprises an HCDR1 that comprises the amino acid sequence set forth in SEQ ID NO: 124, an HCDR2 that comprises the amino acid sequence set forth in SEQ ID NO: 126, and an HCDR3 that comprises the amino acid sequence set forth in SEQ ID NO: 128, and a light chain variable region that comprises an LCDR1 that comprises the amino acid sequence set forth in SEQ ID NO: 132, an LCDR2 that comprises the amino acid sequence set forth in SEQ ID NO: 34, and an LCDR3 that comprises the amino acid sequence set forth in SEQ ID NO: 134;
(h) a heavy chain variable region that comprises an HCDR1 that comprises the amino acid sequence set forth in SEQ ID NO: 142, an HCDR2 that comprises the amino acid sequence set forth in SEQ ID NO: 144, and an HCDR3 that comprises the amino acid sequence set forth in SEQ ID NO: 146, and a light chain variable region that comprises an LCDR1 that comprises the amino acid sequence set forth in SEQ ID NO: 150, an LCDR2 that comprises the amino acid sequence set forth in SEQ ID NO: 14, and an LCDR3 that comprises the amino acid sequence set forth in SEQ ID NO: 153;
(i) a heavy chain variable region that comprises an HCDR1 that comprises the amino acid sequence set forth in SEQ ID NO: 161, an HCDR2 that comprises the amino acid sequence set forth in SEQ ID NO: 163, and an HCDR3 that comprises the amino acid sequence set forth in SEQ ID NO: 165, and a light chain variable region that comprises an LCDR1 that comprises the amino acid sequence set forth in SEQ ID NO: 150, an LCDR2 that comprises the amino acid sequence set forth in SEQ ID NO: 14, and an LCDR3 that comprises the amino acid sequence set forth in SEQ ID NO: 153;
(j) a heavy chain variable region that comprises an HCDR1 that comprises the amino acid sequence set forth in SEQ ID NO: 171, an HCDR2 that comprises the amino acid sequence set forth in SEQ ID NO: 173, and an HCDR3 that comprises the amino acid sequence set forth in SEQ ID NO: 175, and a light chain variable region that comprises an LCDR1 that comprises the amino acid sequence set forth in SEQ ID NO: 150, an LCDR2 that comprises the amino acid sequence set forth in SEQ ID NO: 14, and an LCDR3 that comprises the amino acid sequence set forth in SEQ ID NO: 153;
(k) a heavy chain variable region that comprises an HCDR1 that comprises the amino acid sequence set forth in SEQ ID NO: 181, an HCDR2 that comprises the amino acid sequence set forth in SEQ ID NO: 183, and an HCDR3 that comprises the amino acid sequence set forth in SEQ ID NO: 185, and a light chain variable region that comprises an LCDR1 that comprises the amino acid sequence set forth in SEQ ID NO: 189, an LCDR2 that comprises the amino acid sequence set forth in SEQ ID NO: 191, and an LCDR3 that comprises the amino acid sequence set forth in SEQ ID NO: 193;
(l) a heavy chain variable region that comprises an HCDR1 that comprises the amino acid sequence set forth in SEQ ID NO: 201, an HCDR2 that comprises the amino acid sequence set forth in SEQ ID NO: 203, and an HCDR3 that comprises the amino acid sequence set forth in SEQ ID NO: 205, and a light chain variable region that comprises an LCDR1 that comprises the amino acid sequence set forth in SEQ ID NO: 209, an LCDR2 that comprises the amino acid sequence set forth in SEQ ID NO: 211, and an LCDR3 that comprises the amino acid sequence set forth in SEQ ID NO: 213;
and/or
(m) a heavy chain variable region that comprises an HCDR1 that comprises the amino acid sequence set forth in SEQ ID NO: 221, an HCDR2 that comprises the amino acid sequence set forth in SEQ ID NO: 223, and an HCDR3 that comprises the amino acid sequence set forth in SEQ ID NO: 225, and a light chain variable region that comprises an LCDR1 that comprises the amino acid sequence set forth in SEQ ID NO: 32, an LCDR2 that comprises the amino acid sequence set forth in SEQ ID NO: 34, and an LCDR3 that comprises the amino acid sequence set forth in SEQ ID NO: 76.

4. The isolated antibody or antigen-binding fragment thereof of claim 1, comprising a heavy chain variable region that comprises the amino acid sequence set forth in SEQ ID NO: 2, 22, 42, 62, 82, 102, 122, 140, 159, 169, 179, 199 or 219, and a light chain variable region that comprises the amino acid sequence set forth in SEQ ID NO: 10, 30, 50, 70, 90, 110, 130, 148, 187, 207, or 227.

5. The isolated antibody or antigen-binding fragment thereof of claim 1, comprising:
(a) a heavy chain variable region that comprises the amino acid sequence set forth in SEQ ID NO: 2, and a light chain variable region that comprises the amino acid sequence set forth in SEQ ID NO: 10;
(b) a heavy chain variable region that comprises the amino acid sequence set forth in SEQ ID NO: 22, and a light chain variable region that comprises the amino acid sequence set forth in SEQ ID NO: 30;
(c) a heavy chain variable region that comprises the amino acid sequence set forth in SEQ ID NO: 42, and a light chain variable region that comprises the amino acid sequence set forth in SEQ ID NO: 50;
(d) a heavy chain variable region that comprises the amino acid sequence set forth in SEQ ID NO: 62, and a light chain variable region that comprises the amino acid sequence set forth in SEQ ID NO: 70;
(e) a heavy chain variable region that comprises the amino acid sequence set forth in SEQ ID NO: 82, and a light chain variable region that comprises the amino acid sequence set forth in SEQ ID NO: 90;
(f) a heavy chain variable region that comprises the amino acid sequence set forth in SEQ ID NO: 102, and a light chain variable region that comprises the amino acid sequence set forth in SEQ ID NO: 110;
(g) a heavy chain variable region that comprises the amino acid sequence set forth in SEQ ID NO: 122, and a light chain variable region that comprises the amino acid sequence set forth in SEQ ID NO: 130;
(h) a heavy chain variable region that comprises the amino acid sequence set forth in SEQ ID NO: 140, and a light chain variable region that comprises the amino acid sequence set forth in SEQ ID NO: 148;
(i) a heavy chain variable region that comprises the amino acid sequence set forth in SEQ ID NO: 159, and a light chain variable region that comprises the amino acid sequence set forth in SEQ ID NO: 148;
(j) a heavy chain variable region that comprises the amino acid sequence set forth in SEQ ID NO: 169, and a light chain variable region that comprises the amino acid sequence set forth in SEQ ID NO: 148;
(k) a heavy chain variable region that comprises the amino acid sequence set forth in SEQ ID NO: 179, and a light chain variable region that comprises the amino acid sequence set forth in SEQ ID NO: 187;
(l) a heavy chain variable region that comprises the amino acid sequence set forth in SEQ ID NO: 199, and a light chain variable region that comprises the amino acid sequence set forth in SEQ ID NO: 207;
and/or
(m) a heavy chain variable region that comprises the amino acid sequence set forth in SEQ ID NO: 219, and a light chain variable region that comprises the amino acid sequence set forth in SEQ ID NO: 227.

6. The isolated antibody or antigen-binding fragment thereof of claim 1, comprising (a) a heavy chain that comprises the amino acid sequence set forth in SEQ ID NO: 18, 38, 58, 78, 98, 118, 136, 155, 167, 177, 195, 215 or 229, and a light chain that comprises the amino acid sequence set forth in SEQ ID NO: 20, 40, 60, 80, 100, 120, 138, 157, 197, 217, or 231.

7. The isolated antibody or antigen-binding fragment thereof of claim 1, comprising:
(a) a heavy chain that comprises the amino acid sequence set forth in SEQ ID NO: 18, and a light chain that comprises the amino acid sequence set forth in SEQ ID NO: 20;
(b) a heavy chain that comprises the amino acid sequence set forth in SEQ ID NO: 38, and a light chain that comprises the amino acid sequence set forth in SEQ ID NO: 40;
(c) a heavy chain that comprises the amino acid sequence set forth in SEQ ID NO: 58, and a light chain that comprises the amino acid sequence set forth in SEQ ID NO: 60;
(d) a heavy chain that comprises the amino acid sequence set forth in SEQ ID NO: 78, and a light chain that comprises the amino acid sequence set forth in SEQ ID NO: 80;
(e) a heavy chain that comprises the amino acid sequence set forth in SEQ ID NO: 98, and a light chain that comprises the amino acid sequence set forth in SEQ ID NO: 100;
(f) a heavy chain that comprises the amino acid sequence set forth in SEQ ID NO: 118, and a light chain that comprises the amino acid sequence set forth in SEQ ID NO: 120;
(g) a heavy chain that comprises the amino acid sequence set forth in SEQ ID NO: 136, and a light chain that comprises the amino acid sequence set forth in SEQ ID NO: 138;
(h) a heavy chain that comprises the amino acid sequence set forth in SEQ ID NO: 155, and a light chain that comprises the amino acid sequence set forth in SEQ ID NO: 157;
(i) a heavy chain that comprises the amino acid sequence set forth in SEQ ID NO: 167, and a light chain that comprises the amino acid sequence set forth in SEQ ID NO: 157;
(j) a heavy chain that comprises the amino acid sequence set forth in SEQ ID NO: 177, and a light chain that comprises the amino acid sequence set forth in SEQ ID NO: 157;
(k) a heavy chain that comprises the amino acid sequence set forth in SEQ ID NO: 195, and a light chain that comprises the amino acid sequence set forth in SEQ ID NO: 197;
(l) a heavy chain that comprises the amino acid sequence set forth in SEQ ID NO: 215, and a light chain that comprises the amino acid sequence set forth in SEQ ID NO: 217;
and/or
(m) a heavy chain that comprises the amino acid sequence set forth in SEQ ID NO: 229, and a light chain that comprises the amino acid sequence set forth in SEQ ID NO: 231.

8. An isolated antibody or antigen-binding fragment thereof that specifically binds to FGFR3 or an antigenic fragment thereof, wherein the antibody or antigen-binding fragment binds to the same FGFR3 epitope as or competes for binding to FGFR3 with the antibody or antigen-binding fragment of claim 1.

9. An isolated antibody or antigen-binding fragment thereof that specifically binds to FGFR3 or an antigenic fragment thereof, wherein the antibody or antigen-binding fragment thereof binds to one or more epitopes of FGFR3 selected from:
a. an epitope comprising the sequence GPTVWVK (SEQ ID NO: 260) and/or an epitope comprising the sequence TQR;
b. an epitope comprising the sequence ADVR (SEQ ID NO: 258) and/or an epitope comprising the sequence IGVAEK (SEQ ID NO: 259);
c. an epitope comprising the sequence HCKVY (SEQ ID NO: 261), and/or an epitope comprising the sequence KSWISE (SEQ ID NO: 262), and/or an epitope comprising the sequence ADVR (SEQ ID NO: 258);
d. an epitope comprised within or overlapping with the sequence GPTVWVK (SEQ ID NO: 260) and/or an epitope comprised within or overlapping with the sequence TQR;
e. an epitope comprised within or overlapping with the sequence ADVR (SEQ ID NO: 258) and/or an epitope comprised within or overlapping with the sequence IGVAEK (SEQ ID NO: 259); and
f. an epitope comprised within or overlapping with the sequence HCKVY (SEQ ID NO: 261), and/or an epitope comprised within or overlapping with the sequence KSWISE (SEQ ID NO: 262), and/or an epitope comprised within or overlapping with the sequence ADVR (SEQ ID NO: 258).

10. (canceled)

11. An isolated antibody or antigen-binding fragment thereof that specifically binds to FGFR3 or an antigenic fragment thereof, wherein the antibody or antigen-binding fragment thereof binds to one or more epitopes of FGFR3 selected from:
a. an epitope comprising the sequence SCPPPGGGPMGPTVWVKDGTGLVPSER (SEQ ID NO: 245) and/or an epitope comprising the sequence YSCRQRLTQRVL (SEQ ID NO: 246);
b. an epitope comprising the sequence LLAVPAAN (SEQ ID NO: 247), and/or an epitope comprising the sequence VLERSPHRPILQAG (SEQ ID NO: 248) and/or an epitope comprising the sequence YVTVLKSWISE (SEQ ID NO: 249) and/or or an epitope comprising the sequence ADVRLR (SEQ ID NO: 250) and/or an epitope comprising the sequence LCRATNFIGVAEKAFW (SEQ ID NO: 251);
c. an epitope comprising the sequence GQQEQLVFGSGDAVE (SEQ ID NO: 252) and/or an epitope comprising the sequence VLVGPQRL (SEQ ID NO: 253);
d. an epitope comprising the sequence VLERSPHRPILQAG (SEQ ID NO: 254) and/or an epitope comprising the sequence HCKVYSDAQP (SEQ ID NO: 255) and/or an epitope comprising the sequence YVTVLKSWISESVEADVRLR (SEQ ID NO: 256) and/or an epitope comprising the sequence LCRATNFIGVAEKAF (SEQ ID NO: 257);
e. an epitope comprised within or overlapping with the sequence SCPPPGGGPMGPTVWVKDGTGLVPSER (SEQ ID NO: 245) and/or an epitope comprised within or overlapping with the sequence YSCRQRLTQRVL (SEQ ID NO: 246);
f. an epitope comprised within or overlapping with the sequence LLAVPAAN (SEQ ID NO: 247), and/or an epitope comprised within or overlapping with the sequence VLERSPHRPILQAG (SEQ ID NO: 248) and/or an epitope comprised within or overlapping with the sequence YVTVLKSWISE (SEQ ID NO: 249) and/or or an epitope comprised within or overlapping with the sequence ADVRLR (SEQ ID NO: 250) and/or an epitope comprised within or overlapping with the sequence LCRATNFIGVAEKAFW (SEQ ID NO: 251);
g. an epitope comprised within or overlapping with the sequence GQQEQLVFGSGDAVE (SEQ ID NO: 252) and/or an epitope comprised within or overlapping with the sequence VLVGPQRL (SEQ ID NO: 253); and
h. an epitope comprised within or overlapping with the sequence VLERSPHRPILQAG (SEQ ID NO: 254) and/or an epitope comprised within or overlapping with the sequence HCKVYSDAQP (SEQ ID NO: 255) and/or an epitope comprised within or overlapping with the sequence YVTVLKSWISESVEADVRLR (SEQ ID NO: 256) and/or an epitope comprised within or overlapping with the sequence LCRATNFIGVAEKAF (SEQ ID NO: 257).

12. (canceled)

13. The antibody or antigen-binding fragment of claim 1, wherein FGFR3 is monomeric or dimeric FGFR3b.

14. The isolated antibody or antigen-binding fragment thereof of claim 1, wherein the antibody or antigen-binding fragment is characterized by one or more of the following:
Binds to monomeric human FGFR3b tagged the C-terminus, with myc-myc-His₆ at 25° C. with an affinity (K_{D}) of about 16 nM or greater affinity in a surface plasmon resonance assay;
Binds to monomeric Cynomolgous monkey FGFR3b tagged at the C-terminus with myc-myc-His₆ at 25° C. with an affinity (K_{D}) of about 20 nM or greater affinity in a surface plasmon resonance assay;
Binds to monomeric murine FGFR3b tagged at the C-terminus with myc-myc-His₆ at 25° C. with an affinity (K_{D}) of about 70 nM or greater affinity in a surface plasmon resonance assay;
Does not bind significantly to monomeric human FGFR3c tagged at the C-terminus with myc-myc-His₆ at 25° C. in a surface plasmon resonance assay;
Binds to dimeric human FGFR3b tagged at the C-terminus, with a mouse Fc at 25° C. with an affinity of about 0.6 nM of greater affinity in a surface plasmon resonance assay;
Blocks about 68% or more binding of FGF1 acidic to human FGFR3b-mFc at 200 nM antibody;
Binds to monomeric Cynomolgous and monomeric mouse FGFR3b with a K_{D} that is within about 0.1 nM of the K_{D} for binding to monomeric or dimeric human FGFR3b at 25° C. in a surface plasmon resonance assay;
Blocks binding of 4 nM human FGFR3b-mFc to human FGF1 acidic protein with an IC₅₀ of about 15 nM or a lower concentration;
Blocks intracellular signaling in an engineered IL-3-dependent Ba/F3 murine hematopoietic cell line genetically modified to stably express wild-type or a S249C mutant human fibroblast growth factor receptor 3b that has been stimulated with human heparin at about 5 micrograms/ml, and human FGF1 ligand at about 1 nM, with an IC₅₀ of about 18 nM or a lower concentration;
Blocks intracellular signaling in an engineered IL-3-dependent Ba/F3 murine hematopoietic cell line genetically modified to stably express a S249C mutant human fibroblast growth factor receptor 3b that has been stimulated with human heparin at about 5 micrograms/ml and human FGF1 ligand at about 1 nM, with an IC₅₀ that is lower than for blocking intracellular signaling in an engineered IL-3-dependent Ba/F3 murine hematopoietic cell line genetically modified to stably express wild-type human fibroblast growth factor receptor 3b that has been stimulated with human FGF1 ligand at about 1 nM and human heparin at about 5 micrograms/ml;
Blocks dimerization of FGFR3 or a S249C mutant thereof with stronger inhibition than that of REGN6331 as measured in a non-reducing SDS-PAGE assay;
Reduces tumor size of a bladder cancer tumor expressing FGFR3 or a S249C mutant thereof in a subject administered the antibody or fragment;
Reduces CD73 expression in a bladder cancer tumor expressing FGFR3 or a S249C mutant thereof in a subject administered the antibody or fragment;
Inhibits FGF1/heparin stimulation induced phosphorylation of MAPK in a BaF3 cell expressing wild-type FGFR3 or a S249C mutant thereof,
Inhibits proliferation of UMUC14 bladder cancer cells expressing endogenous FGFR3 or a S249C mutation thereof in a cancer cell spheroid proliferation assay;
Inhibits tumor growth in a SCID mouse xenograft model of bladder cancer cell line UMUC14 expressing FGFR3 or a S249C mutation thereof,
Inhibits CD73-dependent, adenosine-mediated inhibition of immune cell activation;
Increases the ratio of CD8/CD4 and/or CD8+/T_{reg} in tumor tissue having tumor cells expressing FGFR3 or a S249C mutant thereof,
Inhibits FGF3-mediated activation of CD73 expression and/or adenosine production on a tumor cell expressing FGFR3 or a S249C mutant thereof,
Inhibits FGFR3-dependent, adenosine-mediated immune cell suppression; or
Inhibits growth of BaF3 cells expressing the FGFR3 double mutant S249C and V557M, or S249C and V557L.

15. A complex comprising an antibody or antigen-binding fragment thereof of claim 1 bound to FGFR3 or an antigenic fragment thereof.

16. A pharmaceutical formulation comprising the antibody or antigen-binding fragment of claim 1, and a pharmaceutically acceptable carrier.

17. An isolated polypeptide comprising an amino acid sequence selected from SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229 and 231.

18. An isolated polynucleotide that encodes any one or more of the polypeptides of claim 17.

19. (canceled)

20. A vector that comprises a polynucleotide of claim 18.

21. A host cell that comprises the polynucleotide of claim 18.

22. A method for making an antibody or antigen-binding fragment of claim 1 comprising introducing a polynucleotide that encodes the chains of the antibody or antigen-binding fragment into a host cell and incubating the host cell that comprises the polynucleotide in a culture medium under conditions favorable to expression of the chains and, optionally, isolation of the antibody or antigen-binding fragment from the host cell and/or culture medium.

23. A method for administering the antibody or antigen-binding fragment of claim 1 to a subject comprising introducing the antibody or antigen-binding fragment into the body of the subject.

24-25. (canceled)

26. A method for treating or preventing an FGFR3-mediated condition, in a subject in need thereof, comprising administering a therapeutically effective amount of the antibody or antigen-binding fragment of claim 1 to the subject.

27-29. (canceled)

30. A method for
reducing metastasis of a tumor cell expressing FGFR3,
reducing the concentration of adenosine in a tumor expressing FGFR3,
reducing CD73-dependent catalysis of AMP to adenosine by a tumor expressing FGFR3,
inhibiting adenosine-mediated suppression of T-cell function in a tumor expressing FGFR3, in the body of a subject in need thereof comprising administering a therapeutically effective amount of the antibody or antigen-binding fragment of claim 1 to the subject.

31. (canceled)


SEQUENCE LISTING (263 sequences)

SEQ ID NO: 1 - DNA, 345 bp, synthetic construct
  source 1..345: mol_type = other DNA
caggtgcagctgcaggagtcgggcccaggactggtgaagccttcggagaccctgtccctcacctgcactgtctctggtgactccattaatagttacttctggagctggatccggcagttgccagggaaggaactggagtggattggccatatctattctagtgggagtaccagatacaacccctccctccagagtcgagtcaccatatcaatagacacgtccaagaaccagttctccctgaagctgagttctgtgaccgctgcggacacggccgtatattactgtgcgaggggcgccagcgcagttgactactggggccagggaaccctggtcaccgtctcctca

SEQ ID NO: 2 - protein, 115 aa, synthetic construct
QVQLQESGPGLVKPSETLSLTCTVSGDSINSYFWSWIRQLPGKELEWIGHIYSSGSTRYNPSLQSRVTISIDTSKNQFSLKLSSVTAADTAVYYCARGASAVDYWGQGTLVTVSS

SEQ ID NO: 3 - DNA, 24 bp, synthetic construct
  source 1..24: mol_type = other DNA
ggtgactccattaatagttacttc

SEQ ID NO: 4 - protein, 8 aa, synthetic construct
GDSINSYF

SEQ ID NO: 5 - DNA, 21 bp, synthetic construct
  source 1..21: mol_type = other DNA
atctattctagtgggagtacc

SEQ ID NO: 6 - protein, 7 aa, synthetic construct
IYSSGST

SEQ ID NO: 7 - DNA, 27 bp, synthetic construct
  source 1..27: mol_type = other DNA
gcgaggggcgccagcgcagttgactac

SEQ ID NO: 8 - protein, 9 aa, synthetic construct
ARGASAVDY

SEQ ID NO: 9 - DNA, 324 bp, synthetic construct
  source 1..324: mol_type = other DNA
gaaattgtgttgacgcagtctccaggcaccctgtctttgtctccaggggaaagagccaccctctcctgcaggaccagtcagagtattagcagcggctatttagcctggtaccagcagaaacctggccaggctcccaggctcctcatctatggtgcatccagaagggccactggcatcccagacaggttcagtggcagtgggtctgggacagacttcactctcaccatcagcagactggagcctgaagactttgtagtgtattactgtcaacaatatggtagctcaccatacacttttggccaggggaccaagctggagatcaaa

SEQ ID NO: 10 - protein, 108 aa, synthetic construct
EIVLTQSPGTLSLSPGERATLSCRTSQSISSGYLAWYQQKPGQAPRLLIYGASRRATGIPDRFSGSGSGTDFTLTISRLEPEDFVVYYCQQYGSSPYTFGQGTKLEIK

SEQ ID NO: 11 - DNA, 21 bp, synthetic construct
  source 1..21: mol_type = other DNA
cagagtattagcagcggctat

SEQ ID NO: 12 - protein, 7 aa, synthetic construct
QSISSGY

SEQ ID NO: 13-14 - skipped (2 entries)

SEQ ID NO: 15 - DNA, 27 bp, synthetic construct
  source 1..27: mol_type = other DNA
caacaatatggtagctcaccatacact

SEQ ID NO: 16 - protein, 9 aa, synthetic construct
QQYGSSPYT

SEQ ID NO: 17 - DNA, 1329 bp, synthetic construct
  source 1..1329: mol_type = other DNA
caggtgcagctgcaggagtcgggcccaggactggtgaagccttcggagaccctgtccctcacctgcactgtctctggtgactccattaatagttacttctggagctggatccggcagttgccagggaaggaactggagtggattggccatatctattctagtgggagtaccagatacaacccctccctccagagtcgagtcaccatatcaatagacacgtccaagaaccagttctccctgaagctgagttctgtgaccgctgcggacacggccgtatattactgtgcgaggggcgccagcgcagttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 18 - protein, 442 aa, synthetic construct
QVQLQESGPGLVKPSETLSLTCTVSGDSINSYFWSWIRQLPGKELEWIGHIYSSGSTRYNPSLQSRVTISIDTSKNQFSLKLSSVTAADTAVYYCARGASAVDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 648 bp, synthetic construct
  source 1..648: mol_type = other DNA
gaaattgtgttgacgcagtctccaggcaccctgtctttgtctccaggggaaagagccaccctctcctgcaggaccagtcagagtattagcagcggctatttagcctggtaccagcagaaacctggccaggctcccaggctcctcatctatggtgcatccagaagggccactggcatcccagacaggttcagtggcagtgggtctgggacagacttcactctcaccatcagcagactggagcctgaagactttgtagtgtattactgtcaacaatatggtagctcaccatacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 20 - protein, 215 aa, synthetic construct
EIVLTQSPGTLSLSPGERATLSCRTSQSISSGYLAWYQQKPGQAPRLLIYGASRRATGIPDRFSGSGSGTDFTLTISRLEPEDFVVYYCQQYGSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 378 bp, synthetic construct
  source 1..378: mol_type = other DNA
caggtgcagctggtacagtctggggctgaggtgaagaagcctggggcctcagtgaaggtctcctgcaaggtttccggatacaccctcactgaattatccatgcactgggtgcgacaagctcctggaaaagggcttgagtggatgggaggttttgatcctgaagatggtgaaataatctacgcacagaagttccagggcagagtcaccatgaccgaggacacatctacagacacagcctacatggacctgagcagtctgacatctgaagacacggccgtgtattactgtgcaacggagaagcagcaactggtacgaaaatactacttctactacggtttggccgtctggggccaagggaccacggtcaccgtctcctca

SEQ ID NO: 22 - protein, 126 aa, synthetic construct
QVQLVQSGAEVKKPGASVKVSCKVSGYTLTELSMHWVRQAPGKGLEWMGGFDPEDGEIIYAQKFQGRVTMTEDTSTDTAYMDLSSLTSEDTAVYYCATEKQQLVRKYYFYYGLAVWGQGTTVTVSS

SEQ ID NO: 23 - DNA, 24 bp, synthetic construct
  source 1..24: mol_type = other DNA
ggatacaccctcactgaattatcc

SEQ ID NO: 24 - protein, 8 aa, synthetic construct
GYTLTELS

SEQ ID NO: 25 - DNA, 24 bp, synthetic construct
  source 1..24: mol_type = other DNA
tttgatcctgaagatggtgaaata

SEQ ID NO: 26 - protein, 8 aa, synthetic construct
FDPEDGEI

SEQ ID NO: 27 - DNA, 57 bp, synthetic construct
  source 1..57: mol_type = other DNA
gcaacggagaagcagcaactggtacgaaaatactacttctactacggtttggccgtc

SEQ ID NO: 28 - protein, 19 aa, synthetic construct
ATEKQQLVRKYYFYYGLAV

SEQ ID NO: 29 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagcagttatttaaattggtatcagcagaaaccagggaaagtccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtcccccattcactttcggccctgggaccaaagtggatatcaaa

SEQ ID NO: 30 - protein, 107 aa, synthetic construct
DIQMTQSPSSLSASVGDRVTITCRASQSISSYLNWYQQKPGKVPKLLIYAASSLQSGVPSRFSGSGSGTDFTLTISSLQPEDFATYYCQQSYSPPFTFGPGTKVDIK

SEQ ID NO: 31 - DNA, 18 bp, synthetic construct
  source 1..18: mol_type = other DNA
cagagcattagcagttat

SEQ ID NO: 32 - protein, 6 aa, synthetic construct
QSISSY

SEQ ID NO: 33-34 - skipped (2 entries)

SEQ ID NO: 35 - DNA, 27 bp, synthetic construct
  source 1..27: mol_type = other DNA
caacagagttacagtcccccattcact

SEQ ID NO: 36 - protein, 9 aa, synthetic construct
QQSYSPPFT

SEQ ID NO: 37 - DNA, 1362 bp, synthetic construct
  source 1..1362: mol_type = other DNA
caggtgcagctggtacagtctggggctgaggtgaagaagcctggggcctcagtgaaggtctcctgcaaggtttccggatacaccctcactgaattatccatgcactgggtgcgacaagctcctggaaaagggcttgagtggatgggaggttttgatcctgaagatggtgaaataatctacgcacagaagttccagggcagagtcaccatgaccgaggacacatctacagacacagcctacatggacctgagcagtctgacatctgaagacacggccgtgtattactgtgcaacggagaagcagcaactggtacgaaaatactacttctactacggtttggccgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 38 - protein, 453 aa, synthetic construct
QVQLVQSGAEVKKPGASVKVSCKVSGYTLTELSMHWVRQAPGKGLEWMGGFDPEDGEIIYAQKFQGRVTMTEDTSTDTAYMDLSSLTSEDTAVYYCATEKQQLVRKYYFYYGLAVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - DNA, 645 bp, synthetic construct
  source 1..645: mol_type = other DNA
gacatccagatgacccagtctccatcctccctgtctgcatctgtaggagacagagtcaccatcacttgccgggcaagtcagagcattagcagttatttaaattggtatcagcagaaaccagggaaagtccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtcccccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 40 - protein, 214 aa, synthetic construct
DIQMTQSPSSLSASVGDRVTITCRASQSISSYLNWYQQKPGKVPKLLIYAASSLQSGVPSRFSGSGSGTDFTLTISSLQPEDFATYYCQQSYSPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - DNA, 369 bp, synthetic construct
  source 1..369: mol_type = other DNA
caggtacagctgcagcagtcaggtccaggactggtgaagccctcgcagaccctctcactcacctgtgccatctccggggacagtgtctctaggaacagtgctgcttggaactggatcaggcagtccccatcgagtggccttgagtggctgggaaggacatactacaggtccaagtggttttatgattatgcaatatctgtgaaaagtcgaataaccgtcaacccagacacatccaagaaccaattctcccttcacctgaactctgtgactcccgaagacacggctgtctattactgtgcgagaggctacggtggctacgaggactactttgactactggggccagggaaccctggtcaccgtctcctca

SEQ ID NO: 42 - protein, 123 aa, synthetic construct
QVQLQQSGPGLVKPSQTLSLTCAISGDSVSRNSAAWNWIRQSPSSGLEWLGRTYYRSKWFYDYAISVKSRITVNPDTSKNQFSLHLNSVTPEDTAVYYCARGYGGYEDYFDYWGQGTLVTVSS

SEQ ID NO: 43 - DNA, 30 bp, synthetic construct
  source 1..30: mol_type = other DNA
ggggacagtgtctctaggaacagtgctgct

SEQ ID NO: 44 - protein, 10 aa, synthetic construct
GDSVSRNSAA

SEQ ID NO: 45 - DNA, 27 bp, synthetic construct
  source 1..27: mol_type = other DNA
acatactacaggtccaagtggttttat

SEQ ID NO: 46 - protein, 9 aa, synthetic construct
TYYRSKWFY

SEQ ID NO: 47 - DNA, 39 bp, synthetic construct
  source 1..39: mol_type = other DNA
gcgagaggctacggtggctacgaggactactttgactac

SEQ ID NO: 48 - protein, 13 aa, synthetic construct
ARGYGGYEDYFDY

SEQ ID NO: 49 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatggtgcatccagtttgcaaagtggggtcccatcaaggttcagcggcagtggatctgggacagatttcactctcaccatcagcagcctgcagcctgaagattttgcaacttacttttgtcaacagggtagcagtttcccgtacacttttggccaggggaccaagctggagatcaaa

SEQ ID NO: 50 - protein, 107 aa, synthetic construct
DIQMTQSPSSVSASVGDRVTITCRASQGISSWLAWYQQKPGKAPKLLIYGASSLQSGVPSRFSGSGSGTDFTLTISSLQPEDFATYFCQQGSSFPYTFGQGTKLEIK

SEQ ID NO: 51 - DNA, 18 bp, synthetic construct
  source 1..18: mol_type = other DNA
cagggtattagcagctgg

SEQ ID NO: 52 - protein, 6 aa, synthetic construct
QGISSW

SEQ ID NO: 53-54 - skipped (2 entries)

SEQ ID NO: 55 - DNA, 27 bp, synthetic construct
  source 1..27: mol_type = other DNA
caacagggtagcagtttcccgtacact

SEQ ID NO: 56 - protein, 9 aa, synthetic construct
QQGSSFPYT

SEQ ID NO: 57 - DNA, 1353 bp, synthetic construct
  source 1..1353: mol_type = other DNA
caggtacagctgcagcagtcaggtccaggactggtgaagccctcgcagaccctctcactcacctgtgccatctccggggacagtgtctctaggaacagtgctgcttggaactggatcaggcagtccccatcgagtggccttgagtggctgggaaggacatactacaggtccaagtggttttatgattatgcaatatctgtgaaaagtcgaataaccgtcaacccagacacatccaagaaccaattctcccttcacctgaactctgtgactcccgaagacacggctgtctattactgtgcgagaggctacggtggctacgaggactactttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 58 - protein, 450 aa, synthetic construct
QVQLQQSGPGLVKPSQTLSLTCAISGDSVSRNSAAWNWIRQSPSSGLEWLGRTYYRSKWFYDYAISVKSRITVNPDTSKNQFSLHLNSVTPEDTAVYYCARGYGGYEDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - DNA, 645 bp, synthetic construct
  source 1..645: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatggtgcatccagtttgcaaagtggggtcccatcaaggttcagcggcagtggatctgggacagatttcactctcaccatcagcagcctgcagcctgaagattttgcaacttacttttgtcaacagggtagcagtttcccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 60 - protein, 214 aa, synthetic construct
DIQMTQSPSSVSASVGDRVTITCRASQGISSWLAWYQQKPGKAPKLLIYGASSLQSGVPSRFSGSGSGTDFTLTISSLQPEDFATYFCQQGSSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - DNA, 357 bp, synthetic construct
  source 1..357: mol_type = other DNA
gaggtgcagctggtggagtctgggggaggcttggtacagcctggagggtccctgagactctcctgtatagtctctggattcatcttcagtagttatgaaatgagctggctccgccaggctccagggaagggcctggagtggatttcatatattagtagtagtggtcgtgtcatatactatgcagactctgtgaagggccgattcaccatctccagagacagcgccaagaattcactgtatctggaaatgaatagtctgagagccgaagacacggctatatattattgtacgagaaagtgggatagtagtggcccatttgacttctggggccagggaaccctggtcaccgtctcctca

SEQ ID NO: 62 - protein, 119 aa, synthetic construct
EVQLVESGGGLVQPGGSLRLSCIVSGFIFSSYEMSWLRQAPGKGLEWISYISSSGRVIYYADSVKGRFTISRDSAKNSLYLEMNSLRAEDTAIYYCTRKWDSSGPFDFWGQGTLVTVSS

SEQ ID NO: 63 - DNA, 24 bp, synthetic construct
  source 1..24: mol_type = other DNA
ggattcatcttcagtagttatgaa

SEQ ID NO: 64 - protein, 8 aa, synthetic construct
GFIFSSYE

SEQ ID NO: 65 - DNA, 24 bp, synthetic construct
  source 1..24: mol_type = other DNA
attagtagtagtggtcgtgtcata

SEQ ID NO: 66 - protein, 8 aa, synthetic construct
ISSSGRVI

SEQ ID NO: 67 - DNA, 36 bp, synthetic construct
  source 1..36: mol_type = other DNA
acgagaaagtgggatagtagtggcccatttgacttc

SEQ ID NO: 68 - protein, 12 aa, synthetic construct
TRKWDSSGPFDF

SEQ ID NO: 69 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 70 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 71 - DNA, 18 bp, synthetic construct
  source 1..18: mol_type = other DNA
cagagcattagcagctat

SEQ ID NO: 72 - protein, 6 aa, synthetic construct
QSISSY

SEQ ID NO: 73-74 - skipped (2 entries)

SEQ ID NO: 75 - DNA, 30 bp, synthetic construct
  source 1..30: mol_type = other DNA
caacagagttacagtacccctccgatcacc

SEQ ID NO: 76 - protein, 10 aa, synthetic construct
QQSYSTPPIT

SEQ ID NO: 77 - DNA, 1341 bp, synthetic construct
  source 1..1341: mol_type = other DNA
gaggtgcagctggtggagtctgggggaggcttggtacagcctggagggtccctgagactctcctgtatagtctctggattcatcttcagtagttatgaaatgagctggctccgccaggctccagggaagggcctggagtggatttcatatattagtagtagtggtcgtgtcatatactatgcagactctgtgaagggccgattcaccatctccagagacagcgccaagaattcactgtatctggaaatgaatagtctgagagccgaagacacggctatatattattgtacgagaaagtgggatagtagtggcccatttgacttctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 78 - protein, 446 aa, synthetic construct
EVQLVESGGGLVQPGGSLRLSCIVSGFIFSSYEMSWLRQAPGKGLEWISYISSSGRVIYYADSVKGRFTISRDSAKNSLYLEMNSLRAEDTAIYYCTRKWDSSGPFDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 79 - DNA, 648 bp, synthetic construct
  source 1..648: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 80 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - DNA, 375 bp, synthetic construct
  source 1..375: mol_type = other DNA
caggtgcagctggtggagtctgggggaggcttggtcaagcctggagggtccctaagactctcctgtgcagcctctggattcaccttcaatgactacccaatgagctggatccgccaggctccagggaagggactggagtgggtttcatacattactagcagtagtggtagtaccatatactacgcagactctgtgaagggccgattcaccatctccagggacaacgccaagaactcactgtatctgcaaatgaacagcctgagagccgaggacacggccgtgtattactgtgcgagagaggttgtagtggctacgattgggggctactacggtatggacgtctggggccaagggaccacggtcaccgtctcctca

SEQ ID NO: 82 - protein, 125 aa, synthetic construct
QVQLVESGGGLVKPGGSLRLSCAASGFTFNDYPMSWIRQAPGKGLEWVSYITSSSGSTIYYADSVKGRFTISRDNAKNSLYLQMNSLRAEDTAVYYCAREVVVATIGGYYGMDVWGQGTTVTVSS

SEQ ID NO: 83 - DNA, 24 bp, synthetic construct
  source 1..24: mol_type = other DNA
ggattcaccttcaatgactaccca

SEQ ID NO: 84 - protein, 8 aa, synthetic construct
GFTFNDYP

SEQ ID NO: 85 - DNA, 27 bp, synthetic construct
  source 1..27: mol_type = other DNA
attactagcagtagtggtagtaccata

SEQ ID NO: 86 - protein, 9 aa, synthetic construct
ITSSSGSTI

SEQ ID NO: 87 - DNA, 51 bp, synthetic construct
  source 1..51: mol_type = other DNA
gcgagagaggttgtagtggctacgattgggggctactacggtatggacgtc

SEQ ID NO: 88 - protein, 17 aa, synthetic construct
AREVVVATIGGYYGMDV

SEQ ID NO: 89 - DNA, 324 bp, synthetic construct; identical to SEQ ID NO: 69
  source 1..324: mol_type = other DNA

SEQ ID NO: 90 - protein, 108 aa, synthetic construct; identical to SEQ ID NO: 70

SEQ ID NO: 91 - DNA, 18 bp, synthetic construct
  source 1..18: mol_type = other DNA
cagagcattagcagctat

SEQ ID NO: 92 - protein, 6 aa, synthetic construct
QSISSY

SEQ ID NO: 93-94 - skipped (2 entries)

SEQ ID NO: 95 - DNA, 30 bp, synthetic construct; identical to SEQ ID NO: 75
  source 1..30: mol_type = other DNA

SEQ ID NO: 96 - protein, 10 aa, synthetic construct
QQSYSTPPIT

SEQ ID NO: 97 - DNA, 1359 bp, synthetic construct
  source 1..1359: mol_type = other DNA
caggtgcagctggtggagtctgggggaggcttggtcaagcctggagggtccctaagactctcctgtgcagcctctggattcaccttcaatgactacccaatgagctggatccgccaggctccagggaagggactggagtgggtttcatacattactagcagtagtggtagtaccatatactacgcagactctgtgaagggccgattcaccatctccagggacaacgccaagaactcactgtatctgcaaatgaacagcctgagagccgaggacacggccgtgtattactgtgcgagagaggttgtagtggctacgattgggggctactacggtatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 98 - protein, 452 aa, synthetic construct
QVQLVESGGGLVKPGGSLRLSCAASGFTFNDYPMSWIRQAPGKGLEWVSYITSSSGSTIYYADSVKGRFTISRDNAKNSLYLQMNSLRAEDTAVYYCAREVVVATIGGYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - DNA, 648 bp, synthetic construct; identical to SEQ ID NO: 79
  source 1..648: mol_type = other DNA

SEQ ID NO: 100 - protein, 215 aa, synthetic construct; identical to SEQ ID NO: 80

SEQ ID NO: 101 - DNA, 378 bp, synthetic construct
  source 1..378: mol_type = other DNA
gaggtgcagctggtggagtctgggggagacttggtacagcctggagggtccctgagactctcctgtgcagcctctggattcaccttcagtagttatgaaatgaactgggtccgccaggctccagggaaggggctggagtgggtttcatacattagtaatagtggttctaccatatactacgcagactctgtgaagggccgattcaccatctccagagacaacgccgagacctcactgtatctgcaaatgaacagcctgagagccgaggacacggctgtttattactgtgcgagagagggatggggcgcatattgtgctggtgattgctattctggttttgatatttggggccaagggacaatggtcaccgtctcttca

SEQ ID NO: 102 - protein, 126 aa, synthetic construct
EVQLVESGGDLVQPGGSLRLSCAASGFTFSSYEMNWVRQAPGKGLEWVSYISNSGSTIYYADSVKGRFTISRDNAETSLYLQMNSLRAEDTAVYYCAREGWGAYCAGDCYSGFDIWGQGTMVTVSS

SEQ ID NO: 103 - DNA, 24 bp, synthetic construct
  source 1..24: mol_type = other DNA
ggattcaccttcagtagttatgaa

SEQ ID NO: 104 - protein, 8 aa, synthetic construct
GFTFSSYE

SEQ ID NO: 105 - DNA, 24 bp, synthetic construct
  source 1..24: mol_type = other DNA
attagtaatagtggttctaccata

SEQ ID NO: 106 - protein, 8 aa, synthetic construct
ISNSGSTI

SEQ ID NO: 107 - DNA, 57 bp, synthetic construct
  source 1..57: mol_type = other DNA
gcgagagagggatggggcgcatattgtgctggtgattgctattctggttttgatatt

SEQ ID NO: 108 - protein, 19 aa, synthetic construct
AREGWGAYCAGDCYSGFDI

SEQ ID NO: 109 - DNA, 324 bp, synthetic construct; identical to SEQ ID NO: 69
  source 1..324: mol_type = other DNA

SEQ ID NO: 110 - protein, 108 aa, synthetic construct; identical to SEQ ID NO: 70

SEQ ID NO: 111 - DNA, 18 bp, synthetic construct
  source 1..18: mol_type = other DNA
cagagcattagcagctat

SEQ ID NO: 112 - protein, 6 aa, synthetic construct
QSISSY

SEQ ID NO: 113-114 - skipped (2 entries)

SEQ ID NO: 115 - DNA, 30 bp, synthetic construct; identical to SEQ ID NO: 75
  source 1..30: mol_type = other DNA

SEQ ID NO: 116 - protein, 10 aa, synthetic construct
QQSYSTPPIT

SEQ ID NO: 117 - DNA, 1362 bp, synthetic construct
  source 1..1362: mol_type = other DNA
gaggtgcagctggtggagtctgggggagacttggtacagcctggagggtccctgagactctcctgtgcagcctctggattcaccttcagtagttatgaaatgaactgggtccgccaggctccagggaaggggctggagtgggtttcatacattagtaatagtggttctaccatatactacgcagactctgtgaagggccgattcaccatctccagagacaacgccgagacctcactgtatctgcaaatgaacagcctgagagccgaggacacggctgtttattactgtgcgagagagggatggggcgcatattgtgctggtgattgctattctggttttgatatttggggccaagggacaatggtcaccgtctcttcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 118 - protein, 453 aa, synthetic construct
EVQLVESGGDLVQPGGSLRLSCAASGFTFSSYEMNWVRQAPGKGLEWVSYISNSGSTIYYADSVKGRFTISRDNAETSLYLQMNSLRAEDTAVYYCAREGWGAYCAGDCYSGFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 119 - DNA, 648 bp, synthetic construct; identical to SEQ ID NO: 79
  source 1..648: mol_type = other DNA

SEQ ID NO: 120 - protein, 215 aa, synthetic construct; identical to SEQ ID NO: 80

SEQ ID NO: 121 - DNA, 357 bp, synthetic construct
  source 1..357: mol_type = other DNA
gaggtgcagctggtggagtctggaggaggcttggtccagcctggggggtccctgagactctcctgtgcagcctctgggttcaccgtcagtagcaactacatgagctgggtccgccaggctccagggaaggggctggagtgggtctcaattatttatagcggtggtcacacatactactcagactccgtgaagggccgattcaccatctccagacacaattccaagaacactctgtatcttcaaatgaacagcctgagaggtggggacacggccgtgtattactgtgcgagagggtataccagtggctggtacggatttgacttctggggccagggaaccctggtcaccgtctcctca

SEQ ID NO: 122 - protein, 119 aa, synthetic construct
EVQLVESGGGLVQPGGSLRLSCAASGFTVSSNYMSWVRQAPGKGLEWVSIIYSGGHTYYSDSVKGRFTISRHNSKNTLYLQMNSLRGGDTAVYYCARGYTSGWYGFDFWGQGTLVTVSS

SEQ ID NO: 123 - DNA, 24 bp, synthetic construct
  source 1..24: mol_type = other DNA
gggttcaccgtcagtagcaactac

SEQ ID NO: 124 - protein, 8 aa, synthetic construct
GFTVSSNY

SEQ ID NO: 125 - DNA, 21 bp, synthetic construct
  source 1..21: mol_type = other DNA
atttatagcggtggtcacaca

SEQ ID NO: 126 - protein, 7 aa, synthetic construct
IYSGGHT

SEQ ID NO: 127 - DNA, 39 bp, synthetic construct
  source 1..39: mol_type = other DNA
gcgagagggtataccagtggctggtacggatttgacttc

SEQ ID NO: 128 - protein, 13 aa, synthetic construct
ARGYTSGWYGFDF

SEQ ID NO: 129 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcacctggttagcctggtatcagcagaaaccagggaaagcccctaagctcctgatctatgctgcatccagtttgcaaagtggggtcccgtcaagattcagcggcactggatctgggacagatttcactctcaccatcagcagcctgcagcctgaagattttgcaacttattattgtcagcagactaacagtttcccgtggacgttcggccaagggaccaaggtggaaatcaaa

SEQ ID NO: 130 - protein, 107 aa, synthetic construct
DIQMTQSPSSVSASVGDRVTITCRASQGISTWLAWYQQKPGKAPKLLIYAASSLQSGVPSRFSGTGSGTDFTLTISSLQPEDFATYYCQQTNSFPWTFGQGTKVEIK

SEQ ID NO: 131 - DNA, 18 bp, synthetic construct
  source 1..18: mol_type = other DNA
cagggtattagcacctgg

SEQ ID NO: 132 - protein, 6 aa, synthetic construct
QGISTW

SEQ ID NO: 133 - DNA, 27 bp, synthetic construct
  source 1..27: mol_type = other DNA
cagcagactaacagtttcccgtggacg

SEQ ID NO: 134 - protein, 9 aa, synthetic construct
QQTNSFPWT

SEQ ID NO: 135 - DNA, 1341 bp, synthetic construct
  source 1..1341: mol_type = other DNA
gaggtgcagctggtggagtctggaggaggcttggtccagcctggggggtccctgagactctcctgtgcagcctctgggttcaccgtcagtagcaactacatgagctgggtccgccaggctccagggaaggggctggagtgggtctcaattatttatagcggtggtcacacatactactcagactccgtgaagggccgattcaccatctccagacacaattccaagaacactctgtatcttcaaatgaacagcctgagaggtggggacacggccgtgtattactgtgcgagagggtataccagtggctggtacggatttgacttctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 136 - protein, 446 aa, synthetic construct
EVQLVESGGGLVQPGGSLRLSCAASGFTVSSNYMSWVRQAPGKGLEWVSIIYSGGHTYYSDSVKGRFTISRHNSKNTLYLQMNSLRGGDTAVYYCARGYTSGWYGFDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 137 - DNA, 645 bp, synthetic construct
  source 1..645: mol_type = other DNA
gacatccagatgacccagtctccatcttccgtgtctgcatctgtaggagacagagtcaccatcacttgtcgggcgagtcagggtattagcacctggttagcctggtatcagcagaaaccagggaaagcccctaagctcctgatctatgctgcatccagtttgcaaagtggggtcccgtcaagattcagcggcactggatctgggacagatttcactctcaccatcagcagcctgcagcctgaagattttgcaacttattattgtcagcagactaacagtttcccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 138 - protein, 214 aa, synthetic construct
DIQMTQSPSSVSASVGDRVTITCRASQGISTWLAWYQQKPGKAPKLLIYAASSLQSGVPSRFSGTGSGTDFTLTISSLQPEDFATYYCQQTNSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - DNA, 357 bp, synthetic construct
  source 1..357: mol_type = other DNA
gaggtgcagctggtgcagtctggagcagaggtgaaaaagcccggggagtctctgaagatctcctgtaagggttctggatacagctttaccagctactggatcgcctgggtgcgccagatgcccgggaaaggcctggagtggatggggatcatctatcctggtgactctgataccagatacagcccgtccttccaaggccaggtcaccatgtcagccgacaagtccatcaggatcgcctacctgcagtggagcagcctgaaggcctcggacaccgccatgtattattgtgcgagacttgattatagcggcagctggtttgactactggggccagggaaccctggtcaccgtctcctca

SEQ ID NO: 140 - protein, 119 aa, synthetic construct
EVQLVQSGAEVKKPGESLKISCKGSGYSFTSYWIAWVRQMPGKGLEWMGIIYPGDSDTRYSPSFQGQVTMSADKSIRIAYLQWSSLKASDTAMYYCARLDYSGSWFDYWGQGTLVTVSS

SEQ ID NO: 141 - DNA, 24 bp, synthetic construct
  source 1..24: mol_type = other DNA
ggatacagctttaccagctactgg

SEQ ID NO: 142 - protein, 8 aa, synthetic construct
GYSFTSYW

SEQ ID NO: 143 - DNA, 24 bp, synthetic construct
  source 1..24: mol_type = other DNA
atctatcctggtgactctgatacc

SEQ ID NO: 144 - protein, 8 aa, synthetic construct
IYPGDSDT

SEQ ID NO: 145 - DNA, 36 bp, synthetic construct
  source 1..36: mol_type = other DNA
gcgagacttgattatagcggcagctggtttgactac

SEQ ID NO: 146 - protein, 12 aa, synthetic construct
ARLDYSGSWFDY

SEQ ID NO: 147 - DNA, 324 bp, synthetic construct
  source 1..324: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 148 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 149 - DNA, 21 bp, synthetic construct
  source 1..21: mol_type = other DNA
cagtcagtctctagctcttat

SEQ ID NO: 150 - protein, 7 aa, synthetic construct
QSVSSSY

SEQ ID NO: 151 - skipped

SEQ ID NO: 152 - DNA, 27 bp, synthetic construct
  source 1..27: mol_type = other DNA
caacagtacggaagcagcccgtggacg

SEQ ID NO: 153 - protein, 9 aa, synthetic construct
QQYGSSPWT

SEQ ID NO: 154 - DNA, 1341 bp, synthetic construct
  source 1..1341: mol_type = other DNA
gaggtgcagctggtgcagtctggagcagaggtgaaaaagcccggggagtctctgaagatctcctgtaagggttctggatacagctttaccagctactggatcgcctgggtgcgccagatgcccgggaaaggcctggagtggatggggatcatctatcctggtgactctgataccagatacagcccgtccttccaaggccaggtcaccatgtcagccgacaagtccatcaggatcgcctacctgcagtggagcagcctgaaggcctcggacaccgccatgtattattgtgcgagacttgattatagcggcagctggtttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 155 - protein, 446 aa, synthetic construct
EVQLVQSGAEVKKPGESLKISCKGSGYSFTSYWIAWVRQMPGKGLEWMGIIYPGDSDTRYSPSFQGQVTMSADKSIRIAYLQWSSLKASDTAMYYCARLDYSGSWFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 156 - DNA, 648 bp, synthetic construct
  source 1..648: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 157 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - DNA, 366 bp, synthetic construct
  source 1..366: mol_type = other DNA
caggtccagctggtgcagtctggggctgaggtgaagaagcctggggcctcagtgaaggtctcctgcaaggcttctggatacaccttcaacagttatgatgtcaactgggtgcgacaggccactggacaagggcttgagtggatgggatggatgaaccctcacagtggtaacacaggctacgcacagaagttccagggcagagtcactatgaccagggacacctccacaagcacaagctatatggagttgagcagcctgacatctgaggacacggccgtatattactgtgcgagaggccctttttctctactttcagctgaattcttccagcactggggccagggcaccctggtcaccgtctcctca

SEQ ID NO: 159 - protein, 122 aa, synthetic construct
QVQLVQSGAEVKKPGASVKVSCKASGYTFNSYDVNWVRQATGQGLEWMGWMNPHSGNTGYAQKFQGRVTMTRDTSTSTSYMELSSLTSEDTAVYYCARGPFSLLSAEFFQHWGQGTLVTVSS

SEQ ID NO: 160 - DNA, 24 bp, synthetic construct
  source 1..24: mol_type = other DNA
ggatacaccttcaacagttatgat

SEQ ID NO: 161 - protein, 8 aa, synthetic construct
GYTFNSYD

SEQ ID NO: 162 - DNA, 24 bp, synthetic construct
  source 1..24: mol_type = other DNA
atgaaccctcacagtggtaacaca

SEQ ID NO: 163 - protein, 8 aa, synthetic construct
MNPHSGNT

SEQ ID NO: 164 - DNA, 45 bp, synthetic construct
  source 1..45: mol_type = other DNA
gcgagaggccctttttctctactttcagctgaattcttccagcac

SEQ ID NO: 165 - protein, 15 aa, synthetic construct
ARGPFSLLSAEFFQH

SEQ ID NO: 166 - DNA, 1350 bp, synthetic construct
  source 1..1350: mol_type = other DNA
caggtccagctggtgcagtctggggctgaggtgaagaagcctggggcctcagtgaaggtctcctgcaaggcttctggatacaccttcaacagttatgatgtcaactgggtgcgacaggccactggacaagggcttgagtggatgggatggatgaaccctcacagtggtaacacaggctacgcacagaagttccagggcagagtcactatgaccagggacacctccacaagcacaagctatatggagttgagcagcctgacatctgaggacacggccgtatattactgtgcgagaggccctttttctctactttcagctgaattcttccagcactggggccagggcaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 167 - protein, 449 aa, synthetic construct
QVQLVQSGAEVKKPGASVKVSCKASGYTFNSYDVNWVRQATGQGLEWMGWMNPHSGNTGYAQKFQGRVTMTRDTSTSTSYMELSSLTSEDTAVYYCARGPFSLLSAEFFQHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 168 - DNA, 357 bp, synthetic construct
  source 1..357: mol_type = other DNA
caggtccagctggtacagtctggggctgaggtgaagaagcctgggtcctcggtgaaggtctcctgcaaggcttctggagacaccttcagtaactatgttatcggctgggtgcgacaggcccctggacaagggcttgagtggatgggagggatcatccctatctttggtacaacaaactacgcacagcagttccagggcagagtcacgattaccacggacgaatccacgagcacggcctacatggagctgagcagcctgagatctgaggacacggccgtgtattactgtgcgagagatgggaactacggtgactactttgactactggggccagggaaccctggtcaccgtctcctca

SEQ ID NO: 169 - protein, 119 aa, synthetic construct
QVQLVQSGAEVKKPGSSVKVSCKASGDTFSNYVIGWVRQAPGQGLEWMGGIIPIFGTTNYAQQFQGRVTITTDESTSTAYMELSSLRSEDTAVYYCARDGNYGDYFDYWGQGTLVTVSS

SEQ ID NO: 170 - DNA, 24 bp, synthetic construct
  source 1..24: mol_type = other DNA
ggagacaccttcagtaactatgtt

SEQ ID NO: 171 - protein, 8 aa, synthetic construct
GDTFSNYV

SEQ ID NO: 172 - DNA, 24 bp, synthetic construct
  source 1..24: mol_type = other DNA
atcatccctatctttggtacaaca

SEQ ID NO: 173 - protein, 8 aa, synthetic construct
IIPIFGTT

SEQ ID NO: 174 - DNA, 36 bp, synthetic construct
  source 1..36: mol_type = other DNA
gcgagagatgggaactacggtgactactttgactac

SEQ ID NO: 175 - protein, 12 aa, synthetic construct
ARDGNYGDYFDY

SEQ ID NO: 176 - DNA, 1341 bp, synthetic construct
  source 1..1341: mol_type = other DNA
caggtccagctggtacagtctggggctgaggtgaagaagcctgggtcctcggtgaaggtctcctgcaaggcttctggagacaccttcagtaactatgttatcggctgggtgcgacaggcccctggacaagggcttgagtggatgggagggatcatccctatctttggtacaacaaactacgcacagcagttccagggcagagtcacgattaccacggacgaatccacgagcacggcctacatggagctgagcagcctgagatctgaggacacggccgtgtattactgtgcgagagatgggaactacggtgactactttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 177 - protein, 446 aa, synthetic construct
QVQLVQSGAEVKKPGSSVKVSCKASGDTFSNYVIGWVRQAPGQGLEWMGGIIPIFGTTNYAQQFQGRVTITTDESTSTAYMELSSLRSEDTAVYYCARDGNYGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 178 - DNA, 351 bp, synthetic construct
  source 1..351: mol_type = other DNA
caggttcagctggtgcagtctggagctgaggtgaagaagcctggggcctcagtgaaggtctcctgcaagacttctggttacagattcgtcaactatggtttcagctgggtgcgccaggcccctggacaaggccttgaatggatgggatggatcagcccttataatggtaacacaaactatatacagaatctccaggacagaatcaccatgaccacagacacctctacgaacacagcctacatggaactgacgaacctgagatctgacgacacggccgtatattactgtgcgaccttaactggggttcactttgactactggggccagggaaccctggtcaccgtctcctca

SEQ ID NO: 179 - protein, 117 aa, synthetic construct
QVQLVQSGAEVKKPGASVKVSCKTSGYRFVNYGFSWVRQAPGQGLEWMGWISPYNGNTNYIQNLQDRITMTTDTSTNTAYMELTNLRSDDTAVYYCATLTGVHFDYWGQGTLVTVSS

SEQ ID NO: 180 - DNA, 24 bp, synthetic construct
  source 1..24: mol_type = other DNA
ggttacagattcgtcaactatggt

SEQ ID NO: 181 - protein, 8 aa, synthetic construct
GYRFVNYG

SEQ ID NO: 182 - DNA, 24 bp, synthetic construct
  source 1..24: mol_type = other DNA
atcagcccttataatggtaacaca

SEQ ID NO: 183 - protein, 8 aa, synthetic construct
ISPYNGNT

SEQ ID NO: 184 - DNA, 30 bp, synthetic construct
  source 1..30: mol_type = other DNA
gcgaccttaactggggttcactttgactac

SEQ ID NO: 185 - protein, 10 aa, synthetic construct
ATLTGVHFDY

SEQ ID NO: 186 - DNA, 321 bp, synthetic construct
  source 1..321: mol_type = other DNA
gccatccagatgacccagtctccatcctccctgtctgcatctgtaggagacagagtcaccatcacttgccgggcaagtcagggcattagaaatgatttaggctggtatcagcagaaaccagggaaagcccctaatctcctgatctttgaaacatctcgtttacaaagtggggtcccttcgaggttcagcggcagtggttctggcacagatttcactctcaccatcaacagccttcagcctgaagattttgcaacttattactgtctacaagattacaattacccgtggacgttcggccaagggaccaaggtggaaatcaaa

SEQ ID NO: 187 - protein, 107 aa, synthetic construct
AIQMTQSPSSLSASVGDRVTITCRASQGIRNDLGWYQQKPGKAPNLLIFETSRLQSGVPSRFSGSGSGTDFTLTINSLQPEDFATYYCLQDYNYPWTFGQGTKVEIK

SEQ ID NO: 188 - DNA, 18 bp, synthetic construct
  source 1..18: mol_type = other DNA
cagggcattagaaatgat

SEQ ID NO: 189 - protein, 6 aa, synthetic construct
QGIRND

SEQ ID NO: 190-191 - skipped (2 entries)

SEQ ID NO: 192 - DNA, 27 bp, synthetic construct
  source 1..27: mol_type = other DNA
ctacaagattacaattacccgtggacg

SEQ ID NO: 193 - protein, 9 aa, synthetic construct
LQDYNYPWT

SEQ ID NO: 194 - DNA, 1335 bp, synthetic construct
  source 1..1335: mol_type = other DNA
caggttcagctggtgcagtctggagctgaggtgaagaagcctggggcctcagtgaaggtctcctgcaagacttctggttacagattcgtcaactatggtttcagctgggtgcgccaggcccctggacaaggccttgaatggatgggatggatcagcccttataatggtaacacaaactatatacagaatctccaggacagaatcaccatgaccacagacacctctacgaacacagcctacatggaactgacgaacctgagatctgacgacacggccgtatattactgtgcgaccttaactggggttcactttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 195 - protein, 444 aa, synthetic construct
QVQLVQSGAEVKKPGASVKVSCKTSGYRFVNYGFSWVRQAPGQGLEWMGWISPYNGNTNYIQNLQDRITMTTDTSTNTAYMELTNLRSDDTAVYYCATLTGVHFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 196 - DNA, 645 bp, synthetic construct
  source 1..645: mol_type = other DNA
gccatccagatgacccagtctccatcctccctgtctgcatctgtaggagacagagtcaccatcacttgccgggcaagtcagggcattagaaatgatttaggctggtatcagcagaaaccagggaaagcccctaatctcctgatctttgaaacatctcgtttacaaagtggggtcccttcgaggttcagcggcagtggttctggcacagatttcactctcaccatcaacagccttcagcctgaagattttgcaacttattactgtctacaagattacaattacccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 197 - protein, 214 aa, synthetic construct
AIQMTQSPSSLSASVGDRVTITCRASQGIRNDLGWYQQKPGKAPNLLIFETSRLQSGVPSRFSGSGSGTDFTLTINSLQPEDFATYYCLQDYNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - DNA, 351 bp, synthetic construct
  source 1..351: mol_type = other DNA
gaggtgcagctggtggagtctgggggaggcttggcccagcctggggggtccctgaaactctcttgtgaagcctctggattcacgttcagtgattctgcaatgcactgggtccgccaggcttccggaaaagggctggagtgggttggtcgtattagaagcaaagctaatagttacgcgacaggatatgctgcgtcggtgaaaggcaggttcaccatctccagagatgattcaaagaacatggcgtttctggaaatgaacagcctgaaaaccgaagacacggccgtatattactgtctccgacaaacttacggtgacccctggggccagggaaccctggtcaccgtctcctca

SEQ ID NO: 199 - protein, 117 aa, synthetic construct
EVQLVESGGGLAQPGGSLKLSCEASGFTFSDSAMHWVRQASGKGLEWVGRIRSKANSYATGYAASVKGRFTISRDDSKNMAFLEMNSLKTEDTAVYYCLRQTYGDPWGQGTLVTVSS

SEQ ID NO: 200 - DNA, 24 bp, synthetic construct
  source 1..24: mol_type = other DNA
ggattcacgttcagtgattctgca

SEQ ID NO: 201 - protein, 8 aa, synthetic construct
GFTFSDSA

SEQ ID NO: 202 - DNA, 30 bp, synthetic construct
  source 1..30: mol_type = other DNA
attagaagcaaagctaatagttacgcgaca

SEQ ID NO: 203 - protein, 10 aa, synthetic construct
IRSKANSYAT

SEQ ID NO: 204 - DNA, 24 bp, synthetic construct
  source 1..24: mol_type = other DNA
ctccgacaaacttacggtgacccc

SEQ ID NO: 205 - protein, 8 aa, synthetic construct
LRQTYGDP

SEQ ID NO: 206 - DNA, 336 bp, synthetic construct
  source 1..336: mol_type = other DNA
gatgttgtgatgactcagtctccactctccctgtccgtcacccttggacagccggcctccatctcctgcaggtctagtctaagcctcgtatacagtgatggaaacaactacttgaattggtttcagcagaggccaggccaatctccaaggcgcctactttataaagtttttaaccgggactctggggtcccagacagattcagcggcagtgggtcaggcactgatttcacactgaaaatcagcagggtggaggctgaggatgttggggtttattactgcatgcaaggaacacactggccgtggacgttcggccaagggaccaaggtggaaatcaaa

SEQ ID NO: 207 - protein, 112 aa, synthetic construct
DVVMTQSPLSLSVTLGQPASISCRSSLSLVYSDGNNYLNWFQQRPGQSPRRLLYKVFNRDSGVPDRFSGSGSGTDFTLKISRVEAEDVGVYYCMQGTHWPWTFGQGTKVEIK

SEQ ID NO: 208 - DNA, 33 bp, synthetic construct
  source 1..33: mol_type = other DNA
ctaagcctcgtatacagtgatggaaacaactac

SEQ ID NO: 209 - protein, 11 aa, synthetic construct
LSLVYSDGNNY

SEQ ID NO: 210-211 - skipped (2 entries)

SEQ ID NO: 212 - DNA, 27 bp, synthetic construct
  source 1..27: mol_type = other DNA
atgcaaggaacacactggccgtggacg

SEQ ID NO: 213 - protein, 9 aa, synthetic construct
MQGTHWPWT

SEQ ID NO: 214 - DNA, 1335 bp, synthetic construct
  source 1..1335: mol_type = other DNA
gaggtgcagctggtggagtctgggggaggcttggcccagcctggggggtccctgaaactctcttgtgaagcctctggattcacgttcagtgattctgcaatgcactgggtccgccaggcttccggaaaagggctggagtgggttggtcgtattagaagcaaagctaatagttacgcgacaggatatgctgcgtcggtgaaaggcaggttcaccatctccagagatgattcaaagaacatggcgtttctggaaatgaacagcctgaaaaccgaagacacggccgtatattactgtctccgacaaacttacggtgacccctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 215 - protein, 444 aa, synthetic construct
EVQLVESGGGLAQPGGSLKLSCEASGFTFSDSAMHWVRQASGKGLEWVGRIRSKANSYATGYAASVKGRFTISRDDSKNMAFLEMNSLKTEDTAVYYCLRQTYGDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 216 - DNA, 660 bp, synthetic construct
  source 1..660: mol_type = other DNA
gatgttgtgatgactcagtctccactctccctgtccgtcacccttggacagccggcctccatctcctgcaggtctagtctaagcctcgtatacagtgatggaaacaactacttgaattggtttcagcagaggccaggccaatctccaaggcgcctactttataaagtttttaaccgggactctggggtcccagacagattcagcggcagtgggtcaggcactgatttcacactgaaaatcagcagggtggaggctgaggatgttggggtttattactgcatgcaaggaacacactggccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 217 - protein, 219 aa, synthetic construct
DVVMTQSPLSLSVTLGQPASISCRSSLSLVYSDGNNYLNWFQQRPGQSPRRLLYKVFNRDSGVPDRFSGSGSGTDFTLKISRVEAEDVGVYYCMQGTHWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 218 - DNA, 366 bp, synthetic construct
  source 1..366: mol_type = other DNA
caggttcagctggtgcagtctggagttgaggtgaagaagcctggggcctcagtgaaggtctcctgcaaggcttctggttacacctttaagttttatggtatcagttgggtgcgacaggcccctggacaagggcttgagtggatgggctggatcagtgtttacaatggtaaaacaaagtatgcacagaagctccagggcagagtcaccatgacaacagacacatccacgagcacagcctacatggagctgaggagcctgagatctgacgacacggccgtgtatttttgtgcgagagatggggactatgagagtagtggttatccgtttgactactggggccagggaaccctggtcaccgtctcctca

SEQ ID NO: 219 - protein, 122 aa, synthetic construct
QVQLVQSGVEVKKPGASVKVSCKASGYTFKFYGISWVRQAPGQGLEWMGWISVYNGKTKYAQKLQGRVTMTTDTSTSTAYMELRSLRSDDTAVYFCARDGDYESSGYPFDYWGQGTLVTVSS

SEQ ID NO: 220 - DNA, 24 bp, synthetic construct
  source 1..24: mol_type = other DNA
ggttacacctttaagttttatggt

SEQ ID NO: 221 - protein, 8 aa, synthetic construct
GYTFKFYG

SEQ ID NO: 222 - DNA, 24 bp, synthetic construct
  source 1..24: mol_type = other DNA
atcagtgtttacaatggtaaaaca

SEQ ID NO: 223 - protein, 8 aa, synthetic construct
ISVYNGKT

SEQ ID NO: 224 - DNA, 45 bp, synthetic construct
  source 1..45: mol_type = other DNA
gcgagagatggggactatgagagtagtggttatccgtttgactac

SEQ ID NO: 225 - protein, 15 aa, synthetic construct
ARDGDYESSGYPFDY

SEQ ID NO: 226 - DNA, 324 bp, synthetic construct; identical to SEQ ID NO: 69
  source 1..324: mol_type = other DNA

SEQ ID NO: 227 - protein, 108 aa, synthetic construct; identical to SEQ ID NO: 70

SEQ ID NO: 228 - DNA, 1350 bp, synthetic construct
  source 1..1350: mol_type = other DNA
caggttcagctggtgcagtctggagttgaggtgaagaagcctggggcctcagtgaaggtctcctgcaaggcttctggttacacctttaagttttatggtatcagttgggtgcgacaggcccctggacaagggcttgagtggatgggctggatcagtgtttacaatggtaaaacaaagtatgcacagaagctccagggcagagtcaccatgacaacagacacatccacgagcacagcctacatggagctgaggagcctgagatctgacgacacggccgtgtatttttgtgcgagagatggggactatgagagtagtggttatccgtttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 229 - protein, 449 aa, synthetic construct
QVQLVQSGVEVKKPGASVKVSCKASGYTFKFYGISWVRQAPGQGLEWMGWISVYNGKTKYAQKLQGRVTMTTDTSTSTAYMELRSLRSDDTAVYFCARDGDYESSGYPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 230 - DNA, 648 bp, synthetic construct; identical to SEQ ID NO: 79
  source 1..648: mol_type = other DNA

SEQ ID NO: 231 - protein, 215 aa, synthetic construct; identical to SEQ ID NO: 80

SEQ ID NO: 232 - protein, 353 aa, Homo sapiens
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAG

SEQ ID NO: 233 - protein, 355 aa, Homo sapiens
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKSWISESVEADVRLRLANVSERDGGEYLCRATNFIGVAEKAFWLSVHGPRAAEEELVEADEAGSVYAG

SEQ ID NO: 234 - protein, 12 aa, synthetic construct
EQKLISEEDLGG

SEQ ID NO: 235 - protein, 6 aa, synthetic construct
HHHHHH

SEQ ID NO: 236 - protein, 28 aa, synthetic construct
EQKLISEEDLGGEQKLISEEDLHHHHHH

SEQ ID NO: 237 - protein, 233 aa, synthetic construct
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 238 - protein, 383 aa, synthetic construct
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKSWISESVEADVRLRLANVSERDGGEYLCRATNFIGVAEKAFWLSVHGPRAAEEELVEADEAGSVYAGEQKLISEEDLGGEQKLISEEDLHHHHHH

SEQ ID NO: 239 - protein, 588 aa, synthetic construct
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKSWISESVEADVRLRLANVSERDGGEYLCRATNFIGVAEKAFWLSVHGPRAAEEELVEADEAGSVYAGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 240 - protein, 379 aa, synthetic construct
EPPGPEQRVVRRAAEVPGPEPSQQEQVAFGSGDTVELSCHPPGGAPTGPTVWAKDGTGLVASHRILVGPQRLQVLNASHEDAGVYSCQHRLTRRVLCHFSVRVTDAPSSGDDEDGEDVAEDTGAPYWTRPERMDKKLLAVPAANTVRFRCPAAGNPTPSISWLKNGKEFRGEHRIGGIKLRHQQWSLVMESVVPSDRGNYTCVVENKFGSIRQTYTLDVLERSPHRPILQAGLPANQTAILGSDVEFHCKVYSDAQPHIQWLKHVEVNGSKVGPDGTPYVTVLKSWISENVEADARLRLANVSERDGGEYLCRATNFIGVAEKAFWLRVHGPQAAEEELMETDEAGSVYAGEQKLISEEDLGGEQKLISEEDLHHHHHH

SEQ ID NO: 241 - protein, 381 aa, synthetic construct
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGEQKLISEEDLGGEQKLISEEDLHHHHHH

SEQ ID NO: 242 - protein, 383 aa, synthetic construct
ESLGTEQRVVGRVAEVSGPEPSQQEQLVFGSGDAVELSCPPPGGGPMGPTVWVKDGAGLVPSERVLVGPQRLQVLNASHEDSGAYSCRQRLTQLVLCHFSVRVTDAPSSGDDEDGEDEAEDTGVDTGAPYWTRPERMDKKLLAVPAANTVRFRCPAAGNPTPSISWLKNGKEFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKSWISESVEADVRLRLANVSERDGGEYLCRATNFIGVAEKAFWLSVHGPRAAEEELVEADEAGSVYAGEQKLISEEDLGGEQKLISEEDLHHHHHH

SEQ ID NO: 243 - protein, 457 aa, synthetic construct
EVQLVESGGGLVQPGGSLRLSCAASGFTFTSTGISWVRQAPGKGLEWVGRIYPTSGSTNYADSVKGRFTISADTSKNTAYLQMNSLRAEDTAVYYCARTYGIYDLYVDYTEYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 244 - protein, 214 aa, synthetic construct
DIQMTQSPSSLSASVGDRVTITCRASQDVDTSLAWYKQKPGKAPKLLIYSASFLYSGVPSRFSGSGSGTDFTLTISSLQPEDFATYYCQQSTGHPQTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 245 - protein, 27 aa, Homo sapiens
SCPPPGGGPMGPTVWVKDGTGLVPSER

SEQ ID NO: 246 - protein, 12 aa, Homo sapiens
YSCRQRLTQRVL

SEQ ID NO: 247 - protein, 8 aa, Homo sapiens
LLAVPAAN

SEQ ID NO: 248 - protein, 14 aa, Homo sapiens
VLERSPHRPILQAG

SEQ ID NO: 249 - protein, 11 aa, Homo sapiens
YVTVLKSWISE

SEQ ID NO: 250 - protein, 6 aa, Homo sapiens
ADVRLR

SEQ ID NO: 251 - protein, 16 aa, Homo sapiens
LCRATNFIGVAEKAFW

SEQ ID NO: 252 - protein, 15 aa, Homo sapiens
GQQEQLVFGSGDAVE

SEQ ID NO: 253 - protein, 8 aa, Homo sapiens
VLVGPQRL

SEQ ID NO: 254 - protein, 14 aa, Homo sapiens
VLERSPHRPILQAG

SEQ ID NO: 255 - protein, 10 aa, Homo sapiens
HCKVYSDAQP

SEQ ID NO: 256 - protein, 20 aa, Homo sapiens
YVTVLKSWISESVEADVRLR

SEQ ID NO: 257 - protein, 15 aa, Homo sapiens
LCRATNFIGVAEKAF

SEQ ID NO: 258 - protein, 4 aa, Homo sapiens
ADVR

SEQ ID NO: 259 - protein, 6 aa, Homo sapiens
IGVAEK

SEQ ID NO: 260 - protein, 7 aa, Homo sapiens
GPTVWVK

SEQ ID NO: 261 - protein, 5 aa, Homo sapiens
HCKVY

SEQ ID NO: 262 - protein, 6 aa, Homo sapiens
KSWISE

SEQ ID NO: 263 - protein, 4 aa, Homo sapiens
ADVR